(19) United States
(12) Patent Application Publication
(10) Pub. No.: US 2026/0250704 A1
(43) Pub. Date: Aug. 27, 2026

(54) ASIAN SOYBEAN RUST RESISTANCE GENES

(71) Applicant: Two Blades Foundation, Evanston, IL (US)

(72) Inventors: Yogesh Kumar Gupta, Norwich (GB); Cynara Cassandri Teixeira Romero, Norwich (GB); Nadine Ilk-Maintz, Nußloch (DE); Hendrikus Pieter van Esse, Norwich (GB)

(21) Appl. No.: 19/129,612

(22) PCT Filed: Nov. 14, 2023
(86) PCT No.: PCT/US2023/079564

§ 371 Date: May 13, 2025

Related U.S. Application Data
(60) Provisional application No. 63/383,775, filed on Nov. 15, 2022.

(51) Int. Cl. C12N 15/82; C07K 14/415; C12Q 1/6895

(52) U.S. Cl. CPC C12N 15/8282; C07K 14/415; C12Q 1/6895; C12Q 2600/13

(57) ABSTRACT

Compositions and methods and for enhancing the resistance of soybean plants to Asian soybean rust caused by one or more races of Phakopsora pachyrhizi are provided. The compositions comprise nucleic acid molecules encoding resistance (R) gene products and/or modulator gene products and variants thereof as well as plants, seeds, and plant cells comprising such nucleic acid molecules. The methods for enhancing the resistance of a plant to Asian soybean rust caused by one or more races of P. pachyrhizi comprise introducing a nucleic acid molecule encoding an R gene product into a plant cell and can further comprise introducing a nucleic acid molecule encoding a modulator gene product. Additionally provided are methods for using the soybean plants in agriculture to limit plant disease.

### CROSS-REFERENCE TO RELATED APPLICATION

This application claims the benefit of U.S. Provisional Patent Application No. 63/383,775 filed Nov. 15, 2022, which is hereby incorporated herein in its entirety by reference.

### REFERENCE TO AN ELECTRONIC SEQUENCE LISTING

The contents of the electronic sequence listing (070294-0219SEQLST.xml; Size: 322,337 bytes; and Date of Creation: Nov. 10, 2023) is herein incorporated by reference in its entirety.

### FIELD OF THE INVENTION

The present invention relates to compositions and methods for identifying, selecting, and producing enhanced disease and/or pathogen-resistant soybean plants.

### BACKGROUND OF THE INVENTION

Soybeans (Glycine max (L.) Merr,) are one of the most economically important crops on a worldwide basis. The Food and Agriculture Organization of the United Nations (FAO) estimate for the 2020 global gross production value of soybean crop is in excess of $120 billion dollars (USD) and is only exceeded by rice, maize, and wheat (FAOSTAT, available on the World Wide Web at fao.org/faostat/en/#data/QV).

As is the case with other crop plants, soybean production is reduced by pests and pathogens including, for example, insects, nematodes, viruses, bacteria, and fungi. To limit the damage caused by such pests and pathogens, farmers used agricultural chemicals, cultural practices, and/or plants comprising resistance (R) genes against such pests and pathogens.

Asian soybean rust (ASR) caused by the fungus Phakopsora pachyrhizi is an economically devastating foliar disease of soybeans. While ASR has been a serious disease in Asia for many decades, ASR has spread to other soybean-growing regions of the world (Rupe and Sconyers (2008) Plant Health Instructor DOI: 10.1094/PHI-I-2008-0401-01). ASR was first detected in Africa in 1997, in South America in 2001, and in the United States in 2004. Id.

The Asian soybean rust fungus (Phakopsora pachyrhizi) is not only a pathogen of soybean but can grow on many legumes in the specific subfamily of legumes that have keeled flowers (subfamily Papilionoideae, which is also know as Faboideae) (College of Agriculture and Biological Sciences, South Dakota State University, “Asian Soybean Rust” (2005), College of Agriculture and Biological Sciences Publications. Paper 2; available on the World Wide Web at: openprairie.sdstate.edu/coabs_pubs/2). This subfamily of legumes includes, for example, peas, common bean (Phaseolus spp.), and many forages including sweet clover and red clover. Id.

Over 90 plant species are known to be hosts for Phakopsora pachyrhizi including, but are not limited to, common, dry bean, (e.g. field, kidney, navy, pinto; Phaseolus vulgaris var. vulgaris), common, succulent bean, (e.g. garden, green, snap, and wax; Phaseolus vulgaris var. vulgaris), fava or broad bean (Vicia faba), lablab or hyacinth bean (Lablab purpureus), lima bean (Phaseolus lunatus var. lunatus), mung bean (Vigna radiata), scarlet runner bean (Phaseolus coccineus), winged or goa bean (Psophocarpus tetragonolobus), yam bean (Pachyrhizus ahipa, P. erosus), black-eyed pea, cowpea or yardlong bean (Vigna unguiculata), calopo (Calopogonium mucunoides), alyce or oneleaf clover (Alysicarpus vaginalis), crimson clover (Trifolium incarnatum), hop clover (Trifolium aureum), lappa clover (Trifolium lappaceum), white clover (Trifolium repens), crotalaria (Crotalaria anagyroides, C. spectabilis)), crownvetch (Securigera varia), fenugreek (Trigonella foenum-graicum), florida beggarweed (Desmodium tortuosum), kudzu (Pueraria montana var. lobata), lespedeza (Lespedeza spp, Kummerowia striata, K. stipulaceae), lupines (Lupinus spp.), medic (Medicago spp.), milk vetch (Astragalus cicer, A. glycyphyllos), garden and field pea (Pisum sativum), peatree or colorado river hemp (Sesbania exaltata), pigeon pea (Cajanus cajan), siratro (Macroptilium atropurpureum), soybean (Glycine max), sword bean (Canavalia gladiata), trefoil (Lotus spp.), black gram (Vigna mungo), wild soybean (Neonotonia wightii), woolypod vetch (Vicia villosa subsp. varia), and yellow sweet clover (Melilotus officinalis) (Rupe & Sconyers, 2008, “Soybean Rust,” Plant Health Instr. DOI: 10.1094/PHI-I-2008-0401-01).

The sustainable intensification of agriculture will require increased use of genetic solutions instead of chemical solutions (e.g. pesticides) to protect crops against pathogens and pests (Jones et al. (2014) Philos. T. Roy. Soc. B 369:20130087). Wild relatives of domesticated crops, such as soybeans, contain an immense diversity of useful R genes that are a valuable resource for sustainable disease control.

### BRIEF SUMMARY OF THE INVENTION

The present invention provides nucleic acid molecules that are capable of conferring to a leguminous plant, particularly a soybean plant, resistance to Asian soybean rust (ASR) caused by one or more races of Phakopsora pachyrhizi. Such nucleic acid molecules comprise one or more resistance (R) genes and can optionally comprise one or more other genes such as, for example, a gene that modulates the expression of the R gene (i.e. a modulator gene).

The present invention further provides leguminous plants comprising in their genomes one or more of the nucleic acid molecules of the present invention and enhanced resistance to ASR caused by one or more races of P. pachyrhizi, when compared to control plant that does not comprise the one or more of the nucleic acid molecules. Further provided are leguminous plant cells, plant parts, and seeds comprising one or more of the nucleic acid molecules.

The present invention provides methods for enhancing the resistance of a leguminous plant to at least one race of P. pachyrhizi that is known to cause ASR in a leguminous plant. Such methods comprise introducing into at least one leguminous plant cell a heterologous polynucleotide comprising a nucleic acid molecule of the present invention. Preferably, the heterologous polynucleotide or part thereof is stably incorporated into the genome of the leguminous plant cell. The methods can optionally further comprise regenerating the leguminous plant cell into a leguminous plant that comprises in its genome the heterologous polynucleotide. Preferably, such a leguminous plant comprises enhanced resistance to ASR caused by at least one race of P. pachyrhizi, relative to the resistance of a control leguminous plant not comprising the heterologous polynucleotide. More preferably, such a leguminous plant comprises enhanced resistance to ASR caused by at least two, three, four, five, or more races of P. pachyrhizi, relative to the resistance of a control leguminous plant not comprising the heterologous polynucleotide.

Preferably, such a soybean plant comprises enhanced resistance to ASR caused by at least one race of P. pachyrhizi, relative to the resistance of a control soybean plant not comprising the heterologous polynucleotide. More preferably, such a soybean plant comprises enhanced resistance to ASR caused by at least two, three, four, five, or more races of P. pachyrhizi, relative to the resistance of a control soybean plant not comprising the heterologous polynucleotide.

The present invention additionally provides methods for identifying a soybean or other plant that comprises a nucleic acid molecule that is capable of conferring to the soybean or other plant enhanced resistance to ASR caused at least one race of P. pachyrhizi. The methods comprise detecting in the plant the presence of at least one nucleotide acid molecule of the present invention.

Methods of using the soybean plants of the present invention in agricultural crop production to limit ASR are also provided. The methods comprise planting a soybean seed produced by a plant of the present invention in a field or outdoor growing area or indoors in a greenhouse, wherein the seed comprises at least one nucleic acid molecule of the present invention. The methods further comprise growing the soybean plant under conditions favorable for the growth and development of the soybean plant and particularly in environments in which ASR is known to occur, and optionally harvesting at least one seed or plant part from the soybean plant. Such methods can limit the economic loss caused by ASR.

### BRIEF DESCRIPTION OF THE DRAWINGS

FIGS. 1A-1B. Glycine argyrea accessions 14 days post inoculation (dpi) with P. pachyrhizi. FIG. 1A. Resistant genotype PI 653478 A: formation of lesions but no sporulation. FIG. 1B. Susceptible genotype PI 595798: abundant sporulation 14 dpi.

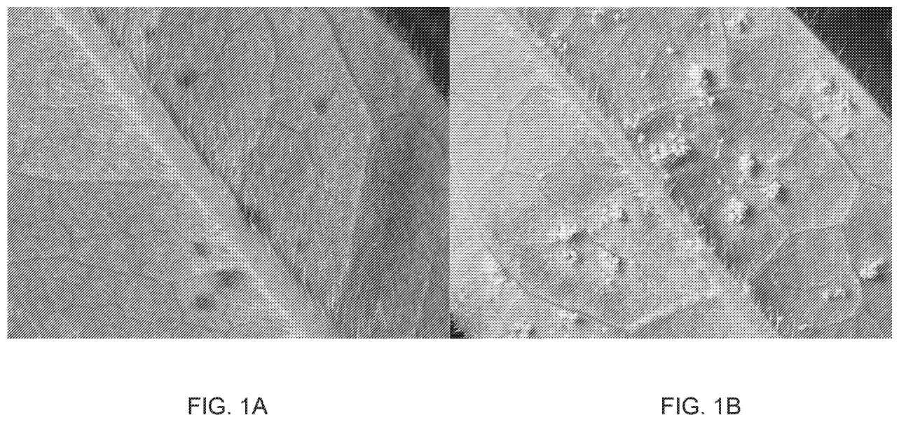

FIGS. 2A-2C. Fine mapping of GaRpp1 resistance and overview of resistance locus containing candidate genes. Fine mapping steps are illustrated by critical loss-of-function recombinants. FIG. 2A. QTL mapping on the F₂ population resulted in the identification of a resistance interval between markers GA_155 and GA_206 (2.8 Mbp). FIG. 2B. Fine mapping steps on F₂ and F₃ recombinants narrowed down the resistance interval to the region between markers CAPS_CG2 and GA_204 (109.6 Kb). FIG. 2C. Overview of GaRpp1 resistance interval containing four candidate genes: GaRpp1_CG1, GaRpp1_CG4, GaRpp1_CG5 and GaRpp1_CG10. Gene GaRpp1_CG1 (in black) is required for resistance, as validated by VIGS experiments. The region upstream of CAPS_CG2 (outside the resistance interval defined by fine mapping) is represented in the figure to show the presence of neighboring genes that are paralogs to GaRpp1_CG1 or GaRpp1_CG4.

FIGS. 2A-3H. Detached leaf assay results of VIGS plants challenged with PPUFV02. FIGS. 3A-3B: Leaves of Gargy_1900 plants silenced with BPMV:cg1 showing sporulation 13 dpi; FIGS. 3C-3D: Leaves of Gargy_1900 plants silenced with BPMV:cg1-2 showing sporulation 13 dpi; FIGS. 3E-3F: Leaves of Gargy_1900 plants silenced with BPMV:cg3-9 remain resistant 13 dpi; FIG. 3G Gargy_1900 leaf inoculated with BPMV: EV (empty vector) remain resistant after rust inoculation; FIG. 3H PI 595798, susceptible control of the experiment, showing sporulation 13 dpi.

FIG. 4. Percent Disease Severity (top) and mRNA expression (bottom) of soybean transformation events from Constructs-4 (light gray bars), -6 (dark gray bars), and -7 (black bars).

FIG. 5. Images of the abaxial side of leaves take from three different transgenic soybean events comprising Construct 7 and one wild-type soybean plant. The images highlight the differences in both disease severity and sporulation observed across transgenic soybean events comprising this construct.

FIG. 6. Genomic interval comprising the sequences of genes GaRpp1_CG1 and GaRpp1_CG4.

FIG. 7. Agrobacterium-mediated transient expression of GaRpp1 candidate genes in N. benthamiana, 4 dpi. Circles indicate the leaf areas infiltrated with a particular construct. Pictures were taken under UV light. PsCRN63=positive control for HR.

FIG. 8. Promoter analysis of the GaRpp1 intergenic region based on the PlantCARE database. The predicted cis-acting elements are annotated in the sequence.

FIG. 9. GaRpp1-mediated cell death in N. benthamiana is independent of major NLR and PRR pathways signalling components. GaRpp1_CG1, GaRpp1_CG4 or GaRpp1_CG1+GaRpp1_CG4 candidate genes were transiently overexpressed in wild-type N. benthamiana (top left), BAK1 overexpressing plants (Nb BAK, top middle), bak1 mutant (Nb bak1, top right), N. benthamiana quadruple knockout mutant of EDS1-family genes EDS1a, PAD4, SAG101a, and SAG101b (Nb epss; bottom left), NRG1 and ADR1 mutant (Nb nrg1/adr1 bottom middle) and N. benthamina plants with VIGS silenced SOBIR1 (Nb TRV::SOBIR1, bottom right) using Agrobacterium system. PsCRN63 and HopQ1=positive controls for HREmpty Vector was used as a negative control. Four-week-old plants were infiltrated at OD 0.8 and imaged 4 dpi.

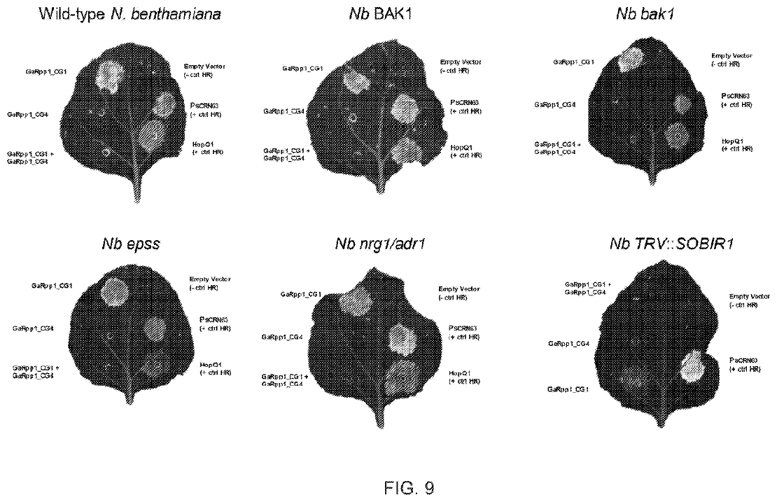

FIG. 10. Schematic representation of GaRPP1_CG1 truncated proteins. Top line represents full-length GaRPP1_CG1. GaRPP1_CG1_v1, GaRPP1_CG1_v2, GaRPP1_CG1_v3 are truncated proteins with removed 1, 2 and 3 transmembrane domains (TMhelix on the graph), respectively. GaRPP1_CG1_v4 is a truncated protein with removed first 200 amino acids in the N-terminal. All truncations have ATG added for translation initiation.

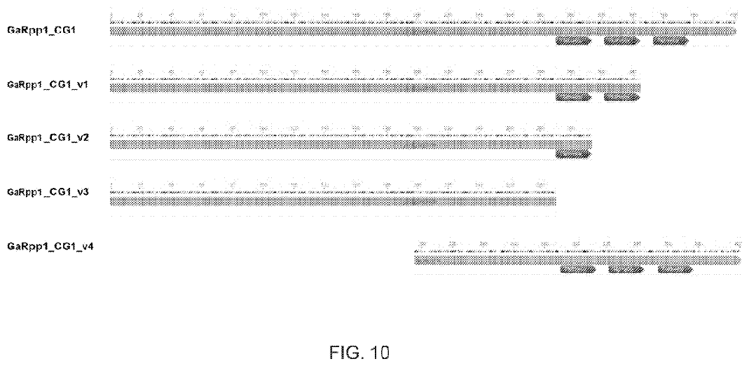

FIG. 11. GaRpp1_CG1-mediated cell death requires all three TM domains, while GaRpp1_CG1 N-terminus is required for GaRpp1_CG4-mediated cell death suppression of GaRpp1_CG1. Agrobacterium-mediated expression system was used to transiently overexpress full length GaRpp1_CG1, truncated proteins GaRPP1_CG1_v1, GaRPP1_CG1_v2 (left leaf), GaRPP1_CG1_v3, GaRPP1_CG1_v4 and GaRPP1_CG1_v4+GaRpp1_CG4 (right leaf). PsCRN63 and HopQ1=positive controls for HR. Empty Vector was used as a negative control.

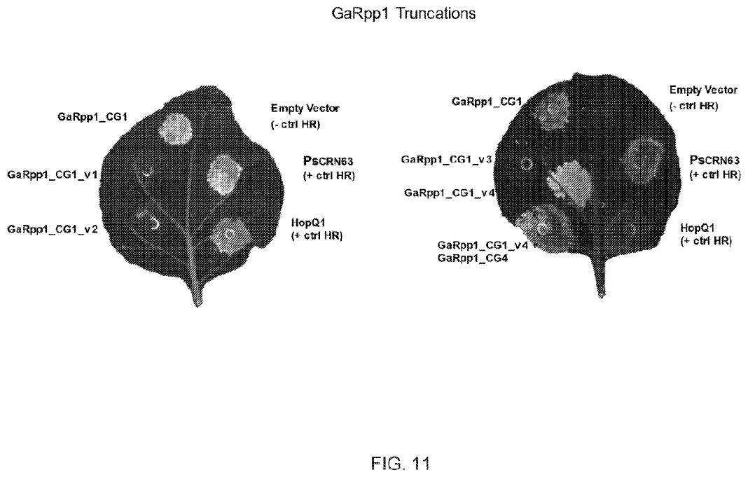

### SEQUENCE LISTING

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO: 1 sets forth the nucleotide sequence of a genomic interval comprising GaRpp1_CG1, the 306 bp intergenic region, and GaRpp1_CG4 from Glycine argyea accession PI 653478 A.

SEQ ID NO: 2 sets forth the genomic nucleotide sequence of GaRpp1_CG1 that is contained in SEQ ID NO: 1.

SEQ ID NO: 3 sets forth the genomic nucleotide sequence of the intergenic region between GaRpp1_CG1 and GaRpp1_CG4 that is contained in SEQ ID NO: 1.

SEQ ID NO: 4 sets forth the genomic nucleotide sequence of GaRpp1_CG4 that is contained in SEQ ID NO: 1.

SEQ ID NO: 5 sets forth the cDNA sequence of GaRpp1_CG1. If desired, a stop codon (e.g. TAA, TAG, or TGA) can be operably linked to the 3′ end of a nucleic acid molecule comprising or consisting of SEQ ID NO: 5. The native stop codon of this cDNA is TGA.

SEQ ID NO: 6 sets forth the amino acid sequence of the protein encoded by GaRpp1_CG1 (SEQ ID NO: 5 and SEQ ID NO: 32).

SEQ ID NO: 7 sets forth the cDNA sequence of GaRpp1_CG4. If desired, a stop codon (e.g. TAA, TAG, or TGA) can be operably linked to the 3′ end of a nucleic acid molecule comprising or consisting of SEQ ID NO: 8. The native stop codon of this cDNA is TAA.

SEQ ID NO: 8 sets forth the amino acid sequence of the protein encoded by GaRpp1_CG4 (SEQ ID NO: 7).

SEQ ID NO: 9 sets forth the nucleotide sequence of a genomic interval comprising GaRpp1_CG2, a 130 bp intergenic region, GaRpp1_CG5, GaRpp1_CG1, the 306 bp intergenic region, and GaRpp1_CG4 from Glycine argyea accession PI 653478 A.

SEQ ID NO: 10 sets forth the nucleotide sequence of a genomic interval comprising GaRpp1_CG2, the 130 bp intergenic region, and GaRpp1_CG5 from Glycine argyea accession PI 653478 A.

SEQ ID NO: 11 sets forth the genomic nucleotide sequence of GaRpp1_CG2 that is contained in SEQ ID NO: 9.

SEQ ID NO: 12 sets forth the genomic nucleotide sequence of the intergenic region between GaRpp1_CG2 and GaRpp1_CG5 that is contained in SEQ ID NO: 9.

SEQ ID NO: 13 sets forth the genomic nucleotide sequence of GaRpp1_CG5 that is contained in SEQ ID NO: 9.

SEQ ID NO: 14 sets forth the cDNA sequence of GaRpp_CG2. If desired, a stop codon (e.g. TAA, TAG, or TGA) can be operably linked to the 3′ end of a nucleic acid molecule comprising or consisting of SEQ ID NO: 14. The native stop codon of this cDNA is TGA.

SEQ ID NO: 15 sets forth the amino acid sequence of the protein encoded by GaRpp1_CG2 (SEQ ID NO: 14).

SEQ ID NO: 16 sets forth the cDNA sequence of GaRpp1_CG5. If desired, a stop codon (e.g. TAA, TAG, or TGA) can be operably linked to the 3′ end of a nucleic acid molecule comprising or consisting of SEQ ID NO: 16. The native stop codon of this cDNA is TAA.

SEQ ID NO: 17 sets forth the amino acid sequence of the protein encoded by GaRpp1_CG5 (SEQ ID NO: 16).

SEQ ID NO: 18 sets forth the nucleotide sequence of a genomic interval comprising MSTRG.57, a 433 bp intergenic region, GaRpp1_CG3, GaRpp1_CG2, the 130 bp intergenic region, GaRpp1_CG5, GaRpp1_CG1, the 306 bp intergenic region, and GaRpp1_CG4 from Glycine argyea accession PI 653478 A.

SEQ ID NO: 19 sets forth the nucleotide sequence of a genomic interval comprising MSTRG.57, the 433 bp intergenic region, and GaRpp1_CG3 from Glycine argyea accession PI 653478 A.

SEQ ID NO: 20 sets forth the genomic nucleotide sequence of MSTRG.57 that is contained in SEQ ID NO: 18.

SEQ ID NO: 21 sets forth the genomic nucleotide sequence of the intergenic region between MSTRG.57 and GaRpp1_CG3 that is contained in SEQ ID NO: 18.

SEQ ID NO: 22 sets forth the genomic nucleotide sequence of GaRpp1_CG3 that is contained in SEQ ID NO: 18.

SEQ ID NO: 23 sets forth the cDNA sequence of MSTRG.57. If desired, a stop codon (e.g. TAA, TAG, or TGA) can be operably linked to the 3′ end of a nucleic acid molecule comprising or consisting of SEQ ID NO: 23. The native stop codon of this cDNA is TGA.

SEQ ID NO: 24 sets forth the amino acid sequence of the protein encoded by MSTRG.57 (SEQ ID NO: 21) beginning at the first start codon in the cDNA sequence.

SEQ ID NO: 25 sets forth the cDNA sequence of GaRpp1_CG3. If desired, a stop codon (e.g. TAA, TAG, or TGA) can be operably linked to the 3′ end of a nucleic acid molecule comprising or consisting of SEQ ID NO: 25. The native stop codon of this cDNA is TAA.

SEQ ID NO: 26 sets forth the amino acid sequence of the protein encoded by GaRpp1_CG3 (SEQ ID NO: 25).

SEQ ID NO: 27 sets forth the nucleotide sequence of a genomic interval comprising GaRpp1_CG1 and the 306 bp intergenic region from Glycine argyea accession PI 653478 A.

SEQ ID NO: 28 sets forth the nucleotide sequence of a genomic interval comprising the 306 bp intergenic region and GaRpp1_CG4 from Glycine argyea accession PI 653478 A.

SEQ ID NO: 29 sets forth the nucleotide sequence of a fragment of the intergenic region between GaRpp1_CG1 and GaRpp1_CG4 that consists of nucleotides 1 to 106 of SEQ ID NO: 3.

SEQ ID NO: 30 sets forth the nucleotide sequence of a fragment of the intergenic region between GaRpp1_CG1 and GaRpp1_CG4 that consists of nucleotides 103 to 306 of SEQ ID NO: 3.

SEQ ID NO: 31 sets forth the nucleotide sequence of a fragment of the intergenic region between GaRpp1_CG1 and GaRpp1_CG that consists of nucleotides 103 to 190 of SEQ ID NO: 3.

SEQ ID NO: 32 sets forth the coding sequence (CDS) of GaRpp1_CG1. If desired, a stop codon (e.g. TAA, TAG, or TGA) can be operably linked to the 3′ end of a nucleic acid molecule. The native stop codon of this cDNA is TGA. The native stop codon of this cDNA is TGA. The amino acid sequence encoded by SEQ ID NO: 32 is set forth in SEQ ID NO: 6.

SEQ ID NO: 33 sets forth the nucleotide sequence of a fragment of the GaRpp1_CG1 CDS that consists of nucleotides 1 to 1032 of SEQ ID NO: 32. If desired, a stop codon (e.g. TAA, TAG, or TGA) can be operably linked to the 3′ end of a nucleic acid molecule comprising or consisting of SEQ ID NO: 33. TAG was used as the stop codon for expression of this fragment as described below in Example 10.

SEQ ID NO: 34 sets forth the amino acid sequence of the protein encoded by SEQ ID NO: 33.

SEQ ID NO: 35 sets forth the nucleotide sequence of a fragment of the GaRpp1_CG CDS that consists of nucleotides 1 to 939 of SEQ ID NO: 32. If desired, a stop codon (e.g. TAA, TAG, or TGA) can be operably linked to the 3′ end of a nucleic acid molecule comprising or consisting of SEQ ID NO: 35. TAG was used as the stop codon for expression of this fragment as described below in Example 10.

SEQ ID NO: 36 sets forth the amino acid sequence of the protein encoded by SEQ ID NO: 35.

SEQ ID NO: 37 sets forth the nucleotide sequence of a fragment of the GaRpp1_CG CDS that consists of nucleotides 1 to 870 of SEQ ID NO: 32. If desired, a stop codon (e.g. TAA, TAG, or TGA) can be operably linked to the 3′ end of a nucleic acid molecule comprising or consisting of SEQ ID NO: 37. TGA was used as the stop codon for expression of this fragment as described below in Example 10.

SEQ ID NO: 38 sets forth the amino acid sequence of the protein encoded by SEQ ID NO: 37.

SEQ ID NO: 39 sets forth the nucleotide sequence of a fragment of the GaRpp1_CG1 CDS that consists of nucleotides 580 to 1224 of SEQ ID NO: 32. An ATG codon was added to the 5′ end as a start codon. If desired, a stop codon (e.g. TAA, TAG, or TGA) can be operably linked to the 3′ end of a nucleic acid molecule comprising or consisting of SEQ ID NO: 39. The native stop codon of GaRpp1_CG1 (TGA) was used as the stop codon for expression of this fragment as described below in Example 10.

SEQ ID NO: 40 sets forth the amino acid sequence of the protein encoded by SEQ ID NO: 39.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention relates to the identification of certain genomic intervals in the genome an accession of Glycine argyea, a wild relative of domesticated soybean (Glycine max). The G. argyea accession (PI 653478 A) is known to be resistant to ASR caused by multiple races of P. pachyrhizi. The identified genomic intervals correspond to a locus that comprises the trait of resistance to ASR caused by multiple races of P. pachyrhizi. The present invention further relates to the isolation of nucleic acid molecules corresponding to the genomic intervals. Such nucleic acid molecules can comprise an entire genomic interval or a portion or portions thereof. For example, a portion can comprise a single gene such as, for example, an R gene, two adjacent genes and the intergenic region between them, or even a part of a single gene (e.g. a promoter).

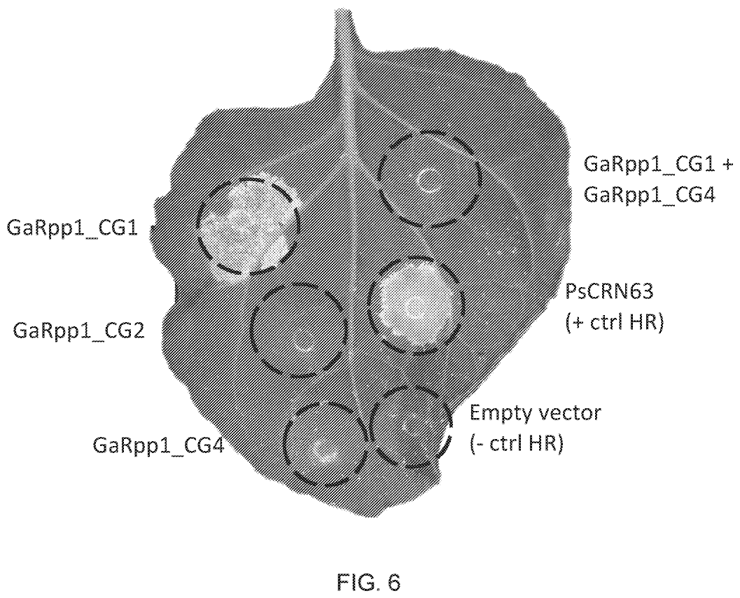

As disclosed hereinbelow, transgenic soybean plants expressing GaRpp1_CG1 (“CG1”) under the control of an operably linked heterologous constitutive promoter displayed enhanced resistance to ASR caused by P. pachyrhizi. In the genome of Glycine argyea accession PI 653478 A, CG1 and GaRpp1_CG4 (“CG4”) are arranged in a head-to-head orientation, sharing a short intergenic region (306 bp from the start of the transcription start site from one gene to the other, FIG. 6). Such an arrangement coupled with the short intergenic region likely indicates that the short intergenic region comprises a bidirectional promoter that drives the expression of both genes and that may be regulated by pathogen infection. While the present invention is not bound by a particular biological mechanism, CG4 may act as a modulator of CG1 that limits the auto-activity of CG4 in plant tissue as disclosed in Example 6 below. Therefore, in certain embodiments of the invention, it may be desirable to co-express CG4 and CG1 in a transgenic plant.

The present invention provides nucleic acid molecules comprising the nucleotide sequences of the R gene, CG1, and its paralogs GaRpp1_CG2 (“CG2”) and MSTRG.57 and naturally occurring (e.g. alleles, orthologs, paralogs) and synthetic or artificial (i.e. non-naturally occurring) variants thereof. The present invention further provides nucleic acid molecules comprising the nucleotide sequences of the modulator gene, CG4, and its paralogs GaRpp1_CG5 (“CG5”), and GaRpp1_CG3 (“CG3”) and naturally occurring and synthetic or artificial variants thereof. Additionally, the present invention provides nucleic acid molecules comprising intergenic regions between the gene pairs CG1 and CG4, CG2 and CG5, and MSTRG.57 and CG3, and naturally occurring and synthetic or artificial variants of such intergenic sequences. Examples of such R gene, modulator gene, and intergenic region nucleotide sequences include the nucleotide sequences set forth in SEQ ID NOS: 2-5, 7, 11-14, 16, 20-23, 25, 27, 32, 33, 35, 37, and 39. In a preferred embodiment of the invention, the nucleic acid molecules comprise a CG1 nucleotide sequence or a variant thereof. If desired, such nucleic acid molecules can further comprise a CG4 nucleotide sequence or variant thereof.

The R gene and modulator nucleotide sequences of the invention include, but are not limited to, the nucleotide sequences of the wild-type or native CG1, CG4, CG2, CG5, MSTRG.57, and CG3 genes comprising a native promoter and the native 3′ adjacent region comprising the coding region; cDNA sequences; and nucleotide sequences comprising only the coding region. Examples of such R gene and modulator nucleotide sequences include the nucleotide sequences set forth in SEQ ID NOS: 1, 2, 4, 5, 7, 9-11, 13, 14, 16, 18-20, 22, 23, 25, 27, 28, 32, 33, 35, 37, and 39, and variants thereof, and nucleotide sequences encoding the amino acid sequences set forth in SEQ ID NOS: 6, 8, 15, 17, 24, 2634, 36, 38, and 40. In embodiments in which the native promoter is not used to drive the expression of the nucleotide sequence encoding the R protein or modulator protein, a heterologous promoter can be operably linked a nucleotide sequence encoding an R protein or modulator protein of the invention to drive the expression of nucleotide sequence encoding the protein in a plant.

Preferably, the R proteins encoded by the R nucleotide sequences of the invention are functional R proteins, or part(s), or domain(s) thereof, which are capable of conferring to a plant, particularly a soybean plant or other plant in the genus Glycine, resistance to one, two, three, four, five or more races of P. pachyrhizi that is/are known to cause ASR.

Preferably, the modulator proteins encoded by the modulator nucleotide sequences of the invention are functional modulator proteins, or part(s), or domain(s) thereof. Such a modulator protein is encoded by a modulator gene that, when co-expressed with an R gene of the present invention is capable of modulating the activity of at least one R gene of the present invention in a plant.

As used herein, a modulator gene is gene that capable of modulating the activity of the R protein encoded by the R gene, when the modulator gene and the R gene are co-expressed in a plant. While such a modulator gene is typically not capable by itself of conferring to a plant, resistance to a plant disease caused by a plant pathogen, the modulator gene when co-expressed in a plant with at least one R gene of the present invention can, for example, enhance or increase the resistance conferred by R gene alone or ameliorate at least one deleterious or otherwise undesirable phenotype that results when the R gene is expressed in a plant. Such a deleterious or otherwise undesirable phenotypes can be any deleterious or otherwise undesirable phenotypic change in a plant that is caused or otherwise associated with the expression of an R gene in a plant, particularly a soybean plant, more particularly a soybean cultivar. Examples of such deleterious or otherwise undesirable phenotypes (as compared to same plant without the R gene) include, but are not limited to, reduced agronomic yield, reduced plant height and/or bushy appearance, leaves with a mosaic pattern with patches of crinkled tissue, negative transmission bias of the resistance allele, delayed plant growth and development and an auto-catalytic hypersensitive response (HR).

The present invention further provides plants comprising a heterologous polynucleotide which comprises an R gene nucleotide sequence and/or a modulator gene nucleotide sequence of the present invention. Preferably, such an R gene nucleotide sequence encodes a full-length R protein of the present invention, or at least a functional part(s) or domain(s) thereof. Preferably, such a modulator gene nucleotide sequence encodes a full-length modulator protein of the present invention, or at least a functional part(s) or domain(s) thereof. In some embodiments, such a heterologous polynucleotide of the present invention is stably incorporated into the genome of the plant, and in other embodiments, the plant is transformed by a transient transformation method and the heterologous polynucleotide is not stably incorporated into the genome of the plant.

In other embodiments, a plant comprising a heterologous polynucleotide which comprises an R gene nucleotide sequence and/or a modulator gene of the present invention is produced using a method of the present invention that involves genome editing to modify the nucleotide sequence of a native or non-native gene in the genome of the plant. The native or non-native gene comprises a nucleotide sequence that is different from (i.e. not identical to) an R gene or modulator gene nucleotide sequence of the present invention, and after modification by methods disclosed in further detail hereinbelow, the modified native or non-native gene comprises an R gene or modulator nucleotide sequence of the present invention. Generally, such methods comprise the use of a plant comprising in its genome a native or non-native gene wherein the native or non-native gene comprises a nucleotide sequence that is homologous to an R gene or modulator nucleotide sequence of the present invention and further comprises introducing into the plant a nucleic acid molecule comprising at least part of an R gene or modulator nucleotide sequence of the present invention. Preferably, a nucleotide sequence of native or non-native gene comprises about 70%, 75% 80%, 85%, 90%, 95%, 96%, 97%, 98%, 99% or greater nucleotide sequence identity to at least one R gene or modulator nucleotide sequence of the present invention. Such a native or non-native gene can be, for example an R gene, or a non-functional homolog of such an R gene that is not, or is not known to be, capable of conferring to a plant, resistance to a plant disease. It is recognized that a plant produced by genome engineering as disclosed herein is a stably transformed plant when the native or non-native gene that is modified is stably incorporated in the genome of the plant.

Methods for both the stable and transient transformation of plants and genome editing are disclosed elsewhere herein or otherwise known in the art. In one embodiment of the invention, the plants are stably transformed soybean plants comprising a heterologous polynucleotide of the present invention stably incorporated into their respective genomes and further comprising enhanced resistance to at least one race of P. pachyrhizi that is known to cause ASR. In preferred embodiments of the invention, such stably transformed plants comprise resistance to multiple races of P. pachyrhizi that are known to cause ASR.

In certain embodiments, a plant of the invention comprises a heterologous polynucleotide which comprises a nucleotide sequence encoding an R protein and/or modulator protein of the present invention and a heterologous promoter that is operably linked for expression of the nucleotide sequence encoding an R protein or a modulator protein. The choice of heterologous promoter can depend on a number of factors such as, for example, the desired timing, localization, and pattern of expression as well as responsiveness to particular biotic or abiotic stimulus. Promoters of interest include, but are not limited to, pathogen-inducible, constitutive, tissue-preferred, wound-inducible, and chemical-regulated promoters.

The present invention further provides methods for enhancing the resistance of a soybean plant to ASR caused by P. pachyrhizi. The methods comprise introducing a heterologous polynucleotide of the invention into at least one soybean plant cell. In certain embodiments, the heterologous polynucleotide is stably incorporated into the genome of the soybean plant cell. If desired, the methods can further comprise regenerating the plant cell into a plant comprising in its genome the heterologous polynucleotide. Preferably, such a regenerated soybean plant comprises enhanced resistance to ASR caused by at least one race of P. pachyrhizi. More preferably, such a regenerated soybean plant comprises enhanced resistance to ASR caused by two, three, four, five, six, seven, eight, nine, ten or more races of P. pachyrhizi.

In other embodiments, a plant comprising a heterologous polynucleotide which comprises an R gene nucleotide sequence and/or a modulator gene of the present invention is produced using a method of the present invention that involves genome editing to modify the nucleotide sequence of a native or non-native gene in the genome of the plant. The native or non-native gene comprises a nucleotide sequence that is different from (i.e. not identical to) an R gene or modulator gene nucleotide sequence of the present invention, and after modification by methods disclosed in further detail hereinbelow, the modified native or non-native gene comprises an R gene or modulator nucleotide sequence of the present invention. Generally, such methods comprise the use of a plant comprising in its genome a native or non-native gene wherein the native or non-native gene comprises a nucleotide sequence that is homologous to an R gene or modulator nucleotide sequence of the present invention and further comprises introducing into the plant a nucleic acid molecule comprising at least part of an R gene or modulator nucleotide sequence of the present invention. Preferably, a nucleotide sequence of native or non-native gene comprises about 70%, 75% 80%, 85%, 90%, 95%, 96%, 97%, 98%, 99% or greater nucleotide sequence identity to at least one R gene or modulator nucleotide sequence of the present invention. Such a native or non-native gene can be, for example an R gene, or a non-functional homolog of such an R gene that is not, or is not known to be, capable of conferring to a plant, resistance to a plant disease. It is recognized that a plant produced by genome engineering as disclosed herein is a stably transformed plant when the native or non-native gene that is modified is stably incorporated in the genome of the plant.

Methods for both the stable and transient transformation of plants and genome editing are disclosed elsewhere herein or otherwise known in the art. In one embodiment of the invention, the plants are stably transformed soybean plants comprising a heterologous polynucleotide of the present invention stably incorporated into their respective genomes and further comprising enhanced resistance to at least one race of P. pachyrhizi that is known to cause ASR. In preferred embodiments of the invention, such stably transformed plants comprise resistance to multiple races of P. pachyrhizi that are known to cause ASR.

The plants disclosed herein find use in methods for limiting ASR caused by at least one race of P. pachyrhizi in agricultural crop production, particularly in regions where ASR is prevalent and is known to negatively impact, or at least has the potential to negatively impact, agricultural yield. The methods of the invention comprise planting a soybean seed of the present invention, wherein the seed comprises at least one R gene nucleotide sequence of the present invention, and optionally comprises at least one modulator gene nucleotide sequence of the present invention. The methods further comprise growing the plant that is derived from the seed under conditions favorable for the growth and development of the plant, and optionally harvesting at least one seed from the plant.

The present invention additionally provides methods for identifying a plant, particularly a soybean plant that comprises an R gene or modulator gene nucleotide sequence of the present invention. The methods find use in breeding soybean plants for resistance to ASR caused by P. pachyrhizi. Such resistant plants find use in the agricultural production of soybean seeds for human or livestock consumption or other use. The methods comprise detecting in a plant, or in at least one part or cell thereof, the presence of an R gene and/or modulator gene nucleotide sequence of the present invention. In some embodiments of the invention, detecting the presence of the R gene and/or modulator gene nucleotide sequence comprises detecting the entire R gene or modulator gene nucleotide sequence in genomic DNA isolated from a soybean plant. In preferred embodiments, however, detecting the presence of a R gene or modulator gene nucleotide sequence comprises detecting the presence of at least one marker within the R gene or modulator gene nucleotide sequence, respectively. In other embodiments of the invention, detecting the presence of the R gene or modulator gene comprises detecting the presence of the R protein encoded by the R gene nucleotide sequence or the modulator protein encoded by the modulator gene nucleotide sequence using, for example, immunological detection methods involving an antibody preparation that specifically binds to the R protein or the modulator protein.

In the methods for identifying a plant, particularly a soybean plant that comprises an R gene or modulator gene nucleotide sequence of the present invention, detecting the presence of the R gene nucleotide sequence and/or the modulator gene nucleotide sequence in the soybean plant can involve one or more of the following molecular biology techniques that are disclosed elsewhere herein or otherwise known in the art including, but not limited to, isolating genomic DNA and/or RNA from the plant, amplifying nucleic acid molecules comprising the R gene nucleotide sequence and/or the modulator gene nucleotide sequence and/or marker(s) therein by PCR amplification, sequencing nucleic acid molecules comprising the R gene nucleotide sequence and/or the modulator gene nucleotide sequence and/or marker(s), identifying the R gene nucleotide sequence and/or the modulator gene nucleotide sequence, the marker(s), or a transcript or transcripts of the R gene nucleotide sequence and/or a transcript of the modulator gene nucleotide sequence by nucleic acid hybridization, and conducting an immunological assay for the detection of the R protein(s) encoded by the R gene nucleotide sequence and/or the modulator gene nucleotide sequence. It is recognized that oligonucleotide probes and PCR primers can be designed to identity the R gene nucleotide sequence and/or the modulator gene nucleotide sequence of the present invention and that such probes and PCR primers can be utilized in methods disclosed elsewhere herein or otherwise known in the art to rapidly identify in a population of plants one or more plants comprising the presence of an R gene nucleotide sequence and/or an modulator gene nucleotide sequence of the present invention.

Depending on the desired outcome, the heterologous polynucleotides of the invention can be stably incorporated into the genome of the plant cell or not stably incorporated into genome of the plant cell. If, for example, the desired outcome is to produce a stably transformed plant with enhanced resistance to ASR caused by P. pachyrhizi, then the heterologous polynucleotide can be, for example, fused into a plant transformation vector suitable for the stable incorporation of the heterologous polynucleotide into the genome of the plant cell. Typically, the stably transformed plant cell will be regenerated into a transformed plant that comprises in its genome the heterologous polynucleotide. Such a stably transformed plant is capable of transmitting the heterologous polynucleotide to progeny plants in subsequent generations via sexual and/or asexual reproduction. Plant transformation vectors, methods for stably transforming plants with an introduced heterologous polynucleotide and methods for plant regeneration from transformed plant cells and tissues are generally known in the art for both monocotyledonous and dicotyledonous plants or described elsewhere herein.

In other embodiments of the invention in which it is not desired to stably incorporate the heterologous polynucleotide in the genome of the plant, transient transformation methods can be utilized to introduce the heterologous polynucleotide into one or more plant cells of a plant. Such transient transformation methods include, for example, viral-based methods which involve the use of viral particles or at least viral nucleic acids. Generally, such viral-based methods involve constructing a modified viral nucleic acid comprising a heterologous polynucleotide of the invention operably linked to the viral nucleic acid and then contacting the plant either with a modified virus comprising the modified viral nucleic acid or with the viral nucleic acid or with the modified viral nucleic acid itself. The modified virus and/or modified viral nucleic acids can be applied to the plant or part thereof, for example, in accordance with conventional methods used in agriculture, for example, by spraying, irrigation, dusting, or the like. The modified virus and/or modified viral nucleic acids can be applied in the form of directly sprayable solutions, powders, suspensions or dispersions, emulsions, oil dispersions, pastes, dustable products, materials for spreading, or granules, by means of spraying, atomizing, dusting, spreading or pouring. It is recognized that it may be desirable to prepare formulations comprising the modified virus and/or modified viral nucleic acids before applying to the plant or part or parts thereof. Methods for making pesticidal formulations are generally known in the art or described elsewhere herein.

The present invention provides nucleic acid molecules comprising at least one R gene nucleotide sequence, nucleic acid molecules comprising at least one modulator gene nucleotide sequence, and nucleic acid molecules comprising at least one R gene nucleotide sequence and at least one modulator gene nucleotide sequences. Preferably, the nucleic acid molecules comprising at least one R gene nucleotide sequence are capable of conferring upon a host plant, particularly a soybean plant, resistance to ASR caused by at least one race of P. pachyrhizi. Preferably, the nucleic acid molecules comprising at least one modulator gene nucleotide sequence are—when co-expressed in a plant with an R gene of the present invention—capable of modulating the activity of the R protein encoded by the R gene. Thus, such nucleic acid molecules find use in limiting a plant disease caused by ASR caused by at least one race of P. pachyrhizi in agricultural production. The nucleic acid molecules of the present invention include, but are not limited to, nucleic acid molecules comprising the R gene and/or modulator gene nucleotide sequences disclosed herein but also additional paralogs, orthologs, and other variants of the R gene and modulator gene nucleotide sequences. Preferably, such paralogs, orthologs, and other variants of an R gene of the present invention are capable of conferring to a plant resistance to a plant disease caused by ASR caused by at least one race of P. pachyrhizi. Preferably, such paralogs, orthologs, and other variants of a modulator gene of the present invention are capable of modulating in a plant the activity of the R protein encoded by at least one R gene of the present invention.

Methods are known in the art or disclosed elsewhere herein for determining the resistance of a soybean plant to ASR caused by at least one race of P. pachyrhizi and for determining the capability of a modulator gene to modulate the activity of an R protein encoded by an R gene including, for example, the assays described hereinbelow.

Additionally provided are methods for introducing one or more nucleic acid molecules of the present invention into a plant, particularly a soybean plant. The methods comprise crossing (i.e. cross-pollinating) a first plant comprising in its genome at least one copy of a nucleic acid molecule of the present invention with a second plant lacking in its genome the nucleic acid molecule. Such methods, either the first plant or the second plant can be the pollen donor plant. For example, if the first plant is the pollen donor plant, then the second plant is the pollen-recipient plant. Likewise, if the second plant is the pollen donor plant, then the first plant is the pollen-recipient plant. Following the crossing, the pollen-recipient plant is grown under conditions favorable for the growth and development of the plant and for a sufficient period of time for seed to mature or to achieve an otherwise desirable growth stage for use in a subsequent in vitro germination procedure such as, for example, embryo rescue that is described below. The seed can then be harvested and those seed comprising the nucleic acid molecule identified by any method known in the art including, for example, the methods for identifying a plant that comprises an R gene or modulator gene nucleotide sequence of the present invention that are described elsewhere herein.

Additionally provided are methods for introducing a nucleic acid molecule of the present invention into a leguminous plant lacking in its genome the nucleic acid molecule. Such a nucleic acid molecule can include, for example, any one or more of the aforementioned genomic sequences or intervals comprising an R gene, particularly CG1. Such nucleic acid molecules include, but are not limited to, nucleic acid molecules comprising a nucleotide sequence set forth in SEQ ID NO: 1, 2, 3, 4, 9, 10, 11, 12, 13, 18, 19, 20, 21, 22, and/or 27. Preferred nucleic acid molecules are those comprising a nucleotide sequence encoding CG1 including, but not limited to, the nucleotide sequences set forth in SEQ ID NOS: 1, 2, 9, 18, and 27. The methods comprise crossing (i.e. cross-pollinating) a first leguminous plant comprising in its genome at least one copy of nucleic acid molecule of the present invention with a second leguminous plant lacking in its genome nucleic acid molecule. The first and second leguminous plants can be the same leguminous species or can be different leguminous species. For example, the first leguminous plant can be a Glycine argyea accession PI 653478 and the second leguminous plant can be Glycine max. Such a crossing of a first species of a plant to a second species of a plant is known as an interspecific hybridization and can be used to introgress a gene or genes of interest (e.g. CG1) from one species into a related species lacking the gene or genes of interest and typically involves multiple generations of backcrossing of the progeny with the related species and selection at each generation of progeny comprising the gene or genes of interest. Such interspecific hybridization, introgression, and backcrossing methods are well known in the art and can be used in the methods of the present invention and has been reported for G. argyea and G. max (Grant et al., 1986, J. Heredity 77 (6): 423-426, doi.org/10.1093/oxfordjournals.jhered.a110274). See “Principals of Cultivar Development,” Fehr, 1993, Macmillan Publishing Company, New York; and “Fundamentals of Plant Genetics and Breeding,” Welsh, 1981, John Wiley & Sons, Inc., New York.

In methods of the present invention for introducing nucleic acid molecule of the present invention into a leguminous plant lacking in its genome nucleic acid molecule, either the first leguminous plant or the second leguminous plant can be the pollen donor plant. For example, if the first leguminous plant is the pollen donor plant, then the second leguminous plant is the pollen-recipient plant. Likewise, if the second leguminous plant is the pollen donor plant, then the first leguminous plant is the pollen-recipient plant. Following the crossing, the pollen-recipient plant is grown under conditions favorable for the growth and development of the plant and for a sufficient period of time for seed to mature or to achieve an otherwise desirable growth stage for use in a subsequent in vitro germination procedure such as, for example, embryo rescue that is described below. The seed can then be harvested and those seed comprising nucleic acid molecule of the present invention identified by any method known in the art including, for example, the methods for identifying a leguminous plant that comprises an R gene for a plant disease caused by ASR that are described elsewhere herein.

It is recognized, however, that in certain embodiments of the invention involving interspecific hybridizations, it may be advantageous to harvest the seed resulting from such interspecific hybridizations at an immature growth stage and then to germinate the immature seeds in culture (i.e. in vitro), whereby the seeds are allowed germinate in culture using methods known in art as “embryo rescue” methods. See Reed (2005) “Embryo Rescue,” in Plant Development and Biotechnology, Trigiano and Gray, eds. CRC Press, Boca Raton, pp. 235-239; and Sharma et al. (1996) Euphytica 89:325-337. It is further recognized that “embryo rescue methods are typically used when mature seeds produced by an interspecific cross display little or no germination, whereby few or no interspecific hybrid plants are produced.

Plants of interest are plant species that are susceptible to Asian soybean rust fungus caused by Phakopsora pachyrhizi. Such plants of interest include, for example, leguminous plants (Fabaceae family; also know as the Leguminosae family) in the subfamily Papilionoideae. Preferred plants are plants in the subfamily Papilionoideae that find use as food for humans and/or other animals (e.g. livestock, fish) including, but not limited to, common, dry bean (e.g. field, kidney, navy, pinto), common, succulent bean, fava bean, hyacinth bean, lima bean, mung bean, scarlet runner bean, winged bean, jicama, black-eyed pea, cowpea, crimson clover, white clover, fenugreek, lupines, garden pea, field pea, pigeon pea, soybean, sword bean, black gram, yellow sweet clover, and soybean. In preferred embodiments of the invention, the plant of interest is soybean.

Leguminous plants of the present invention include, for example, leguminous plants that are hosts for P. pachyrhizi. Such leguminous plants include, but are not limited to, common, dry bean, (e.g. field, kidney, navy, pinto; Phaseolus vulgaris var. vulgaris), common, succulent bean, (e.g. garden, green, snap, and wax; Phaseolus vulgaris var. vulgaris), fava or broad bean (Vicia faba), lablab or hyacinth bean (Lablab purpureus), lima bean (Phaseolus lunatus var. lunatus), mung bean (Vigna radiata), scarlet runner bean (Phaseolus coccineus), winged or goa bean (Psophocarpus tetragonolobus), yam bean (Pachyrhizus ahipa, P. erosus), black-eyed pea, cowpea or yardlong bean (Vigna unguiculata), calopo (Calopogonium mucunoides), alyce or oneleaf clover (Alysicarpus vaginalis), crimson clover (Trifolium incarnatum), hop clover (Trifolium aureum), lappa clover (Trifolium lappaceum), white clover (Trifolium repens), crotalaria (Crotalaria anagyroides, C. spectabilis)), crownvetch (Securigera varia), fenugreek (Trigonella foenum graicum), florida beggarweed (Desmodium tortuosum), kudzu (Pueraria montana var. lobata), lespedeza (Lespedeza spp, Kummerowia striata, K. stipulaceae), lupines (Lupinus spp.), medic (Medicago spp.), milk vetch (Astragalus cicer, A. glycyphyllos), garden and field pea (Pisum sativum), peatree or colorado river hemp (Sesbania exaltata), pigeon pea (Cajanus cajan), siratro (Macroptilium atropurpureum), soybean (Glycine max), sword bean (Canavalia gladiata), trefoil (Lotus spp.), black gram (Vigna mungo), wild soybean (Neonotonia wightii), woolypod vetch (Vicia villosa subsp. varia), and yellow sweet clover (Melilotus officinalis).

The methods of the present invention find use in producing plants with enhanced resistance to a plant disease caused by ASR caused by at least one race of P. pachyrhizi. Typically, the methods of the present invention will enhance or increase the resistance of the subject plant to the plant disease by at least 25%, 50%, 75%, 100%, 150%, 200%, 250%, 500% or more when compared to the resistance of a control plant to the same race(s) of P. pachyrhizi. Unless stated otherwise or apparent from the context of a use, a control plant for the present invention is a plant that does not comprise an R gene and/or modulator gene nucleotide sequence of the present invention. Preferably, the control plant is essentially identical (e.g. same species, subspecies, and variety) to the plant comprising the R gene and/or modulator gene nucleotide sequence except the control does not comprise the R gene and/or modulator gene nucleotide sequence. In some embodiments, the control will comprise a heterologous, control polynucleotide (e.g. vector control) that does comprise the one or more the R gene and/or modulator gene nucleotide sequences.

Additionally, the present invention provides transformed plants, seeds, and plant cells produced by the methods of present invention and/or comprising a heterologous polynucleotide of the present invention. Such a heterologous polynucleotide comprises at least one R gene or modulator gene nucleotide sequences of the present invention. Also provided are progeny plants and seeds thereof comprising a heterologous polynucleotide of the present invention. The present invention also provides fruits, seeds, leaves, stems, roots, and other plant parts produced by the transformed plants and/or progeny plants of the invention as well as food products and other agricultural products comprising, or produced or derived from, the plants or any part or parts thereof including, but not limited to, fruits, leaves, stems, roots, and seed. It is recognized that such food products can be consumed or used by humans and other animals including, but not limited to, pets (e.g., dogs and cats), livestock (e.g., pigs, cows, chickens, turkeys, and ducks), and animals produced in freshwater and marine aquaculture systems (e.g. fish, shrimp, prawns, crayfish, and lobsters).

The term “plant” is intended to encompass plants at any stage of maturity or development, as well as any cells, tissues or organs (plant parts) taken or derived from any such plant unless otherwise clearly indicated by context. Plant parts include, but are not limited to, fruits, stems, tubers, roots, flowers, ovules, stamens, leaves, embryos, meristematic regions, callus tissue, anther cultures, gametophytes, sporophytes, pollen, microspores, protoplasts, and the like. The present invention also includes seeds produced by the plants of the present invention.

The present invention provides nucleic acid molecules that are capable of conferring to a plant, particularly a soybean plant, resistance to ASR caused by at least one race of P. pachyrhizi, plants and plants cells comprising such nucleic acid molecules and related methods.

In preferred embodiments of the present invention, a nucleic acid molecule comprising at least one R gene nucleotide sequence is capable of conferring to a soybean plant resistance to ASR caused by at least two races of P. pachyrhizi. In more preferred embodiments, a nucleic acid molecule comprising at least one R gene nucleotide sequence is capable of conferring to a plant resistance to a plant disease caused by three, four, five, six, seven, eight, nine, ten or more races of P. pachyrhizi.

In one embodiment of the invention, the nucleotide sequences encoding R proteins have at least about 75%, 80%, 85%, 90%, 91%, 92%, 93%, 94%, 95%, 96%, 97%, 98%, 99% or more sequence identity to the entire nucleotide sequence set forth in SEQ ID NO: 2, 5, 11, 14, 20, 23, and 32, or to a fragment thereof.

In another embodiment of the invention, the nucleotide sequences encoding modulator proteins have at least about 75%, 80%, 85%, 90%, 91%, 92%, 93%, 94%, 95%, 96%, 97%, 98%, 99% or more sequence identity to the entire nucleotide sequence set forth in SEQ ID NO: 4, 7, 13, 16, 22, 25, and 28, or to a fragment thereof.

The present invention encompasses isolated or substantially purified polynucleotide (also referred to herein as “nucleic acid molecule”, “nucleic acid” and the like) or protein (also referred to herein as “polypeptide”) compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” is intended a portion of the polynucleotide or a portion of the amino acid sequence and hence protein encoded thereby. Fragments of polynucleotides comprising coding sequences may encode protein fragments that retain biological activity of the full-length or native protein. Fragments of polynucleotides comprising promoter sequences may retain the biological activity (i.e. capable of driving the expression of an operably linked nucleotide sequence) of the full-length or native promoter. Fragments of polynucleotides comprising bidirectional promoter sequences may retain the biological activity of the full-length or native promoter in one or both directions. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the invention.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the R proteins of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode an R protein of the invention. Generally, variants of a particular polynucleotide of the invention will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. In certain embodiments of the invention, variants of a particular polynucleotide of the invention will have at least about 75%, 80%, 85%, 90%, 91%, 92%, 93%, 94%, 95%, 96%, 97%, 98%, 99% or more sequence identity to at least one nucleotide sequence selected from the group consisting of SEQ ID NOS: 1-5, 7, 9-14, 16, 18-23, 25, 27-32, and 33, 35, 37, and 39, and optionally comprise a non-naturally occurring nucleotide sequence that differs from the nucleotide sequence set forth in SEQ ID NO: 1-5, 7, 9-14, 16, 18-23, 25, 27-32, and 33, 35, 37, and 39 by at least one nucleotide modification selected from the group consisting of the substitution of at least one nucleotide, the addition of at least one nucleotide, and the deletion of at least one nucleotide. It is understood that the addition of at least one nucleotide can be the addition of one or more nucleotides within a nucleotide sequence of the present invention (e.g. SEQ ID NO: 1-5, 7, 9-14, 16, 18-23, 25, 27-32, and 33, 35, 37, and 39), the addition of one or more nucleotides to the 5′ end of a nucleotide sequence of the present invention, and/or the addition of one or more nucleotides to the 3′ end of a nucleotide sequence of the present invention.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to at least one polypeptide having the amino acid sequence selected from the group consisting of SEQ ID NOS: 6, 8, 15, 17, 24, 26, 34, 36, 38, and 40, is disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity. In certain embodiments of the invention, variants of a particular polypeptide of the invention will have at least about 60%, 65%, 70%, 75%, 80%, 85%, 90%, 91%, 92%, 93%, 94%, 95%, 96%, 97%, 98%, 99% or more sequence identity to at least one amino acid sequence set forth in SEQ ID NO: 6, 8, 15, 17, 24, 26, 34, 36, 38, and 40, and optionally comprises a non-naturally occurring amino acid sequence that differs from at least one amino acid sequence selected from the group consisting of SEQ ID NO: 6, 8, 15, 17, 24, 26, 34, 36, 38, and 40 by at least one amino acid modification selected from the group consisting of the substitution of at least one amino acid, the addition of at least one amino acid, and the deletion of at least one amino acid. It is understood that the addition of at least one amino acid can be the addition of one or more amino acids within an amino acid sequence of the present invention (e.g. SEQ ID NO: 6, 8, 15, 17, 24, 26, 34, 36, 38, and 40), the addition of one or more amino acids to the N-terminal end of an amino acid sequence of the present invention, and/or the addition of one or more amino acids to the C-terminal end of an amino acid sequence of the present invention.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Such variants may result from, for example, genetic polymorphism or from human manipulation. Biologically active variants of an R protein or a modulator protein will have at least about 75%, 80%, 85%, 90%, 91%, 92%, 93%, 94%, 95%, 96%, 97%, 98%, 99% or more sequence identity to the amino acid sequence for the native protein (e.g. the amino acid sequence set forth in SEQ ID NO: 6, 8, 15, 17, 24, 26, 34, 36, 38, or 40) as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant and other variant forms. Likewise, the proteins of the invention encompass naturally occurring proteins as well as variations and modified forms thereof. More preferably, variants of an R protein confer to a plant or part thereof comprising the variant resistance to ASR caused by at least one race of P. pachyrhizi, and variants of a modulator protein are capable of modulating in a plant the activity of an R protein. In some embodiments, the mutations that will be made in the DNA encoding the variant will not place the sequence out of reading frame. Optimally, the mutations will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by assays that are disclosed herein below.

Variant polynucleotides and proteins also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The polynucleotides of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire sequences set forth herein or to variants and fragments thereof are encompassed by the present invention. Such sequences include sequences that are orthologs and paralogs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 75%, 80%, 85%, 90%, 91%, 92%, 93%, 94%, 95%, 96%, 97%, 98%, 99%, or greater sequence identity. Functions of orthologs are often highly conserved among species. “Paralogs” are homologous genes that arise from gene duplication events in a species. Paralogues often share similar structures and functions in related pathways and protein complexes. Genes found in the same species are considered paralogues when their nucleotide sequences and/or their encoded protein sequences share at least 75%, 80%, 85%, 90%, 91%, 92%, 93%, 94%, 95%, 96%, 97%, 98%, 99%, or greater sequence identity.

In one embodiment, the orthologs of the present invention have coding sequences comprising at least 80%, 85%, 90%, 91%, 92%, 93%, 94%, 95%, 96%, 97%, 98%, 99%, or greater nucleotide sequence identity to at least one nucleotide sequence selected from the group consisting of the nucleotide sequences set forth in SEQ ID NOS: 1, 2, 4, 5, 7, 9-11, 13, 14, 16, 18-20, 22, 23, 25, 27, 28, 32, 33, 35, 37, and 40 and/or encode proteins comprising least 80%, 85%, 90%, 91%, 92%, 93%, 94%, 95%, 96%, 97%, 98%, 99%, or greater amino acid sequence identity to at least one amino acid sequence selected from the group consisting of the amino acid sequences set forth in SEQ ID NO: 6, 8, 15, 17, 24, 26, 34, 36, 38, and 40.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as 32P, or any other detectable marker. Thus, for example, probes for hybridization can be made by labeling synthetic oligonucleotides based on the polynucleotides of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, New York).

For example, an entire polynucleotide disclosed herein, or one or more portions thereof, may be used as a probe capable of specifically hybridizing to corresponding polynucleotide and messenger RNAs. To achieve specific hybridization under a variety of conditions, such probes include sequences that are unique among the sequence of the gene or cDNA of interest sequences and are optimally at least about 10 nucleotides in length, and most optimally at least about 20 nucleotides in length. Such probes may be used to amplify corresponding polynucleotides for the particular gene of interest from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, New York). An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 90%, 95%, 96%, 97%, 98% or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. BLAST, Gapped BLAST, and PSI-Blast, XBLAST and NBLAST are available on the World Wide Web at ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22 (22): 4673-4680, 1994) using the program AlignX included in the software package Vector NTI Suite Version 7 (InforMax, Inc., Bethesda, MD, USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website on the World Wide Web at ebi.ac.uk/Tools/clustalw/index).

The use of the term “nucleic acid molecule” and the equivalent terms “polynucleotide molecule” and “polynucleotide” is not intended to limit the present invention to nucleic acid molecules, polynucleotide molecules, and polynucleotides comprising DNA. Those of ordinary skill in the art will recognize that nucleic acid molecules, polynucleotide molecules, and polynucleotides, can comprise ribonucleotides and combinations of ribonucleotides and deoxyribonucleotides. Such deoxyribonucleotides and ribonucleotides include both naturally occurring molecules and synthetic analogues. The nucleic acid molecules, polynucleotide molecules, and polynucleotides of the invention also encompass all forms of sequences including, but not limited to, single-stranded forms, double-stranded forms, hairpins, stem-and-loop structures, and the like.

The heterologous polynucleotides or polynucleotide constructs comprising R protein or modulator protein coding regions can be provided in expression cassettes for expression in the plant or other organism or non-human host cell of interest. The cassette will include 5′ and 3′ regulatory sequences operably linked to the R protein or modulator protein coding region. “Operably linked” is intended to mean a functional linkage between two or more elements. For example, an operable linkage between a polynucleotide or gene of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the coding region to be under the transcriptional regulation of the regulatory regions. The expression cassette may additionally contain selectable marker genes.

The expression cassette will include in the 5′-3′ direction of transcription, a transcriptional and translational initiation region (i.e., a promoter), an R protein or modulator protein coding region of the invention, and a transcriptional and translational termination region (i.e., termination region) functional in plants or other organism or non-human host cell. The regulatory regions (i.e., promoters, transcriptional regulatory regions, and translational termination regions) and/or the coding region of the invention may be native/analogous to the host cell or to each other. Alternatively, the regulatory regions and/or the protein coding region of the invention may be heterologous to the host cell or to each other.

As used herein, “heterologous” in reference to a nucleic acid molecule, polynucleotide, nucleotide sequence, or polynucleotide construct is a nucleic acid molecule, polynucleotide, nucleotide sequence, or polynucleotide construct that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

As used herein, a “native gene” is intended to mean a gene that is a naturally-occurring gene in its natural or native position in the genome of a plant. Such a native gene has not been genetically engineered or otherwise modified in nucleotide sequence and/or position in the genome the plant through human intervention, nor has such a native gene been introduced into the genome of the plant via artificial methods such as, for example, plant transformation.

As used herein, a “non-native gene” is intended to mean a gene that has been introduced into a plant by artificial means and/or comprises a nucleotide sequence that is not naturally occurring in the plant. Non-native genes include, for example, a gene (e.g. an R gene) that is introduced into the plant by a plant transformation method. Additionally, when a native gene in the genome of a plant is modified, for example by a genome-editing method, to comprise a nucleotide sequence that is different (i.e. non-identical) from the nucleotide sequence of native gene, the modified gene is a non-native gene.

The present invention provides host cells comprising at least of the nucleic acid molecules, expression cassettes, and vectors of the present invention. In preferred embodiments of the invention, a host cells is plant cell. In other embodiments, a host cell is selected from the group consisting of a bacterium, a fungal cell, and an animal cell. In certain embodiments, a host cell is non-human animal cell. However, in some other embodiments, the host cell is an in-vitro cultured human cell.

While it may be optimal to express the R protein or modulator protein using heterologous promoters, the native promoter of the corresponding R gene or modulator gene may be used.

The termination region may be native with the transcriptional initiation region, may be native with the operably linked protein coding region of interest, may be native with the plant host, or may be derived from another source (i.e., foreign or heterologous to the promoter, the protein of interest, and/or the plant host), or any combination thereof. Convenient termination regions are available from the Ti-plasmid of A. tumefaciens, such as the octopine synthase (OCS) and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165 (2): 233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the R protein coding sequences within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12 (2): 255-265; Kawamata et al. (1997) Plant Cell Physiol. 38 (7): 792-803; Hansen et al. (1997) Mol. Gen Genet. 254 (3): 337-343; Russell et al. (1997) Transgenic Res. 6 (2): 157-168; Rinehart et al. (1996) Plant Physiol. 112 (3): 1331-1341; Van Camp et al. (1996) Plant Physiol. 112 (2): 525-535; Canevascini et al. (1996) Plant Physiol. 112 (2): 513-524; Yamamoto et al. (1994) Plant Cell Physiol. 35 (5): 773-778; Lam (1994) Results Probl. Cell Differ. 20:181-196; Orozco et al. (1993) Plant Mol Biol. 23 (6): 1129- 1138; Matsuoka et al. (1993) Proc Natl. Acad. Sci. USA 90 (20): 9586-9590; and Guevara-Garcia et al. (1993) Plant J. 4 (3): 495-505. Such promoters can be modified, if necessary, for weak expression.

Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used in the heterologous polynucleotides of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6 (2): 141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14 (2): 247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFPT from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschmidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not intended to be limiting. Any selectable marker gene can be used in the present invention.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet.76:767-774; Hinchee, et al. (1990) Stadler. Genet. Symp.203212.203-212; Cousins, et al. (1991) Aust. J. Plant Physiol. 18:481-494; Chee, P. P. and Slightom, J. L. (1992) Gene.118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P: 119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5:17-27; Eapen et al. (1994) Plant Cell Rep. 13:582-586; Hartman, et al. (1994) Bio-Technology 12:919923; Ritala, et al. (1994) Plant. Mol. Biol. 24:317-325; and Wan, Y. C. and Lemaux, P. G. (1994) Plant Physiol. 104:3748.

The methods of the invention involve introducing a heterologous polynucleotide or polynucleotide construct into a plant. By “introducing” is intended presenting to the plant the heterologous polynucleotide or polynucleotide construct in such a manner that the construct gains access to the interior of a cell of the plant. The methods of the invention do not depend on a particular method for introducing a heterologous polynucleotide or polynucleotide construct to a plant, only that the heterologous polynucleotide or polynucleotide construct gains access to the interior of at least one cell of the plant. Methods for introducing heterologous polynucleotides or polynucleotide constructs into plants are known in the art including, but not limited to, stable transformation methods, transient transformation methods, and virus-mediated methods.

By “stable transformation” is intended that the heterologous polynucleotide or polynucleotide construct introduced into a plant integrates into the genome of the plant and is capable of being inherited by progeny thereof. By “transient transformation” is intended that a heterologous polynucleotide or polynucleotide construct introduced into a plant does not integrate into the genome of the plant. It is recognized that stable and transient transformation methods comprise introducing one or more nucleic acid molecules (e.g. DNA), particularly one or more recombinant nucleic acid molecules (e.g. recombinant DNA) into a plant, plant cell, or other host cell or organism.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100:247-250; Scheid et al., (1991) Mol. Gen. Genet., 228:104-112; Guerche et al., (1987) Plant Science 52:111-116; Neuhause et al., (1987) Theor. Appl Genet. 75:30-36; Klein et al., (1987) Nature 327:70-73; Howell et al., (1980) Science 208:1265; Horsch et al., (1985) Science 227:1229-1231; DeBlock et al., (1989) Plant Physiology 91:694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a heterologous polynucleotide or polynucleotide construct of the invention within a viral DNA or RNA molecule. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

In specific embodiments, the polynucleotides, polynucleotide constructs, and expression cassettes of the invention can be provided to a plant using a variety of transient transformation methods known in the art. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) PNAS Sci. 91:2176-2180 and Hush et al. (1994) J. Cell Science 107:775-784, all of which are herein incorporated by reference. Alternatively, the polynucleotide can be transiently transformed into the plant using techniques known in the art. Such techniques include viral vector system and Agrobacterium tumefaciens-mediated transient expression as described elsewhere herein.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a heterologous polynucleotide or polynucleotide construct of the invention, for example, an expression cassette of the invention, stably incorporated into their genome.

Any methods known in the art for modifying DNA in the genome of a plant can be used to modify genomic nucleotide sequences in planta, for example, to create or insert a resistance gene or even to replace or modify an endogenous resistance gene or allele thereof. Such methods include, but are not limited to, genome-editing (or gene-editing) techniques, such as, for example, methods involving targeted mutagenesis, homologous recombination, and mutation breeding. Targeted mutagenesis or similar techniques are disclosed in U.S. Pat. Nos. 5,565,350; 5,731,181; 5,756,325; 5,760,012; 5,795,972, 5,871,984, and 8,106,259; all of which are herein incorporated in their entirety by reference. Methods for gene modification or gene replacement comprising homologous recombination can involve inducing double breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered endonucleases to make double-strand breaks at specific recognition sequences in the genome of a plant, other organism, or host cell. See, for example, Durai et al., (2005) Nucleic Acids Re.s 33:5978-90; Mani et al. (2005) Biochem. Biophys. Res. Comm. 335:447-57; U.S. Pat. Nos. 7,163,824, 7,001,768, and 6,453,242; Arnould et al. (2006) J. Mol. Biol. 355:443-58; Ashworth et al., (2006) Nature 441:656-9; Doyon et al. (2006) J. Am. Chem. Soc. 128:2477-84; Rosen et al., (2006) Nucleic Acids Res. 34:4791-800; and Smith et al., (2006) Nucleic Acids Res. 34: e149; U.S. Pat. App. Pub. No. 2009/0133152; and U.S. Pat. App. Pub. No. 2007/0117128; all of which are herein incorporated in their entirety by reference.

Unless stated otherwise or apparent from the context of a use, the term “gene replacement” is intended to mean the replacement of any portion of a first polynucleotide molecule or nucleic acid molecule (e.g. a chromosome) that involves homologous recombination with a second polynucleotide molecule or nucleic acid molecule using a genome-editing technique as disclosed elsewhere herein, whereby at least a part of the nucleotide sequence of the first polynucleotide molecule or nucleic acid molecule is replaced with the second polynucleotide molecule or nucleic acid molecule. It is recognized that such gene replacement can result in additions, deletions, and/or modifications in the nucleotide sequence of the first polynucleotide molecule or nucleic acid molecule and can involve the replacement of an entire gene or genes, the replacement of any part or parts of one gene, or the replacement of non-gene sequences in the first polynucleotide molecule or nucleic acid molecule.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi: 10.1093/nar/gkq704; and Miller et al. (2011) Nat. Biotechnol. 29:143-148; all of which are herein incorporated by reference.

The CRISPR/Cas nuclease system can also be used to make double-strand breaks at specific recognition sequences in the genome of a plant for gene modification or gene replacement through homologous recombination. The CRISPR/Cas nuclease is an RNA-guided (simple guide RNA, sgRNA in short) DNA endonuclease system performing sequence-specific double-stranded breaks in a DNA segment homologous to the designed RNA. It is possible to design the specificity of the sequence (Cho S.W. et al., Nat. Biotechnol. 31:230-232, 2013; Cong L. et al., Science 339:819-823, 2013; Mali P. et al., Science 339:823-826, 2013; Feng Z. et al., Cell Research 1-4, 2013).

In addition, a ZFN can be used to make double-strand breaks at specific recognition sequences in the genome of a plant for gene modification or gene replacement through homologous recombination. The Zinc Finger Nuclease (ZFN) is a fusion protein comprising the part of the FokI restriction endonuclease protein responsible for DNA cleavage and a zinc finger protein which recognizes specific, designed genomic sequences and cleaves the double-stranded DNA at those sequences, thereby producing free DNA ends (Urnov et al. (2010) Nat. Rev. Genet. 11:636-46; Carroll (2011) Genetics. 188:773-82).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

The nucleic acid molecules, expression cassettes, vectors, and heterologous polynucleotides of the present invention may be used for transformation and/or genome editing of any plant species, including, but not limited to, monocots and dicots.

As used herein, the term “plant” includes seeds, plant cells, plant protoplasts, plant cell tissue cultures from which plants can be regenerated, plant calli, plant clumps, and plant cells that are intact in plants or parts of plants such as embryos, pollen, ovules, seeds, tubers, propagules, leaves, flowers, branches, fruits, roots, root tips, anthers, and the like. Progeny, variants, and mutants of the regenerated plants are also included within the scope of the invention, provided that these parts comprise the introduced polynucleotides. As used herein, “progeny” and “progeny plant” comprise any subsequent generation of a plant whether resulting from sexual reproduction and/or asexual propagation, unless it is expressly stated otherwise or is apparent from the context of usage.

As used herein, the terms “transgenic plant” and “transformed plant” are equivalent terms that refer to a “plant” as described above, wherein the plant comprises a heterologous nucleic acid molecule, heterologous polynucleotide, or heterologous polynucleotide construct that is introduced into a plant by, for example, any of the stable and transient transformation methods disclosed elsewhere herein or otherwise known in the art. Such transgenic plants and transformed plants also refer, for example, the plant into which the heterologous nucleic acid molecule, heterologous polynucleotide, or heterologous polynucleotide construct was first introduced and also any of its progeny plants that comprise the heterologous nucleic acid molecule, heterologous polynucleotide, or heterologous polynucleotide construct.

In certain embodiments of the invention, the methods involve the planting of seedlings and then growing such seedlings so as to produce plants derived therefrom and optionally harvesting from the plants a plant part or parts. As used herein, a “seedling” refers to a less than fully mature plant that is typically grown in greenhouse or other controlled-or semi-controlled (e.g. a cold frame) environmental conditions before planting or replanting outdoors or in a greenhouse for the production a harvestable plant part, such as, for example, a seed.

In some embodiments of the present invention, a plant cell is transformed with a heterologous polynucleotide encoding an R protein and/or modulator protein of the present invention. The term “expression” as used herein refers to the biosynthesis of a gene product, including the transcription and/or translation of said gene product. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide. Examples of heterologous polynucleotides and nucleic acid molecules that encode R proteins and modulator proteins are described elsewhere herein.

The use of the terms “DNA” or “RNA” herein is not intended to limit the present invention to polynucleotide molecules comprising DNA or RNA. Those of ordinary skill in the art will recognize that the methods and compositions of the invention encompass polynucleotide molecules comprised of deoxyribonucleotides (i.e., DNA), ribonucleotides (i.e., RNA) or combinations of ribonucleotides and deoxyribonucleotides. Such deoxyribonucleotides and ribonucleotides include both naturally occurring molecules and synthetic analogues including, but not limited to, nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The polynucleotide molecules of the invention also encompass all forms of polynucleotide molecules including, but not limited to, single-stranded forms, double-stranded forms, hairpins, stem-and-loop structures, and the like. Furthermore, it is understood by those of ordinary skill in the art that the nucleotide sequences disclosed herein also encompasses the complement of that exemplified nucleotide sequence.

The invention is drawn to compositions and methods for enhancing the resistance of a plant to plant disease, particularly to compositions and methods for enhancing the resistance of a plant to ASR caused by at least one race of P. pachyrhizi. By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen is minimized or lessened.

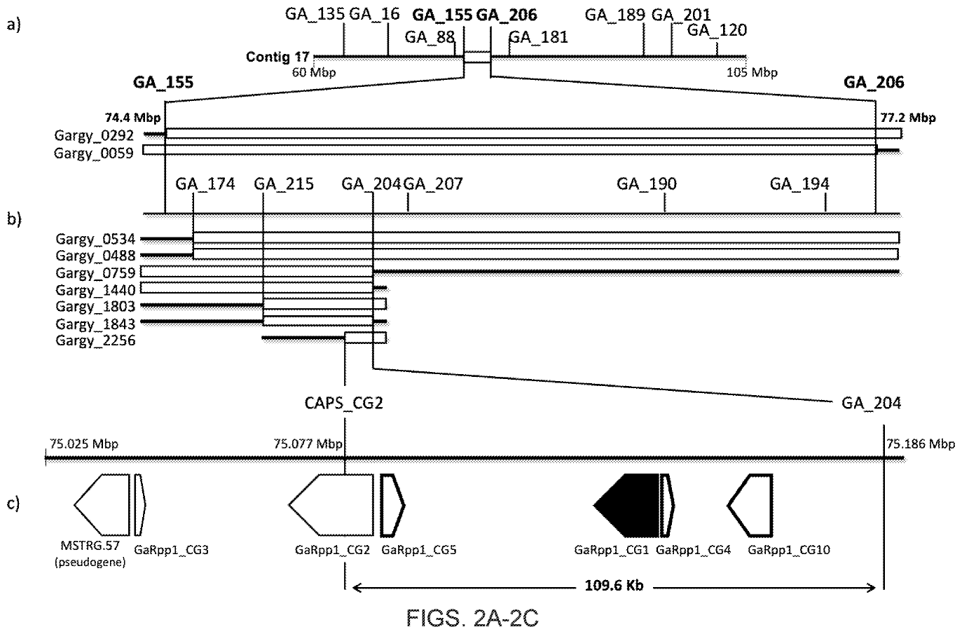

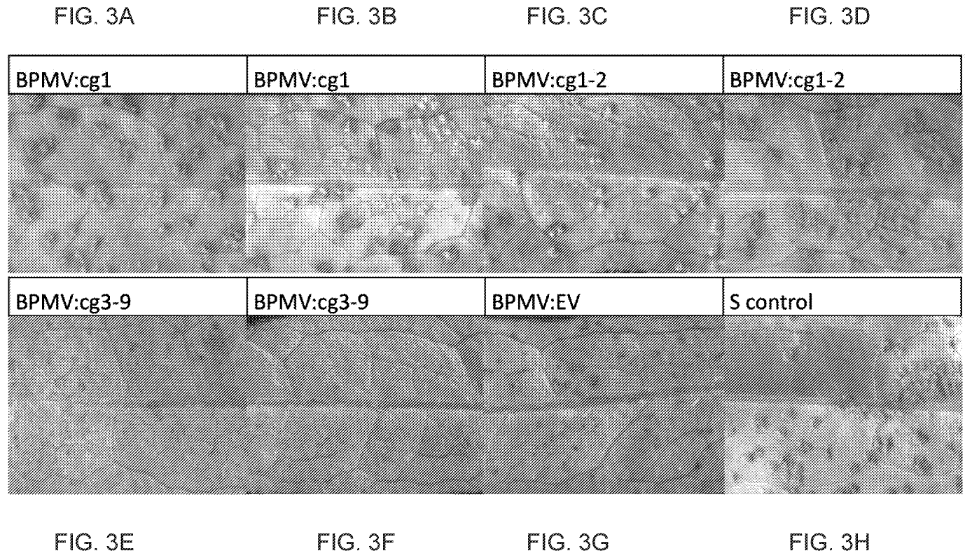

The present invention further provides promoters comprising the nucleotide sequences of the intergenic regions between the gene pairs CG1 and CG4, CG2 and CG5, and MSTRG.57 and CG3, and naturally occurring and synthetic or artificial variants of such intergenic sequences. It is believed that the intergenic regions are promoters, particularly bidirectional promoters that can drive the expression of both paired R genes, which are arranged in a head-to-head orientation in the genome of Glycine argyea accession PI 653478 A (FIG. 2C). Such promoters include, for example, promoters comprising a nucleotide sequence selected from the group consisting of SEQ ID NOS: 3, 12, and 21, and naturally occurring and synthetic or artificial variants of such intergenic sequences or fragments of such promoters that are capable of driving the expression of an operably linked nucleotide sequence in a plant cell. Examples of such fragments include, but are not limited to, fragments comprising the nucleotides sequences set forth in SEQ ID NOS: 29. 30, and 31.

Preferably, the promoters of the present invention are bidirectional promoters or pathogen-inducible promoters. More preferably, the promoters of the present invention are bidirectional and pathogen-inducible promoters. Methods are known in the art or disclosed elsewhere herein for determining promoter activity, including, for example, bidirectional promoter activity and pathogen-inducible promoter activity.

The following examples are offered by way of illustration and not by way of limitation.

### EXAMPLES

### Example 1: Identification of Candidate ASR Resistance Genes

An F₂ population was generated from a cross of two Glycine argyrea accessions: PI 653478 A, accession that is known to be resistant to Phakopsora pachyrhizi, and PI 595798 an accession that is known to be susceptible to P. pachyrhizi). PI 653478 A has been reported to be resistant to nine Australian races of P. pachyrhizi (Jarosz and Burdon (1990) Heredity 64:347-353, doi.org/10.1038/hdy.1990.43). In addition, PI 653478 A and PI 595798 have been tested for resistance against P. pachyrhizi isolates PPUFV02, Aus-1, K1-2, and T1-4. PI 653478 A was resistant to each of isolates, and PI 595798 PI 653478 A was resistant to each of isolates (data not shown).

Bulked Segregant Analyses and QTL mapping were performed on the resulting F₂ segregating population, identifying a major resistance QTL, named GaRpp1. The resistance region was mapped to linkage group 3 of G. argyrea, which is syntenic to G. max Chr03.

Using PacBio Single Molecule Real-Time (SMRT) sequencing (available on the World Wide Web at: pacb.com) and Dovetail Genomics (Santa Cruz, CA, US) Hi-C scaffolding method (Genome Res. 2016 March; 26 (3): 342-350. doi: 10.1101/gr.193474.115), a high-quality genome was assembled for the resistant genotype PI 653478 A, resulting in a 1.02 Gb genome in 690 large scaffolds. All markers associated with the resistance locus were mapped on the Contig 17, providing a complete overview of the physical map.

The first round of fine mapping on additional F₂ recombinants using the P. pachyrhizi isolate PPUFV02 delimited the resistance interval to the region between markers GA_155 and GA_206 (FIG. 2A, represented by the two loss-of-function recombinants Gargy_0292 and Gargy_0059). Screenings on the progenies of F₂ and F₃ recombinants for this genomic interval have ultimately narrowed down the GaRpp1 resistance locus to the 109.6 Kb region between flanking markers CAPS_CG2 and GA_204 (FIG. 2B). The presence of a single copy of the resistance allele in this interval is sufficient to confer resistance to the plant 14 days after inoculation with the PPUFV02 isolate. Recombinants that do not carry any copy of the resistance allele for this interval are susceptible.

Four candidate genes are present in this interval, named: GaRpp1_CG1, GaRpp1_CG4, GaRpp1_CG5 and GaRpp1_CG10. The marker CAPS_CG2 was developed on the sequence of candidate gene GaRpp1_CG2, therefore excluding this gene as a resistance candidate (FIG. 2C).

### Example 2: Candidate Gene Validation by Virus-Induced Gene Silencing

The virus-induced gene silencing (VIGS) protocol using Bean Pod Mottle Virus (BPMV) described by Whitham et al. ((2016) Curr. Protoc. Plant Biol. 1:263-283, doi: 10.1002/cppb.20012) was optimized for G. argyrea. With the aim of silencing candidate genes for functional validation, fragments of the GaRpp1 candidates were cloned into the BPMV vectors to develop four constructs: BPMV:cg1 (targeting GaRpp1_CG1), BPMV:cg2 (targeting GaRpp1_CG2), BPMV:cg1-2 (targeting CG1 and CG2) and BPMV:cg3-9 (targeting CGs 3, 4, and 5 simultaneously).

VIGS assays were performed using the resistant genotype Gargy_1900, derived from the cross between PI 653478 A and PI 595798, and known to carry the resistance allele GaRpp1. The Gargy_1900 genotype was used for the VIGS experiments instead of the resistant accession PI 653478 A to avoid the possibility that PI 653478 A had other genetic resistance gene(s) to P. pachyrhizi that could interfere with the results of silencing GaRpp1.

VIGS assays were initially performed using constructs BPMV:cg2 and BPMV:cg3-9 on the resistant genotype Gargy_1900. Upon P. pachyrhizi inoculation, the silenced leaves remained resistant. However, the silencing experiments targeting GaRpp1_CG1 with two different constructs, BPMV:cg1 and BPMV:cg1-2, resulted in clear sporulation of silenced leaves 13 dpi (FIG. 3).

At 13 dpi with PPUFV02, leaves from Gargy_1900 plants silenced with BPMV:cg1 and BPMV:cg1-2 showed a susceptible phenotype (FIGS. 3A-3D), similar to the susceptible control PI 595798 (FIG. 3H). Leaves from Gargy_1900 plants infected with BPMV:cg3-9 as well as with the BPMV: EV constructs remained resistant to soybean rust (FIG. 3E-G). These results indicate that GaRpp1_CG1 is the resistance gene GaRpp1.

### Example 3: Validation by Stable Transformation of GaRpp1_CG1 in G. max

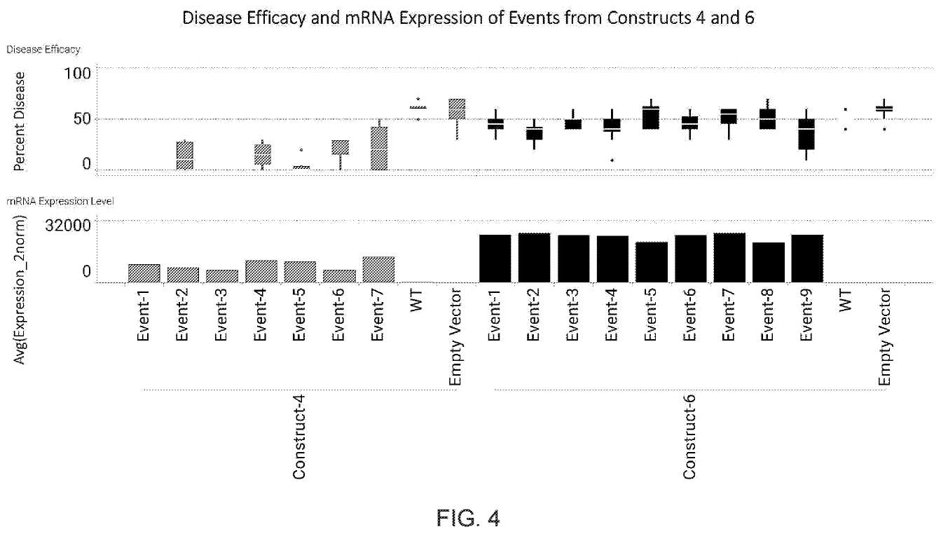

Transgenic soybean plants were generated by transforming the susceptible genotype Williams 82 with the coding sequence (CDS) of the resistance gene GaRpp1_CG1 driven by high expression promoters. Transgenic plants transformed with three constructs, “Construct-4”, “Construct-6”, and “Construct-7” showed a significant decrease in disease severity (FIG. 4).

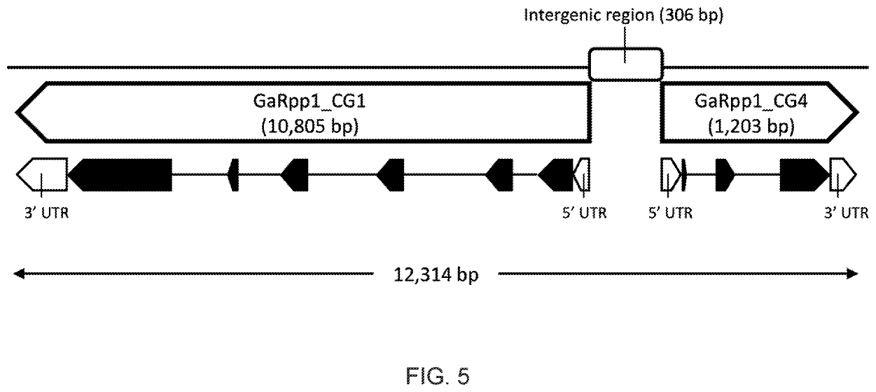

All the events from transformation with Construct-4 showed a “slow rusting” phenotype, characterized by a delayed latency period, smaller lesion size and reduced sporulation at 14 dpi. Transformants with Construct-6 displayed a 36%-50% reduction in disease severity in three events (FIG. 4, medium gray bars). Events transformed with Construct-7 ranged in both severity of disease phenotype (ranging from high to low) as well as level of sporulation caused by the disease (ranging from normal sporulation to none, FIG. 5).

Transformation with Construct-4 resulted in medium expression of the transgene (FIG. 4, light gray bars), Construct 6 resulted in high expression (FIG. 4, medium gray bars), and Construct-7 resulted in medium, targeted, expression (FIG. 4, black bars).

Constitutive expression of GaRpp1_CG1 at medium to high-level resulted in stunted, oddly branched plants with leaf malformations and a strong transmission bias, with negative selection on transgene transmission.

The deleterious effects of GaRpp1_CG1 overexpression indicate that it requires tight regulation of transcription, which might involve GaRpp1_CG4 and the intergenic region between CG1 and CG4 (bi-directional promoter). Additional constructs were generated containing the genomic sequence spanning GaRpp1_CG1 and CG4, including the native intergenic region. Transgenic soybean plants were generated by transforming the susceptible genotype Williams 82 with a construct comprising a genomic sequence containing GaRpp1_CG1, the intergenic region, and GaRpp1_CG4. When the transgenic plants were tested for ASR resistance, the transgenic plants displayed enhanced resistance to ASR relative to susceptible, control plants.

### Example 4: The Genomic Orientation of the GaRpp1_CG1 and GaRpp1_CG4 Indicates that these Genes are Expressed from a Shared Bidirectional Promoter

Genes GaRpp1_CG1 and GaRpp1_CG4 are arranged in a head-to-head orientation in the genome, sharing a short intergenic region (306 bp from the start of the transcription start site from one gene to the other, FIG. 6). Such an arrangement coupled with that short intergenic region likely indicates that short intergenic region comprises a bidirectional promoter that drives the expression of both gene and that may be regulated by pathogen infection.

A bidirectional promoter comprises the intergenic region of two adjacent genes located on complementary DNA strands, driving their transcription in opposite directions (Arnaiz et al., 2019). Bidirectional promoters are prevalent in eukaryotes, including plants. A genome-wide analysis of bidirectional promoters in Arabidopsis resulted in the identification of 2471 bidirectional gene pairs and found that they are often co-expressed and tend to be involved in the same biological function (Wang et al., 2009).

### Example 5: GaRpp1 Locus in Other G. argyrea Accessions

Five additional G. arygrea accessions (PI509452, PI599400, PI595794, and PI595795) showing contrasting phenotypes upon P. pachyrhizi inoculation were sequenced, and the reads were mapped to the GaRpp1 locus of the reference assembly of PI 653478 A. We observed that the region upstream of GaRpp1_CG1, comprising the intergenic sequence and the GaRpp1_CG4 gene is conserved in the two resistant G. argyrea accessions, but not conserved in the three susceptible accessions tested (data not shown). The fact that GaRpp1_CG4 sequence, as well as the intergenic sequence are conserved in resistant accessions but not in the susceptible accessions may indicate their importance for the proper activity of GaRpp1_CG1.

### Example 6: Transient Expression of GaRpp1 Genes in N. benthamiana

Agrobacterium-mediated transient expression of GaRpp1_CG1 in N. benthamiana elicits a hypersensitive response (HR) at 2 to 3 dpi. HR is a common and effective mechanism of plant defence against pathogens, but the fact that CG1 expression triggers HR in the absence of pathogen (auto-activity) indicates that CG1 may require a modulator to limit its auto-activity.

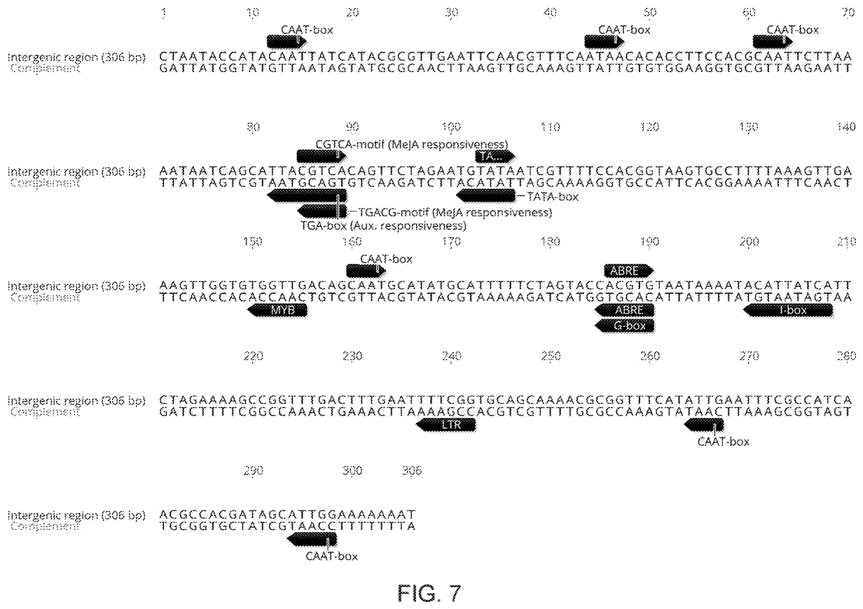

Given that GaRpp1_CG1 and GaRpp1_CG4 are arranged in a head-to-head orientation in the genome and likely share a bidirectional promoter as noted above in Example 2 (see also FIG. 6), GaRpp1_CG4 was co-infiltrated with GaRpp1_CG1. The results can be visualized in FIG. 7. CG1 triggers HR response when infiltrated alone, which is not the case for GaRpp1_CG2 and CG4 (no auto-activity was observed). Upon co-expression with CG4, CG1 does not elicit an HR response which indicates that CG4 can modulate the activity of CG1 in planta.

For the infiltrations, the Phytophthora sojae effector PsCRN63 was used as a positive control for the HR response, and empty vector was used as negative control. All genes are under the regulation of the Lotus japonicus LjUBI promoter.

While the present invention does not depend on a particular biological mechanism, the transient expression results are consistent with a biological mechanism for which GaRpp1_CG1 and GaRpp1_CG4 work together in conferring ASR resistance to a plant. The above-mentioned VIGS results for BPMV:cg3-9 constructs are also consistent with such a biological mechanism because silencing a positive regulator of a resistance gene would not result in susceptibility.

### Example 7: Paralogs of GaRpp1_CG1 and GaRpp1_CG4

The region upstream of GaRpp1 interval contains two other gene pairs that are paralogous to the CG1+CG4 pair (FIG. 2C): GaRpp1_CG2 and MSTRG.57 are paralogous to CG1, while GaRpp1_CG5 and GaRpp1_CG3 are paralogous to CG4. These gene pairs are also arranged in a head-to-head layout in the genome and share up to 91.9% identity at the protein level in the case of MSTRG.57 as compared to GaRpp1_CG1 (Table 1). GaRpp1_CG3 is 89.7% identical to GaRpp1_CG4 at protein level (Table 2).

The ortholog gene to GaRpp1_CG1 in G. max is Glyma.03G108000.1, which is 79.6% identical at the nucleotide sequence level (comparison of CDS sequences) and 64.6% at the amino acid sequence level (Table 1). The ortholog to GaRpp1_CG4 is Glyma.03G107900.1, which and encodes a protein that longer (705 bp, compared to 456 bp for CG4), 60.4% identical at the nucleotide sequence level (according to comparison of CDS sequences), and 86.8% identical at the amino acid sequence level (Table 2).

TABLE 1
Distance Matrix Protein Alignments for GaRpp1_CG1 Paralogs
(% Amino Acid Sequence Identity)

| | GaRpp1_ | GaRpp1_ | | G. max |
|---|---|---|---|---|
| | CG1 | CG2 | MSTRG.57 | ortholog |
| GaRpp1_CG1 | | 55.1 | 91.9* | 64.6 |
| GaRpp1_CG2 | 55.1 | | 53.7 | 47.3 |
| MSTRG.57* | 91.9* | 53.7 | | 68.3 |
| G. max ortholog | 64.6 | 47.3 | 68.3 | |

*MSTRG.57 is a pseudogene (i.e. lacks a start codon). The predicted protein sequence of MSTRG.57 starting from the first Met in the sequence encodes a peptide that is 100 bp shorter than GaRpp1_CG1.

TABLE 2
Distance Matrix Protein Alignments for GaRpp1_CG4 Paralogs
(% Amino Acid Sequence Identity)

| | GaRpp1_ | GaRpp1_ | GaRpp1_ | G. max |
|---|---|---|---|---|
| | CG4 | CG5 | CG3 | ortholog |
| GaRpp1_CG4 | | 81.5 | 89.7 | 86.8 |
| GaRpp1_CG5 | 81.5 | | 80.7 | 77.3 |
| GaRpp1_CG3 | 89.7 | 80.7 | | 84.6 |
| G. max ortholog | 86.8 | 77.3 | 84.6 | |

### Example 8: Promoter Analysis of the Intergenic Region Between GaRpp1_CG1 and GaRpp1_CG4

The 306 bp intergenic region between GaRpp1_CG1 and GaRpp1_CG4 was used as a query for the prediction of cis-acting elements using the PlantCARE (available on the World Wide Web at bioinformatics.psb.ugent.be/webtools/plantcare/html/) online database and the detection of transcription factor binding sites (TFBSs) using the Plant Promoter Analysis Navigator (PlantPAN 2.0; available on the World Wide Web at: PlantPAN2.itps.ncku.edu.tw).

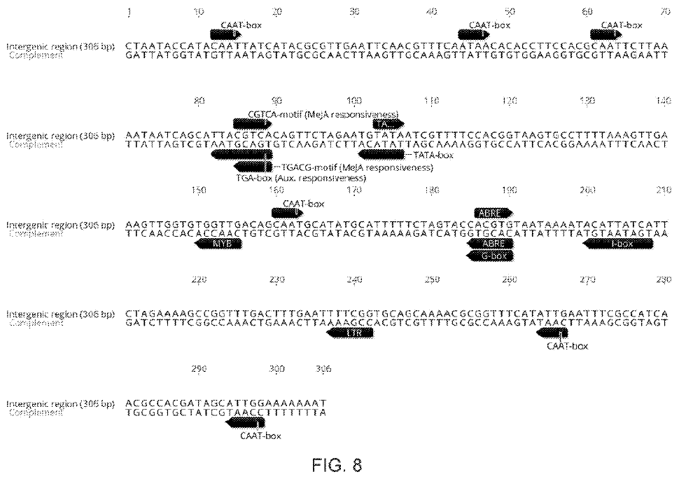

The core promoter element TATA-box is present in the sequence in both the forward and reverse orientation around position 101 (Table 3). The GaRpp1 intergenic region also has multiple putative cis-acting elements present in both orientations, such as CAAT-boxes, CGTCA/TGACG-motif (methyl jasmonate responsive element), and ABRE (abscisic acid responsive-element). A putative TGA-box element (auxin responsive) is present in the reverse strand from positions 82-89 (Table 3; FIG. 8).

TABLE 3
Promoter analysis of GaRpp1 intergenic region using PlantCARE online database.

| Motif | Direction | Sequence | Minimum | Maximum | Length | Function |
|---|---|---|---|---|---|---|
| CAAT-box | forward | CAAT | 12 | 15 | 4 | common cis-acting element in promoter |
| | | | | | | and enhancer regions |
| CAAT-box | forward | ATAA | 44 | 47 | 4 | common cis-acting element in promoter |
| | | | | | | and enhancer regions |
| CAAT-box | forward | CAAT | 61 | 64 | 4 | common cis-acting element in promoter |
| | | TGACGT | | | | and enhancer regions |
| TGA-box | reverse | AA | 82 | 89 | 8 | part of an auxin-responsive element |
| CGTCA- | forward | CGTCA | 85 | 89 | 5 | cis-acting regulatory element involved in |
| motif | | | | | | the MeJA-responsiveness |
| TGACG- | reverse | TGACG | 85 | 89 | 5 | cis-acting regulatory element involved in |
| motif | | | | | | the MeJA-responsiveness |
| TATA-box | reverse | TATACA | 101 | 106 | 6 | core promoter element around -30 of |
| | | | | | | transcription start |
| TATA-box | forward | TATA | 103 | 106 | 4 | core promoter element around -30 of |
| | | | | | | transcription start |
| MYB | reverse | CAACCA | 150 | 155 | 6 | MYB transcription factor binding site |
| CAAT-box | forward | CAAT | 160 | 163 | 4 | common cis-acting element in promoter |
| | | | | | | and enhancer regions |
| G-box | reverse | CACGTG | 185 | 190 | 6 | cis-acting regulatory element involved in |
| | | | | | | light responsiveness |
| ABRE | reverse | CACGTG | 185 | 190 | 6 | cis-acting element involved in the |
| | | | | | | abscisic acid responsiveness |
| ABRE | forward | ACGTG | 186 | 190 | 5 | cis-acting element involved in the |
| | | TGATAA | | | | abscisic acid responsiveness |
| I-box | reverse | TGT | 200 | 208 | 9 | part of a light responsive element |
| LTR | reverse | CCGAAA | 237 | 242 | 6 | cis-acting element involved in low- |
| | | | | | | temperature responsiveness |
| CAAT-box | reverse | CAAT | 264 | 267 | 4 | common cis-acting element in promoter |
| | | | | | | and enhancer regions |
| CAAT-box | reverse | CCAAT | 294 | 298 | 5 | common cis-acting element in promoter |
| | | | | | | and enhancer regions |

The automatic promoter analysis using the PlantPAN 2.0 platform returned a long list of transcription factors binding sites in both DNA strands (Table 4). Two regions in the reverse strand represent putative WRKY DNA-binding domain (positions 153-157 and 225-229). WRKY transcription factors are known to play an important role in plant defense responses (Cheng et al., 2012, Plant Physiol. 159 (2): 810-825, doi.org/10.1104/U.S. Plant Pat. No. 112,196816).

Putative CG-1 DNA binding domains are also present in both DNA strands, and are associated with CAMTA (for CAIModulin-binding Transcription Activator) transcription factors. CAMTAs belong to a family of transcription factors that plays critical roles in plant responses to biotic and abiotic stresses (Bouche et al., 2022, J. Biol. Chem. 277 (24): 21851-2186, doi.org/10.1074/jbc.M200268200).

TABLE 4
Promoter analysis of GaRpp1 intergenic region using PlantPAN 2.0.

| Motif | Direction | Sequence | Minimum | Maximum | Length |
|---|---|---|---|---|---|
| CG-1 | reverse | ACGCG | 23 | 27 | 5 |
| CG-1 | forward | CGCGT | 23 | 27 | 5 |
| NAC; NAM | reverse | GCGTG | 57 | 61 | 5 |
| TF motif seq 0419; aux inducibility | forward | CACGC | 57 | 61 | 5 |
| NAC; NAM | reverse | TGCGT | 58 | 62 | 5 |
| AT-Hook | forward | AAATA | 70 | 74 | 5 |
| AT-Hook | forward | ATAAT | 72 | 76 | 5 |
| TF motif seq 0450; Palindromic C-box | forward | TTACG | 82 | 86 | 5 |
| NAC; NAM | reverse | ACGTA | 83 | 87 | 5 |
| bZIP | forward | ACGTC | 84 | 88 | 5 |
| TF motif seq 0449; TGA-box, aux responsive | reverse | TGACG | 85 | 89 | 5 |
| TF motif seq 0450 | reverse | TGACG | 85 | 89 | 5 |
| TBP | reverse | TAAAA | 129 | 133 | 5 |
| WRKY | reverse | GTCAA | 153 | 157 | 5 |
| homeodomain | forward | TGACA | 154 | 158 | 5 |
| EIN3 | forward | ATGCA | 162 | 166 | 5 |
| EIN3 | reverse | ATGCA | 163 | 167 | 5 |
| TF motif seq 0303, aux response | forward | CATATG | 165 | 170 | 6 |
| TF motif seq 0303 | reverse | CATAT | 166 | 170 | 5 |
| EIN3 | forward | ATGCA | 168 | 172 | 5 |
| EIN3 | reverse | ATGCA | 169 | 173 | 5 |
| bHLH | reverse | ACGTG | 185 | 189 | 5 |
| bZIP | forward | CACGT | 185 | 189 | 5 |
| bHLH | forward | ACGTG | 186 | 190 | 5 |
| bZIP | reverse | CACGT | 186 | 190 | 5 |
| AT-Hook | reverse | TATTA | 191 | 195 | 5 |
| AT-Hook | forward | AAAAT | 195 | 199 | 5 |
| TF_motif_seq_0083 | reverse | TATTT | 196 | 200 | 5 |
| EIN3 | reverse | TGTAT | 198 | 202 | 5 |
| NAC; NAM | reverse | GTCAA | 225 | 229 | 5 |
| WRKY | reverse | TGACT | 225 | 229 | 5 |
| WRKY | forward | TGACT | 226 | 230 | 5 |
| TCR | forward | TTTGA | 230 | 234 | 5 |
| AP2 | reverse | CACCG | 240 | 244 | 5 |
| CG-1 | reverse | CGCGT | 252 | 256 | 5 |
| CG-1 | forward | ACGCGG | 252 | 257 | 6 |
| TF motif seq 0303 | forward | CATAT | 261 | 265 | 5 |
| TF_motif_seq_0315; GT-1 motif* | reverse | TTTTC | 298 | 302 | 5 |
| AT-Hook | forward | AAAAT | 302 | 306 | 5 |

### Example 9: GaRpp1-Mediated Cell Death in N. benthamiana is Independent of Major NLR and PRR Pathways SDignalling Components

NLR signaling components, including EDS1, PAD4, SAG101, NRG, and ADR1, form a vital network in plant immunity. EDS1 acts as a central integrator, collaborating with PAD4 and SAG101 to regulate defense gene expression and coordinate immune responses. PAD4 plays a critical role in controlling the expression of defense-related genes, while SAG101 aids in signal transduction during NLR-mediated immune responses. NRG enhances resistance against diverse pathogens by working alongside EDS1 and PAD4. ADR1 serves as a transcriptional coactivator, further fortifying the plant's defense response. In parallel, PRR signaling components like BAK1 and SOBIR1 are pivotal in initiating immune responses upon detection of pathogen-associated molecular patterns (PAMPs). BAK1 acts as a coreceptor, amplifying PRR activation, while SOBIR1 interacts with both PRRs and coreceptors, transmitting signals that lead to the activation of robust defense mechanisms (Reviewed in Ramirez-Zavaleta et al., 2022, doi.org/10.3390/ijms232112974). Together, these components form intricate signaling networks crucial for plant immunity, allowing plants to effectively ward off a wide array of pathogens. To test if any of these components are involved in GaRpp1 signaling, we have taken advantage of GaRpp1_CG1-mediated cell death in N. benhamiana. We have tested various mutants (FIG. 8) in different PRR and NLR signaling components and showed that GaRpp1_CG1 triggers cell death independently of all of them. This suggests that GaRpp1 functions via different, possibly novel mechanism.

### Example 10: GaRpp1-CG1-Mediated Cell Death Requires All Three Transmembrane Domains

To further study possible mechanism of cell death induction by GaRpp1_CG1 we generated truncated versions of the protein: GaRpp1_CG1_v1, GaRpp1_CG1_v2, GaRpp1_CG1_v3 with one, two and three transmembrane domains truncated, respectively, as well as GaRpp1_CG1_v4 with the N-terminus truncated until the three transmembrane domains (amino acids 1-200). The truncations were cloned under LjUBI promoter. All constructs, including two positive controls (PsCRN63 and HopQ1) and one negative control (Empty vector) were Agro-infiltrated into four-week-old wild-type N. benthamiana plants at 0.8OD and imaged 4 dpi. Cell death imaging indicates that truncation of even one transmembrane domain leads to loss of cell death as compared to full length GaRpp1_CG1 transient expression. However, GaRpp1_v4 transient expression exhibits strong cell death. This experiment shows that transmembrane domains are sufficient and necessary for cell-death phenotype in N. benthamiana.

As GaRpp1_CG1_v4 can induce cell death, it was coinfiltrated with GaRpp1_CG4 to investigate whether the N-terminus truncation would result in cell death suppression. Strong cell death phenotype indicates that GaRpp1_CG4 requires the GaRpp1_CG1 N-terminal region to modulate or suppress cell death phenotype and possibly pathogen resistance.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A nucleic acid molecule comprising a nucleotide sequence selected from the group consisting of:
(a) the nucleotide sequence set forth in SEQ ID NO: 1, 2, 4, 5, 7, 9, 10, 11, 13, 14, 16, 18, 19, 20, 22, 23, 25, 27, 28, or 32;
(b) a nucleotide sequence encoding the amino acid sequence set forth in SEQ ID NO: 6, 8, 15, 17, 24, or 26;
(c) a nucleotide sequence having at least 80% sequence identity to at least one of the nucleotide sequences set forth in SEQ ID NOS: 2, 5, 11, 14, 20, 23, 27, and 32, wherein the nucleic acid molecule is capable of conferring resistance to Asian soybean rust (ASR) caused by at least one race of Phakopsora pachyrhizi, relative to a control plant not comprising the nucleic acid molecule;
(d) a nucleic acid molecule comprising a nucleotide sequence encoding an amino acid sequence having at least 80% sequence identity to at least one of the amino acid sequences set forth in SEQ ID NO: 6, 15, and 24, wherein the nucleic acid molecule is capable of conferring resistance to ASR caused by at least one race of P. pachyrhizi, relative to a control plant not comprising the nucleic acid molecule;
(e) a nucleotide sequence having at least 80% sequence identity to at least one of the nucleotide sequences set forth in SEQ ID NOS: 4, 7, 13, 16, 22, 25, and 28, wherein the nucleic acid molecule is capable of modulating in a plant the activity of at least one R gene therein, relative to a control plant not comprising the nucleic acid molecule; and
(f) a nucleic acid molecule comprising a nucleotide sequence encoding an amino acid sequence having at least 80% sequence identity to at least one of the amino acid sequences set forth in SEQ ID NO: 8, 17, and 26, wherein the nucleic acid molecule is capable of modulating in a plant the activity of at least one R gene therein, relative to a control plant not comprising the nucleic acid molecule.

2. The nucleic acid molecule of claim 1, wherein the nucleic acid molecule is an isolated nucleic acid molecule.

3. An expression cassette or vector comprising the nucleic acid molecule of claim 1 or 2.

4. A host cell transformed with the nucleic acid molecule of claim 1 or 2 or the expression cassette or vector of claim 3.

5. A plant or plant cell comprising the nucleic acid molecule of claim 1 or 2 or the expression cassette or vector of claim 3.

6. The plant or plant cell of claim 5, wherein the plant is a soybean plant and the plant cell is a soybean plant cell.

7. The plant of claim 6, wherein the soybean plant comprises enhanced resistance to ASR caused by at least one race of P. pachyrhizi, relative to the resistance of a control soybean plant.

8. A soybean plant, plant cell, or seed comprising stably incorporated in its genome a heterologous polynucleotide comprising a nucleotide sequence selected from the group consisting of:
(a) the nucleotide sequence set forth in SEQ ID NO: 1, 2, 4, 5, 7, 9, 10, 11, 13, 14, 16, 18, 19, 20, 22, 23, 25, 27, 28, or 32;
(b) a nucleotide sequence encoding the amino acid sequence set forth in SEQ ID NO: 6, 8, 15, 17, 24, or 26;
(c) a nucleotide sequence having at least 80% sequence identity to at least one of the nucleotide sequences set forth in SEQ ID NOS: 2, 5, 11, 14, 20, 23, 27, and 32, wherein the nucleic acid molecule is capable of conferring resistance to Asian soybean rust (ASR) caused by at least one race of Phakopsora pachyrhizi, relative to a control plant not comprising the nucleic acid molecule;
(d) a nucleic acid molecule comprising a nucleotide sequence encoding an amino acid sequence having at least 80% sequence identity to at least one of the amino acid sequences set forth in SEQ ID NO: 6, 15, and 24, wherein the nucleic acid molecule is capable of conferring resistance to ASR caused by at least one race of P. pachyrhizi, relative to a control plant not comprising the nucleic acid molecule;
(e) a nucleotide sequence having at least 80% sequence identity to at least one of the nucleotide sequences set forth in SEQ ID NOS: 4, 7, 13, 16, 22, 25, and 28, wherein the nucleic acid molecule is capable of modulating in a plant the activity of at least one R gene therein, relative to a control plant not comprising the nucleic acid molecule; and
(f) a nucleic acid molecule comprising a nucleotide sequence encoding an amino acid sequence having at least 80% sequence identity to at least one of the amino acid sequences set forth in SEQ ID NO: 8, 17, and 26, wherein the nucleic acid molecule is capable of modulating in a plant the activity of at least one R gene therein, relative to a control plant not comprising the nucleic acid molecule.

9. The soybean plant, plant cell, or seed of claim 8, wherein the heterologous polynucleotide further comprises a promoter operably linked for the expression of the nucleotide sequence in a plant.

10. The soybean plant, plant cell, or seed of claim 8, wherein the heterologous polynucleotide comprises the nucleotide sequence set forth in SEQ ID NO: 1.

11. The soybean plant, plant cell, or seed of claim 8 or 9, wherein the heterologous polynucleotide comprises the nucleotide sequence set forth in SEQ ID NO: 2 or 5 or a nucleotide sequence encoding the amino acid sequence set forth in SEQ ID NO: 6.

12. The soybean plant, plant cell, or seed of claim 11, wherein the heterologous polynucleotide further comprises the nucleotide sequence set forth in SEQ ID NO: 4 or 7, or wherein the soybean plant, plant cell, or seed further comprises an additional heterologous polynucleotide comprising the nucleotide sequence set forth in SEQ ID NO: 4 or 7.

13. The soybean plant of any one of claims 8-12, wherein the soybean plant comprises enhanced resistance to ASR caused by at least one race of P. pachyrhizi, relative to the resistance of a control soybean plant.

14. A method for enhancing the resistance of a soybean plant to ASR caused by at least one race of P. pachyrhizi, the method comprising introducing into at least one soybean plant cell a heterologous polynucleotide and regenerating a soybean plant comprising in its genome the heterologous polynucleotide, wherein the heterologous polynucleotide comprising a nucleotide sequence selected from the group consisting of:
(a) the nucleotide sequence set forth in SEQ ID NO: 1, 2, 5, 9, 10, 11, 14, 18, 19, 20, 23, 27, or 32;
(b) a nucleotide sequence encoding the amino acid sequence set forth in SEQ ID NO: 6, 15, and 24;
(c) a nucleotide sequence having at least 80% sequence identity to at least one of the nucleotide sequences set forth in SEQ ID NOS: 2, 5, 11, 14, 20, 23, 27, and 32, wherein the nucleic acid molecule is capable of conferring resistance to Asian soybean rust (ASR) caused by at least one race of Phakopsora pachyrhizi, relative to a control plant not comprising the nucleic acid molecule; and
(d) a nucleic acid molecule comprising a nucleotide sequence encoding an amino acid sequence having at least 80% sequence identity to at least one of the amino acid sequences set forth in SEQ ID NO: 6, 15, and 24, wherein the nucleic acid molecule is capable of conferring resistance to ASR caused by at least one race of P. pachyrhizi, relative to a control plant not comprising the nucleic acid molecule.

15. The method of claim 14, wherein the heterologous polynucleotide further comprises an additional nucleotide sequence selected from the group consisting of:
(e) the nucleotide sequence set forth in SEQ ID NO: 4, 7, 13, 16, 22, 25 or 28;
(f) a nucleotide sequence encoding the amino acid sequence set forth in SEQ ID NO: 8, 17, or 26;
(g) a nucleotide sequence having at least 80% sequence identity to at least one of the nucleotide sequences set forth in SEQ ID NOS: 4, 7, 13, 16, 22, 25, and 28, wherein the nucleic acid molecule is capable of modulating in a plant the activity of at least one R gene therein, relative to a control plant not comprising the nucleic acid molecule; and
(h) a nucleic acid molecule comprising a nucleotide sequence encoding an amino acid sequence having at least 80% sequence identity to at least one of the amino acid sequences set forth in SEQ ID NO: 8, 17, and 26, wherein the nucleic acid molecule is capable of modulating in a plant the activity of at least one R gene therein, relative to a control plant not comprising the nucleic acid molecule.

16. The method of claim 14, wherein the regenerated soybean plant comprises an additional nucleotide sequence selected from the group consisting of:
(e) the nucleotide sequence set forth in SEQ ID NO: 4, 7, 13, 16, 22, 25 or 28;
(f) a nucleotide sequence encoding the amino acid sequence set forth in SEQ ID NO: 8, 17, or 26;
(g) a nucleotide sequence having at least 80% sequence identity to at least one of the nucleotide sequences set forth in SEQ ID NOS: 4, 7, 13, 16, 22, 25, and 28, wherein the nucleic acid molecule is capable of modulating in a plant the activity of at least one R gene therein, relative to a control plant not comprising the nucleic acid molecule; and
(h) a nucleic acid molecule comprising a nucleotide sequence encoding an amino acid sequence having at least 80% sequence identity to at least one of the amino acid sequences set forth in SEQ ID NO: 8, 17, and 26, wherein the nucleic acid molecule is capable of modulating in a plant the activity of at least one R gene therein, relative to a control plant not comprising the nucleic acid molecule.

17. The method of any one of claims 14-16, wherein the heterologous polynucleotide is stably incorporated into the genome of the soybean plant cell.

18. The method of any one of claims 14-17, wherein the heterologous polynucleotide comprises further comprises a promoter operably linked to the nucleotide sequence for the expression of the nucleotide sequence in a plant.

19. The method of any one of claims 14-18, wherein the soybean plant comprising the heterologous polynucleotide comprises enhanced resistance to ASR caused by at least one race of P. pachyrhizi, relative to the resistance of a control soybean plant.

20. A soybean plant producible or produced by the method of any one of claims 14-19.

21. A seed of the plant of any one of claims 5-13 and 20, wherein the seed comprises the heterologous polynucleotide.

22. A method of limiting ASR caused by at least one race of P. pachyrhizi in agricultural crop production, the method comprising planting a seed of the plant of any one of claims 5-13 and 20 and growing the seed under conditions favorable for the growth and development of a plant resulting therefrom, wherein the seed comprises the nucleic acid molecule, expression cassette, vector, or heterologous polynucleotide.

23. A method for identifying a plant that comprises an R gene and/or a modulator gene, the method comprising detecting in the plant, or in at least one part or cell thereof, the presence of a nucleotide sequence selected from the group consisting of:
the nucleotide sequence set forth in SEQ ID NO: 1, 2, 4, 5, 7, 9, 10, 11, (a) 13, 14, 16, 18, 19, 20, 22, 23, 25, 27, 28, or 32;
(b) a nucleotide sequence encoding the amino acid sequence set forth in SEQ ID NO: 6, 8, 15, 17, 24, or 26;
(c) a nucleotide sequence having at least 80% sequence identity to at least one of the nucleotide sequences set forth in SEQ ID NOS: 2, 5, 11, 14, 20, 23, 27, and 32, wherein the nucleic acid molecule is capable of conferring resistance to Asian soybean rust (ASR) caused by at least one race of Phakopsora pachyrhizi, relative to a control plant not comprising the nucleic acid molecule;
(d) a nucleic acid molecule comprising a nucleotide sequence encoding an amino acid sequence having at least 80% sequence identity to at least one of the amino acid sequences set forth in SEQ ID NO: 6, 15, and 24, wherein the nucleic acid molecule is capable of conferring resistance to ASR caused by at least one race of P. pachyrhizi, relative to a control plant not comprising the nucleic acid molecule;
(e) a nucleotide sequence having at least 80% sequence identity to at least one of the nucleotide sequences set forth in SEQ ID NOS: 4, 7, 13, 16, 22, 25, and 28, wherein the nucleic acid molecule is capable of modulating in a plant the activity of at least one R gene therein, relative to a control plant not comprising the nucleic acid molecule; and
(f) a nucleic acid molecule comprising a nucleotide sequence encoding an amino acid sequence having at least 80% sequence identity to at least one of the amino acid sequences set forth in SEQ ID NO: 8, 17, and 26, wherein the nucleic acid molecule is capable of modulating in a plant the activity of at least one R gene therein, relative to a control plant not comprising the nucleic acid molecule.

24. The method of claim 23, wherein the presence of the R gene or modulator gene is detected by detecting at least one marker nucleotide sequence.

25. The method of claim 23 or 24, wherein the nucleotide sequence comprises or consists of the nucleotide sequence of (a).

26. The method of any one of claims 23-25, wherein detecting the presence of the nucleotide sequence comprises a member selected from the group consisting of PCR amplification, nucleic acid sequencing, nucleic acid hybridization, and an immunological assay for the detection of the R protein encoded by the R gene or modulator protein encoded by the modulator gene.

27. Use of the plant or seed of any one of claims 5-13, 20, and 21 in agriculture.

28. A human or animal food product comprising, or produced using, the plant, and/or seed of any one of claims 5-13, 20, and 21, or a part or parts of the plant and/or seed.

29. A polypeptide comprising an amino acid sequence selected from the group consisting of:
(a) the amino acid sequence set forth in SEQ ID NO: 6, 15, and 24;
(b) the amino acid sequence set forth in SEQ ID NO: 8, 17, and 26;
(c) an amino acid sequence having at least 80% sequence identity to at least one of the amino acid sequences set forth in SEQ ID NO: 6, 15, and 24, wherein a polypeptide comprising the amino acid sequence is capable of conferring resistance to ASR caused by at least one race of P. pachyrhizi, relative to a control plant not comprising the polypeptide; and
(d) an amino acid sequence having at least 80% sequence identity to at least one of the amino acid sequences set forth in SEQ ID NO: 8, 17, and 26, wherein a polypeptide comprising the amino acid sequence is capable of modulating in a plant the activity of at least one R gene therein, relative to a control plant not comprising the polypeptide.

30. A promoter comprising a nucleotide sequence selected from the group consisting of:
(a) the nucleotide sequence set forth in SEQ ID NO: 3, 12, 21, 29, 30, and 31; and
(b) a nucleotide sequence having at least 90% sequence identity to at least one of the nucleotide sequences set forth in SEQ ID NOS: 3, 12, 21, 29, 30, and 31, wherein the promoter is capable of driving the expression on an operably linked polynucleotide in a plant cell.

31. The promoter of claim 30, wherein the promoter is a bidirectional promoter that is capable of driving the expression of two operably linked polynucleotide sequences.

32. The promoter of claim 30, wherein the promoter is a pathogen-inducible promoter that is capable of driving the expression of the operably linked polynucleotide sequence in response to an infection by a pathogen.

33. The promoter of claim 31, wherein the promoter is a pathogen-inducible promoter that is capable of driving the expression of the two operably linked polynucleotide sequences in response to an infection by a pathogen.

34. The promoter of claim 30 or 31, wherein the promoter is inducible by at least one race of P. pachyrhizi.

35. The promoter of claim 30 or 31, wherein the promoter is inducible by two, three, four, five, or more races of P. pachyrhizi.

36. A method for introducing CG1 into a leguminous plant, the method comprising:
(a) crossing a first leguminous plant comprising in its genome at least one copy of CG1, with a second leguminous plant lacking in its genome CG1whereby at least one progeny plant is produced; and
(b) selecting at least one progeny plant comprising in its genome CG1 by detecting in the progeny plant the presence of CG1.

37. The method of claim 36, wherein the first leguminous plant is a G. argyrea plant and the second leguminous plant is a G. max plant lacking in its genome CG1.

38. The method of claim 36 or 37, wherein the first leguminous plant further comprises in its genome at least one copy of CG4.

39. The method of claim 38, wherein CG4 is linked to CG1.

40. A progeny plant obtainable using the method of any one of claims 36-39.

41. A seed of the progeny plant of claim 40.


SEQUENCE LISTING (40 sequences)

SEQ ID NO: 1 - DNA, 12314 bp, Glycine argyea
  source 1..12314: mol_type = genomic DNA
agaaaaaataacaagtattaatgtgatatttaaaacaaaagagaatttcaatagtaagatttttcatttaaaaagcgtcttgggccacttctaaagaagttgcaaagaaactgcttttcagaaatttcatttcaaagaccaattactatcaaatactcaaactttgatatttcaaaggcaaccacgacctaaaaagaaataaaacagataaattgtttctggatccaaaataccatattccagacttacattcaagtgcgtcatttcaggtatcctggatcttgatgatacactcaggcgagtgtttctgagttacttctcctccaacatcatttggataggagcaccatatgcagctagcatagagcacccccatgattatgatgctaagaagtagcattaaggatgtggcccctccaaacacaaacaataggcctgagattttagctgaaccaccccatgaaacactatatccagacactgcaactattatcgctaaaatgtaatagagtaaattaaatcccagcattgagtatgcaggtatacgatgaggcaatgacaaggtaagcatggctgcaaaaataaagttcaggaaaaatattgatgcccatactgtgatggaacatgctttccccatacttaattccattgggtcaatgcctctggattgggataacttctcaagaggattgcccaaaacagatagttgggcccattgcattccatataaggcagcccatgtagtaagacgatttgcatcagtagaatgtgattggaatagcctgttcccataaaacttcaaaaatgtgctcaaactcttcgtctttcctttatctacttttaacaccccattatcgtcaatctttacaacatggtttgggggatgagaacggcgattttccttttcttcctgttgatagtcaatcacaatggagaaatatggttggtgagtgagaattttgatgaaataatagatggtctttctatggaactttatttttggtattttcttgtaaatgtaaagatctcatgcaatggaaatgtcattgtaggggacaaaataacaaaatgatatatggctcattttgcacccatgttgagatatgtgacacacatgcacaccatatatataattttagatatgttgaggtatgtgaaagatgtgagatatttcaaaaataaaattgtgagatatttgaaagacatatcctctctatgtgagcaaaaagtgtgtctaaagcttaatgacttgttattattacctaattctatatgttaccttgcattttttcagttgctgatatagttctttctgtggctgtacaaaaaagcaacaaatgttaggtacttgagaagtaaagatccaatcaagttacattaaggaaaaatatgcatatgcatctctttattaaattttcaagccaattagtattgatcatcaaaatggacaaaaggaaaagacaatgattttccctacatggctcattttgctcctatgttgagttatgtgagatatttcaagccgagaaaaaacaaaaatatatttgaaagacacatctttttatgtgagtagaaagttagtctaacaatatggagttagacaccaaatgtttcgttacgagatgacactacactttgcgccattgaaatataatgtgtcttttctcccagtgcataagacgattttgcaaataattacataagacataaattgtactttttttgtcacgagacaaggtcattgaattagcttttttacatcaaaattgttattataatgttccatttgacaataaaataataatgttaatgataagtcccaaaatttttttgttttggatgaagaaaacacaataacctgtaattaattagttctatatgtaccttgttctcctcttgactctcgcatgcttgtttttgtgcataaaagcaaggaaggttaggtacttgatcatatcattagatattatgttaaattaaggtgcaattcaacttatttgttaaaataaaataccatgattttccctagcttatggatcatatcattagatattatgttgagatacatcaaaaacaaaaatgtgacatatttgaaggatatactttgtattgtgagtagaaattctgtctaaaaatgtaagatgatttgatttctgcactagacaaagttttgtgtttttcttgatcacatctctatatcaaatttgttattacaatgttccatttgtcattaaaataataatggtattgataagacttgggaaaaattatgtacctttgatattatttcattctctggccttttcacaaaacaacgttctaggtcaatgtcagtatatacttgcagccgcttctcctgtacataaaagaaatagaggttaattaggcacttagtagaggtgaaattcaaatacattaaggaaaaataagcatgcctttattatatttaatctcatgtcaattagtattgagcattaaaaatggacaaaaggaagagaaagaaaattatatgattcatttagctcccgctttggatacaaacggaagaaaggcaaattaaatgtttaaatttatttgttaaaaaaaactaaaaataaacaactttttatttttagtgtgattgtctaaattgcttatgcttaaaaaataactttcctcttattttaagaaactaattatatctacttaaaaaaatgctgataaaaaatgcttattttaaaattgattttctaactttaaaaacaaacacacccaatgccttaccctcggtagaaattgatgtttttttgtagtttggtcctgttttcataaatttggatacttaaatgctcaattacctctttaagatactacacttttatttattttatctaaaatgtaactttcgtcttcttatttttttaaatccattattttttctcataatttttaattgagacattttgttttttatttttaattaattgcaattttagtcctcctatttttaattgagacatttcgcttttaaaaatttgtgattttagtccctcactttcaagacttgtaaaaataatatgaactatgattgtacttagatagtggatagaattaacaataataaaccatggttgtactttaatagtggatagaaagaaattaatagaaaaaaattgaacggatccaatggaataaaataaattaatatttattgtgtcaatattttaatgataaaaggatcaaagagattgattccatctcatagtagctaaattagaaaagaagaaaaaataatagtattgggtgatatgaaataaaatacgtataataaagtcatgtttagttctattccaacatatttaaaaccaatcatgactcaaaagaaaaaaaatagcaccttccattttattgtatttctaggtgtcaattggaacacaactccgaatatttcaggaattaatgattataaatattgaaatcattttgcaactcaaagtaatctaggtaattgatttaactttattttactacttttacaagagaaaacgaaaatatgacattttatcgatagtttctttttctatattggtagatatgttatagatatttgaaagatgtgagatatttaaaaaaaaagttagattttgaaagacacacaagaaattaacttttattacgagaaactagctaagaaacttgttaaaaaataattacttgtcaaccaagttataataaggcataactattatttctaacaagcacaagcctttatgtatgggtaaagagtgtgtttgacaatgtagacttaggcaacatgtgtttagttatgatgacactacattttgttgaaaacataattatgtctattttccttaatgtgagacaaattttccaatcatctatacaagtaacaagactaaggttgtgtttttcttgttcacaaagtggttgcattatcttctctatatcaaatttgttattataatgatccaattgacagtaaaatattaatagcaatgaatagcactatagaaaattatgttttgaacgaagagatcataacttgttgtatcaattagttctatacttacatgatttttcaccatttgcatcggataatactgatcaagatatattccattaaggtaacgttgtagtatgactctctgtacataaaagcaataaaagttaggtacctaacaagtgaaggtgcaattcaattactgtaaggaaaaacatgcatatttttagtaaacttctatgccaattagtattgatcataaaaatggacaacaggaagagaatcacaattacttgccttggtttatttcgttgccatgtctggacaaaaggcaaacaacgacaaggacttgcccttggtagaaaaattgttgtttttcgtagcttggtacaggtttcacaaagttggaaacttaaaaagataaattatttctttaagatactatccattaatgtaatgtcactaattataatcaatgtaatttgggtgatatttgcataattaagagacttagtattaataaaagagaattaatgaaagaatggacattttggtatttgtttagaggttagggtgtgaataagagattgctgaatgcacccccctcctcatgttaaaaactgtggctgaccatggttgtctcactttctccttcttcttcctttatgtccatgtctctccttcacacccaagcaccatttctcttcactccttgattagtggctggaaacataaatcaagcaagaaaggaagctcttctccttgtcctttattgttgttgagttttggttgtgagcaatttgaggaaaagagtagagctaggagctgcccacttcattcggagtagctgggcagaattggaagctatagagagctggtccactctccaatttgatcaattgaactctaaggttggtactctcctcctatttctccttgattcttagaaatcataaggttagggtttggtttgataaatgttgagtattggaagggtgaaacagttttaaggtgtaatttggagtgtgtagaagttgattcaaggttgaaatggtgttccaagcatgaaacatgcatgttcatgacatcaaggaacatcgatctggtcaaccctgatgttttgtgcataacaggagctatcggtatccaaatggtctaaaaacagtgtcatttgaaagttaagagattcaagtaggttctggaattttcgtgctatttttggaggttcctagaatttattgtgcagagttataacaggctgttatactctgtttttagcagattttactggtgtttagcagattttactggtgcagagggcaatcttgaagaaattttatctcagtctaggatgatccaaatttagtgatttcagtggaaatagaaagatgtgtatgtttaatttatacctgtaaaatctcaggtcatttggatgagtatcctggtgttggcgggtgtttgaaaattgctggttgaactgatatgttattgtcagcactgttatagtgcgtaacagttctggtattttaagtgttacagaggttgtggtttatgaaattggatgaaaccagttttgtttgaaagataggagataaatatagatgcctctaaattcccttgattttaggagcttcctacagggatttatgatttttataagtgggtacaaaatctgcataattttatgcagaaaattctgtttttccgtgtgttttggtataattaaatgccttgtacggataggatgattgttgattcatgcctgtgtgcctttatatagggcatgacaacgagggtgttagtggcggagaccacagttcagggattgaatagcagagcacggccttgcctggtaagttcgtggttcttgggagtgttttccactgcttgagtttcaccacactccgagggagtgatggttgcatacatgattcatatgcattaaattggatgttacatctttcctcatgataatatgtatgcatattctgtgctttgtgtttggttgcctttcggatagatcttctggcaagttagatagatatagacctccctaggagcagagcacaactcctagttggtttgctcacgtaaagggtatatgatcccttaacaccagagatacgtggtcccgtcgtggttgaagcctattctcggaggtgtcatatacgtgcatccatgcgtaaaggggtatctcccgatgggatgacaagggtgtgagttccggcctaacactgaggactgagagtcctgatgctgatgatgatgcatctttggcaggcttagtaccgcagactcctgatgctgatgatgatgcatcattggcagcgtagttccgcagatacacattagagctaaagaatgtttagttgatttaccacggtctggacccgggtgatgaaaccggataccccttctccttccttcattttcattccttccaactctccacggagagtttgaagtcgaacctgtctaactcgatcggctcccttgtagacatttgcaagaatgtcccacgcctcctttgaatttgttgcaccagcgatcttctaaaacccagaatcatcaacagcccggaataacatgtacaacacccctttgtctttcaacctcgtctccttcaatgtcttgttctgcgccatcgtatatcctgctgtggatgttggtttctcgaaaccaccatggacaacttcccaactatcatgggctcccaaaagagctttcatatggatactccaattttcataattagcctttgtaaggcggggtagtggcagactactacttgacccactctccattttctcaagctctgaaaccaatttgtgatatcacatgtcctcacacacactggtgcactcacataaacacacaacaatctctctagtagaatacttctgatgtatttagataatggtctgccaagccacacattggcctcttacatgagatgctatttataagtaatagcaagtgagatatgactcttcacatgagtaacttatttacctagggacatgactcttcacatgagtaacttcttcacctagggacatgactcttcacatgagtaacttcttcacctatctaatttattattattaaagtttaatccaacaaaatctccccataaactttaaatatttctttcgtctttcatccccaacttttttttttctttttctcttataaacccatttaactccaatgggctgacttccttttggcagttcggtcaactcccaagtttcattccgttcaatgaccttgattttttcattcatggcggttttccatttttcatctcggattgcctctttaaatgttatttcttcagaatctgtcaacaaacaaataaggtaagcttcattagttgaatcatacaggtcatgcaaactcctcattcggggttgtctcatctcgtcttcatcagatgtctcacttcttgccgggatccaacttggtgattctccttccttctcttcttgtcgagtaagcgtctgtccgggatctgaggtgaccttggatgaagtgctccagttccattcatcttcttcattaactttcacatctcggcttatcattactttcttggtgccgggatccaacaacttgtatccctttgtcttttcgtcatatccaatgaaaataaatttcctgctcttgtcttccaacttggttctttgctgatcagggatgtgtgcataagccacactcccgaagactctgaaatggcctactgatggcttctgtcctgtccatgcttcctgtggggtctttccatttagactggaatgtggacaacggttctgcacatacactgcacacaatacggcctgcccccaaaactccttaggcattttcttcgttttcaacattgaccttaccatatcaagaatgatacggtttttctctcagccactccattttgctgtggtgagtacggtgcagttaaaaacctttttatcccatgctcctcgcaataccttttaaatgcaattgatgtgtactccccacctcggtcagaacggattgacttgattcaccttccagttgcattttctatcatcatcttaaaactcttgaatacttcaagcactttggatttttcttccagcaaatacacccaagttttacgactaaaatcgtctataaacgagacaaaatacctctttccactgaaagacccaggagagatgggtccacacaagtcggtatgaattagttctagtggttgttgagatctgtatttggccgtcttttgaaatggactcctaacatgcttgcttagcacacattcttcacaaatctcttttcacagttcaaactcggcaactcttccaccatattcttgttcatcatctcgaccaggcctccgtagtgaagatgcccaaatcggcgatgccataaggatacttcatcactcatctcggcttgcaaacagtcgacctgcacactcctcaacattagtttgaacattctatttttcttcatctgaacttgaactatcaaacatccttgtttatccttcaagaagagaactcggtctgtcatgagaaccgagtatccattctccatcagttgtcccacactaagaatattactttttagatcgggcacgtaatagacatcttggatgcgcccaattcgtccatctttctgcaagaaacaaatcgtacctcagcctttgacatcaacctttgaaacatctccaaacgaaacatgtatggcttctatttgtctcagattactgaacaaatgcttatgcccacacatatgattgcttgcactagtgtctagataccattccatctcgaatccctggttgacgtcacttctagtcatcatcagaattcctccatctcttacttctccttctgtaacaaggtttgaagtttcatttcttctttcatcttggcaatccttcgcaaaatgtccgactcgaccacagttgtaacatctgtccgagttgcaatctcttgagtaatgtccatactttcctcatttaaaacattctacatttgattgatttgaatgtcggcctcttccacgaccttggcctcgccaattctgccgacttaatttagcctgctccttattccttccttcttgaccgcggcctctactaccgcgaccgtctcctcgacctccatagtcatgccctctacctcgaaagttttgagaataaagtaccttttcgtccttgattgaagcttttgtctggagtgcttgctccagtgactcttctttcttctttttctttctttgttcatgcgcctccaaggaaccagcaagttcgtcaacggtgagcgttgctaggtcctttgactcctcaatcgcacatacaacattctcgaagtcgtcagtgagagacctcagaattttctccaccactttggcatcggacaacacctctccatttctgtttagttgatttaccacggtctagacccgggtgatgaaaccggataccccttctccttccttcattttcattccttccaactctccacggagagtttgaagtcgaacctgtctaactcgatcggctcccttgtagaaatttgcaagaatgtcccacgcctcctttgaagttgttgcaccagcgatcttctcgaacccggaatcatcaacagtccagaataacatgtacaacgcccccttgtctttcaacctcatctccttcaatgtcttgttctgcaccgccgtatatcctgctatggatgttggttccttgaaaccaccatggacaacttcccaactatcatgggctcccaaaagagctttcatctggatactccagttttcgtaattagcctttgtaaggcggggtagtggtagactactacttggcccactctccattttctcaagctctgataccaatttgtgatatcacaggtcctcacacacactggtgcactcacacaaacacacaacaatctctctggtagaatacttctgatatatttagataatggtctgccaagccacacattgacctcttacatgagatgttatttataagtaatagcaagtgggacatgactcttcacatgagtaacttctttacctagggacatgactcttcacatgagtaacttcttcacctatctaatttattattattaaagtttaatccaacataaattacttaatcaggatcgtgatggcaattaagagttgatgagagttataacttacagtgtcaaatttccggaaaggcgaatccactttaggcgcaaacttggattcccaaactgaatcagcaatcacaatccccatgttaggcactgcacacaaatcaaagaaagaatatgcatacatgttagaaattatgatggcggtataaacagaaactgctgctgagttatatcattatctactacttccacttcaatcctaatatctggatattaatttaccttgtataacccatagttctggatacagttcctctatagtttcctcgagtatccatgaaagcatattcttaaaccaaatctgtgtgtccagttctgctatgcattgcaaaattttgtttgtcagctctaccacagagtcgtcgccactgatcacgtttatttcttctatcaaatgcgcaatattctcggcttccataactgaataacatggtagatttttcaaatgagttttcatccgatctataatggcttctccacgatccgatggtggtgcaggctcattgatctcgtccatttcttctgttcctctcagtctcagcaagtttacaaactactcactttcttttttgctcaagagacacataacttgcttctacctaataccatacaattatcatacgcgttgaattcaacgtttcaataacacaccttccacgcaattcttaaaataatcagcattacgtcacagttctagaatgtataatcgttttccacggtaagtgccttttaaagttgaaagttggtgtggttgacagcaatgcatatgcatttttctagtaccacgtgtaataaaatacattatcattctagaaaagccggtttgactttgaattttcggtgcagcaaaacgcggtttcatattgaatttcgccatcaacgccacgatagcattggaaaaaaatgttttgagaaggtgtgtttggtttcagactttcacatatcatccaggtggagcaaaatttgcagtacgagagagttggattcttgggctgggaactactaccaaatcatgtatatgcctaatttctcctcggtaagttgtatatgcttacttctatgctgaactgcatcatgatcaaactccattcaactcaaacaaaccgagctaatttgttcttaatcattcttatttattatcgtttttcacatcgattttgaggaatctgaatatgccaaagtcagcagttgacacatgtatagctatattaattatattttgcagtttccatcgacaaatcttgttgcgaaaatcttgaaccaaaggcagaatatccatgaaaaggaggagcatggagatgatgagaagggcaagctggtatcattaggacaaatgaatagtttaagtgtaagtttccttcttttcccctctaaactatataagttgtaattattgcagaaatgtaatgccattatattatgctaatactaattaaactgcagagcaagcaagatgttccaatgttagacttagttagagccatgtgcactgtccatgatgagaaatcaggacaacctttagcttttgagtacacattgaacacaattatgcattttgttctggttgtgtgtagtgagttatttaactaagctaatatatggtgtgatgtatattatgtgtattccagaggaaagaggatagtagaacatcaatgaacattagttctgaagttgaaagaagaacaccagaaatcatgggaagatgtgcatcgtggtgggccacctccacattttttttgtgtttgtgttcattaaaattcagcaagtcacttctccaaccacaaggacccaaatcactatggcttgccatagtaggaatgttcagttttactgtgtttgtatctgcttcttctgttatgctagttgttggagcacagttcactcgtatgcagataactgttatgctcagtgctttcttggctatgctgttagcttaattagatagttttcatatatgtacttatgttttcttcaggcagggttgacaagttgttttaatcttgttatttacataatttcattcgcataacattaaaacaaagtttgggtatgtgatatatcaatatcaacgattaaccagacagaacatattcacggatgaacattga

SEQ ID NO: 2 - DNA, 10805 bp, Glycine argyea
  source 1..10805: mol_type = genomic DNA
gtagaagcaagttatgtgtctcttgagcaaaaaagaaagtgagtagtttgtaaacttgctgagactgagaggaacagaagaaatggacgagatcaatgagcctgcaccaccatcggatcgtggagaagccattatagatcggatgaaaactcatttgaaaaatctaccatgttattcagttatggaagccgagaatattgcgcatttgatagaagaaataaacgtgatcagtggcgacgactctgtggtagagctgacaaacaaaattttgcaatgcatagcagaactggacacacagatttggtttaagaatatgctttcatggatactcgaggaaactatagaggaactgtatccagaactatgggttatacaaggtaaattaatatccagatattaggattgaagtggaagtagtagataatgatataactcagcagcagtttctgtttataccgccatcataatttctaacatgtatgcatattctttctttgatttgtgtgcagtgcctaacatggggattgtgattgctgattcagtttgggaatccaagtttgcgcctaaagtggattcgcctttccggaaatttgacactgtaagttataactctcatcaactcttaattgccatcacgatcctgattaagtaatttatgttggattaaactttaataataataaattagataggtgaagaagttactcatgtgaagagtcatgtccctaggtaaagaagttactcatgtgaagagtcatgtcccacttgctattacttataaataacatctcatgtaagaggtcaatgtgtggcttggcagaccattatctaaatatatcagaagtattctaccagagagattgttgtgtgtttgtgtgagtgcaccagtgtgtgtgaggacctgtgatatcacaaattggtatcagagcttgagaaaatggagagtgggccaagtagtagtctaccactaccccgccttacaaaggctaattacgaaaactggagtatccagatgaaagctcttttgggagcccatgatagttgggaagttgtccatggtggtttcaaggaaccaacatccatagcaggatatacggcggtgcagaacaagacattgaaggagatgaggttgaaagacaagggggcgttgtacatgttattctggactgttgatgattccgggttcgagaagatcgctggtgcaacaacttcaaaggaggcgtgggacattcttgcaaatttctacaagggagccgatcgagttagacaggttcgacttcaaactctccgtggagagttggaaggaatgaaaatgaaggaaggagaaggggtatccggtttcatcacccgggtctagaccgtggtaaatcaactaaacagaaatggagaggtgttgtccgatgccaaagtggtggagaaaattctgaggtctctcactgacgacttcgagaatgttgtatgtgcgattgaggagtcaaaggacctagcaacgctcaccgttgacgaacttgctggttccttggaggcgcatgaacaaagaaagaaaaagaagaaagaagagtcactggagcaagcactccagacaaaagcttcaatcaaggacgaaaaggtactttattctcaaaactttcgaggtagagggcatgactatggaggtcgaggagacggtcgcggtagtagaggccgcggtcaagaaggaaggaataaggagcaggctaaattaagtcggcagaattggcgaggccaaggtcgtggaagaggccgacattcaaatcaatcaaatgtagaatgttttaaatgaggaaagtatggacattactcaagagattgcaactcggacagatgttacaactgtggtcgagtcggacattttgcgaaggattgccaagatgaaagaagaaatgaaacttcaaaccttgttacagaaggagaagtaagagatggaggaattctgatgatgactagaagtgacgtcaaccagggattcgagatggaatggtatctagacactagtgcaagcaatcatatgtgtgggcataagcatttgttcagtaatctgagacaaatagaagccatacatgtttcgtttggagatgtttcaaaggttgatgtcaaaggctgaggtacgatttgtttcttgcagaaagatggacgaattgggcgcatccaagatgtctattacgtgcccgatctaaaaagtaatattcttagtgtgggacaactgatggagaatggatactcggttctcatgacagaccgagttctcttcttgaaggataaacaaggatgtttgatagttcaagttcagatgaagaaaaatagaatgttcaaactaatgttgaggagtgtgcaggtcgactgtttgcaagccgagatgagtgatgaagtatccttatggcatcgccgatttgggcatcttcactacggaggcctggtcgagatgatgaacaagaatatggtggaagagttgccgagtttgaactgtgaaaagagatttgtgaagaatgtgtgctaagcaagcatgttaggagtccatttcaaaagacggccaaatacagatctcaacaaccactagaactaattcataccgacttgtgtggacccatctctcctgggtctttcagtggaaagaggtattttgtctcgtttatagacgattttagtcgtaaaacttgggtgtatttgctggaagaaaaatccaaagtgcttgaagtattcaagagttttaagatgatgatagaaaatgcaactggaaggtgaatcaagtcaatccgttctgaccgaggtggggagtacacatcaattgcatttaaaaggtattgcgaggagcatgggataaaaaggtttttaactgcaccgtactcaccacagcaaaatggagtggctgagagaaaaaccgtatcattcttgatatggtaaggtcaatgttgaaaacgaagaaaatgcctaaggagttttgggggcaggccgtattgtgtgcagtgtatgtgcagaaccgttgtccacattccagtctaaatggaaagaccccacaggaagcatggacaggacagaagccatcagtaggccatttcagagtcttcgggagtgtggcttatgcacacatccctgatcagcaaagaaccaagttggaagacaagagcaggaaatttattttcattggatatgacgaaaagacaaagggatacaagttgttggatcccggcaccaagaaagtaatgataagccgagatgtgaaagttaatgaagaagatgaatggaactggagcacttcatccaaggtcacctcagatcccggacagacgcttactcgacaagaagagaaggaaggagaatcaccaagttggatcccggcaagaagtgagacatctgatgaagacgagatgagacaaccccgaatgaggagtttgcatgacctgtatgattcaactaatgaagcttaccttatttgtttgttgacagattctgaagaaataacatttaaagaggcaatccgagatgaaaaatggaaaaccgccatgaatgaaaaaatcaaggtcattgaacggaatgaaacttgggagttgaccgaactgccaaaaggaagtcagcccattggagttaaatgggtttataagagaaaaagaaaaaaaaaagttggggatgaaagacgaaagaaatatttaaagtttatggggagattttgttggattaaactttaataataataaattagataggtgaagaagttactcatgtgaagagtcatgtccctaggtgaagaagttactcatgtgaagagtcatgtccctaggtaaataagttactcatgtgaagagtcatatctcacttgctattacttataaatagcatctcatgtaagaggccaatgtgtggcttggcagaccattatctaaatacatcagaagtattctactagagagattgttgtgtgtttatgtgagtgcaccagtgtgtgtgaggacatgtgatatcacaaattggtttcagagcttgagaaaatggagagtgggtcaagtagtagtctgccactaccccgccttacaaaggctaattatgaaaattggagtatccatatgaaagctcttttgggagcccatgatagttgggaagttgtccatggtggtttcgagaaaccaacatccacagcaggatatacgatggcgcagaacaagacattgaaggagacgaggttgaaagacaaaggggtgttgtacatgttattccgggctgttgatgattctgggttttagaagatcgctggtgcaacaaattcaaaggaggcgtgggacattcttgcaaatgtctacaagggagccgatcgagttagacaggttcgacttcaaactctccgtggagagttggaaggaatgaaaatgaaggaaggagaaggggtatccggtttcatcacccgggtccagaccgtggtaaatcaactaaacattctttagctctaatgtgtatctgcggaactacgctgccaatgatgcatcatcatcagcatcaggagtctgcggtactaagcctgccaaagatgcatcatcatcagcatcaggactctcagtcctcagtgttaggccggaactcacacccttgtcatcccatcgggagatacccctttacgcatggatgcacgtatatgacacctccgagaataggcttcaaccacgacgggaccacgtatctctggtgttaagggatcatataccctttacgtgagcaaaccaactaggagttgtgctctgctcctagggaggtctatatctatctaacttgccagaagatctatccgaaaggcaaccaaacacaaagcacagaatatgcatacatattatcatgaggaaagatgtaacatccaatttaatgcatatgaatcatgtatgcaaccatcactccctcggagtgtggtgaaactcaagcagtggaaaacactcccaagaaccacgaacttaccaggcaaggccgtgctctgctattcaatccctgaactgtggtctccgccactaacaccctcgttgtcatgccctatataaaggcacacaggcatgaatcaacaatcatcctatccgtacaaggcatttaattataccaaaacacacggaaaaacagaattttctgcataaaattatgcagattttgtacccacttataaaaatcataaatccctgtaggaagctcctaaaatcaagggaatttagaggcatctatatttatctcctatctttcaaacaaaactggtttcatccaatttcataaaccacaacctctgtaacacttaaaataccagaactgttacgcactataacagtgctgacaataacatatcagttcaaccagcaattttcaaacacccgccaacaccaggatactcatccaaatgacctgagattttacaggtataaattaaacatacacatctttctatttccactgaaatcactaaatttggatcatcctagactgagataaaatttcttcaagattgccctctgcaccagtaaaatctgctaaacaccagtaaaatctgctaaaaacagagtataacagcctgttataactctgcacaataaattctaggaacctccaaaaatagcacgaaaattccagaacctacttgaatctcttaactttcaaatgacactgtttttagaccatttggataccgatagctcctgttatgcacaaaacatcagggttgaccagatcgatgttccttgatgtcatgaacatgcatgtttcatgcttggaacaccatttcaaccttgaatcaacttctacacactccaaattacaccttaaaactgtttcacccttccaatactcaacatttatcaaaccaaaccctaaccttatgatttctaagaatcaaggagaaataggaggagagtaccaaccttagagttcaattgatcaaattggagagtggaccagctctctatagcttccaattctgcccagctactccgaatgaagtgggcagctcctagctctactcttttcctcaaattgctcacaaccaaaactcaacaacaataaaggacaaggagaagagcttcctttcttgcttgatttatgtttccagccactaatcaaggagtgaagagaaatggtgcttgggtgtgaaggagagacatggacataaaggaagaagaaggagaaagtgagacaaccatggtcagccacagtttttaacatgaggaggggggtgcattcagcaatctcttattcacaccctaacctctaaacaaataccaaaatgtccattctttcattaattctcttttattaatactaagtctcttaattatgcaaatatcacccaaattacattgattataattagtgacattacattaatggatagtatcttaaagaaataatttatctttttaagtttccaactttgtgaaacctgtaccaagctacgaaaaacaacaatttttctaccaagggcaagtccttgtcgttgtttgccttttgtccagacatggcaacgaaataaaccaaggcaagtaattgtgattctcttcctgttgtccatttttatgatcaatactaattggcatagaagtttactaaaaatatgcatgtttttccttacagtaattgaattgcaccttcacttgttaggtacctaacttttattgcttttatgtacagagagtcatactacaacgttaccttaatggaatatatcttgatcagtattatccgatgcaaatggtgaaaaatcatgtaagtatagaactaattgatacaacaagttatgatctcttcgttcaaaacataattttctatagtgctattcattgctattaatattttactgtcaattggatcattataataacaaatttgatatagagaagataatgcaaccactttgtgaacaagaaaaacacaaccttagtcttgttacttgtatagatgattggaaaatttgtctcacattaaggaaaatagacataattatgttttcaacaaaatgtagtgtcatcataactaaacacatgttgcctaagtctacattgtcaaacacactctttacccatacataaaggcttgtgcttgttagaaataatagttatgccttattataacttggttgacaagtaattattttttaacaagtttcttagctagtttctcgtaataaaagttaatttcttgtgtgtctttcaaaatctaactttttttttaaatatctcacatctttcaaatatctataacatatctaccaatatagaaaaagaaactatcgataaaatgtcatattttcgttttctcttgtaaaagtagtaaaataaagttaaatcaattacctagattactttgagttgcaaaatgatttcaatatttataatcattaattcctgaaatattcggagttgtgttccaattgacacctagaaatacaataaaatggaaggtgctattttttttcttttgagtcatgattggttttaaatatgttggaatagaactaaacatgactttattatacgtattttatttcatatcacccaatactattattttttcttcttttctaatttagctactatgagatggaatcaatctctttgatccttttatcattaaaatattgacacaataaatattaatttattttattccattggatccgttcaatttttttctattaatttctttctatccactattaaagtacaaccatggtttattattgttaattctatccactatctaagtacaatcatagttcatattatttttacaagtcttgaaagtgagggactaaaatcacaaatttttaaaagcgaaatgtctcaattaaaaataggaggactaaaattgcaattaattaaaaataaaaaacaaaatgtctcaattaaaaattatgagaaaaaataatggatttaaaaaaataagaagacgaaagttacattttagataaaataaataaaagtgtagtatcttaaagaggtaattgagcatttaagtatccaaatttatgaaaacaggaccaaactacaaaaaaacatcaatttctaccgagggtaaggcattgggtgtgtttgtttttaaagttagaaaatcaattttaaaataagcattttttatcagcatttttttaagtagatataattagtttcttaaaataagaggaaagttattttttaagcataagcaatttagacaatcacactaaaaataaaaagttgtttatttttagttttttttaacaaataaatttaaacatttaatttgcctttcttccgtttgtatccaaagcgggagctaaatgaatcatataattttctttctcttccttttgtccatttttaatgctcaatactaattgacatgagattaaatataataaaggcatgcttatttttccttaatgtatttgaatttcacctctactaagtgcctaattaacctctatttcttttatgtacaggagaagcggctgcaagtatatactgacattgacctagaacgttgttttgtgaaaaggccagagaatgaaataatatcaaaggtacataatttttcccaagtcttatcaataccattattattttaatgacaaatggaacattgtaataacaaatttgatatagagatgtgatcaagaaaaacacaaaactttgtctagtgcagaaatcaaatcatcttacatttttagacagaatttctactcacaatacaaagtatatccttcaaatatgtcacatttttgtttttgatgtatctcaacataatatctaatgatatgatccataagctagggaaaatcatggtattttattttaacaaataagttgaattgcaccttaatttaacataatatctaatgatatgatcaagtacctaaccttccttgcttttatgcacaaaaacaagcatgcgagagtcaagaggagaacaaggtacatatagaactaattaattacaggttattgtgttttcttcatccaaaacaaaaaaattttgggacttatcattaacattattattttattgtcaaatggaacattataataacaattttgatgtaaaaaagctaattcaatgaccttgtctcgtgacaaaaaaagtacaatttatgtcttatgtaattatttgcaaaatcgtcttatgcactgggagaaaagacacattatatttcaatggcgcaaagtgtagtgtcatctcgtaacgaaacatttggtgtctaactccatattgttagactaactttctactcacataaaaagatgtgtctttcaaatatatttttgttttttctcggcttgaaatatctcacataactcaacataggagcaaaatgagccatgtagggaaaatcattgtcttttccttttgtccattttgatgatcaatactaattggcttgaaaatttaataaagagatgcatatgcatatttttccttaatgtaacttgattggatctttacttctcaagtacctaacatttgttgcttttttgtacagccacagaaagaactatatcagcaactgaaaaaatgcaaggtaacatatagaattaggtaataataacaagtcattaagctttagacacactttttgctcacatagagaggatatgtctttcaaatatctcacaattttatttttgaaatatctcacatctttcacatacctcaacatatctaaaattatatatatggtgtgcatgtgtgtcacatatctcaacatgggtgcaaaatgagccatatatcattttgttattttgtcccctacaatgacatttccattgcatgagatctttacatttacaagaaaataccaaaaataaagttccatagaaagaccatctattatttcatcaaaattctcactcaccaaccatatttctccattgtgattgactatcaacaggaagaaaaggaaaatcgccgttctcatcccccaaaccatgttgtaaagattgacgataatggggtgttaaaagtagataaaggaaagacgaagagtttgagcacatttttgaagttttatgggaacaggctattccaatcacattctactgatgcaaatcgtcttactacatgggctgccttatatggaatgcaatgggcccaactatctgttttgggcaatcctcttgagaagttatcccaatccagaggcattgacccaatggaattaagtatggggaaagcatgttccatcacagtatgggcatcaatatttttcctgaactttatttttgcagccatgcttaccttgtcattgcctcatcgtatacctgcatactcaatgctgggatttaatttactctattacattttagcgataatagttgcagtgtctggatatagtgtttcatggggtggttcagctaaaatctcaggcctattgtttgtgtttggaggggccacatccttaatgctacttcttagcatcataatcatgggggtgctctatgctagctgcatatggtgctcctatccaaatgatgttggaggagaagtaactcagaaacactcgcctgagtgtatcatcaagatccaggatacctgaaatgacgcacttgaatgtaagtctggaatatggtattttggatccagaaacaatttatctgttttatttctttttaggtcgtggttgcctttgaaatatcaaagtttgagtatttgatagtaattggtctttgaaatgaaatttctgaaaagcagtttctttgcaacttctttagaagtggcccaagacgctttttaaatgaaaaatcttactattgaaattctcttttgttttaaatatcacattaatacttgttattttttct

SEQ ID NO: 3 - DNA, 306 bp, Glycine argyea
  source 1..306: mol_type = genomic DNA
ctaataccatacaattatcatacgcgttgaattcaacgtttcaataacacaccttccacgcaattcttaaaataatcagcattacgtcacagttctagaatgtataatcgttttccacggtaagtgccttttaaagttgaaagttggtgtggttgacagcaatgcatatgcatttttctagtaccacgtgtaataaaatacattatcattctagaaaagccggtttgactttgaattttcggtgcagcaaaacgcggtttcatattgaatttcgccatcaacgccacgatagcattggaaaaaaat

SEQ ID NO: 4 - DNA, 1203 bp, Glycine argyea
  source 1..1203: mol_type = genomic DNA
gttttgagaaggtgtgtttggtttcagactttcacatatcatccaggtggagcaaaatttgcagtacgagagagttggattcttgggctgggaactactaccaaatcatgtatatgcctaatttctcctcggtaagttgtatatgcttacttctatgctgaactgcatcatgatcaaactccattcaactcaaacaaaccgagctaatttgttcttaatcattcttatttattatcgtttttcacatcgattttgaggaatctgaatatgccaaagtcagcagttgacacatgtatagctatattaattatattttgcagtttccatcgacaaatcttgttgcgaaaatcttgaaccaaaggcagaatatccatgaaaaggaggagcatggagatgatgagaagggcaagctggtatcattaggacaaatgaatagtttaagtgtaagtttccttcttttcccctctaaactatataagttgtaattattgcagaaatgtaatgccattatattatgctaatactaattaaactgcagagcaagcaagatgttccaatgttagacttagttagagccatgtgcactgtccatgatgagaaatcaggacaacctttagcttttgagtacacattgaacacaattatgcattttgttctggttgtgtgtagtgagttatttaactaagctaatatatggtgtgatgtatattatgtgtattccagaggaaagaggatagtagaacatcaatgaacattagttctgaagttgaaagaagaacaccagaaatcatgggaagatgtgcatcgtggtgggccacctccacattttttttgtgtttgtgttcattaaaattcagcaagtcacttctccaaccacaaggacccaaatcactatggcttgccatagtaggaatgttcagttttactgtgtttgtatctgcttcttctgttatgctagttgttggagcacagttcactcgtatgcagataactgttatgctcagtgctttcttggctatgctgttagcttaattagatagttttcatatatgtacttatgttttcttcaggcagggttgacaagttgttttaatcttgttatttacataatttcattcgcataacattaaaacaaagtttgggtatgtgatatatcaatatcaacgattaaccagacagaacatattcacggatgaacattga

SEQ ID NO: 5 - DNA, 1571 bp, Glycine argyea
  source 1..1571: mol_type = unassigned DNA
gtagaagcaagttatgtgtctcttgagcaaaaaagaaagtgagtagtttgtaaacttgctgagactgagaggaacagaagaaatggacgagatcaatgagcctgcaccaccatcggatcgtggagaagccattatagatcggatgaaaactcatttgaaaaatctaccatgttattcagttatggaagccgagaatattgcgcatttgatagaagaaataaacgtgatcagtggcgacgactctgtggtagagctgacaaacaaaattttgcaatgcatagcagaactggacacacagatttggtttaagaatatgctttcatggatactcgaggaaactatagaggaactgtatccagaactatgggttatacaagtgcctaacatggggattgtgattgctgattcagtttgggaatccaagtttgcgcctaaagtggattcgcctttccggaaatttgacactagagtcatactacaacgttaccttaatggaatatatcttgatcagtattatccgatgcaaatggtgaaaaatcatgagaagcggctgcaagtatatactgacattgacctagaacgttgttttgtgaaaaggccagagaatgaaataatatcaaagccacagaaagaactatatcagcaactgaaaaaatgcaaggaagaaaaggaaaatcgccgttctcatcccccaaaccatgttgtaaagattgacgataatggggtgttaaaagtagataaaggaaagacgaagagtttgagcacatttttgaagttttatgggaacaggctattccaatcacattctactgatgcaaatcgtcttactacatgggctgccttatatggaatgcaatgggcccaactatctgttttgggcaatcctcttgagaagttatcccaatccagaggcattgacccaatggaattaagtatggggaaagcatgttccatcacagtatgggcatcaatatttttcctgaactttatttttgcagccatgcttaccttgtcattgcctcatcgtatacctgcatactcaatgctgggatttaatttactctattacattttagcgataatagttgcagtgtctggatatagtgtttcatggggtggttcagctaaaatctcaggcctattgtttgtgtttggaggggccacatccttaatgctacttcttagcatcataatcatgggggtgctctatgctagctgcatatggtgctcctatccaaatgatgttggaggagaagtaactcagaaacactcgcctgagtgtatcatcaagatccaggatacctgaaatgacgcacttgaatgtaagtctggaatatggtattttggatccagaaacaatttatctgttttatttctttttaggtcgtggttgcctttgaaatatcaaagtttgagtatttgatagtaattggtctttgaaatgaaatttctgaaaagcagtttctttgcaacttctttagaagtggcccaagacgctttttaaatgaaaaatcttactattgaaattctcttttgttttaaatatcacattaatacttgttattttttct

SEQ ID NO: 6 - protein, 407 aa, Glycine argyea
MDEINEPAPPSDRGEAIIDRMKTHLKNLPCYSVMEAENIAHLIEEINVISGDDSVVELTNKILQCIAELDTQIWFKNMLSWILEETIEELYPELWVIQVPNMGIVIADSVWESKFAPKVDSPFRKFDTRVILQRYLNGIYLDQYYPMQMVKNHEKRLQVYTDIDLERCFVKRPENEIISKPQKELYQQLKKCKEEKENRRSHPPNHVVKIDDNGVLKVDKGKTKSLSTFLKFYGNRLFQSHSTDANRLTTWAALYGMQWAQLSVLGNPLEKLSQSRGIDPMELSMGKACSITVWASIFFLNFIFAAMLTLSLPHRIPAYSMLGFNLLYYILAIIVAVSGYSVSWGGSAKISGLLFVFGGATSLMLLLSIIIMGVLYASCIWCSYPNDVGGEVTQKHSPECIIKIQDT

SEQ ID NO: 7 - DNA, 734 bp, Glycine argyea
  source 1..734: mol_type = unassigned DNA
gttttgagaaggtgtgtttggtttcagactttcacatatcatccaggtggagcaaaatttgcagtacgagagagttggattcttgggctgggaactactaccaaatcatgtatatgcctaatttctcctcgtttccatcgacaaatcttgttgcgaaaatcttgaaccaaaggcagaatatccatgaaaaggaggagcatggagatgatgagaagggcaagctggtatcattaggacaaatgaatagtttaagtaggaaagaggatagtagaacatcaatgaacattagttctgaagttgaaagaagaacaccagaaatcatgggaagatgtgcatcgtggtgggccacctccacattttttttgtgtttgtgttcattaaaattcagcaagtcacttctccaaccacaaggacccaaatcactatggcttgccatagtaggaatgttcagttttactgtgtttgtatctgcttcttctgttatgctagttgttggagcacagttcactcgtatgcagataactgttatgctcagtgctttcttggctatgctgttagcttaattagatagttttcatatatgtacttatgttttcttcaggcagggttgacaagttgttttaatcttgttatttacataatttcattcgcataacattaaaacaaagtttgggtatgtgatatatcaatatcaacgattaaccagacagaacatattcacggatgaacattga

SEQ ID NO: 8 - protein, 151 aa, Glycine argyea
MYMPNFSSFPSTNLVAKILNQRQNIHEKEEHGDDEKGKLVSLGQMNSLSRKEDSRTSMNISSEVERRTPEIMGRCASWWATSTFFLCLCSLKFSKSLLQPQGPKSLWLAIVGMFSFTVFVSASSVMLVVGAQFTRMQITVMLSAFLAMLLA

SEQ ID NO: 9 - DNA, 68382 bp, Glycine argyea
  source 1..68382: mol_type = genomic DNA
attcaagtgcatgatttcaggtatcctggatcgtgatgatacactcaggcgagtgcttcagttagttctcctccaacatcatttagagaggagcaccatgcagcatagagcaccccgatgattatgatgctaagaagtagtagcattaagcctgtggcccctccaaacacaaacaataggcctgatattttagctgaaccaccccatgaaacactatatccagacgttgcaactattatctctaaaatgtaatagagtaaagaaaatcccagcaatgcgtatgccgatatatgaaaaggcaatgacgaggtatgcactgctgcataaataatgttcaggaaaaatagtgatgcccatactgggctggaacatgctttccccatacttaattccattgggtcaatgcttgtggattgggacaacttgtcaagaggattgcccaaagcagatagttgggtccatagcagtccatataaggcaacacataaagtacgatcagtagaatgtgatcggaatagcctgttcccataaaacttcagaaatctgttcaaactcttcttctttcctttatctactttcaacaccccattatcgtcaatctttacaacatggtttgggggatgagaatggcgatttttcttttcttcctgttgatagtcaatcacaatggagaaatatggttggtgagtgagaattttgatgaaataatatatggtctttctatggaactttatttttggtattttcatgcaaatttaaagatcttatattttttagatgatgttaaaaatttaggttaaaatatatttgtgattcttaataaatattcaaattttgttgtgtttacttcccaataaatgttttctttgtatttgatccttgatatttgaatttttttgtttctggtccctaacattaggttaagaatgttaagtgtctgttaaatgtgatttctgatgatttctaaaacaaccagtatttactttaaattttaaatgacaatttatggcatctaaaaatgactagaatttgcttcaccgaggcacaccaacaacttgccatcacgatccatttcaggggacaaaaagaacaaaatgatacatggctcattttgctcccatgttgagatatgtgacacacatgcacaccatatatatataattttagatatgttgaggcagcaacggatccaggaatattcttttatgttcgcacacttagtatttttttgtaaatttttttaaaaaaaacactttaaatcaactaaaaatggaatcaacaaattgttagtgaaactaaacagtgacaacaatcacatcggcacatactggagattatgcaaacacagttagaggaacttacattatattgatcaatcttttttatcatctcatccagatgataacgtaggctgaagtccctaaaagcctgtagaagctcgtcactgcttatgtgaccactatcatctttatcaaaataggcaaaagcagcagccaaatcctcccctccttccatcttattcaagtgcagtgtagcagcaggaagttcaccatagtctattgatccattgttgtctataccagcctgccaaagttcatacataaggcaaggcaattaatgatcacccaactcagcttacacaaaagaagacttatttgaaataacattaaaacactatgtgttacattctatgataatggaaatctacctaccgcttccataagggatttaatttcatattccatgagattagagcccacacttttcaaccccttcttgagttcctcaaaagctattgtcccactattgttttaatgtatcagggtgtgatttatatgaataccagactatatataaataacagagtataaaactttataaaattgatatgaattgatattctgaaacgaaaagactatataatatatttaatcaccattaacgggaacaaacataaatcttacaaaggataaaaatatccatcaatgcagaaagctctaacctctaatatgagagaaatggatccatagaaattgacaaataaataactagctgacatgacatctgtattcagatacccacattttcttatgtaatatactttcatcatatctcttgacctcacaatccttagatttatgatatcacaaaagcattctaagatctacaatcctatatgaaagtattggctcataggaattcagaacaaatcatgtggtaattaataataattcagaacaaatcatgtgaaaaaagcttgaatcagcttcaatggcggtgacgacgcaggcacgaacgcagaacgcagaacgcagattggaggtcgaggccgaggccaagcaagccgtgagcgtgacgacggcgcggccaacacgagggtgtcgacgcagggaggtgttgcacggtggacacgtgacgacggggtggcggcagatgactcggtgcggctcgacgtgaggctcccgtttttggacaaacagaagacaaaggcaaattagatgtttaaatttatttgtaaaaaaaaatacttaaaataaacaacttttttatttttagtgtgtttgtctaaattgattgtgcttaaaaaaccagtttcctcttattttaagaaactaattatatctacttagaaaaaatgctgataaaaaacgcttattttaaaaggattttctaactttaaaaacaaacgcacccaatgccttaccctcggtataaattggtgttttatgtagtttggtcctgttttcataaatttggatacttaaatgctcaattaattctttaagatactacacttttattttacctaaaatgtaacttttgtcttcctattttttaaaatttattatttttgtcctcataatttttaattgagacattttattttttattttaaaaaaaaattataattttagtctccctatttttgattgagacattttgcttttcacttttaaaattttgtgattttagtccctttttgtcactttcaagacttgtaaaaataatatgaaccaccatggttgtacttaaacagtggatagaatgaaattaatagaaacaaattgaacggatccaatgaaataaaataaattaatatttattgtgtcaatattttaatgataaaatgatcaaagagattgattccatctcatagtagctaaattagaaaagaagaaaaaataatagtattggatgatatgaaataaaatacgtataacaaagtataacaaagtcatgtttagttctattccgaacatatttaaaaccaatcatgactcaaaagaaagagaaaaaaatagcaccttccattttattgtatttctaggtgtcaattgggaacacaactccgaatatttcaggaattaatgattacaaatattgaaatcattttgcaactcaaagtaatctagttaattgatttaactttattttactacttttacaagagaaaatcaaaggaaaacaaaaggagggcattttcattagacaacattatcacgaacactgttagcaaaatcgcgatctggatcgtaagatcggacgattttacgatctttgcggccaacggcaatccggatcggtgctaggatcgtagccgagtgggatcgtggtgagatcgcgagtttgatcgtaggatcgtaagatcctcgatccgtcacgatcctgtttcagcactgaaattaaaagccctaaccttggattcgaaaacgacccaaatcacccagtagctgcgcgacccagcaacgccgcaacatcccgacctccccccgcgcgatttcagacgacggtggcacctcccgacctcccctcttctgcgatcccgaacgatccaaggtcgacgacccgaaggaggaggagaaggaggagatcgacgccgattttgacggaggagtggaaaacttgttcggcgatctcggcggcgagaggaaggctctgattttcagcaacgaaacgcggaggacggaggtgcttcacagaggcgccgcagagctcctctagtttcagcaataacacacgcgaccaggagaagatgaaacacgcgaccaggagcagaggagaagaggaaacatgcggggagaagagaagaggaaacacgctacaggagaagaggagaagaaaagaaaatagggttttctactaatttgttttttgtactcactttttttctttctgcagtatcatataatatttttgaatggacttttgagttttaatttacgtaatttctatttaagtctttccatacccacttttttatttctgcagtttcatagtaggatctacgatccgatctacgatccgatcttacgatcctcgatttacgatctttcatcccccctcccgatcctaagtaaaatctcgattttaacaacaatgatcacgaatcaaattatttttttatttctcttccgattttgaatgaaaaactatatttaagcattttttttttgtctacacacgaggaacaaaaaccctcggtcaattttgaggatcattgaagaataaaggaaaaatctagggaggataaaataattaaattagggttactaattataatttgaaattaaagataatagtgacgatttttcaaaattgtcaaaaattgtattttgaatttttaacggtcatgtcatcatttaataaaggaatccttacaacagggactatgaataaattttttcaaatatcaaagatcaaacacaaagaaaaaataaattaggaatcaaacacaaaatttaaatatttattagggatcgaaaatatattttaaccaaaaatttattataaaaaaattgactaatgcaactttcaaaatcttggagaacttgcaaatatttattattatttcttacgataaatgttagctaaaaaagcaaaaaaaaaatatcaaacaatcaacataataaaaaggacaagcacatggtaaacgtttaagtttgtcttggtttaatccaaggtccattttattaatgtatggagaaggatctcctatagtgaaagttaacatgagaggatcatgtgaactaatcacatccatctatatcttttaaatatgagtttataataattttttataaaataaattaaataatttcaatggtttagatctcatgcaatggaaatgtcattgtaggggacaaaagaacaaaatgatacatggctcattttgctcccatgttgagatatgtgacacacatgcacaccatatatataattttagatatgttgaggtatgtgaaagatgtgagatatttcaaaaataaaattgtgagatatttgaaagacatatcctctctatgtgagcaaaaagtgtgtctaaagctcaatgacttgttatttttaactaattctatatgtaccttgcacattctctctaccaactttgcatgttcatacccttcatccggttgccgatatagttctttgacttctttcggttgttgtagttgcagttgctgtacaaaaaagtaacaaatgttaggtacttgagaagtaaaggtccaattaagttacattaaggaaaaatatgcatatgcatctctttattaaatttccaagccagttagtattgatcatcaaaatggacaaaaagaaaagacaatgatttttcctacatggctcattttgctcccatgttgagttatgtgggatgtttcaagccgagcaaaaaataaaaatatacttgaaagacacatcttgtttatgtgagtagaaagtttgtctaacaatatgtagttagacaacaaatgtttcgttatgagatgacactacactttgcgccattgaaaatataatgtgtcttttctcttaatgtataagacgactttgcaaataatctacgtaagacaaaaattgtactttttttttcacaagacaaggccattgcattagcttttttacatcaaaattgttattataatgttccatttgacaataaaataataatgttaaatgataagtccaaaaaaaattatgttttggatgaagaaaactcaatgacttgtaattaattagttctatatgtatcttgtttttctccttttgagtctcacttgcttgtttttgtgcataaaagcaagaaaggttaggtacttgagaagtaaattaaggtgcaattcaacttatttgttcaaataaatagaatgatttgccctagcttatggatcatatagttagatattatgttgagatacgtcaaaaaaaatgtgagatatttgaaggatatactttgtattgtggatttgtgagtagatattatgtctaaaaatttaagatgatttgatttctgcactagacaaagttttgtgtatttcttggtcatatctctttatcaaatttgttattacgatgttccatttgtgattaaaataataatggtattgataagacttgggaaaaattatgttttggattaagagataacaatggttggctgttataattagttctatatgtaccttaaactttctttcatcctcttgccacttcacaaaacaacgttctacgtcaatgtcagtatatacttgcagccacttctgtacataaaagcaatagagattaattaggcacttagtagaggtgcaattcaaatacattaaggaaaaataagcatgcctttattatatttaatctcatgtcacttagtatcgagcattaaaaatggacaaaaggtagagaaagaaaattatatgattcatttagcttccgtttttggacaaacggaagacaaaggcaaattagatgtttaaatttatttgttaaaaaaacttaaaataaacaactttttatctttagtgtatttgtctaaattgcttatgcttaaaaaatcagtttcctcttattttaagaaactaattatatctacttaaaaaaatgctgataaaaaatgcttattttaaaattgattttctaactttaaaaacaaacacacccaatgccttaccctcggtagaaattggtgttttatgtagtttggtcctgttttcataaatttggatacttaaatgctcaattacttctttaagatactacacttttatttattttacctaaaatgtaatttcgtcttcctattttttaattcattatttttgtcctcataatttttaattgagacattttgttttttttttttaaattacaattttagtccctttgtcactttcaagatttgtaaaaataatatgaaccatgattgtacttaaatagtggatagaattaacaattatgaaccatgattgtacttaaatagtggatagaattaacaacaatgaaccatggttgtacttaaatagtggatagaatgaaattaatagaaaaaaattgaacggatccaatgaaataaaataaattaatatttattgtgtcaatattttaatgataaaatgatcaaagagattgatgccatctcatagtagctaaattagaaaagaagaaaaaataatagtattggatgatatgaaataaaatacttataataaagtcatgtttagttctgttccaacatatttaaaaccaatcatgactcaaaagaaaaaaatagcaccttccatttaattgtatttctaggtgtcaattgggaacacaactccgaatatttcaggaattaatgattataaatattgaaatcattttgcaactcaaagtaatctagttaattgatttaactttattttactactattacaagagaaaatgaaaatatgacatttcatcgatagtttctttttcaatattggtagatatgttatagatatttgaaagatgtgagatatttaaaagaaaaagtgagattttgaaagacacacaagaaattaacttttattacaagaaactagctaggaaacttgttaaaaaataattacttgtcaaccaagttataataaggcatagctattatttctaacaggcagaagcctttatgtatgggtaaagagtgtgtttgacaatgtagagttaggcaacacgtgtttagttatgatgacactgcattttgttgaaaacataattatgtctatttgccttaatgtgagacaaattttccaatcatctatacaagtaacaagactaaggttgtgtttttcttgtttacaaagtggttgcattatcttctctagctatatcaaatttgttattataatgatccaattgacagtaaaatattaatagcaatgaatagcactatagaaattatgatttgaacgaagagatcataacttgttgtatcaattagttctatacttacatgatttttcacctgaccaagatatatttgataaaggtaaccttgtagtatcaatccctgtacataaaagcaataaaagttaggtacctaacaagtgaaggtgcaattcaattactgtaaggaaaaacatgcgtatttttagtaaacttctatgccaattagtatttaataggaactggttcagaaaatgcagagaacgactgaagttttattcaatagtgaagaacacagattacgagcacagtgattttcgaggcaatcacaacctcccaactctccaagagagtctaaccttaccttctctccccttcttatacattcacgcccccctctcagttagttataactattcctccaacattgttactgtaatgttagtggcattaaaatacaaaggtggtatgtttggtggatagcactgacatggagcatttggttccaccgcaacaggatcgtcttccacaatgaaaacttcaatggatctaagttgatggaggatgctgtatttttttgttggtcatggctagttaatctggagaaaggatttaatatacctttctaccattggtctagtcagttgagtgttgggttctgctagatagttttttatcttgttttttaacttccttatgagtctaaccttaggctgttctttggcatcttaatgcgggtcgttatcatgtattaggctgccaaagttctggttggctccctgggatggtttctttttcagtattacttgtactcagctctatatatatatatatatatatattatctttgctgatcaaaaaaaaaaaattatcttttagttatactaactgcctaactattaaaatgctatctgaaccctctgtgatatgtttcaccccacctagcactcctatcaataccctcctcttcaagctggaccttgtccccaaggtcctcttcagaaaaagcagtcttcaactcttccagcagctcccagctgttctcatgatgtggcagtccttgccatttgatcttgacagaaaattaatgatgaattaaaagttatcaaaaaaaattgagggactaaaatcttaattaaccctttagattaatatttattacaaataattctgaaattaaaaaaaaaaaaattgaatccctgataagggccaagaaaaatttgaatggagccaataataattctcaaaagtgtgacatttacttgtcattttaagatatcccttcacatctcaaaatatttcacaaattaaaggttacagctccagttcaaagtaatttacatagcataattgcaaatttgaatgcaagaaggaaaaaattattgctttagaagtaaaattacttttgagttcttccaactgaaatctaaatatgcagtaaactacttaatgaggatcgtgatagcaattaagagtttatgagagttataacttacagcgtaaaatttcgggaaagacgaattcacattaggcgcaaacttggattcccaaactgaatcagcaatcacaatccccatgccatgcactgcacacaaatcaaagaaagaatatgcatacatgttagaaattatgatggcgtattaacacaaactgctgctgagttaatttaccatgtacaacccagagttctggatacagttccgttatagttttctcgagtgcccgtaaaagataaggctccaaagtccttgtttccgtttctgatatggattgcaaaattttgcgtatcagctcttccagagagtcgtctccactcacttctatttcttctataaaatgaaacttcttttcggcttccccaactggaaagtaccgtagtagatttttcaaatgaaatttcatccgatctataatggcttctctacgatcggatggaggtgcctgttcattgatctcgttcatttcttctgttcctctcagcctcagctagtttacaaactaatcaatttcggttttgctcaagagttgcttctactgccagaggcggcagaagtagggtggaaaaccctaatttgcgggtgaagttacctatatacccttcattttttatatttttcccaaagtaccctaagacttggtcagaaaatatcagcccatttttttgttagtgcctctgttctcctcgctgttctctcatttcacgctttccccctggccctttctctcgtttcgctgcagcgccgcctgtgcctgttgcctgctgcccacggcgctccgcgcctccgcctgctgcctgcgcctccgcggatctccagcttccaggttcacttaatcacttttatttttcaattttccccaatccaatttacaatcactcaacgataacaaaaataaaaaagtagggataaaaaggaactgcaatgtgctttgtttgaagctatagaacatgtgaacaaattttcattgctttatacatttttaaagcttttaattagtagtttggttggtacttggtaataactagaatatcacattttagtttctatttttcttataaaaaactgacacatacactgatacagtgatacacatgaaatctcatttctttagaaacatattatgagttatgaattaatattcaatgtaagatggataccttaaaattaaacaaatgccacaaaactaaagtgtgacaaaactatgaataaaatgcagtacttttttttttggtttcccacaagtatcatcctttaaaagatgtatttgaaagcattgataataacgtaattatgaaggaattccaaaaaataaggcatagaatgatgatattgtaattatttttctggatatgtatttgaaattttgagtgcccccttgatttattttctggatctgtccctgcctaacaccatacaagtttgagaaggtgatgagaaatcaggacaaccttttagcttttgagtacacattgaacacaattatgcattttgttctggttgcgtgtgtaagtgagttatttaacttatgcaatgatatatggtgtgatgtatattatgtgtattccaggggaaagaggatagtagaacatcaatgaacattagttctgaagttgaaagaagaacaccagaaatcatgggaagatgtgcatcgtggtgggccaccttcacattttttttgtgtttgtgttcattaaaattgagcaagccacttctccaaccacaaggacccaattcactatgccttgcgatagtaggaatgttcagttttaccgtgtttgtatctgcttcttctgttatgcttgttgttggagcacagttcactcgtatgcagataactgttatgctcagtgctttcttggctatgctgttagtttaattagacagttttcatatatgtagttatgtttccttcaggcagggttgaacacaagttgttgtaatcttgttatttacataatttcattcgcataacaataaaacaaagtttgggtatgtgatatatcaatatcaacgattaaccagacagaacatattcacggatgaacattgaatgacaggaacatcccttcgtatgaacttttctttctttttcttttaagtttatttagaatcttatgaagcataagcataatacaataagatattaaaagaataacaccagaaatcacaggaagatatgcatcgtggttggcaaccccggatttttttggtgcttgtcattaaaattaggcaaaccacttctccaaccacaaggacctcattcactatggcttgccatcctaggaatgttcagtttcctttgtttgtgtctgcggtttctgttatgcttgttgttctagcacgcttcaatctgatgcaaataactgttattgtcgttgccttcttcgatctgctgttagctttatcacatgttttcatatatcccttccttaacccactagaatatagtgatggatagggaattgtataatttgcatgggattgctttcaattctataggctgcgtggacacatgagatatatgtttcttgtattttgttatttacagagtttcattagcataccaatgttttatgtatgtgatatatcacaagactcgacttgaagttgtttaaggggataagtctagtaccactcgaaccaatgacttaaacatttaatttcataaaagaacattttgtaaatagagaggagttggaagagagtttttttggggtcaaagataacatgtgtcttccaatccaccaggtcaaagactaatctagttcatgatgtgtgactatctctaatccaatgaaattttgcacaagataaaaatcaaacacgaattttcctattaaatagatcgttcatctaaacaaagtgaaatatgataccattatgtcaattcttttggatttaaagaaagttttgtttacctaaaaaaaagatgaccaaaaaaaaagttttgtttagatagttttaattttttataatttaattgtatatgttatcaatgtaaaactttttttatataaatatttaataaaaattactttatcaccatatcatattaacatataagaagatatgataaaatattaacatgatccaattttattaaaaagtagtgtgattaaaaaaaagagtttgttttttttcaaatgctaatccaaatcgcattatgtaactgtgtcactgatccaaaaaaaattgcgatagaaatcaaatatgattctttttttcattaaataaatttttctcgtaaatagaatcggacattatattaactctaaaatcagttggctttaacttcctacacccgtctaagttctaatgcacccagcagttatttgtgaaataccacaaataacctttgttaaaataataaaacataagcgttttgattttttctaatacaccagtcctttgcgcttctgcttcttctgcttacttccgcttctgcttctgaaacctgggattttggttctccgtcgcattcaaccgtgtgcgatgctgttctctcgggctgtggacggagcttgtggtggtcggcgacgaagctgttctggttttccggcgcgacgaggtaagtgactggctttgagtcgtttcgtcttcggttttgcatgcgtacggattgctaatctgtaacctctgaagtctacggattatggatccgtattaaagattggaaatccgtaataagaaatttgaaatccgtaggagccttacggattgccaatctgtatcatccttacggattgtcaatccgtaaggctcctacggattgtcaatccgtaaggctcctacggatttatgatccgtaagtcacttacggattgacgatccgtaaggcttacggattgccaatccgtatgggtatttgcttaatttttgacttttttgaaattagaagattaagtgtgatgactgaggtattttttacatgaatgaactgaactggtttaatgaattgctatgtgaaaagcatgttgtctaatggtatggacttgctagattgtgtttgatcctccacatgcctatggtttgatccacatttgctaggtaatttgaaatgtcatgagcaaaatgaactgtttaaatgaatttttatgtgaaaaccatgttgtctaatggtatggacttggtagattgttgttgatccacatggctatggtttgatccaagattgtttttgaattgttatgagtaaagtgaattgttttattcaattgttatttgaaaagcatgttgtctgatggtatggacctggtagttggtttctgatccacatgggtatgttttaatccagttgttaggtatcgttatctgaaaggtttattttgattaagatggctatcattgtaatgtaactacttgggttcaatgtcagattatggttaggactataggattacgacgggctttggcaagagatgccagtgaagaagaaccccctgtacagcgaaggcctacagcatcagcacggagacaacgcgagaggactagtgttgttgaggacgtgcctgctgaggtagtcgctgacgatgttgaccttgtggcacctgttgaggctgaggtcgagggaagcatcaatgcctctgatattgagggatttcctggtgggccccaggacacttcagtgttgactcattacgctgaacatattgcacctagggtttggaggggagaggtacgattgtatttaaatataattcaatttgagaattattttggcgttagggtgtgcctatgtaactaaccactaataaaggaacggcccgagcttaagttggcatctcatgggaggaaggttcataaatttggcaggcttgcgcctgagattgaaggcttggtcgcaggtaccggattgagcccactgatcacctgttcggtatatacaggtgaccggggtttaatctcggcatttgtggagaggtggcataaggagactagcagtttccatttgccgattggagagatgacgatcacgctggacgacgtcgcatctctatttcatctaccagtcatgggtgcgttgcagacgtacgaccttttggacgttgatgacgcgattgcgttacttgtcgagttgcttgaggttacccccgaccaggttgcagcagagacgaaatagtgccatggagcttatgttcgtctgtcatggttgcgggagttgtatgctgagaggtgtgaccagatgcagtggacggtggcagctagagcttatttgctccattcgatggcagttagagctcctcctgccgtgccagatcccgcacctgatccaccagctagtatggcagagcccacggttgacactggagatgatcagcctgacatgcctcgacatgcagtggtaaatacaattaaaacttctttctagtattcagaaatgtttaaacttttatgattataatgacctcttacacagttttgtgtaactttttgcagggcgcatgccaaaccattgctgagaggttacagaggctgatagattttcgcatggtgaccgctggcaccgatgcgttcgacattgttgatcactgtatcacattagcacgcagtgtatgcgatgatcagagagtttatgtgcggtcgagacgtcctaggagtggaccgcggcgagattgatttgttacattatttgtacttcactatataaacatatgtattaacattattatattgttgaaacatttcatttcatatatgtacatggtttgaacatttcatgttggttatatgtacatggtttgaacattatccaactgcgtcagtattttctttagtgaaataaaataatccggcagttatacaattacataaccccctgtcatataggtgaacacacatgaaaatactacataaagtcacaatgtatgccagtgttgagaccccctatcacagcccagatcaacataagttggtggcaatgttgtcaagctggtatacttctgcattcgactaatgtaactagctggccactcttttgcttgaggtcgacaatgtcttgaccataataaagcaattggtggcataggacagccgtcttgcaatgcaagctgaacaaaatgatcacaattacattgatttgttaatgacatgtgttataatcatctacacaaattaaagcattttgatattacctgtacaaaatggtttccatacacatgcccaataaaaatgacgcgatgtacagcagaatctctaggcggttgggttctaagggggaaaaaggtcatactttgttgcttagaaagacaaacaatgacaaggttataacgtgaaacaattacataacccatgtcagtgatgtccatccatttatcctgtgttacctgcaattttacaaatacatcagtatcattatgggcaatttaacaaatactaaattatggaatgcaaatgtaaacaacgaaaaaaacccttacatgtccaattgcttctaccaaaagtgacctctttaactcctcatatctttgtgaccccccgaataaggcaatatagtcttcagaccattctgcaagttcctttaacaaatggttgcgtaccacagcccacgcgtcttcaccataaccaagtaaacccgcaatggcacgatagtcacagttaccgtcagctttaacatcacatactccaataatataattcctcatgcatggcaaaaattgatctagcattgggatattacccatcggtatacgttctgaagactttgatgcagacttttgttgagatgagcaactgtcatgctgagaatataacgcatccacatattcaaaatacaatggatcccgctttgttgatctttcgtgtttggtgacatttttcttttgagcccctttagtcttgaccttttcaggaggaggacacatggaattttgttcaggatatgctatttctttgagcttagtctttaaagttatcttgccacacatgtcaagactttcgaatcgcttagaaattgcttccatctcgtcagttatcgtcacttcacagtcatcaacaccttggtcagagaaatgtaacctccgccaaaacatgtgtatggtacctagtggtatgctggaaagattatatttggctagctcacacgtacaaggcaaaccacacgtgcttctcaacacacatccacaacgagaactatcaacgcctgcataatgaactcggtcaaactcctcagcaattagatttaacgcataccttgaaaccatgtcgacgagtcttttgtacaatgtcactttaaaggaatgtcctacgacatgcgtacttgtctcaaaagaagcctttatttcattgtgttgcaaggtgatcatgttgctcatggcatcccaaacattacacagatctccaacgctattttgcagtagccgctttaaggtccaatgagcagactcaaccctgcagtgaaaggtgcacaaatattaatttggaaaacatgatgtcattcaggtattgcaaaggatttacttgtttgtagtagtgtttccaagatgcatgactttattggtccatgctgtaacaaatctttctttgtggggaatcaaccatgtgtctttaacatactgaacaaatattggccatggtgcgcaggcacactcaaacttcataagacagtcttcatatgcattttcagaagcacaatctacaagacttccccacgcttccatgacatagtcccatgcattgttttgtcccacaagcgttttacacttagccttcacattcttgtcaatgtggaattgacacaacaagttggtagtgtcaaggaaaacctgttggatgacattcatcagggctgcatctctgtctgtgacaatgactttaggaaaaacctcagtctttaaaaaaggcctctaaatcgttcaagggcccatacaacattattgatacgttcaccctccaaatatgcataagcagctgaaaatgtcatcccagtaggtggcacaccaactatgtccaacaaagggagtctatacatgttcgtcttgtatgtgctgtcaatgagaaataccaaattacatgaatttatcaacttcactgaatcagggtgtgcccagaatatgtctcgtagaacatcttcatccttcagtctatgccaatggatatatctatcgcattcaagaagcttcatcaattgttgcattcagtattgttgcctctaacagaagtacggtacgcatatcttgcattgtatacttgtttcattgttgtataactttcgggattgcgatcttttaaactcagcataatattctttggtttaactcttgactttgtcatttcagcaacaattaccttctcttccgcacttaaacgaccagcataaggatgtccagtcattgacttggccaaacgatgattatgtaacccacatactaacttcatcatccagccttcacctcctacgactggtttccctcgcaacttgaaaggacaaccacactttctcgtgctagtggtcgtccgtactaaatctttgttgagtgctctatactttccactcctttcacaacctatcaaaagaaatgacgttcttcccctctttccagttgctgtgtccgaccgcacaaccacagctacaaatccaatctcatgcaacactacgagcccattccaataactcctcacgacacttaaacacctacaaaatttgataatacatattttaacagtggttaagtattaaatttactagcttttatgaaatataacatgatacaaatacctttgaagtattgaatgcctccgaacaatcaattgaagagttattattgtcacacgcaaatggttcccctcccttgtcttcactcatattcatttcttgatacatcatatcatcatgcatcaaattatcattatccatcttaacaaacaaattaaaaaataaccatcattaaaaaactaaatgcataataattatactcacaaaaatatcctacaaaaaatattaacttcacaatatatttttaaatgaaaataggacaaatacattaattaataatacttttaacttattaaaaataattttttttaaataaactaccatacggattggcaatctgtaagaatcgtactgattggcaatccgtaaggattatacggattagcaatccgtatgatccttacggattagcaatccgaatgatccttacggattgacaatccgtatgatccttacggattgacaatccgtaaggttcgtacggattgctaatccgtaaggtccatacggattgctaatccgtaaggatcgtacggattgctaattcgtatcacctttacggattgccaattcgtacgatccttacggatcgccaatccgtacgatcattatggattgccaatcaaacctccgacgacacagaacgtacctctggcgacgccagcggccaacggaacctccaccaatgctaaccaccacttccgcactcccgacgaagcgataccaagcgccaacgaataaccacagcagttcacgaacagcgaagaagaagaatgaagaaaacgcatggccatactgcctggtgtatgttaaaaagcaacgaggggtattatcggtatttgatgaaaatgctgggtgtattagaaatcctactggtgtaggaagttaatgccaaatcagtttggtgttgtgcttcatacaccagatataattattctacaccattttttttcataataccaaaactgcccttaatgaatgaaaaaacaaataaatggtgaaggcgctttacttactgtgtttggtttgttggaataccgtttttgaggcggaagcttggttggaacggattgtggggtggtttttttgcatttggaagtcgttttgaggtgtgcattccacatttggaaggaaaagctgggctgggtgtcgccggagtgaaagcgccgcagttgcaggtgagttttggtagttttttgggccgtacggatccgagaatccgtaagaattgatggtgcatgaccttacggattgggcaatccgtataggtagtacggattacacaatccgtataggtcatacggattgtgtaatccgtactacctatacggattgcctaatccgtaaggtaaaaaaagtttaaaaaagtgtaaaaaatgaaatttaatttgcaaaaaattaaaattaaatgtaaataaggacattttaattataagaagtgttttaaaaatgttatgtgaaagaatgacaaagcatgtagggtgtgtcaggcatgtgttgtgttttggatttgtttgcccatcagtgttttgaatgtcattactgtttaatgaatacgattttggtgtgtgattttaatgttagtaaatgttattatttagagtatatgtgtttttacatggtatgttaaattcttttttgttttgttagaatggacagcgatgagtggatgtacgagagtataatggctgatgatgtttacatgaatgatgacattggactagagcctgatgacttggacgagaatgattgttttgttgattgttctgcagcgttccaaacaaatgaggtgattatgtggattttaatgtagttgaagttgttaaatgattatattttaaacattgatgagtaatgacttggctgatgatgtggatttcaaacaaatgacttggtgtaggtgtttgaatcgcgtgatgccgtattgaagtgggctcgtaccgttgcgtttgacaatggttttgtggcggttatcatgaggagtgacaccaaaactggaaaaataggaagaacgtcatttgtgttaattggttgtgaaagaagtggaaagtatagagcatggaaaaaaggtttcacaccttcaatcactagtagtagaaaatgcgagtgtccatttaagttgcgggccaaaccaattacgggaggtgaagggtggatggtgaagcgactatgtggtaatcataatcatgtattggccaagacatttttcggacacccatatgctggtcgaataactgaggaggaaaagaccattgttgctgatatgacaagggcgatggtcaaaccgaaaaatatcctcctaacattgaaggagcacaatgcagataatgtaacaagtttgaaacaaatttacaatgcaagatatgcataccggacacgtacaagaggcggtaatactgaaatgcaacagctaatgaaacttcttgaacgcgacatgtatattcattggcatcgattgaaagatgaaagtgttgtacgtgacttgttttgggcacttctaggtgcagtgaaactgagcaatgcatatcatatggtgttcttgatagacagtacctataaaacaaacaagtatagacttcctttactagacattgttggtgtaacaccaactggcatgacattttcagctgcctttgcctatttagaggttgaacgtataaataatcttgtgtgggctcttgaacggtttataggccttttttataagattgaattttcccaaaggtgattgttacagataaggatgcagcattgatgagtgctgtgaaaactgtttttccagaggccaaaaatttattgtgtcggtttcacattgacaaaaatgtgaaggccaaatgtaaaacttatgttaggcagaataatgcatgggattatattatggaagcatggggaagtctacttgattgtgctactgagaagcagtatgatgatacccttatgcaatttgaagttttttgtgcaccgtggcctacttttgttgattatgttaaggacacttggcttaatccccacaaagagaaatttgtgactgcatggactaataaagttatgcattttgggaatacaacaacaaataggtaactttgttatttcacattgttcaatctgacatttgtttatgtttattggcttgcaaaataatttatttgtttgtcatacaatacagggttgagtctgctcattggaccttaaaaatgcttttgcagaatagtgttggagacttatgcactgtttgggatgccatgaataatatgatcacctagcaacatactgagatcaaggcatctttagagaaaagcacacatgtggtaggacatgtttttaaagttactttatacaagaaacttgttggcatggtgtctaggtacgcgttaaatgagattgctgctgagtatgagcgagtccattatgcaggaaaaaatccttctcgttgtgggtgtgtaatgaggacaacgcatggtttgccttgtgcatgtgacctagcgaaatatgtacttgggagcattccgattgatactgttcatgttttttggcggagattaaacttttcagatgaatgtatggatgaatctgaagtcaccatcactaaagagtgggaagtcatatctacccggtttgaaaaacttgatttgtgtgggaaaatgacactaaagactaagcttaaagaaatcgcgtaccctgattcaaattcgatgtgtcctcctcctgataaggtaaaaactaaaggtgccccaaagaagaaaatgacaaagcacgaaaggtcaacgagaagggacccgtcttactttgagtatgtagacgagttacattccgtgcataatagttgccctagtccaagacaatccgtttctgaaactgatgttatgcagcaacacatgatggttccaatgttagatcaatttcatccatttatgcagaagtttatccagaatatcatcgatgtcaaagcggatggtaactgtggatatcgtgtcattgctgccttattaggttatggtgaagaatcttggtctttggtacgcaatcatttgctcaaagagcttacagcatggactgatggatatatagccttgttcggcgggatacatagatttgaaagcttgaagcggtcgcttttggttgatggattgtctaaggtaagacaactaaagatgtcggtttcgtaagcctttactaccaaatgcatgggatctttaatgtttttcattaactgtatatttacaggtaactaaggataaatggatggatctcacggatatgggtcatgttattgcatcaagatataacgtgattctagtctcgctttcacttaaacaaagtatgacattcttccctctacatagccaacctccgagggatctgcaagttcatcgcatactatgcatcggtcatgtttttggtaaccactttgttcaggtatgattgacattttttgccttcatttgttgttactttaattacgttcaacctacctaattttaatatgatatgaattgcaaacaggtatttttacgagacaattgtccgttgccatgcacggccttgttgtgggctactcattgtaccatggaagctgaatcgtggcctagttattatattgggcgtatgcaaatgtatacaaaatgtatgaaagtacgtacagaacacgttgacttaggagatgcttagacaatttctggttaatgaaagttctatttaaattcataaaagttcaaatattaaaatgctacaattcatatgcatccataaatgttcaactgcaaagtacatgaataatataaacaaaggtcatagtcgcctgcgctgtctctgtctaggccgtgtagtgcgcgtatacactaagccgtcacctgtcacacctcgagcaatacgcagcagttgctccaccatagagtatgcatcagtgccctctatgactatctcagagtcaagcatatgttgcaacccctcaaccatgcgttcgcaagcaacctgaaataagcacatatattattatttaaatgttactatatcattagacgggttctgcattgcatatacaaaaaaaagttaaatttactaccgctgcatgtgcatgggtatgccctgtcgaagatatagtagactctaagcctgctgcagcatcgtcacgaggctgatgcaccaggggagccacaggtactggtgtactagttgcctctgaacaaaaacgtgaacgagcagcctcatactgtgcaggagtcatgattggatgtgacagacggtaaaaccactccatatagccagcaacatgttgacccgaatgatcaaccaagtccccagtcgcaacaatgtaatcagtgaagtgcatccatctgtcatccacatccgctgcagaaggcatgtcgcacgaaggtacctctggaatgtgctgtacatatccaaactgcctcaggacccggtctggacaatacatgacactatgtggaccccaacggatggccccaaagtataaacatatcgtctcaaactctctgtgcaaacggtggtcttcatacggagtccagcaaacatcagcagaagtcaacctatctatacgcaatcgataggttgacagaggtaacgccttcgccatcttccaccggcaggcacgtggctgaacctcctgatagtcatcgctcggaacaacattccctaacatagggaaatgctcgtatatccagcactgtatatgacaataaaatataaattaacacataatcaataacataaattaacaaatataaattaacacatcatcattactaaccacctatacctgtaacagtgtaatgtagcctgccagatgcttcccatggtgcttacatgcatagttcaagttttcatacatgtgcatcagagcaccagctccccaagcgtatctggcagcctcatcgatgtcgcggaaagcttccaagaataccacacctatatgtgtggcactcttgttagcaaacagagtgcaccccaccagatgaagcaaataagccctcgcagctaatgtccactgcctggcttcacacttctgataatacaactctcgtagccacgataggcgaacgtatgccccatggcattgggtcgtctcagcagtggcttcgtcaactgtcacatgtagcaaatcaaccagcatgctaatggcctctcctacgtcgataacctcaaaggactgcagcgctcccataaccggaagatgcaagatcgaagccacgtcatccagcgtcactgtcagctcccccataggcaggtggaaactactggtttcagggtgccatctctcaacaaaagcagatagcaatcccctgtccccagtgtccacacagcaagaaacaagaggagccagccccgatgcagccactatgccctggatctctgcagctggcttcccaaacttaaccactttccgtccgtgagaagccaacttcagctcaggacgctcctgcagtaaagactatatcagttttcatgtatgttatcaataattgaatatgcaaaaccgatgaaaattataaaaatgtacctctccggcccagactctaacggcaatatgatctgcataagatgtcaacaacgaacaatcctctagtccccctggaaatgaatcaatctgatcatcaacatcctgtgcctcagcatcaacatgctgtgcctcagcatcaacatcctgtgtccgatcatcctgtgtcggagggatatccgtatccacctgaggctcctcaataacgggatcctcgacagcaacctcctctaacctttgcctacggacggatgccgtaggcctacgcctctgcccaggatcatcttcactagctcgactaactccacccactttctgaggcattcctctagtcctagccatgatctctgcgtatcatttgtacacatgtcagccatcaatccataaccatcacatgtcattcatgactagaaaccaaaccaaaccaagattggtacacagccaccagatgtcattcatcaattcaaaccacacaaaaccaaacaaacatcaatccataactattacatatcattcatcacatactacataaccactacatctaatacttcacttcataaccactacataaccactacatcaatccaacattactacataaccactacatctaatacttgacttcaacccagaccaagccaaaccaacaataacacttaaatatgcattccagctcatactacacaagcatgaccatatcttaacttactgaaaaagcaaaattatattcaaatatgtccaaaaccaaagcaacaataacccttaaacattgcaagtcatgttacactaaaaaattcacctaaatccaacaaaaaattcaaaaaaaagcatccatggattgaccaatccgtaaacaacctacggatcacataatccgcagtaatccgtaagcttcgtacggattatgtgatccgtatgctgtttacggattgtctgatccaaagcacctttacggattgtctgatccagagcacctttacggattgtctgatccgagagcattgtacggattatctaatccgtatacatattacggattcagtaatccgtaatatgcatacggattacataatccgtacaatgctcccggatcagacaatccgtgcggcctttctccacggattcccgattccgtttccgccatagccggcctgcaacagcctccacacaccaccatacccaagtttcaaccaataaatcactcaccggaagcaggaacgaccaatgcggtgcttgtcggaatacaatgcctcactgaacaataaccctcgccgccgattaaaaacaaaggagcctgcggtgcttgtcggaaaagctcactgaaccaatgttgctcggatagtggtgcaggaaacagggaagagggaagtaaacaacgaattggtgcagcaaatggtgtaggaagtatggcggagacagcgcagagaaggaggaggacgaagtgggaagaagagggaaggacaaatttttaaattttttttatataattaaattttaatttaagggtataaatggaaattaactgtataaatatggtgtataagattttattatggtgtaggaagctgtagccatcagtttgtccttaaagaaaaaaaaataggataaccaaaaaggctaggaaatgaaagcccaatgcttctgtaacaaaccatttcgtgtttttgtatcgtctctggcattctacatccactctgtaacacaccacacaacaatggcttccagttccaaatcggagacttttctgcagcgatacggttactattttctgttgggatcaatcgctgctgtctacgtactcactgtgccttacactaaggtcgaagtgagtttcaacgttcaggtctctctcttcatcttcttcgacctctttgcttatttttattagttatttatttatttataaattataattcctttgtgtttcctaaaggcaatgcacgatattctctatcacaggctgaacttagacaatgtgagttcactgtgtctttgctattttttttattagttggtagtttattgtattttgtatataattgatctttttttttaatgtttctgttaatagtatgatcatctggaattccctggggtggttcctcggactttcctcggtatgtttttgtgttttgtagccattttctctcaaaatatgtatttttatttaagcttattatgtttttcatatctttaatatatctctttttcagtttactacctaaaattatttttttatgattttactatatgacatatccaaacttttgcttttgacacctactattagtcttggtctgttaagtgatgatgtgacaagctaacacactagcaaatcatcagttaagtgatgatctgataagttaacagagtgccacatcatcacttaacaaacttagagtaatggtaagtagtaagtaccaaccaaaagtaaaagtttaaaaatgtcagatagtaaaataaataaataaataaaaaattaggtagtaaactaaaaaaggagtatattacaagtatgaaaaacatatttaagccttttatttattcatttagtttttggttggttaatgttttcatatttagcactagtcagacctttgtgatttattatggaaaatgataatttactttgtgaagaattaagcaaataaggattacggactcgtttatacagttgcattgcttgtgtgcctgatgtgaatgaatccatttaataacctttgagattgtgatttgttactagcatgtgttacaacggttattcaagtttgtttagtttttagtgctgaaaatttaaccgtgatgatgttctattggattgttaagattgagtttttgcaattgccatgcctcctcggtataagctggtgtgtgctagtgtagtacttctctctttgttatattgttatatgttatctaattgtgttattgttattttgttttaggtgctttgcttgtgtcaataattgcatcaccgtttgtgttaactgcaagtttgctgcatttgccaaagttttatgctcttctcataggtatgtatccttctttcatacttgacaatcaatgcatattttagttttctcatttcagggaatgctgtttttatcttttttccacctctggatttctcctttcctttggtttaaattgaaaaaaagactggcgctaatgatcaaacgtgtgtatgtttttttgttgggaggagcccgacatacatatgccaagaagtatctagttattacttttttgtttggatgcctgggaatttgtgcagataagaaaccaatcacactagaagtttaaagtctaaaacactgtctttagctggaaaataatagctcgaaaattaattctgataacgtacttgtttcactgagccattggtctctatgttgttttgcatcattctgttagtctcacaagcattcattacaggaaaatgtctgtgttatatgtacataattgaattgactgatcaaatcaaatcaaataatctacaattcatagttgagtttaaagtcatgaaccatgtcaaaatgctcaaaattaccaagatgatggtgtcaatttttgttctcctgtgctctctgttacttgagaatgtggtgggaagtacattaacatgtcttattgttgatgtctagttcggatggccctagggggcataatactatacacactaagattctttcgacatcaggtatgtacctttggatcatcaataagcaagtaaatttatttatttattttacttatttatgaagtagtgtttattcaactctctgatattatacctatccaattatagctcaagtttcaatacttctatcatgcacccttgcatatttggtgcatggatgcacaaagatgtttaactgttgatcatataaaaataataattcaacagtagagataatttatctcaatttatttggcatgtagtactataatttttaaaattatgaaaatgttctctaaagactaaagcttggccagagcttcagtgagcttcatgactatgaaaaggagattaatcaaacaaaggagtaaattggaaaatttagtgaggaactcaaatgtggttgaaccttctcaagcaatggcaaaaatcttggttatgtaggttgcatatcttttatcagtgtttgttcaagctgtcctaattcatctctataaaaacttaactaggtgtcccaatggaatggttcatgtgcctccttcaccttgtcgtgcctgccagcattggaaaatctttgagcattgattaaaactttggaaaaggataaaactggtaaaatattgagagagaaaaaaatctgaagggtgcatggcaccaaatttgagagcattgtagaattgttccaagtttcataaggaagcaacaaactttcttttcttatcattctaataatgatctttgctttgcagataatgaataagtttggacatcaagtagaatccttctttgtgatattaactgccactcagtttcattttctgttctattgtacccgtccacttcccaacattcttgctctgggcctaggtaaactacctagcttgttttgcttcttaatattcctgatagtgtttttcttgtttttaatgatgaatggcagtgtgaaaccttctagtcaatgttatggtgctgtactttctcttcatttattttgcagtgaatttagcatatggatactggttcgagggatgtttttatgcagctctaaaatctctggtatgtatgttgtggttttatttcaggttcatctcaatactaaatttttcttttgaatattataagctcttatgtattttttcttcacttcatataatatgcatattttgccaagccagaatttcgtgattaaatattctgtctcagaattagttttttaaactagtcttttaccgttaaattactatttttaaaaaagaaaagtccaacttctcatattatcaagtttacaacaaccaagcttttttccattaggtggggccagttacatggatactatttctttattttttttttccagaaatatttacttaatatttgattccaaacctattccattagattaacaaaacttaattctcatttgtatgtgtattgtgtgttgtgtatgtcaatgtgaactagttgcctgtgtgataacacatgtgaattccgtggaggcagtattggttacctcccaatgattattatttagtagggtctatcggcatctatgtttttcattttgttctcacagagacaaattcaatgcccttgatttgaatttctatgattggaatctcttgcacaactaaatgactcctccttttgcatatctacaaagttcagttaatagcttataatttaatgaaaagagattttgaaattaacttcttaagcatataaatatatgcaacttcttagccccaacttttttaaaggaaagggaaattagaaagaaaggggtgaaggtacaattaagatgagaatgaaatgtttgtggtccattttgaccagaatagaggtaaaaataaaatttatccgcaattttataatattttataagtgttaaaagtgaactgtacatggcatttagatgtttttgaaaacttgcaagggcaagaatatcttgtgaatatatatatatatatatatatatatatatatatatatatatatatatatatatatatgatatttttgctcctaatcaactgtgagagaaaaattcatgaactcctaaagagggttgttagagtagtgtaggttgcggtgtgatgacagtagtgcagtgtcagtggtgtgtggttagagtggaggggcaagtggtaaggggtgatcaatgatgtctctctatacattccattctcaataatgaaataaaacccacctccctgcttcactcagaatgtctttagctgaaacacgagaatactgaattatcagggcctataaccttcctgaaatattttttaaaatttgaactagatgtggggattatacaatgtgcataacattcttaattagtagctcacttaccctttatattatttatgctattatctatttaaaatcatagaatttagtgtgatttttttgggtacatattttagcattgttttattgtcaataagtttgaaatgaaatgattttatgaaagtgatttttttttttaaatatttctactagtttaatatatcatcttttcacacacacacacacacacaatttgacaggaaggaatgttttctggaattagttggcccttttaattcttaatatcgtagtatgcttgatatgctttgccttttctgaaataatctctttgcacatttggattatcagatttttgctacaactgtactcagacgtgacatgctgttacttctttgccccattgcgctgcaacttcttttggtatgttgtgcttttctgtaacttgcttttcatttttccgttttctccttaaattcttgagctactgatagagagcttgtcaattctttaatttatttgtttggaggatatctaatgagttatgatgcttccccctacttgtacctgttcttgttttcctgataaactttcttctatatatttaaaaagaaaaggaaaaaatctggctattctcattattttctgttggatgcttgaatttcttattttccaacaccactccgtgtctattaaactattgcatcctattctttatcttgcttttaataatgttatgctttaagtttgacaagtggagcaagtatttcctggtactggaatatatttattccatatcaactttggacttctgatgtcaatatcttgacaatgaaatgaagatcacgttttctgtaaaaacagataagaaattctattgaactgtcctggcaacagttctaatattgatatcatcatttcccagcataatttaaattaaatctgatttggacttttatcttatcgaatatttcaatttaaaggcttccaatttatgagttggttattattgcattcatgagaatttttgttgctttgtcactgtctcagaggctataggatgattggcaaatgtgtttacaatgttcaattgggtcaccttagaatccctatgtttgaaagccacctaaatttcattattgcattcatgaaattttttgttgctttgtcactatctcagaggctataggatgattggcaaatgtgtttgaaatgttcagttgggtcaccatagaatccctttgctggaaagccgccttaatttcattgttgcattcatgagaatttttgttgctttgccactatctcagacactataagatgattggaaaatgtgtttgaaatgttcaattgggtcacaccatagaatccctatgttggaaagctgccttaatttcagtcacccctagcccaccttaattgcggcctctttgggctgtcttaattgtagccttgtggggggggttgactagagatatgacttaaagagagctcataaagcttgagcaatcctcattttacaaaccggttttgtgggtttgagttaagccttaaacccaaattataaatttataaggtggtattagatcctatctaaaatccaatttttggccaacccacatttgtccatgctccagccagatgtgtcttgggtgttattattgcgatacttgcatttgtccatgctctagatggccaatcttgggcatgaggattgtgttggatagctgccttaattgtggcgactcatcacctgccttaattgtggtggcctggcctctttgggctaccttaattgtggcctcaaggggatttgcttagtctcacattgactaaagatatggttataaagcttggacaatcctcatcttacaagttagttttgtaaggttgagttggactctacgccaatttgtaagaccctattgactctgtaattttatttaacatttaaatatagaacttcattgcgttaactatgcttgtgttgttatcaatctttggagaacaatgtttattatatttggcatttgaagttaatacttaatagtgctgtttgatgaattttcttatcctcattttctgaccttgcaattttggttgaattattataaaattgaaatttattctgaaaattccatacttatggtaacatgatcatgctttgtgcctaataaaattaatctcttgaagggaagacaaaaaaaatttcagtgtgaggtgctctaaaacactgcactgggatggctctcttctgcataggttggtgacatgcttttttactttaattttaatctgttatttaaataaaatcaaaattttatgcatgataggatgctattgttaaaatttgattctaacattatggtcttctgattacaggtctaaccatattggttgattcaattatgtggaaaagattattgtggcctgaatttgaagtcttttggtttaactctgtactaaacaagagctctgaatggggagtatccttcaccaagtttatattgaacaactgatagctgtagaatatgaatacatgttatacatatttcatgaccagggtattttttaatttttttctggttctgtttggtttgaaaaaacatttttttttcacaaataattacaaaattctcaccttgttttcactttctttcctgtttccaaagatttgtttgtacaggaaaaaagagaacaaaaacccattttcactattttttgtataaatcattgaaaacaggaaacaaagtgaaaatagaggtattaatttgtgaaagcatataacattttaaccaaacaggcctttgtttgctaccttatatattacttaatgatgaggctaagccctagtttagcaaggagttacaagagaagatcttttgtcacgtttcttgttcactttagaggttgatgttctgaattgaatactttcagttgctgtttgtggatagggatatcattcaggatattagagttggtttaccatttacagtaggttatggtcttcatcttaagtttgctgatgatacaattctctttttagaggataatgatacctttggggatgttctttctattaaatttttggaagttatctttgatttttttaggttaacttttctaagagtgatttggctggtttaattgtggatagctatgttctttcttccattgcttctctggatgttgtgaaactttacagtgccccttcacatacttgggagttcctttgggtggtaatccttttccacttcttttggaaatcctgggattactcagatctttgttttgaagtgaatactaaattgtatctaattctgtgtgggctgactttgaaagccatttatttatgtgacccaagaaggactgttcaaagagatttgtacttcctttgtctcttggggtattttagtttgtttcaaaatatttgtcacttttagaaaatcaggaaagcattatttacttttttcaatcaataccctcatttgagtatctcacctaaatacttgaatgaattcacaaatacattctaactctttaaataagggtagttttctcaaagaaagttctcttatcatagtaatttgtgtaactaataattttcttaagtgtgcattaccttaaaagacataatatgaaaaagaggaattagtaaatttctgcaacaagaggattttggtgtatttggattagggtttgtatgtttagtggctaatacttgtattcttcattgtttgtgaattttctctctgttttgcccatggacaaggcacactaagcttattatcatgacatcatctaaaatctgcatattgtctgttgtctttgataacactgtcctatttgtatttgataaattattatgttccttgacaatatttttgttagacacatgctttccattggtacttcacctcagctcttcctcgttcactactttgctgcatatccactgtcactagtaagtcttggattagcaaatatcctctaacgattgcattgttacttatgtagatttctagcctgacatggtggtttcttccacttccagtgatctggttaatattttgtaacttaaatatgaaatattttgggttttctccttaaaattgaactgtctgaaggtagtacttaaagattaatattttccctatgattaatgtttgaactatgaattaatattgaatatttcttacctgttagctttggaaagtatttgttccctatttgttttcagccagttcaaatatcttaaatgcatacacttagtcatgtatattcttgcaaacccagtttaaaagataataggatatttggattgtggatagtcatggtatattgcactgaagaccaggctagttgcatgattagtgtagctgttgccctttaagaatttatcataactattttttagagaaggaaagaaagaaattattttagattaggagaattatatacacatgacaattaaaattgtagtgttatttcattcacattattggtttaacatttcattcctgttattgggatctcatgtgtaaaggattttttttaattatttaacaattagcatactgatgcacaatgaagcagcattaaggattgttttgttatttacaattttttatgttattctcataaagttttgaatttccttttatttgagagatgtcttactattttagggattttattcttcgttatatgttcttaagaaaatattgtcatgttaaaatttcctgtttaatttaattcgattaggatttaacatttaaggaaattaatactatttaattaggatatagctaggattttatattaaactgggattttcataatctttgggttataaacatcttttgagtttcttatgttattgggacttcattataatacaatatttagattttcatttctttagagaatatttttaaactcaaccagaggagtactcttgccttagatgagttctttaggtgaattgatccacatcacatgccctacagtatacgccactttttctaccttcctcgaacggaaaaaaaataaaataaataaaggaaaagaactgggaaaagaaaaagaagaaagcctccttttcatcctaacttcagttacttgacaattgacatgctgtcttctttgcagtttggcctttttgtagacagaagggtacgatctttcacttttccagttcttgccttcattttgctttattctaagcttccccacaaggtattacaattataatctgaaatccattgcctaatatgtgtttgtcttcaaattgaaacatgtatttagtattattctgattgatattgcaggagcttcgttttatcataagttcaattccaatatttaacttgtctgcttcaattgcttctaacagaatgtaagttctcctaatgagtattgtataggtctgtcaaaactaattgggttctatttgtattttctgaaattatttcattgtgtggtcttatatactaaaatacaactgaaaatggatattctgggaaagcaagtaattgagattttaaaatatgaatgaatgtgcattcctttccttgtttctatggcatttgtatgcattctttctcatttgttattggattatgaacaacattcataacttttttaatgtttatcaacctatgttttggcttcaagaatgatgatgctgtgtaacctgtaaggaagctcattagaccaagggattaagttttgctctcaaatgacaatacttgattatgagcacctgagtggcagttgcccctgctttatttcagacaaacttgaattttgcctttaggatgttcatataaggttgttgcgtcgtctatcaaggatcatgcatattccttgtttaaattatgtaaaatctggattgagaagcatgttttcttcatcatccttagtaactcaaaaaattgaataacttgcagttgctaaaatatattggagcaagttagttcaatatacaaagcatcttcctcagtcttctgtatatatttgttcttttgaactgggcttaatttagtgctccaataatacaatatgggttgtgctctaataatacaatatggctgttctattcattttgttagtcaccaagtgaatgcaacaagacagtttgcttcattgcatatttgcatgatccaaaggtatttgacattttgatggcattcataacattaggtggtttgaggtcatttacatgaaatttcattttgcttattggtattttagttttaaattcaatatgacaacagaaaaaaaaaacctttaacctactagttttaaattcaatatgacaacagaaacaacccttaacctactgggtggtgaggcaatatagtatgaatacattattatgatcattgtcatctagtcattttgatacaggaaaaagttaactagatataaaaacaattggaataatgaatacaatatatccatctaaactagaaatctaaaatcaatcgagaataggaaagagaaagctacgaatgccgcacagctgctcaatttctccatgcatgacacaaactgttgaagttgaagctgctgtgacttgtgagttgagtgccaaaaatttgaagagtggtgattgagctgaactgtatttacaatctaaacagagatatgaaaatttcggatgtttccctctttgaagaattatatctgtttccttttaagttactaaccttcagtaatatgcatcctgaaatgtttcagttacaacaataagaaaaagatgatttggaacttgcttttcctcattttgctgggattacttttaatgaggttagttcaccagtcattttgtttcattgtgatggatatatcattctttttttgtacttactctctatcttgcagtcttgcaggcaccatcacaagtttcatggcatcatactggaactatcctagtggccatgccttaaaagaattgcatggaattggtgacatgagtcctgaaatatccatgcattggctgcaaagtattttgctagaattttcctagagagaaattgctccttgtttgtcccttaatttggtaaaatattggagttgcagtttctatttttcataatcaacagtggcaagttctgttaaaattgttaatactgttgtacccgttggatctctgatgaattactaaacatcaatttttaatatggttcagtactgagcttgatattttggtcatggtttattgttctaaaaattatttccatgcgtgctatctgagtgttcatattgtatgacattctggattaaattaaatgatatgtgactttggggtgctttattgaatgatgtgttgtatgacattctctgaatctcttacccgcattccaaaaatatatctcaattctcaaaaggatttctagaataaactacattattcttcgggcagtggtttgagcaaaaaaattagcatgtcataataaatatgccaaagctttccctttggtgtatagagctgatgagttattttttggctaatttatgcatggaccaatatgcagtactggttggtactggtccttctgattttctcaaggacaatacaaaggcagtgtttgtgatactcatacttattgtgatagccaagatgcattccgtattgtctccaatcctgttttctaggggatgaccaagcacaatgatgtagattgtcactctatcagaaaagatccattctggagacatcataacttgttttgtcaatccaaatgctcaccaatcttttgagtggaccttggacaagttgttacttgttatctttgtaaactaactagctctggtcacatatgagctaatgcactagctttgacaggttgtgttaggaaaatttattattctttgtccattgaatggcaactagaaagattgtatcttttttattattacatttatcatataaatatggagtctgtggtgtagttgaaacacatttatggacatgcttttgttctctctacatgaacaatgtgtcaattttgggaaaagataaaggattgtgcttattgttactgtggtattaaggaagatttttctttagtgtttggttgatgtaacaggatttgttttgatttgtacgatttcttttcttgcccagaattcctttcatctgaattagaagtcaaatgagcagtcattgagacctaaaacaactccaatccagaaaaccccctcatttaaattataaaaagaaggaatttcattaaaagataaaaaaaattgaccactaaagaagatagtaatgtaacgagttttttatttttatctttttgatactgccccttgtcattacttttgttgtctttgttttgattttttaattgttgcaggctttcataatgatacagatgaacggtgggtgcacattgatacattttcagccatgaatggaatatctcgcttttgtgaaagtgaatttccatggaggtatattttgcttctttcttttgcagtgtactaatgatgcatcgagttgttaattgcatgcatccaaacaaacctttctttgactaaatctcatttttaaggatcaatgaagccatgcctttctgagcctttatctatgatttagtatccacagaaagttacagttctctaatttcattgtcctacttggaatgagatcaactatatttattttcagaagttatgttttcaaaagttatcatcagaattgcttctttggctgagtaataaattgatgcaaagaaaaacttcatcgaattcttcgacttgcacattcacatttttcggttccccccttcaaataaaggatagtgtatttgtttatttgattttatcgttccacagtaaactgtaattcagtatgacataggtaccttcctgttgggaaggcaggtagtgtaggccttgataatgttgcttaagtacgtaatctgctttctcaagtccagcttttcttagtagagtgacaaggagctggagctatttatagggttagtgaatgtgtttcttattgcactaatgaaaaaacaataaaccttgatctattttcctgatgctcaccatattcaggttcattagttagcaccttatctctagttgaactgagccatcattgtggttatagggtttacatttttaaacatctacttgtaagtgtcttctataccattcgttgctgtagcaagttcaatactggatttgctacatggtctgtaatgtctacatggaaaaattgtggaaaaactacatatacaaatgattaaataagaatgagatagcatgttatggggaagatttaagtgttaacgaggtattagcagtatacttctcattatttctgcattcattgctttcaatttccaacgatcagactatgctgctttccactattttccgactacaagtaacttatatactattctctggtgcaggtattctaaagaagaacaaattagcttgcaagaatttcagcagagaaattttacttttcttataaagtgagttatccattaacatattgctagtgttatttctagtactttgtctgacaaacatataaattgtatagcacatgctaatgtttatgatgagagatcctacaaactgttgacagaaagtgtcggtattgtcaagtgagtaagtgtgtgaaataaaattgataaacttcatacattagatccgcccgcgaagagtatatgttgttggcaaggactgcatcagcatgtgttgtgtgcataatgatcattgccatgaaccaactatcctaaaaactttagttttaagtgaagacaaatgaatagttttatattacatctcaactacacccattcatgcaaaatcccttatggcttgaagcgtggatagtgtgcaagcccaccaaccttgagctgaaatttaacttcttattacaagaataggtgcagcacagattgaaatctagaacaataagtcatagaatctttgttatcatgtaatggacctactatcccaaaagcttgagctattaaagtgaagacgcatgatgattttatattacatctctaactgcgaacaatttggcatttggatgaataaatgttgtctgacgtctgtcatctttctgtgcttgcatgtgtagaatgtgtgtgtgagagaggaaatgaatgtttggagatattgaattggaagtttatatatgatttttcacatgtcaactcactgactaattgcaagaaatttccccctttatctgacctccttgttgatctttaacagtgaacatcctgcgatcaatggattcaagtgcctatttactgaagatgggttttcaagagtccgcctgaaacctggctatccaccaattttactggttggtaattgctattagtattcccaaccagctcaaacaatgaatgctttgttcattccaaaggtattgatctctgttttgaccctttgcaggttaaagagcccaaagtgtatgttcatggaaatttagaaaaccagaatattttcagccaaaattggcctggctgcccataatgctgtctggtatgatagtctatttgaaatcatcaggtttgaagggtgtgggtatcctttgccacccttttttcttaattaccaatgaggaaggagggggagcaagtgaaagttggtattgtgacccctactaacagttctgtgttgtcaatattaaagagaatgtacatatgggtacaatagtttttcttgtagcatttcaaatattgattctcggatgtagctatatctgatttttgaattacatatagaagattcggttcaacaatctggtttgggataacttcatttatgctggaaaactttttggctcaattctggtaattggtgtatactgtttgctagagtagaactttctgtattaactagcatgatactcgtgtttttgcacgttttatagacaaataaataatataatatatattcaaattattaaggtcattattaataaggaatcaatgtttaagtggtgagtatattatttacaaaagtctaaaatataattttttgtagtcagatataacaataagataaaaaacctttgggtaggcaaataaataatataatatatattcaaattattaaggtcattattaatacggaatcaatgtttaagtggtgagtatattatttacaaaagtctaaaatataattttttgtagtaagatataacaataagacaaaaacctttgggtaggaaaaaatacaatgtgaatttgtatattagaaaaactttttttagtgcaatattaaaaaaacaagataccatgcaagataagagtagtcatatatggagtttaaatacagtatagagcaggggtgtggatggacatgtcttttgtctccattaggggttgggtatacccaccatgtccccgtcctcagagtgtttcttcatacaccattagtaatttctaatacaccattatttttaaaaaaataccaaaactgcccttgctggttgtggacgggtatatatctgagctgagcagcatttgatcaccatttcatacatctctttcataagcattggtgattttccgttagtcccagttgcagttgatacgcattggtgattttggtgccggagtcgtggttgtcggagctgttggtggttggcggagccgcgaagacgcggctgaactgtgcaaggtaagtggaggcagagtggatcgtacggattcggtaatccgtatggatgatacggattaaccaatccgtataacccatacggattacttaatccgtacaatgtgtacggattacttaatccgtacaatgtgtacggattacttaatccgtacaatgtgtacggattaagtaatccgtatgggttatacggattggttaatccgtatcatccatacggattaccgaatccgtacgcgttgtacggattacataatccgtatgtgtggtacggattaaccaatccgtatggattggtttgctgaattttactgttttctaaatgttggaaatatgtgtttgtataaaattgagtttagtttgataaatttgttgtattgaatgaatatattgatttaattgttgtgtaaacataacaataaataaaataaaacaatatgaatgcaaataaacataacttagcagaatgtttcatgctgaatgagtttgaaatcttattggatttgttaggatggacaacgatgaatggatttatgacagtataatgtcagaagaagttaatatggatgaagacattggagcagacgctgatgcgtttgaattcaatgaaaacaacggtgtggaccccgatgcgattgaggataatggttgtttaattgattgttctgcaactttccaaaccaatgaggtaattatgtgtatgttaatgtatttaaagttgttatatcattgtatttgggatattaaatatgaattatccatttgtgtaggtgtttgcatcacgtgaggcggtattgaaatgggctcgtaccactgcgtttgaaaatggttttgtagcggttataatgaggagcgacaccaaaactggaaaaataggaagaacgtcctttgtattaattggttgtgaaaggagtggaaaatatagagcatggaaaaaaggtttcacaccgtcaaacaccagcactagaaaatgtgagtgtccttttaagttgcggggtaaaccgatccctggaggacaggggtggatggtgaaggtaatttgtggcaatcataatcatgaattggcgaagtcattttgtggacacccatatgctggtcgaataactgaggaggagaagaccattgttggtgatatgacaaggtcgatggtgaaaccaagaaatattttgttaactttgaaggggcacaatgccgacaatgtaacaagtttgaagcaagtttataatgcaagatatgcataccgaacgcgtacaagaggcgataattctgaaatgcaacagctaatgaaactcctagaacgcgacatgtatattcattggcatcgagtaaaggatgacaatgttgtacgagatttgttttgggcacatccagatgcagtacagttgactaatgcatatcatatggtgttcctcattgatagtacctacaaaacaaacaggtacagacttcctttacttgacattgttggtgtcactcctactgggctgacattttcagctgcatttgcctatttagaggctgaacgcatcaacaatgttgtctgggcccttgaacggtttagagggatttttatgaaaagtgaattcttcccaaaggtgattgttaccgatagagattcagcgttgatgaatgctgtgaaatgtgtttttccggattcaaccaatttattgtgtcggtttcacatagacaaaaatgttaaggcaaaatgtaaaacttatgttaggcaaagccatgtatgggattatgttatggaagcatggggaagtcttgttgactgtgctactgaaaaacaatttggtgaatgccttatgcagtttgatgttgcatgtgcaccgtggcctacatttgttgattacgtgaaggacacttggttgaatcttcacaaagaaaaatttgtgaaagcatggacaaacaaagtaatgcattttggaaatacgacaacaaacaggtacatctgttattagacattaattacattgacatttccattagttaattgtattttaattaattcattatatgacctatatgcagggtggagtctgcacattccactttaaagcgcctgttgcagaatagcattggagacttatgcactgtttgagatgccatgaacaacatgatcatcttacaacataatgaaataagggcatctttggagaaaagcacgcatgttgttgggcatgcttttaaagttactttatacaagcaacttgttggcactgtatcgaggtatgctttaaatcaaattgctgatgagtatgaccgagtgcattatgcaggaaaaaacccttctcggtgtggatgtgtgctgagaacaacgaatggtttgccttgtgcttgtgaacttgctaagtatactcttcagagcattccaattaacgccgttcatatattttggcggagattaaactttgcaagtgaaggtatgcatgattctgaagtcacgataactgaagagattcaagccatttcaaaccgttttcaaaagcttgatttggttgggaaactgacagttaaaagtaaacttaaagaaattgcgtaccctgatttgaattccatgtgccctccccctgataaggtgaaaactaaaggtgctccgaagaagaagatgacaaagcacagaaggtccactagaagggacccatcatactttgagtatgtagacgaatttcattcagtgaagaatagttgctcaacgccacaacagtccaattcgtcatgtgatgttcagatgcaacacgggatagttccaatgttagatcaatttcatccatgtatgcggcagttcatcttgaacgtgacggatgtcaaagcagatggtaattgcggataccgtgtaattgctgccttattaggttatggtgaagaatcttggtcattagtacgcagtcatttgctccgagaacttggagactggacagatggatatatagccttgtttggtggtatacatagatttgaaacgttgaagagatccctactggttgacggattatccggggtaaaccttttttttatttaatgattatggctttaataacaaagtaattactactcattgtgtctaacagaatatgtgtttatgtacaggttactatggataagtggatggatattacggacatgggtcatgtgattgcatcaagatataacgtcattgtggtgtccctttcacttaaacaaagtatgacattttttcctctacgaagccaaccccctagagacccccacgttcatcgcattttgtgtattggtcatgtctttggtaaccactttgtgcaggtatcatatttatttgtttatttaatgataatacaataatctgaaaatgtagactgattgtatgatgttattatttgtaccaggttttcttaaaggacaactgccctttgccgttgacagcatttttgtgggatagacactgtaccaacgaagcaagacattggccctatcattacattagtagaatgcaaatgtatgttagatgtatgaattttaccactgaacatgtagacttatgcgatgtgtagaaacgtctaaaatgcaataaataaagtgttctggaaagcataactgtatgtacatatatttataagtatccaggtctgtacaatatgaaaaatacgacgcaaaagtaatactaaataagtgttatcaagaaagcatgactagtagtctactgaaggcgcctccgctgtctatctcgtgctcgtgtgcctcggcggtagactaggccgtggtccatcacactacgagctaggagcaacatgtcttgcaccctagagtatgcttcggtgccctctgtgaccatcccgtcatctatgtaagcctgcaaacccccggccaatcgttggcaaataacttgcaattacaatggcttgaacaataaattacccaacgtaatacaatcatatatcaaaatgcgtaaataaatacatatatttttaccaatgcatgtactggatcagaaggaggagctggatcagatacgtgtggtgaagggggatcaacatctgctgaagggggaggtgtagggtcagaaggaggacctggaggagaacgaggcgcgtatggggcctgtgaccgggcctgtgcatactgtgtcggagtgagaaatggatgtgagatccgatagaaccactctaaatatccagcaacacagtgacttggataatgtaccaagtcaccggtcgatataaggtaatctgtgaagtgggcccatcggtcgtcaacatcgtctgcagataggactcccgacaacggcaagtttggaatatgctgaatctgtccaaactgcctcactacccgatccggcaagtacctaaccacgtgcggaccccatctgatgaaacctctgaataaactgatcggctcaaaatctctaaagggtcggtgctccgcatatggtgtccaacatatgtcataaggtctcagcctctctatgcggacacggtacgtcatcaacggcaacgccttcgcacccttccagcgacaggcacgcggctgcgtctcatcgtatgtagccatagcacccccatccaccacagtgggaaaatactcatatatccagcactgcaagtagaaatgattaatctctgaaataattcaaaacaaaacaaacccacacacacagaaagaggcacaacattacctgtaatagtgaaacatatccagctagatgctttccatggtgttttgatgcataactaagattctcatacatatgcaccaaaacaccagctccccatgcaaatcgtccagcctgatcgatgtcccggaaaggctctaagaactggacgcctatatgtgtcgcactcttgttggcaaacaaagtgcagccaacaagatgcaaaagataagcccgggctgccagcagccactgcctagcgccacacagcctcaaatactggtctcgaagccaggacaagcgaacatatgccccgtggcactgtgtcgtctcggcttcagcctcatctacggtgacgttcagcagctcaaccagcaatgtaatggcctctgcaacatctatctggtcatagtcatggactactcccgtgatagggatgtgcaatagagaggcgacgtcatcgagtgtaacagtcaactcgccgactgggagatgaaaactcgaagtctcagcatgccatctctcgacaaatgctgagagcagtccccgatctccagtctccatcgagcaccgtatcagcgggctcaggccagtagcagccactaacccctcaatctcaggagctggcttcccaaatttaataaccttacgcccatgggaagccaacttcagctctgcacgttcctgcaataattaaatgaacatatgtttacaatggctaagaagcacaaatatcattttgtcataaagtgcttgtcttatatttacctgtcctgaccatactctaacagccacatggtcggcataagcagtcaacactgaacagtttgctggacctcctggaaaaccatcagcctccatatcatctgtcaccggtacatcctgcaccagaggatcctgaaccggagcatcctcaaccggaggatcctgaatcagaggatcctgaaccggagcatcctcaatcggaggatcctgaatcggaggatcctgaaccagaggatcctcacgaagactaacacccgccctttcacggcgaaccgatgctgtcggcctacgccgatcatgaagtccacctcgagtcctagccataatctgtacaacatttgtatatatcttacattatcgcttcagtctaaatgacatacacactatctgatttggggtttaccaagttggattcacaggcatacaaaccaataaagtccaattatatgaaaacaatttcatatcggtttcaacaagtatgattcaaagcctcaggaacatataaagacaaattctaacaaaaccatatcatttggggttcaacaagtttgcatcacaggcatagaaaccagtaaagtccaagtatatggaaacaatcccataccggattcaacaactgtgattcaaaaccataatcacctataaagtttgattcacaagtttcttgttcaaatcacatgtaaagaagcacatacaatcaataccatataatcatcaatgcatggataactaaacaatcacatacacttgaaaattattgttcctaattacttcaaaatcatgccatcatcaataattggatacctgttgtcttcaattcttttcataattttcattctactaaattcaatgaaaggaaatacatggccattaccacatacactaatcaacatgtatggctaacaaaatacaaaatcatcttcctaatattttaaaaaccatgtcatcatcaacatttgcataaccatagataaatgatattcctaattattttaaaaagcatgttttccaaaaatatagtctgatacggattacgtgatccgtaaaccccatacggattggacaatccgtacggggtttacggattcggtaatccgtataacccatacggattgtccaatccgtacggggtttacggattatcgaatccgtatcatgcccaagcccccatggacgtaacagaccaagaaaaaaccagagggaagaagaaacttacctcaacgccctcgtcaccagtcgcggaccacgcccccgcccaatgccaccaccaaagccaccaccaaagcctcaccgagaatggatgccggatgaagtgagggagacgaaacggaagaagtgaacagtgagtgagagaagggaagtggagcgggtttttttattttaaaaagctaagggcagaatgggaaatgcactgtaaatgaatggtgtaggagaaattttaatggtgtatgaagttgctctcccgtcctcactgattcataatataaattcagtccattacaataaaaaaaatattttgagcacaattagtcaaaactaaacatgcagtaggtattaaagagatttaactaagttggttaaacacgcgaatgttataaatcctatggcaccatattcaattcctgctcttcaaaaaaaaaaatgcagtaggcgttaagaccattaaattgagagttattccatatatattcacattgaagtaagatgagtctccttcgaattcattcctcacagaactaggatgagtctccttcgaattcattcctctgcgcataaccaaaccctcctttctgtctctttctcatcttccctctcccattcctcctcactctacttcaaccactcttcttcttcttcctccaccactctcacccatgctcacacgtgcttccacccccgcctctcggtgctcacgtgctccatctccaagatccacagctacggcaccgtcgactacgagcgccgccccatcgtgcgctggaacgacgtctaccgcaggatatctctccaccaaaacccccaagtggggtccgcagaggtgctcaaccaatgggagaacgaagggaggcacctcaccaagtgggagctttcaagagtcgttaaagagttgaggaagtacaagagatttcgaagagccctcgaggtcaaaattctatctttaaaaccttttttttttatcatttgggtctctttttttgtatgtttttgctttttactgatgaaatgctgatatatatgtctgtttttggataaacttttctctgtaagtactttttttaggagaagaaaataagaatgaaagattaattaacttctccaataagttaatttatttagctcatgcatagcatggttttgaattgccgttgcggtcgtgttgtgtgagccagaaaacctttacatcactggaaaatgcagctcatgcggcggtcgtgatgcgattgcagagtgacaaaataccttgacgttgcggaccgtaatttaaaaccatgtgcataggtgagaaaacacctttttggagaagctaaatgaagagagcttctacaaattaatttgtgctaagctaatatgagcttaaaaggagaagctaatttgttttttcttctcctaaaagtgcttatggagaagtttatccagacagcctagagtttttttgttcttcataaaactaattatttggtcccaggttttaaattgtggtcatggttttgttgtgattattgatattgtgagaatttgcagttgaggaaattcaaaaaaacattaacgattccagtgtggcaaaaactgcggttgtggaccgtattttaaaccttgatttggtcccatagttgacatttaggaataaataagaattagtcctgtatcagaaaaactaagggtctgtttggtagggtggaacgaaaacaagggagaaatttgaattttgtttgtagaggttttttccctttcctttttctctccaaccaaacaaatagacgaacataccattaggggttgaagtagaaataacaattaaattcttaccattaggggttgaagtagaaataacaattaaattcttagaggaaatacagggtctggtcatacgtcctgaacagcaggatagttggaggtgggagggggccccgaatggtaaatacatggtgggaagtgcgtacaagctattaaatagggtgttggatacccagggtcaggaggaggtgtacaaagatctttggaagctacacatcccttgcaaagcctcttttgcttggaggttactgagagactgtttgccaactaaaaagaatttggttcatagaaatataaatattggtgatcatagttgtcctttctgttgtaactttgttgaggatgcatcacacgtgttcttgagctgccacaagattatgccgctgtggtgggaatctatgagctggataaatgtggtaggggcatccgcggttaacccaattggtcatttccttcaacacagttactgcaactttactggcattaaagttcagaggtggtatatttggtggatagccttgacatggagcatttagtttcatctcaacatgattgttttccataatgaaagctttgatgggtctaagctgttggaggacgctttattcttttactggttctggttagcgaatctggagaaaggatttaatattcctttctaccattggtctagtcatattagagatggtttttgttagtaggggaggttttcattttggctctttttgttttctttcggccaacagggtgagcttttcctagggtgacctttggcaccttaattcgagttgttaccatgtattaggttgccaaagctctgctaggcttactgttagctttgtctttttcagtactacttgtactcctttctcatatatataatatattatctttgctgataaaaaaaaaattaggggttgaagtatgatgtttaaaaactttttttttttggcactgcaggggctggggggattctcaatttgtgttgttttggtgtcaacaaatacaaaatagttatttaaagggtgaaattattaggtgaacttatcattgaattaacatttccggttactaatttctatagttatgcattttcaaagactaaagcaacacaagtggccaccaaattgtataataacacacacatagggcatattcagtgtgaggtaaaaagtgttaatcttgggaatacaactatacatagatggtaaagattgttttagttagtatatataatcaattactgagtggtgggagggttgaatgtttgctgtttgggtatgtttcatttggctttaggaatggttaatatttaaggattttttccttttctgtgttaagaagtttgattttctttggaccctgctaatttagaatttagatggcattcttctttttaggaaaatcctgatccttgagggtgattaacaacttgattcatttctggtttttcattgtgacatgacatgagtatattctacagacttgtgtgtaaataggcagcattcttcaagttcagtagttgataaccatctttaaatacctgagttgaaatatatagcactgctatttgttcatgatagtggattttgctatacagcattgccatttgatttaagttttgcatgtgattcaagttggaatataaaataccacacaacaattgttacacaattccatgacttcctaaaaacatactaattcagatccataacctcctaaagacatagatgctatacaaacattaataatgcaggcctagtgagtctgggtatgtttcttattccttctcacataagctttccaaggtatatcagagcattagcaggaaccgaaaaatctgtgaattgaggtttccttcttccacaacagtttataaaagagaaaaagatttcccgaagaagcttttgcctaccatctatcatttgtaattttatcttactggatgcaaggaaagtccaatatctttgccacaaatttagcaaatcccctcgccccccctgctcaaccttttccctgctgtctttcccatattttctaataactgtaacagaatattcttagctagaattctattttgtcaatggcgtggaggaactgtatatgtttttacatcattggactatcattcactctgaggaaacaaagattgcttcgccccctttggcattgtcagaaaagtgtagcatgtttcaaaagatagtccagggaaaggagtaggggtgggctatatgagactacaacaagacctgattgattccatagaagctatatcagaaatgttctttctttattatttatttggggcatttggttttatgggataaatgaacatatacaataacataatctgtaacagcatttttagttgttaattctagagttgcaaacactgtcagtaaaatttgaataatctggaaaaccattttttgggccactatatttaatggcatgcttcctttggaatgtatggaattatgaattatgaaatcaattactatttctgattatgtttacttatttgcttcaaatgtctttatcaggcatgtacaattttttgaaagcaatgagaaaatgtttcaggtatatgattggatgaacaatagaccagagcggtttagagtttctgagagtgatgcagcaattcagttagatttaattgcaaaagttcgtggagtttctagtgcagaagctttctttctgagtctggaagataagttaaaagataagaagacttatggtgccctgttgaatgtatacgtgcattctagatcgaaggaaaaggcagaatctttatttgatactatgaggagtaaagggtatgtaatacatgccctaccatttaatgtaatgatgactctgtacatgaaccttaatgaatattctaaagttgatatgttgacttctgaaatgatggaaaaaaacatacggctagacatctatacatataatatctggttatcttcgtgcggatctcaaggatcggtagaaaagatggaacaagtgtttgaacaaatggaaaaggatcctaccattgtccctaactggtctacattcagcacaatggcttcgatgtacattaggatggatcagaatgaaaaagcagaaaagtgcttaagaaaagttgagggtagaattaaaggtcgagatagaataccttttcattatctcctgagtttatatggaagtgttggaaagaaagacgaagtgtatcgtgtgtggaatacttacaaatcaatttttccaagtataccaaacttgggataccatgctattatttcttctctggttaaattggatgataatgagggtgcagaaaaattatatgaggaatggatttcagtaaagtcaagttatgatcctagaattggaaaccttctcataggctggtatgttaagaaagatgacacagataaagctttgagtttctttgagcagatgtcgaatgatggctgcattccaaattcaaatacatgggagattctttctgagggccacattgcagataagaggatttctgaagccctgtcctgcttgaaagaagcatttatggttgctggtggttcagagagttggagaccaaagccctcatacttgtctgcattccttgagctttgtcaagagcaagatgacatggaaagtgctgaggttttaattggactgctgaggcagtcaaaatttagtaaaagtaaagtttatgcatctctcattggctcacctgatggtaccattgacaatggtgaattgccaagcaagattgatataactgatagaactgatgatgctgttgacagtgaaaatatggatgatgactctcaaatgttgttgaaccaactagatagtagcttttgatactttatttctttttcttttgactagtggaaaatgatatgctagaacatacaggggattttttgtcaaatatgatctcatcagagtttcatggcttctatgtcttattctcttcatcacagttagaaagtggcagctgcttctatgtcattggtgttttttttaacgatttggtgatttgattatgtgtattgaagtgtgaacgtgtgtgtgtgtcaacagtgaaaatatggatgatgactctcaaatgttgttaaaccaactagatagtagcttttgatactttatttctttttcttttgactagtggaaaatgatatgctagaacatacaggggatttttttgtcaaatatgatgtcatcagagtttcatggcttctatgtcttattctcttcatcacagttagaaagtggcagctgcttctatgtcattggttggtgttttttttaacgatttggtgatttgattatgtgtattgaagtgtgaacgtgtgtgtgtgcgtgtgtgtgtgcgtgtgcgcgtgtgtgtgtgttgtgcgtgcgcgcaccgtgcgtgcgtgcggtttaggttcaatataatgagaggaatgatggattgagcttttggagcattaactctatttgctttagcccctaacgttgcttaaagtattttattttagtcatttttcacttaattgtgcgtgcgcgcgtgcgtgcgcgtgcgtgcgtgtgtgtgcgtgcggtttaggttcaatataatgagaggaatgatggattgagcttttggagcattaactctatttgctttagcccctaacattgctgaaagtattttattttagtcatttttcacttaattctcttctactatttttgtaccaaaaatgataaatgtaaatgccatttatagataggaactaaaatatatttttttaacttttaatttttgatgaattttatttcaataaattaatttggtgcatttaaattttaacttttaaaaaaattatgaattttatcttctgtcattttactcctaatataattacacctttactcaactttttattttttgaaaaaatttatcctaatttttggtaaattttactcctaatttttttgcttggttgggataaaatttacaagtttattatgtcaaattaaaaagggaaaggttcaccgaaaattaaaaagtttggataaaaaatgcaattaagtccgagtaatatagagtttatgctatgaaaggaagattaatagaaaaaataacaagtattaatgtgatatttaaaacaaaagagaatttcaatagtaagatttttcatttaaaaagcgtcttgggccacttctaaagaagttgcaaagaaactgcttttcagaaatttcatttcaaagaccaattactatcaaatactcaaactttgatatttcaaaggcaaccacgacctaaaaagaaataaaacagataaattgtttctggatccaaaataccatattccagacttacattcaagtgcgtcatttcaggtatcctggatcttgatgatacactcaggcgagtgtttctgagttacttctcctccaacatcatttggataggagcaccatatgcagctagcatagagcacccccatgattatgatgctaagaagtagcattaaggatgtggcccctccaaacacaaacaataggcctgagattttagctgaaccaccccatgaaacactatatccagacactgcaactattatcgctaaaatgtaatagagtaaattaaatcccagcattgagtatgcaggtatacgatgaggcaatgacaaggtaagcatggctgcaaaaataaagttcaggaaaaatattgatgcccatactgtgatggaacatgctttccccatacttaattccattgggtcaatgcctctggattgggataacttctcaagaggattgcccaaaacagatagttgggcccattgcattccatataaggcagcccatgtagtaagacgatttgcatcagtagaatgtgattggaatagcctgttcccataaaacttcaaaaatgtgctcaaactcttcgtctttcctttatctacttttaacaccccattatcgtcaatctttacaacatggtttgggggatgagaacggcgattttccttttcttcctgttgatagtcaatcacaatggagaaatatggttggtgagtgagaattttgatgaaataatagatggtctttctatggaactttatttttggtattttcttgtaaatgtaaagatctcatgcaatggaaatgtcattgtaggggacaaaataacaaaatgatatatggctcattttgcacccatgttgagatatgtgacacacatgcacaccatatatataattttagatatgttgaggtatgtgaaagatgtgagatatttcaaaaataaaattgtgagatatttgaaagacatatcctctctatgtgagcaaaaagtgtgtctaaagcttaatgacttgttattattacctaattctatatgttaccttgcattttttcagttgctgatatagttctttctgtggctgtacaaaaaagcaacaaatgttaggtacttgagaagtaaagatccaatcaagttacattaaggaaaaatatgcatatgcatctctttattaaattttcaagccaattagtattgatcatcaaaatggacaaaaggaaaagacaatgattttccctacatggctcattttgctcctatgttgagttatgtgagatatttcaagccgagaaaaaacaaaaatatatttgaaagacacatctttttatgtgagtagaaagttagtctaacaatatggagttagacaccaaatgtttcgttacgagatgacactacactttgcgccattgaaatataatgtgtcttttctcccagtgcataagacgattttgcaaataattacataagacataaattgtactttttttgtcacgagacaaggtcattgaattagcttttttacatcaaaattgttattataatgttccatttgacaataaaataataatgttaatgataagtcccaaaatttttttgttttggatgaagaaaacacaataacctgtaattaattagttctatatgtaccttgttctcctcttgactctcgcatgcttgtttttgtgcataaaagcaaggaaggttaggtacttgatcatatcattagatattatgttaaattaaggtgcaattcaacttatttgttaaaataaaataccatgattttccctagcttatggatcatatcattagatattatgttgagatacatcaaaaacaaaaatgtgacatatttgaaggatatactttgtattgtgagtagaaattctgtctaaaaatgtaagatgatttgatttctgcactagacaaagttttgtgtttttcttgatcacatctctatatcaaatttgttattacaatgttccatttgtcattaaaataataatggtattgataagacttgggaaaaattatgtacctttgatattatttcattctctggccttttcacaaaacaacgttctaggtcaatgtcagtatatacttgcagccgcttctcctgtacataaaagaaatagaggttaattaggcacttagtagaggtgaaattcaaatacattaaggaaaaataagcatgcctttattatatttaatctcatgtcaattagtattgagcattaaaaatggacaaaaggaagagaaagaaaattatatgattcatttagctcccgctttggatacaaacggaagaaaggcaaattaaatgtttaaatttatttgttaaaaaaaactaaaaataaacaactttttatttttagtgtgattgtctaaattgcttatgcttaaaaaataactttcctcttattttaagaaactaattatatctacttaaaaaaatgctgataaaaaatgcttattttaaaattgattttctaactttaaaaacaaacacacccaatgccttaccctcggtagaaattgatgtttttttgtagtttggtcctgttttcataaatttggatacttaaatgctcaattacctctttaagatactacacttttatttattttatctaaaatgtaactttcgtcttcttatttttttaaatccattattttttctcataatttttaattgagacattttgttttttatttttaattaattgcaattttagtcctcctatttttaattgagacatttcgcttttaaaaatttgtgattttagtccctcactttcaagacttgtaaaaataatatgaactatgattgtacttagatagtggatagaattaacaataataaaccatggttgtactttaatagtggatagaaagaaattaatagaaaaaaattgaacggatccaatggaataaaataaattaatatttattgtgtcaatattttaatgataaaaggatcaaagagattgattccatctcatagtagctaaattagaaaagaagaaaaaataatagtattgggtgatatgaaataaaatacgtataataaagtcatgtttagttctattccaacatatttaaaaccaatcatgactcaaaagaaaaaaaatagcaccttccattttattgtatttctaggtgtcaattggaacacaactccgaatatttcaggaattaatgattataaatattgaaatcattttgcaactcaaagtaatctaggtaattgatttaactttattttactacttttacaagagaaaacgaaaatatgacattttatcgatagtttctttttctatattggtagatatgttatagatatttgaaagatgtgagatatttaaaaaaaaagttagattttgaaagacacacaagaaattaacttttattacgagaaactagctaagaaacttgttaaaaaataattacttgtcaaccaagttataataaggcataactattatttctaacaagcacaagcctttatgtatgggtaaagagtgtgtttgacaatgtagacttaggcaacatgtgtttagttatgatgacactacattttgttgaaaacataattatgtctattttccttaatgtgagacaaattttccaatcatctatacaagtaacaagactaaggttgtgtttttcttgttcacaaagtggttgcattatcttctctatatcaaatttgttattataatgatccaattgacagtaaaatattaatagcaatgaatagcactatagaaaattatgttttgaacgaagagatcataacttgttgtatcaattagttctatacttacatgatttttcaccatttgcatcggataatactgatcaagatatattccattaaggtaacgttgtagtatgactctctgtacataaaagcaataaaagttaggtacctaacaagtgaaggtgcaattcaattactgtaaggaaaaacatgcatatttttagtaaacttctatgccaattagtattgatcataaaaatggacaacaggaagagaatcacaattacttgccttggtttatttcgttgccatgtctggacaaaaggcaaacaacgacaaggacttgcccttggtagaaaaattgttgtttttcgtagcttggtacaggtttcacaaagttggaaacttaaaaagataaattatttctttaagatactatccattaatgtaatgtcactaattataatcaatgtaatttgggtgatatttgcataattaagagacttagtattaataaaagagaattaatgaaagaatggacattttggtatttgtttagaggttagggtgtgaataagagattgctgaatgcacccccctcctcatgttaaaaactgtggctgaccatggttgtctcactttctccttcttcttcctttatgtccatgtctctccttcacacccaagcaccatttctcttcactccttgattagtggctggaaacataaatcaagcaagaaaggaagctcttctccttgtcctttattgttgttgagttttggttgtgagcaatttgaggaaaagagtagagctaggagctgcccacttcattcggagtagctgggcagaattggaagctatagagagctggtccactctccaatttgatcaattgaactctaaggttggtactctcctcctatttctccttgattcttagaaatcataaggttagggtttggtttgataaatgttgagtattggaagggtgaaacagttttaaggtgtaatttggagtgtgtagaagttgattcaaggttgaaatggtgttccaagcatgaaacatgcatgttcatgacatcaaggaacatcgatctggtcaaccctgatgttttgtgcataacaggagctatcggtatccaaatggtctaaaaacagtgtcatttgaaagttaagagattcaagtaggttctggaattttcgtgctatttttggaggttcctagaatttattgtgcagagttataacaggctgttatactctgtttttagcagattttactggtgtttagcagattttactggtgcagagggcaatcttgaagaaattttatctcagtctaggatgatccaaatttagtgatttcagtggaaatagaaagatgtgtatgtttaatttatacctgtaaaatctcaggtcatttggatgagtatcctggtgttggcgggtgtttgaaaattgctggttgaactgatatgttattgtcagcactgttatagtgcgtaacagttctggtattttaagtgttacagaggttgtggtttatgaaattggatgaaaccagttttgtttgaaagataggagataaatatagatgcctctaaattcccttgattttaggagcttcctacagggatttatgatttttataagtgggtacaaaatctgcataattttatgcagaaaattctgtttttccgtgtgttttggtataattaaatgccttgtacggataggatgattgttgattcatgcctgtgtgcctttatatagggcatgacaacgagggtgttagtggcggagaccacagttcagggattgaatagcagagcacggccttgcctggtaagttcgtggttcttgggagtgttttccactgcttgagtttcaccacactccgagggagtgatggttgcatacatgattcatatgcattaaattggatgttacatctttcctcatgataatatgtatgcatattctgtgctttgtgtttggttgcctttcggatagatcttctggcaagttagatagatatagacctccctaggagcagagcacaactcctagttggtttgctcacgtaaagggtatatgatcccttaacaccagagatacgtggtcccgtcgtggttgaagcctattctcggaggtgtcatatacgtgcatccatgcgtaaaggggtatctcccgatgggatgacaagggtgtgagttccggcctaacactgaggactgagagtcctgatgctgatgatgatgcatctttggcaggcttagtaccgcagactcctgatgctgatgatgatgcatcattggcagcgtagttccgcagatacacattagagctaaagaatgtttagttgatttaccacggtctggacccgggtgatgaaaccggataccccttctccttccttcattttcattccttccaactctccacggagagtttgaagtcgaacctgtctaactcgatcggctcccttgtagacatttgcaagaatgtcccacgcctcctttgaatttgttgcaccagcgatcttctaaaacccagaatcatcaacagcccggaataacatgtacaacacccctttgtctttcaacctcgtctccttcaatgtcttgttctgcgccatcgtatatcctgctgtggatgttggtttctcgaaaccaccatggacaacttcccaactatcatgggctcccaaaagagctttcatatggatactccaattttcataattagcctttgtaaggcggggtagtggcagactactacttgacccactctccattttctcaagctctgaaaccaatttgtgatatcacatgtcctcacacacactggtgcactcacataaacacacaacaatctctctagtagaatacttctgatgtatttagataatggtctgccaagccacacattggcctcttacatgagatgctatttataagtaatagcaagtgagatatgactcttcacatgagtaacttatttacctagggacatgactcttcacatgagtaacttcttcacctagggacatgactcttcacatgagtaacttcttcacctatctaatttattattattaaagtttaatccaacaaaatctccccataaactttaaatatttctttcgtctttcatccccaacttttttttttctttttctcttataaacccatttaactccaatgggctgacttccttttggcagttcggtcaactcccaagtttcattccgttcaatgaccttgattttttcattcatggcggttttccatttttcatctcggattgcctctttaaatgttatttcttcagaatctgtcaacaaacaaataaggtaagcttcattagttgaatcatacaggtcatgcaaactcctcattcggggttgtctcatctcgtcttcatcagatgtctcacttcttgccgggatccaacttggtgattctccttccttctcttcttgtcgagtaagcgtctgtccgggatctgaggtgaccttggatgaagtgctccagttccattcatcttcttcattaactttcacatctcggcttatcattactttcttggtgccgggatccaacaacttgtatccctttgtcttttcgtcatatccaatgaaaataaatttcctgctcttgtcttccaacttggttctttgctgatcagggatgtgtgcataagccacactcccgaagactctgaaatggcctactgatggcttctgtcctgtccatgcttcctgtggggtctttccatttagactggaatgtggacaacggttctgcacatacactgcacacaatacggcctgcccccaaaactccttaggcattttcttcgttttcaacattgaccttaccatatcaagaatgatacggtttttctctcagccactccattttgctgtggtgagtacggtgcagttaaaaacctttttatcccatgctcctcgcaataccttttaaatgcaattgatgtgtactccccacctcggtcagaacggattgacttgattcaccttccagttgcattttctatcatcatcttaaaactcttgaatacttcaagcactttggatttttcttccagcaaatacacccaagttttacgactaaaatcgtctataaacgagacaaaatacctctttccactgaaagacccaggagagatgggtccacacaagtcggtatgaattagttctagtggttgttgagatctgtatttggccgtcttttgaaatggactcctaacatgcttgcttagcacacattcttcacaaatctcttttcacagttcaaactcggcaactcttccaccatattcttgttcatcatctcgaccaggcctccgtagtgaagatgcccaaatcggcgatgccataaggatacttcatcactcatctcggcttgcaaacagtcgacctgcacactcctcaacattagtttgaacattctatttttcttcatctgaacttgaactatcaaacatccttgtttatccttcaagaagagaactcggtctgtcatgagaaccgagtatccattctccatcagttgtcccacactaagaatattactttttagatcgggcacgtaatagacatcttggatgcgcccaattcgtccatctttctgcaagaaacaaatcgtacctcagcctttgacatcaacctttgaaacatctccaaacgaaacatgtatggcttctatttgtctcagattactgaacaaatgcttatgcccacacatatgattgcttgcactagtgtctagataccattccatctcgaatccctggttgacgtcacttctagtcatcatcagaattcctccatctcttacttctccttctgtaacaaggtttgaagtttcatttcttctttcatcttggcaatccttcgcaaaatgtccgactcgaccacagttgtaacatctgtccgagttgcaatctcttgagtaatgtccatactttcctcatttaaaacattctacatttgattgatttgaatgtcggcctcttccacgaccttggcctcgccaattctgccgacttaatttagcctgctccttattccttccttcttgaccgcggcctctactaccgcgaccgtctcctcgacctccatagtcatgccctctacctcgaaagttttgagaataaagtaccttttcgtccttgattgaagcttttgtctggagtgcttgctccagtgactcttctttcttctttttctttctttgttcatgcgcctccaaggaaccagcaagttcgtcaacggtgagcgttgctaggtcctttgactcctcaatcgcacatacaacattctcgaagtcgtcagtgagagacctcagaattttctccaccactttggcatcggacaacacctctccatttctgtttagttgatttaccacggtctagacccgggtgatgaaaccggataccccttctccttccttcattttcattccttccaactctccacggagagtttgaagtcgaacctgtctaactcgatcggctcccttgtagaaatttgcaagaatgtcccacgcctcctttgaagttgttgcaccagcgatcttctcgaacccggaatcatcaacagtccagaataacatgtacaacgcccccttgtctttcaacctcatctccttcaatgtcttgttctgcaccgccgtatatcctgctatggatgttggttccttgaaaccaccatggacaacttcccaactatcatgggctcccaaaagagctttcatctggatactccagttttcgtaattagcctttgtaaggcggggtagtggtagactactacttggcccactctccattttctcaagctctgataccaatttgtgatatcacaggtcctcacacacactggtgcactcacacaaacacacaacaatctctctggtagaatacttctgatatatttagataatggtctgccaagccacacattgacctcttacatgagatgttatttataagtaatagcaagtgggacatgactcttcacatgagtaacttctttacctagggacatgactcttcacatgagtaacttcttcacctatctaatttattattattaaagtttaatccaacataaattacttaatcaggatcgtgatggcaattaagagttgatgagagttataacttacagtgtcaaatttccggaaaggcgaatccactttaggcgcaaacttggattcccaaactgaatcagcaatcacaatccccatgttaggcactgcacacaaatcaaagaaagaatatgcatacatgttagaaattatgatggcggtataaacagaaactgctgctgagttatatcattatctactacttccacttcaatcctaatatctggatattaatttaccttgtataacccatagttctggatacagttcctctatagtttcctcgagtatccatgaaagcatattcttaaaccaaatctgtgtgtccagttctgctatgcattgcaaaattttgtttgtcagctctaccacagagtcgtcgccactgatcacgtttatttcttctatcaaatgcgcaatattctcggcttccataactgaataacatggtagatttttcaaatgagttttcatccgatctataatggcttctccacgatccgatggtggtgcaggctcattgatctcgtccatttcttctgttcctctcagtctcagcaagtttacaaactactcactttcttttttgctcaagagacacataacttgcttctacctaataccatacaattatcatacgcgttgaattcaacgtttcaataacacaccttccacgcaattcttaaaataatcagcattacgtcacagttctagaatgtataatcgttttccacggtaagtgccttttaaagttgaaagttggtgtggttgacagcaatgcatatgcatttttctagtaccacgtgtaataaaatacattatcattctagaaaagccggtttgactttgaattttcggtgcagcaaaacgcggtttcatattgaatttcgccatcaacgccacgatagcattggaaaaaaatgttttgagaaggtgtgtttggtttcagactttcacatatcatccaggtggagcaaaatttgcagtacgagagagttggattcttgggctgggaactactaccaaatcatgtatatgcctaatttctcctcggtaagttgtatatgcttacttctatgctgaactgcatcatgatcaaactccattcaactcaaacaaaccgagctaatttgttcttaatcattcttatttattatcgtttttcacatcgattttgaggaatctgaatatgccaaagtcagcagttgacacatgtatagctatattaattatattttgcagtttccatcgacaaatcttgttgcgaaaatcttgaaccaaaggcagaatatccatgaaaaggaggagcatggagatgatgagaagggcaagctggtatcattaggacaaatgaatagtttaagtgtaagtttccttcttttcccctctaaactatataagttgtaattattgcagaaatgtaatgccattatattatgctaatactaattaaactgcagagcaagcaagatgttccaatgttagacttagttagagccatgtgcactgtccatgatgagaaatcaggacaacctttagcttttgagtacacattgaacacaattatgcattttgttctggttgtgtgtagtgagttatttaactaagctaatatatggtgtgatgtatattatgtgtattccagaggaaagaggatagtagaacatcaatgaacattagttctgaagttgaaagaagaacaccagaaatcatgggaagatgtgcatcgtggtgggccacctccacattttttttgtgtttgtgttcattaaaattcagcaagtcacttctccaaccacaaggacccaaatcactatggcttgccatagtaggaatgttcagttttactgtgtttgtatctgcttcttctgttatgctagttgttggagcacagttcactcgtatgcagataactgttatgctcagtgctttcttggctatgctgttagcttaattagatagttttcatatatgtacttatgttttcttcaggcagggttgacaagttgttttaatcttgttatttacataatttcattcgcataacattaaaacaaagtttgggtatgtgatatatcaatatcaacgattaaccagacagaacatattcacggatgaacattga

SEQ ID NO: 10 - DNA, 11756 bp, Glycine argyea
  source 1..11756: mol_type = genomic DNA
attcaagtgcatgatttcaggtatcctggatcgtgatgatacactcaggcgagtgcttcagttagttctcctccaacatcatttagagaggagcaccatgcagcatagagcaccccgatgattatgatgctaagaagtagtagcattaagcctgtggcccctccaaacacaaacaataggcctgatattttagctgaaccaccccatgaaacactatatccagacgttgcaactattatctctaaaatgtaatagagtaaagaaaatcccagcaatgcgtatgccgatatatgaaaaggcaatgacgaggtatgcactgctgcataaataatgttcaggaaaaatagtgatgcccatactgggctggaacatgctttccccatacttaattccattgggtcaatgcttgtggattgggacaacttgtcaagaggattgcccaaagcagatagttgggtccatagcagtccatataaggcaacacataaagtacgatcagtagaatgtgatcggaatagcctgttcccataaaacttcagaaatctgttcaaactcttcttctttcctttatctactttcaacaccccattatcgtcaatctttacaacatggtttgggggatgagaatggcgatttttcttttcttcctgttgatagtcaatcacaatggagaaatatggttggtgagtgagaattttgatgaaataatatatggtctttctatggaactttatttttggtattttcatgcaaatttaaagatcttatattttttagatgatgttaaaaatttaggttaaaatatatttgtgattcttaataaatattcaaattttgttgtgtttacttcccaataaatgttttctttgtatttgatccttgatatttgaatttttttgtttctggtccctaacattaggttaagaatgttaagtgtctgttaaatgtgatttctgatgatttctaaaacaaccagtatttactttaaattttaaatgacaatttatggcatctaaaaatgactagaatttgcttcaccgaggcacaccaacaacttgccatcacgatccatttcaggggacaaaaagaacaaaatgatacatggctcattttgctcccatgttgagatatgtgacacacatgcacaccatatatatataattttagatatgttgaggcagcaacggatccaggaatattcttttatgttcgcacacttagtatttttttgtaaatttttttaaaaaaaacactttaaatcaactaaaaatggaatcaacaaattgttagtgaaactaaacagtgacaacaatcacatcggcacatactggagattatgcaaacacagttagaggaacttacattatattgatcaatcttttttatcatctcatccagatgataacgtaggctgaagtccctaaaagcctgtagaagctcgtcactgcttatgtgaccactatcatctttatcaaaataggcaaaagcagcagccaaatcctcccctccttccatcttattcaagtgcagtgtagcagcaggaagttcaccatagtctattgatccattgttgtctataccagcctgccaaagttcatacataaggcaaggcaattaatgatcacccaactcagcttacacaaaagaagacttatttgaaataacattaaaacactatgtgttacattctatgataatggaaatctacctaccgcttccataagggatttaatttcatattccatgagattagagcccacacttttcaaccccttcttgagttcctcaaaagctattgtcccactattgttttaatgtatcagggtgtgatttatatgaataccagactatatataaataacagagtataaaactttataaaattgatatgaattgatattctgaaacgaaaagactatataatatatttaatcaccattaacgggaacaaacataaatcttacaaaggataaaaatatccatcaatgcagaaagctctaacctctaatatgagagaaatggatccatagaaattgacaaataaataactagctgacatgacatctgtattcagatacccacattttcttatgtaatatactttcatcatatctcttgacctcacaatccttagatttatgatatcacaaaagcattctaagatctacaatcctatatgaaagtattggctcataggaattcagaacaaatcatgtggtaattaataataattcagaacaaatcatgtgaaaaaagcttgaatcagcttcaatggcggtgacgacgcaggcacgaacgcagaacgcagaacgcagattggaggtcgaggccgaggccaagcaagccgtgagcgtgacgacggcgcggccaacacgagggtgtcgacgcagggaggtgttgcacggtggacacgtgacgacggggtggcggcagatgactcggtgcggctcgacgtgaggctcccgtttttggacaaacagaagacaaaggcaaattagatgtttaaatttatttgtaaaaaaaaatacttaaaataaacaacttttttatttttagtgtgtttgtctaaattgattgtgcttaaaaaaccagtttcctcttattttaagaaactaattatatctacttagaaaaaatgctgataaaaaacgcttattttaaaaggattttctaactttaaaaacaaacgcacccaatgccttaccctcggtataaattggtgttttatgtagtttggtcctgttttcataaatttggatacttaaatgctcaattaattctttaagatactacacttttattttacctaaaatgtaacttttgtcttcctattttttaaaatttattatttttgtcctcataatttttaattgagacattttattttttattttaaaaaaaaattataattttagtctccctatttttgattgagacattttgcttttcacttttaaaattttgtgattttagtccctttttgtcactttcaagacttgtaaaaataatatgaaccaccatggttgtacttaaacagtggatagaatgaaattaatagaaacaaattgaacggatccaatgaaataaaataaattaatatttattgtgtcaatattttaatgataaaatgatcaaagagattgattccatctcatagtagctaaattagaaaagaagaaaaaataatagtattggatgatatgaaataaaatacgtataacaaagtataacaaagtcatgtttagttctattccgaacatatttaaaaccaatcatgactcaaaagaaagagaaaaaaatagcaccttccattttattgtatttctaggtgtcaattgggaacacaactccgaatatttcaggaattaatgattacaaatattgaaatcattttgcaactcaaagtaatctagttaattgatttaactttattttactacttttacaagagaaaatcaaaggaaaacaaaaggagggcattttcattagacaacattatcacgaacactgttagcaaaatcgcgatctggatcgtaagatcggacgattttacgatctttgcggccaacggcaatccggatcggtgctaggatcgtagccgagtgggatcgtggtgagatcgcgagtttgatcgtaggatcgtaagatcctcgatccgtcacgatcctgtttcagcactgaaattaaaagccctaaccttggattcgaaaacgacccaaatcacccagtagctgcgcgacccagcaacgccgcaacatcccgacctccccccgcgcgatttcagacgacggtggcacctcccgacctcccctcttctgcgatcccgaacgatccaaggtcgacgacccgaaggaggaggagaaggaggagatcgacgccgattttgacggaggagtggaaaacttgttcggcgatctcggcggcgagaggaaggctctgattttcagcaacgaaacgcggaggacggaggtgcttcacagaggcgccgcagagctcctctagtttcagcaataacacacgcgaccaggagaagatgaaacacgcgaccaggagcagaggagaagaggaaacatgcggggagaagagaagaggaaacacgctacaggagaagaggagaagaaaagaaaatagggttttctactaatttgttttttgtactcactttttttctttctgcagtatcatataatatttttgaatggacttttgagttttaatttacgtaatttctatttaagtctttccatacccacttttttatttctgcagtttcatagtaggatctacgatccgatctacgatccgatcttacgatcctcgatttacgatctttcatcccccctcccgatcctaagtaaaatctcgattttaacaacaatgatcacgaatcaaattatttttttatttctcttccgattttgaatgaaaaactatatttaagcattttttttttgtctacacacgaggaacaaaaaccctcggtcaattttgaggatcattgaagaataaaggaaaaatctagggaggataaaataattaaattagggttactaattataatttgaaattaaagataatagtgacgatttttcaaaattgtcaaaaattgtattttgaatttttaacggtcatgtcatcatttaataaaggaatccttacaacagggactatgaataaattttttcaaatatcaaagatcaaacacaaagaaaaaataaattaggaatcaaacacaaaatttaaatatttattagggatcgaaaatatattttaaccaaaaatttattataaaaaaattgactaatgcaactttcaaaatcttggagaacttgcaaatatttattattatttcttacgataaatgttagctaaaaaagcaaaaaaaaaatatcaaacaatcaacataataaaaaggacaagcacatggtaaacgtttaagtttgtcttggtttaatccaaggtccattttattaatgtatggagaaggatctcctatagtgaaagttaacatgagaggatcatgtgaactaatcacatccatctatatcttttaaatatgagtttataataattttttataaaataaattaaataatttcaatggtttagatctcatgcaatggaaatgtcattgtaggggacaaaagaacaaaatgatacatggctcattttgctcccatgttgagatatgtgacacacatgcacaccatatatataattttagatatgttgaggtatgtgaaagatgtgagatatttcaaaaataaaattgtgagatatttgaaagacatatcctctctatgtgagcaaaaagtgtgtctaaagctcaatgacttgttatttttaactaattctatatgtaccttgcacattctctctaccaactttgcatgttcatacccttcatccggttgccgatatagttctttgacttctttcggttgttgtagttgcagttgctgtacaaaaaagtaacaaatgttaggtacttgagaagtaaaggtccaattaagttacattaaggaaaaatatgcatatgcatctctttattaaatttccaagccagttagtattgatcatcaaaatggacaaaaagaaaagacaatgatttttcctacatggctcattttgctcccatgttgagttatgtgggatgtttcaagccgagcaaaaaataaaaatatacttgaaagacacatcttgtttatgtgagtagaaagtttgtctaacaatatgtagttagacaacaaatgtttcgttatgagatgacactacactttgcgccattgaaaatataatgtgtcttttctcttaatgtataagacgactttgcaaataatctacgtaagacaaaaattgtactttttttttcacaagacaaggccattgcattagcttttttacatcaaaattgttattataatgttccatttgacaataaaataataatgttaaatgataagtccaaaaaaaattatgttttggatgaagaaaactcaatgacttgtaattaattagttctatatgtatcttgtttttctccttttgagtctcacttgcttgtttttgtgcataaaagcaagaaaggttaggtacttgagaagtaaattaaggtgcaattcaacttatttgttcaaataaatagaatgatttgccctagcttatggatcatatagttagatattatgttgagatacgtcaaaaaaaatgtgagatatttgaaggatatactttgtattgtggatttgtgagtagatattatgtctaaaaatttaagatgatttgatttctgcactagacaaagttttgtgtatttcttggtcatatctctttatcaaatttgttattacgatgttccatttgtgattaaaataataatggtattgataagacttgggaaaaattatgttttggattaagagataacaatggttggctgttataattagttctatatgtaccttaaactttctttcatcctcttgccacttcacaaaacaacgttctacgtcaatgtcagtatatacttgcagccacttctgtacataaaagcaatagagattaattaggcacttagtagaggtgcaattcaaatacattaaggaaaaataagcatgcctttattatatttaatctcatgtcacttagtatcgagcattaaaaatggacaaaaggtagagaaagaaaattatatgattcatttagcttccgtttttggacaaacggaagacaaaggcaaattagatgtttaaatttatttgttaaaaaaacttaaaataaacaactttttatctttagtgtatttgtctaaattgcttatgcttaaaaaatcagtttcctcttattttaagaaactaattatatctacttaaaaaaatgctgataaaaaatgcttattttaaaattgattttctaactttaaaaacaaacacacccaatgccttaccctcggtagaaattggtgttttatgtagtttggtcctgttttcataaatttggatacttaaatgctcaattacttctttaagatactacacttttatttattttacctaaaatgtaatttcgtcttcctattttttaattcattatttttgtcctcataatttttaattgagacattttgttttttttttttaaattacaattttagtccctttgtcactttcaagatttgtaaaaataatatgaaccatgattgtacttaaatagtggatagaattaacaattatgaaccatgattgtacttaaatagtggatagaattaacaacaatgaaccatggttgtacttaaatagtggatagaatgaaattaatagaaaaaaattgaacggatccaatgaaataaaataaattaatatttattgtgtcaatattttaatgataaaatgatcaaagagattgatgccatctcatagtagctaaattagaaaagaagaaaaaataatagtattggatgatatgaaataaaatacttataataaagtcatgtttagttctgttccaacatatttaaaaccaatcatgactcaaaagaaaaaaatagcaccttccatttaattgtatttctaggtgtcaattgggaacacaactccgaatatttcaggaattaatgattataaatattgaaatcattttgcaactcaaagtaatctagttaattgatttaactttattttactactattacaagagaaaatgaaaatatgacatttcatcgatagtttctttttcaatattggtagatatgttatagatatttgaaagatgtgagatatttaaaagaaaaagtgagattttgaaagacacacaagaaattaacttttattacaagaaactagctaggaaacttgttaaaaaataattacttgtcaaccaagttataataaggcatagctattatttctaacaggcagaagcctttatgtatgggtaaagagtgtgtttgacaatgtagagttaggcaacacgtgtttagttatgatgacactgcattttgttgaaaacataattatgtctatttgccttaatgtgagacaaattttccaatcatctatacaagtaacaagactaaggttgtgtttttcttgtttacaaagtggttgcattatcttctctagctatatcaaatttgttattataatgatccaattgacagtaaaatattaatagcaatgaatagcactatagaaattatgatttgaacgaagagatcataacttgttgtatcaattagttctatacttacatgatttttcacctgaccaagatatatttgataaaggtaaccttgtagtatcaatccctgtacataaaagcaataaaagttaggtacctaacaagtgaaggtgcaattcaattactgtaaggaaaaacatgcgtatttttagtaaacttctatgccaattagtatttaataggaactggttcagaaaatgcagagaacgactgaagttttattcaatagtgaagaacacagattacgagcacagtgattttcgaggcaatcacaacctcccaactctccaagagagtctaaccttaccttctctccccttcttatacattcacgcccccctctcagttagttataactattcctccaacattgttactgtaatgttagtggcattaaaatacaaaggtggtatgtttggtggatagcactgacatggagcatttggttccaccgcaacaggatcgtcttccacaatgaaaacttcaatggatctaagttgatggaggatgctgtatttttttgttggtcatggctagttaatctggagaaaggatttaatatacctttctaccattggtctagtcagttgagtgttgggttctgctagatagttttttatcttgttttttaacttccttatgagtctaaccttaggctgttctttggcatcttaatgcgggtcgttatcatgtattaggctgccaaagttctggttggctccctgggatggtttctttttcagtattacttgtactcagctctatatatatatatatatatatattatctttgctgatcaaaaaaaaaaaattatcttttagttatactaactgcctaactattaaaatgctatctgaaccctctgtgatatgtttcaccccacctagcactcctatcaataccctcctcttcaagctggaccttgtccccaaggtcctcttcagaaaaagcagtcttcaactcttccagcagctcccagctgttctcatgatgtggcagtccttgccatttgatcttgacagaaaattaatgatgaattaaaagttatcaaaaaaaattgagggactaaaatcttaattaaccctttagattaatatttattacaaataattctgaaattaaaaaaaaaaaaattgaatccctgataagggccaagaaaaatttgaatggagccaataataattctcaaaagtgtgacatttacttgtcattttaagatatcccttcacatctcaaaatatttcacaaattaaaggttacagctccagttcaaagtaatttacatagcataattgcaaatttgaatgcaagaaggaaaaaattattgctttagaagtaaaattacttttgagttcttccaactgaaatctaaatatgcagtaaactacttaatgaggatcgtgatagcaattaagagtttatgagagttataacttacagcgtaaaatttcgggaaagacgaattcacattaggcgcaaacttggattcccaaactgaatcagcaatcacaatccccatgccatgcactgcacacaaatcaaagaaagaatatgcatacatgttagaaattatgatggcgtattaacacaaactgctgctgagttaatttaccatgtacaacccagagttctggatacagttccgttatagttttctcgagtgcccgtaaaagataaggctccaaagtccttgtttccgtttctgatatggattgcaaaattttgcgtatcagctcttccagagagtcgtctccactcacttctatttcttctataaaatgaaacttcttttcggcttccccaactggaaagtaccgtagtagatttttcaaatgaaatttcatccgatctataatggcttctctacgatcggatggaggtgcctgttcattgatctcgttcatttcttctgttcctctcagcctcagctagtttacaaactaatcaatttcggttttgctcaagagttgcttctactgccagaggcggcagaagtagggtggaaaaccctaatttgcgggtgaagttacctatatacccttcattttttatatttttcccaaagtaccctaagacttggtcagaaaatatcagcccatttttttgttagtgcctctgttctcctcgctgttctctcatttcacgctttccccctggccctttctctcgtttcgctgcagcgccgcctgtgcctgttgcctgctgcccacggcgctccgcgcctccgcctgctgcctgcgcctccgcggatctccagcttccaggttcacttaatcacttttatttttcaattttccccaatccaatttacaatcactcaacgataacaaaaataaaaaagtagggataaaaaggaactgcaatgtgctttgtttgaagctatagaacatgtgaacaaattttcattgctttatacatttttaaagcttttaattagtagtttggttggtacttggtaataactagaatatcacattttagtttctatttttcttataaaaaactgacacatacactgatacagtgatacacatgaaatctcatttctttagaaacatattatgagttatgaattaatattcaatgtaagatggataccttaaaattaaacaaatgccacaaaactaaagtgtgacaaaactatgaataaaatgcagtacttttttttttggtttcccacaagtatcatcctttaaaagatgtatttgaaagcattgataataacgtaattatgaaggaattccaaaaaataaggcatagaatgatgatattgtaattatttttctggatatgtatttgaaattttgagtgcccccttgatttattttctggatctgtccctgcctaacaccatacaagtttgagaaggtgatgagaaatcaggacaaccttttagcttttgagtacacattgaacacaattatgcattttgttctggttgcgtgtgtaagtgagttatttaacttatgcaatgatatatggtgtgatgtatattatgtgtattccaggggaaagaggatagtagaacatcaatgaacattagttctgaagttgaaagaagaacaccagaaatcatgggaagatgtgcatcgtggtgggccaccttcacattttttttgtgtttgtgttcattaaaattgagcaagccacttctccaaccacaaggacccaattcactatgccttgcgatagtaggaatgttcagttttaccgtgtttgtatctgcttcttctgttatgcttgttgttggagcacagttcactcgtatgcagataactgttatgctcagtgctttcttggctatgctgttagtttaattagacagttttcatatatgtagttatgtttccttcaggcagggttgaacacaagttgttgtaatcttgttatttacataatttcattcgcataacaataaaacaaagtttgggtatgtgatatatcaatatcaacgattaaccagacagaacatattcacggatgaacattgaatgacaggaacatcccttcgtatgaa

SEQ ID NO: 11 - DNA, 10230 bp, Glycine argyea
  source 1..10230: mol_type = genomic DNA
tgccgcctctggcagtagaagcaactcttgagcaaaaccgaaattgattagtttgtaaactagctgaggctgagaggaacagaagaaatgaacgagatcaatgaacaggcacctccatccgatcgtagagaagccattatagatcggatgaaatttcatttgaaaaatctactacggtactttccagttggggaagccgaaaagaagtttcattttatagaagaaatagaagtgagtggagacgactctctggaagagctgatacgcaaaattttgcaatccatatcagaaacggaaacaaggactttggagccttatcttttacgggcactcgagaaaactataacggaactgtatccagaactctgggttgtacatggtaaattaactcagcagcagtttgtgttaatacgccatcataatttctaacatgtatgcatattctttctttgatttgtgtgcagtgcatggcatggggattgtgattgctgattcagtttgggaatccaagtttgcgcctaatgtgaattcgtctttcccgaaattttacgctgtaagttataactctcataaactcttaattgctatcacgatcctcattaagtagtttactgcatatttagatttcagttggaagaactcaaaagtaattttacttctaaagcaataattttttccttcttgcattcaaatttgcaattatgctatgtaaattactttgaactggagctgtaacctttaatttgtgaaatattttgagatgtgaagggatatcttaaaatgacaagtaaatgtcacacttttgagaattattattggctccattcaaatttttcttggcccttatcagggattcaatttttttttttttaatttcagaattatttgtaataaatattaatctaaagggttaattaagattttagtccctcaattttttttgataacttttaattcatcattaattttctgtcaagatcaaatggcaaggactgccacatcatgagaacagctgggagctgctggaagagttgaagactgctttttctgaagaggaccttggggacaaggtccagcttgaagaggagggtattgataggagtgctaggtggggtgaaacatatcacagagggttcagatagcattttaatagttaggcagttagtataactaaaagataattttttttttttgatcagcaaagataatatatatatatatatatatatagagctgagtacaagtaatactgaaaaagaaaccatcccagggagccaaccagaactttggcagcctaatacatgataacgacccgcattaagatgccaaagaacagcctaaggttagactcataaggaagttaaaaaacaagataaaaaactatctagcagaacccaacactcaactgactagaccaatggtagaaaggtatattaaatcctttctccagattaactagccatgaccaacaaaaaaatacagcatcctccatcaacttagatccattgaagttttcattgtggaagacgatcctgttgcggtggaaccaaatgctccatgtcagtgctatccaccaaacataccacctttgtattttaatgccactaacattacagtaacaatgttggaggaatagttataactaactgagaggggggcgtgaatgtataagaaggggagagaaggtaaggttagactctcttggagagttgggaggttgtgattgcctcgaaaatcactgtgctcgtaatctgtgttcttcactattgaataaaacttcagtcgttctctgcattttctgaaccagttcctattaaatactaattggcatagaagtttactaaaaatacgcatgtttttccttacagtaattgaattgcaccttcacttgttaggtacctaacttttattgcttttatgtacagggattgatactacaaggttacctttatcaaatatatcttggtcaggtgaaaaatcatgtaagtatagaactaattgatacaacaagttatgatctcttcgttcaaatcataatttctatagtgctattcattgctattaatattttactgtcaattggatcattataataacaaatttgatatagctagagaagataatgcaaccactttgtaaacaagaaaaacacaaccttagtcttgttacttgtatagatgattggaaaatttgtctcacattaaggcaaatagacataattatgttttcaacaaaatgcagtgtcatcataactaaacacgtgttgcctaactctacattgtcaaacacactctttacccatacataaaggcttctgcctgttagaaataatagctatgccttattataacttggttgacaagtaattattttttaacaagtttcctagctagtttcttgtaataaaagttaatttcttgtgtgtctttcaaaatctcactttttcttttaaatatctcacatctttcaaatatctataacatatctaccaatattgaaaaagaaactatcgatgaaatgtcatattttcattttctcttgtaatagtagtaaaataaagttaaatcaattaactagattactttgagttgcaaaatgatttcaatatttataatcattaattcctgaaatattcggagttgtgttcccaattgacacctagaaatacaattaaatggaaggtgctatttttttcttttgagtcatgattggttttaaatatgttggaacagaactaaacatgactttattataagtattttatttcatatcatccaatactattattttttcttcttttctaatttagctactatgagatggcatcaatctctttgatcattttatcattaaaatattgacacaataaatattaatttattttatttcattggatccgttcaatttttttctattaatttcattctatccactatttaagtacaaccatggttcattgttgttaattctatccactatttaagtacaatcatggttcataattgttaattctatccactatttaagtacaatcatggttcatattatttttacaaatcttgaaagtgacaaagggactaaaattgtaatttaaaaaaaaaaaacaaaatgtctcaattaaaaattatgaggacaaaaataatgaattaaaaaataggaagacgaaattacattttaggtaaaataaataaaagtgtagtatcttaaagaagtaattgagcatttaagtatccaaatttatgaaaacaggaccaaactacataaaacaccaatttctaccgagggtaaggcattgggtgtgtttgtttttaaagttagaaaatcaattttaaaataagcattttttatcagcatttttttaagtagatataattagtttcttaaaataagaggaaactgattttttaagcataagcaatttagacaaatacactaaagataaaaagttgtttattttaagtttttttaacaaataaatttaaacatctaatttgcctttgtcttccgtttgtccaaaaacggaagctaaatgaatcatataattttctttctctaccttttgtccatttttaatgctcgatactaagtgacatgagattaaatataataaaggcatgcttatttttccttaatgtatttgaattgcacctctactaagtgcctaattaatctctattgcttttatgtacagaagtggctgcaagtatatactgacattgacgtagaacgttgttttgtgaagtggcaagaggatgaaagaaagtttaaggtacatatagaactaattataacagccaaccattgttatctcttaatccaaaacataatttttcccaagtcttatcaataccattattattttaatcacaaatggaacatcgtaataacaaatttgataaagagatatgaccaagaaatacacaaaactttgtctagtgcagaaatcaaatcatcttaaatttttagacataatatctactcacaaatccacaatacaaagtatatccttcaaatatctcacattttttttgacgtatctcaacataatatctaactatatgatccataagctagggcaaatcattctatttatttgaacaaataagttgaattgcaccttaatttacttctcaagtacctaacctttcttgcttttatgcacaaaaacaagcaagtgagactcaaaaggagaaaaacaagatacatatagaactaattaattacaagtcattgagttttcttcatccaaaacataattttttttggacttatcatttaacattattattttattgtcaaatggaacattataataacaattttgatgtaaaaaagctaatgcaatggccttgtcttgtgaaaaaaaaagtacaatttttgtcttacgtagattatttgcaaagtcgtcttatacattaagagaaaagacacattatattttcaatggcgcaaagtgtagtgtcatctcataacgaaacatttgttgtctaactacatattgttagacaaactttctactcacataaacaagatgtgtctttcaagtatatttttattttttgctcggcttgaaacatcccacataactcaacatgggagcaaaatgagccatgtaggaaaaatcattgtcttttctttttgtccattttgatgatcaatactaactggcttggaaatttaataaagagatgcatatgcatatttttccttaatgtaacttaattggacctttacttctcaagtacctaacatttgttacttttttgtacagcaactgcaactacaacaaccgaaagaagtcaaagaactatatcggcaaccggatgaagggtatgaacatgcaaagttggtagagagaatgtgcaaggtacatatagaattagttaaaaataacaagtcattgagctttagacacactttttgctcacatagagaggatatgtctttcaaatatctcacaattttatttttgaaatatctcacatctttcacatacctcaacatatctaaaattatatatatggtgtgcatgtgtgtcacatatctcaacatgggagcaaaatgagccatgtatcattttgttcttttgtcccctacaatgacatttccattgcatgagatctaaaccattgaaattatttaatttattttataaaaaattattataaactcatatttaaaagatatagatggatgtgattagttcacatgatcctctcatgttaactttcactataggagatccttctccatacattaataaaatggaccttggattaaaccaagacaaacttaaacgtttaccatgtgcttgtcctttttattatgttgattgtttgatattttttttttgcttttttagctaacatttatcgtaagaaataataataaatatttgcaagttctccaagattttgaaagttgcattagtcaatttttttataataaatttttggttaaaatatattttcgatccctaataaatatttaaattttgtgtttgattcctaatttattttttctttgtgtttgatctttgatatttgaaaaaatttattcatagtccctgttgtaaggattcctttattaaatgatgacatgaccgttaaaaattcaaaatacaatttttgacaattttgaaaaatcgtcactattatctttaatttcaaattataattagtaaccctaatttaattattttatcctccctagatttttcctttattcttcaatgatcctcaaaattgaccgagggtttttgttcctcgtgtgtagacaaaaaaaaaatgcttaaatatagtttttcattcaaaatcggaagagaaataaaaaaataatttgattcgtgatcattgttgttaaaatcgagattttacttaggatcgggaggggggatgaaagatcgtaaatcgaggatcgtaagatcggatcgtagatcggatcgtagatcctactatgaaactgcagaaataaaaaagtgggtatggaaagacttaaatagaaattacgtaaattaaaactcaaaagtccattcaaaaatattatatgatactgcagaaagaaaaaaagtgagtacaaaaaacaaattagtagaaaaccctattttcttttcttctcctcttctcctgtagcgtgtttcctcttctcttctccccgcatgtttcctcttctcctctgctcctggtcgcgtgtttcatcttctcctggtcgcgtgtgttattgctgaaactagaggagctctgcggcgcctctgtgaagcacctccgtcctccgcgtttcgttgctgaaaatcagagccttcctctcgccgccgagatcgccgaacaagttttccactcctccgtcaaaatcggcgtcgatctcctccttctcctcctccttcgggtcgtcgaccttggatcgttcgggatcgcagaagaggggaggtcgggaggtgccaccgtcgtctgaaatcgcgcggggggaggtcgggatgttgcggcgttgctgggtcgcgcagctactgggtgatttgggtcgttttcgaatccaaggttagggcttttaatttcagtgctgaaacaggatcgtgacggatcgaggatcttacgatcctacgatcaaactcgcgatctcaccacgatcccactcggctacgatcctagcaccgatccggattgccgttggccgcaaagatcgtaaaatcgtccgatcttacgatccagatcgcgattttgctaacagtgttcgtgataatgttgtctaatgaaaatgccctccttttgttttcctttgattttctcttgtaaaagtagtaaaataaagttaaatcaattaactagattactttgagttgcaaaatgatttcaatatttgtaatcattaattcctgaaatattcggagttgtgttcccaattgacacctagaaatacaataaaatggaaggtgctatttttttctctttcttttgagtcatgattggttttaaatatgttcggaatagaactaaacatgactttgttatactttgttatacgtattttatttcatatcatccaatactattattttttcttcttttctaatttagctactatgagatggaatcaatctctttgatcattttatcattaaaatattgacacaataaatattaatttattttatttcattggatccgttcaatttgtttctattaatttcattctatccactgtttaagtacaaccatggtggttcatattatttttacaagtcttgaaagtgacaaaaagggactaaaatcacaaaattttaaaagtgaaaagcaaaatgtctcaatcaaaaatagggagactaaaattataatttttttttaaaataaaaaataaaatgtctcaattaaaaattatgaggacaaaaataataaattttaaaaaataggaagacaaaagttacattttaggtaaaataaaagtgtagtatcttaaagaattaattgagcatttaagtatccaaatttatgaaaacaggaccaaactacataaaacaccaatttataccgagggtaaggcattgggtgcgtttgtttttaaagttagaaaatccttttaaaataagcgttttttatcagcattttttctaagtagatataattagtttcttaaaataagaggaaactggttttttaagcacaatcaatttagacaaacacactaaaaataaaaaagttgtttattttaagtatttttttttacaaataaatttaaacatctaatttgcctttgtcttctgtttgtccaaaaacgggagcctcacgtcgagccgcaccgagtcatctgccgccaccccgtcgtcacgtgtccaccgtgcaacacctccctgcgtcgacaccctcgtgttggccgcgccgtcgtcacgctcacggcttgcttggcctcggcctcgacctccaatctgcgttctgcgttctgcgttcgtgcctgcgtcgtcaccgccattgaagctgattcaagcttttttcacatgatttgttctgaattattattaattaccacatgatttgttctgaattcctatgagccaatactttcatataggattgtagatcttagaatgcttttgtgatatcataaatctaaggattgtgaggtcaagagatatgatgaaagtatattacataagaaaatgtgggtatctgaatacagatgtcatgtcagctagttatttatttgtcaatttctatggatccatttctctcatattagaggttagagctttctgcattgatggatatttttatcctttgtaagatttatgtttgttcccgttaatggtgattaaatatattatatagtcttttcgtttcagaatatcaattcatatcaattttataaagttttatactctgttatttatatatagtctggtattcatataaatcacaccctgatacattaaaacaatagtgggacaatagcttttgaggaactcaagaaggggttgaaaagtgtgggctctaatctcatggaatatgaaattaaatcccttatggaagcggtaggtagatttccattatcatagaatgtaacacatagtgttttaatgttatttcaaataagtcttcttttgtgtaagctgagttgggtgatcattaattgccttgccttatgtatgaactttggcaggctggtatagacaacaatggatcaatagactatggtgaacttcctgctgctacactgcacttgaataagatggaaggaggggaggatttggctgctgcttttgcctattttgataaagatgatagtggtcacataagcagtgacgagcttctacaggcttttagggacttcagcctacgttatcatctggatgagatgataaaaaagattgatcaatataatgtaagttcctctaactgtgtttgcataatctccagtatgtgccgatgtgattgttgtcactgtttagtttcactaacaatttgttgattccatttttagttgatttaaagtgttttttttaaaaaaatttacaaaaaaatactaagtgtgcgaacataaaagaatattcctggatccgttgctgcctcaacatatctaaaattatatatatatggtgtgcatgtgtgtcacatatctcaacatgggagcaaaatgagccatgtatcattttgttctttttgtcccctgaaatggatcgtgatggcaagttgttggtgtgcctcggtgaagcaaattctagtcatttttagatgccataaattgtcatttaaaatttaaagtaaatactggttgttttagaaatcatcagaaatcacatttaacagacacttaacattcttaacctaatgttagggaccagaaacaaaaaaattcaaatatcaaggatcaaatacaaagaaaacatttattgggaagtaaacacaacaaaatttgaatatttattaagaatcacaaatatattttaacctaaatttttaacatcatctaaaaaatataagatctttaaatttgcatgaaaataccaaaaataaagttccatagaaagaccatatattatttcatcaaaattctcactcaccaaccatatttctccattgtgattgactatcaacaggaagaaaagaaaaatcgccattctcatcccccaaaccatgttgtaaagattgacgataatggggtgttgaaagtagataaaggaaagaagaagagtttgaacagatttctgaagttttatgggaacaggctattccgatcacattctactgatcgtactttatgtgttgccttatatggactgctatggacccaactatctgctttgggcaatcctcttgacaagttgtcccaatccacaagcattgacccaatggaattaagtatggggaaagcatgttccagcccagtatgggcatcactatttttcctgaacattatttatgcagcagtgcatacctcgtcattgccttttcatatatcggcatacgcattgctgggattttctttactctattacattttagagataatagttgcaacgtctggatatagtgtttcatggggtggttcagctaaaatatcaggcctattgtttgtgtttggaggggccacaggcttaatgctactacttcttagcatcataatcatcggggtgctctatgctgcatggtgctcctctctaaatgatgttggaggagaactaactgaagcactcgcctgagtgtatcatcacgatccaggatacctgaaatcatgcacttgaat

SEQ ID NO: 12 - DNA, 130 bp, Glycine argyea
  source 1..130: mol_type = genomic DNA
gaagtagggtggaaaaccctaatttgcgggtgaagttacctatatacccttcattttttatatttttcccaaagtaccctaagacttggtcagaaaatatcagcccatttttttgttagtgcctctgttc

SEQ ID NO: 13 - DNA, 1396 bp, Glycine argyea
  source 1..1396: mol_type = genomic DNA
tcctcgctgttctctcatttcacgctttccccctggccctttctctcgtttcgctgcagcgccgcctgtgcctgttgcctgctgcccacggcgctccgcgcctccgcctgctgcctgcgcctccgcggatctccagcttccaggttcacttaatcacttttatttttcaattttccccaatccaatttacaatcactcaacgataacaaaaataaaaaagtagggataaaaaggaactgcaatgtgctttgtttgaagctatagaacatgtgaacaaattttcattgctttatacatttttaaagcttttaattagtagtttggttggtacttggtaataactagaatatcacattttagtttctatttttcttataaaaaactgacacatacactgatacagtgatacacatgaaatctcatttctttagaaacatattatgagttatgaattaatattcaatgtaagatggataccttaaaattaaacaaatgccacaaaactaaagtgtgacaaaactatgaataaaatgcagtacttttttttttggtttcccacaagtatcatcctttaaaagatgtatttgaaagcattgataataacgtaattatgaaggaattccaaaaaataaggcatagaatgatgatattgtaattatttttctggatatgtatttgaaattttgagtgcccccttgatttattttctggatctgtccctgcctaacaccatacaagtttgagaaggtgatgagaaatcaggacaaccttttagcttttgagtacacattgaacacaattatgcattttgttctggttgcgtgtgtaagtgagttatttaacttatgcaatgatatatggtgtgatgtatattatgtgtattccaggggaaagaggatagtagaacatcaatgaacattagttctgaagttgaaagaagaacaccagaaatcatgggaagatgtgcatcgtggtgggccaccttcacattttttttgtgtttgtgttcattaaaattgagcaagccacttctccaaccacaaggacccaattcactatgccttgcgatagtaggaatgttcagttttaccgtgtttgtatctgcttcttctgttatgcttgttgttggagcacagttcactcgtatgcagataactgttatgctcagtgctttcttggctatgctgttagtttaattagacagttttcatatatgtagttatgtttccttcaggcagggttgaacacaagttgttgtaatcttgttatttacataatttcattcgcataacaataaaacaaagtttgggtatgtgatatatcaatatcaacgattaaccagacagaacatattcacggatgaacattgaatgacaggaacatcccttcgtatgaa

SEQ ID NO: 14 - DNA, 1568 bp, Glycine argyea
  source 1..1568: mol_type = unassigned DNA
tgccgcctctggcagtagaagcaactcttgagcaaaaccgaaattgattagtttgtaaactagctgaggctgagaggaacagaagaaatgaacgagatcaatgaacaggcacctccatccgatcgtagagaagccattatagatcggatgaaatttcatttgaaaaatctactacggtactttccagttggggaagccgaaaagaagtttcattttatagaagaaatagaagtgagtggagacgactctctggaagagctgatacgcaaaattttgcaatccatatcagaaacggaaacaaggactttggagccttatcttttacgggcactcgagaaaactataacggaactgtatccagaactctgggttgtacatgtgcatggcatggggattgtgattgctgattcagtttgggaatccaagtttgcgcctaatgtgaattcgtctttcccgaaattttacgctggattgatactacaaggttacctttatcaaatatatcttggtcaggtgaaaaatcataagtggctgcaagtatatactgacattgacgtagaacgttgttttgtgaagtggcaagaggatgaaagaaagtttaagcaactgcaactacaacaaccgaaagaagtcaaagaactatatcggcaaccggatgaagggtatgaacatgcaaagttggtagagagaatgtgcaaggctggtatagacaacaatggatcaatagactatggtgaacttcctgctgctacactgcacttgaataagatggaaggaggggaggatttggctgctgcttttgcctattttgataaagatgatagtggtcacataagcagtgacgagcttctacaggcttttagggacttcagcctacgttatcatctggatgagatgataaaaaagattgatcaatataatgaagaaaagaaaaatcgccattctcatcccccaaaccatgttgtaaagattgacgataatggggtgttgaaagtagataaaggaaagaagaagagtttgaacagatttctgaagttttatgggaacaggctattccgatcacattctactgatcgtactttatgtgttgccttatatggactgctatggacccaactatctgctttgggcaatcctcttgacaagttgtcccaatccacaagcattgacccaatggaattaagtatggggaaagcatgttccagcccagtatgggcatcactatttttcctgaacattatttatgcagcagtgcatacctcgtcattgccttttcatatatcggcatacgcattgctgggattttctttactctattacattttagagataatagttgcaacgtctggatatagtgtttcatggggtggttcagctaaaatatcaggcctattgtttgtgtttggaggggccacaggcttaatgctactacttcttagcatcataatcatcggggtgctctatgctgcatggtgctcctctctaaatgatgttggaggagaactaactgaagcactcgcctgagtgtatcatcacgatccaggatacctgaaatcatgcacttgaat

SEQ ID NO: 15 - protein, 478 aa, Glycine argyea
MNEINEQAPPSDRREAIIDRMKFHLKNLLRYFPVGEAEKKFHFIEEIEVSGDDSLEELIRKILQSISETETRTLEPYLLRALEKTITELYPELWVVHVHGMGIVIADSVWESKFAPNVNSSFPKFYAGLILQGYLYQIYLGQVKNHKWLQVYTDIDVERCFVKWQEDERKFKQLQLQQPKEVKELYRQPDEGYEHAKLVERMCKAGIDNNGSIDYGELPAATLHLNKMEGGEDLAAAFAYFDKDDSGHISSDELLQAFRDFSLRYHLDEMIKKIDQYNEEKKNRHSHPPNHVVKIDDNGVLKVDKGKKKSLNRFLKFYGNRLFRSHSTDRTLCVALYGLLWTQLSALGNPLDKLSQSTSIDPMELSMGKACSSPVWASLFFLNIIYAAVHTSSLPFHISAYALLGFSLLYYILEIIVATSGYSVSWGGSAKISGLLFVFGGATGLMLLLLSIIIIGVLYAAWCSSLNDVGGELTEALA

SEQ ID NO: 16 - DNA, 652 bp, Glycine argyea
  source 1..652: mol_type = unassigned DNA
tcctcgctgttctctcatttcacgctttccccctggccctttctctcgtttcgctgcagcgccgcctgtgcctgttgcctgctgcccacggcgctccgcgcctccgcctgctgcctgcgcctccgcggatctccagcttccaggggaaagaggatagtagaacatcaatgaacattagttctgaagttgaaagaagaacaccagaaatcatgggaagatgtgcatcgtggtgggccaccttcacattttttttgtgtttgtgttcattaaaattgagcaagccacttctccaaccacaaggacccaattcactatgccttgcgatagtaggaatgttcagttttaccgtgtttgtatctgcttcttctgttatgcttgttgttggagcacagttcactcgtatgcagataactgttatgctcagtgctttcttggctatgctgttagtttaattagacagttttcatatatgtagttatgtttccttcaggcagggttgaacacaagttgttgtaatcttgttatttacataatttcattcgcataacaataaaacaaagtttgggtatgtgatatatcaatatcaacgattaaccagacagaacatattcacggatgaacattgaatgacaggaacatcccttcgtatgaa

SEQ ID NO: 17 - protein, 149 aa, Glycine argyea
LAVLSFHAFPLALSLVSLQRRLCLLPAAHGAPRLRLLPAPPRISSFQGKEDSRTSMNISSEVERRTPEIMGRCASWWATFTFFLCLCSLKLSKPLLQPQGPNSLCLAIVGMFSFTVFVSASSVMLVVGAQFTRMQITVMLSAFLAMLLV

SEQ ID NO: 18 - DNA, 119155 bp, Glycine argyea
  source 1..119155: mol_type = genomic DNA
aaatactcaaactttgatatttcaaaggcaaccacgacctaaaaagaaataaaacagataaattgtttctggatccaaaataccagattccagacttacattcaagtgcgtcatttcaggtatcctggatcttgatgatacactcaggcgagtgtttctgagttacttctcctccaacatcatttggataggagcaccatatgcagctagcatagagcacccccatgattatgatgctaagaagtagcattaaggatgtggcccctccaaacacaaacaataggcctgagattttagctgaaccaccccatgaaacattatatccagacactgcaactattatcgctaaaatgtaatagagtaaattaaatcccagcattgagtatgcgggtatacgatgaggcaatgacaaggtaagcatggctgcaaaaataaagttcaggaaaaatattgatgcccatactgtgatggaacatgctttccccatacttaattccattgggtcaatgcctctggattgggataacttctcaagaggattgcccaaaacagatagttgggcccattgcattccatataaggcagcccatgtagtaagacgatttgcatcagtagaatgtgattggaatggcctgttcccataaaacttcaaaaatgtgttcaaactcttcgtctttcctttatctacttttaacaccccattatcgtcaatctttacaacatggtttgggggatgagaacggcgattttccttttcttcctgttgatagtcaatcacaatggagaaatatggttggtgagtgagaattttgatgaaataatatatggtctttctatggaactttatttttggtattttcttgtaaatgtaaagatctcatgcaatggaaatgtcattgtaggggacaaaataacaaaatgatatatggctcattttgcacccatgttgagatatgtgacacacatgcacaccatatatataattttagatatgttgaggtatgtgaaagatgtgagatatttcaaaaataaaattgtgagatatttgaaagacatatcctctctatgtgagcaaaaagtgtgtctaaagcttgatgacttgttattattacctaattctatatgttaccttgcattttttcagttgctgatatagttctttctgtggctgtacaaaaaagcaacaaatgttaggtacttgagaagtaaagatccaatcaagttaaattaaggaaaaatatgcatatgcatctctttattaaattttcaacccaattagtattgatcatcaaaatggacaaaaggaaaagacaatgattttccctacatggctcattttgctcctatgttgagttatgtgagatatttcaagccgagaaaaaacaaaaatatatttgaaagacacatcttttttatgtgagtagaaagttagtctaacaatatggagttagacaccaaatgtttcgttacgagatgacactacactttgcgccattgaaaatataatgtgtcttttctccccgtgcataagacgattttgaaaataattacataagacataaattgtactttttttgtcacgagacaaggtcattgaattagcttttttacatcaaaattgttattataatgttccatttgacaataaaataataatgttaatgataagtcccaaaaaaattatgttttggatgaagaaaacacaataacctgtcattaattagttctatatgtaccttgttctcctcttgactctcgcatgcttgtttttgtgcataaaagcaaggaaggttaggtacttgatcatatcattagatattatgttaaattaaggtgcaattcaacttatttgttaaaataaatacaatgattttccctagcttatggatcatatcattagatactatgttgagatacatcaaaaacaaaatgtgacatatttgaaggatatactttgtattgtgagtagaaattctgtctaaagatgtaagatgatttgatttctgcactagacaaagttttgtgtttttcttgatcacatctctatatcaaatttgttattacaatgttccatttgtcattaaaataataatggtattgataagacttgggaaaaattatgtacctttgatattatttcattctctggccttttcacaaaacaactttctaggtcaatgccagtatatacttgcagccgcttctgctgtacataaaagaaatagatgttaattaggcacttagtagaggtgaaattcaaatacattaaggaaaaataagcatgcctttattatatttaatctgatgtcaattagtatcgagcattaaaaatggacaaaaggaagagaaagaaaattatatgattcatttagctcccgcttttggacacaaaccgaagaaaggaaaattaaatgtttaaatttttgttaaaaaaaacaaaaaataaacaactttttatttttagtgtgattgtctaaattacttatgcttaaaaaataactttcctcttattttaagaaactaattatatctacttaaaaaaatgctgataaaaaatgcttattttaaaattgattttctaactttaaaaacaaacacacccaatgccttaccctcggtagaaattgatgttttatgtagtttggtcctgttttcataaatttggatacttaaatgctcaattatctctttaagatactacacttttatttattttacctaaaatgtaactttcgtcttcttatttttttaaatccattatttttctcataatttttaattgagacattttattttttatttttaattaattgtaattttagtcctcctatttttaattgagacatttcacttttcacttttaaaaatttgtgattttagtccctcactttcaagacttgtaaaaataatatgaactatgattgtacttagatagtggatagaaagaaattaatagaaaaaaattgaacggatccaatgaaataaaataaattaatatttattgtgtcaatattttaatgataaaatgatcaaagagattgattccatctcatagtagctaaattagaaaagaagaaaaaataatagtattggatgatatgaaataaaatacgtataataaagtcatgtttagttctattccaacatatttaaaaccaatcatgactcaaaagaaaaaaaatagtaccttccattttattgtatttctaggtgtcaattgggaacacaactccgaatatttcaggaattaatgattataaatattgaaatcattttgcaactcaaagtaatctaggtaattgatttaactttattttactacttttacaagagaaaacgaaaatatgacattttatcgatagtttctttttctatattggtagatatgttatagatatttgaaagatgtgagatatttaaaaaaaaagtgagattttgaaagacacacaagaaattaacttttattacaagaaactagctaggaaacttgttaaaaaataattacttgtcaaccaagttataataaggcatagctattatttctaacaggcacaagcctttatgtatgggtaaagagtgtgtttgacaatgtagacttaggcaacacgtgtttagttatgatgacactacattttgttgaaaacataattatgtctatttgccttaatgtgagacaaattttccaatcatctatacaagtaacaagactaaggttgtgtttttcttgttcacaaagtggttgcattatcttctctatataaaatttgttattataatgatccaattgacagtaaaatattaatagcaatgaatagcactatagaaagttatgttttgaacgaagagatcataacttgttgtatcaattagttctatacttacatgatttttcaccatttgcatcggataatactgatcaagatatattcgattaaggtaacgttgtagtatgactctctgtacataaaagcaataaaagttaggtacctaacaagtgaaggtgcaattcaattactgtaaggaaaaacatgcatatttttagtaaacttctatgccaattagtattgatcataaaaatggacaacaggaagagaatcacaattacttgccctggtttatttcgttgccatgtttggacaaaaggcaaacaacgacaaggacttgcccttggtagaaaaattgttgtttttcgtagcttggtacaggtttcacaaagttggaaacttaaaaagctaaattatttctttaagatactatgcattaatgtaatgtcactaattataatcaatgtaatttgggtgatatttgcataattaagagacttagtattaataaaagaggattaatgaaagaagggacattttggtatttgtttagaggttagggtgtgaataagagattgctgaatgcacccccctcctgatgttaaaaactgtggctgaccatggttctctcactttctccttcttcttcctttatgtccatgtctctccttcacacccaagcaccatttctcttcactccttgattagtggctggaaacataaatcaagcaagaaaggaagctcttctccttgtcctttattgttgttgagtttggttgtgagaaatttgaggaaaagagtaaagctaggagctgcccacttcattcggagtagctgggcagaattggaagctatagagagctggtccactctccaatttgatcaattgaactctaaggttggtactctcctcctatttctccttgattcttagaaatcataaggttagggtttggtttgatgagggtttggagtgaaattgataaatgttgagtattggaagggtgaaacagttttaaggtgtaatttggagtgtgtagaagttgattcaaggttgaaatggtgttccaagcatgaaacatgcatgttcatgacagcaaggaacatcgatctggtcagccctgatgttttgtgcataacaggagctatcggtatccaaatggtctaaaaacagtgtcatttgaaagttaagagattcaagtaggttctggaattttcgtgctatttttggaggttcctagaatttattgtgcagagttataacaggctgttatactctgtttttagcagattttactggtgcagaggcaatcttgcggaaattttatctcagtctaggatgatccaaatttagtgctttcagtggaaatagaaagatgtgtatgtttaatttatacctgtaaaagctcaggtcatttggatgagtatcctggtgttggcgggagtttgaaaatggctggttgaactgatatgttactgtcagcactgttatagtgcgtaacagttctggtattttaagtgttatagaggctgtggtttatgaaattggatgaaaccagttttgtttgaaagataggagataaatatagatgcctctaaattcccttgattttaggagctttctacagggatttatgatttttataagtgggtacaaaatctgcataattttatgcagaaaattctgtttttccgtgtgttttggtataattaaatgccttgtacagataggatgattgttgattcatgcctgtgtgcctttgtataggcgtgacaccgagggtgttagtggcggagaccacagttcagggattgaacagcagagcacggcctcgcctggtaagttcgtggttcttgggagtgtttcccactgcttgagtttcaccacactccgagggagtgatggttgcatacatgattcatatgcattaaattggatgttacagctttcctcatgataatatgtatgcatattctgtgctttgtgtttggttgcctttcggatagatcttctggcaagttagatagatatggacctacctaggagcagagcacaactcctagttggtttgctcacataaagggtatatgatcccttaacaccagagatacgtggtcccgtcgcggttgaagcctattctcggaggtgtcatatacgtgcatccatgcgtaaaagggtatctcccgatgggatgacaagggtgtgagttccggcctcacactgaggactgagagtcctgatgctgatgatgatgcatctttggcaggcttagtaccgcagactcctgatgctgatgatgatgcatcattggcggcgtagttccgcagatacacattagagctaaagaatgtttagttgatttaccacggtctggacccgggtgatgaaaccggataccccttctccttccttcattttcattccttccaactctccacggagagtttgaagtcgaacctgtctaactcgatcagctcccttgtagacatttgcaagaatgtcccacgcctcctttgaatttgttgcaccagcgatcttcttaaacccaaaatcatcaacagcccggaataacatgtacaacgcccctttgtctttcaacctcgtctccttcaatgtcttgttctgcgccgtcgtatatcctactgtggatgttggtttctcaaaaccaccatggacaacttcccaactatcatgggctcccaaaagagctttcatctggatactccagtttttgtaattagcctttgtaaggcggggtagtggcagactactacttggcccactctctattttctcaagctctgaaaccaatttgtgatatcacaggtcctcacacacactagtgcactcacacaaacacacaacaatctctctggtagaatacttctgatgtatttagataatggtctgccaagccacacattggcctcttacatgagatgctatttataagtaatagcaagtgggatatgactcttcacatgagtaacttatttacctagggacatgactcttcacatgagtaacttcttcacctatctaatttattattattaaagtttaatccaacaaaatctctccataaactttaaatatttttttcgtctttcatccccaactttttttttctttttctcttataaacccatttaactccaatgggctgacttccttttggcagttcggtcaactcccaagtttcattccgttcaatggccttgatttcttcattcatggcggttttccatttttcatctcggattgcctctttaaatgttatttcttcagaatctgtcaacaaacaaataaggtaagcttcattagttgaatcatacaggtcctgcaaactcctcattcggggttgtctcatctcgtcttcatcagatgtctcacttcttgccgggatccaacttggtgattctccttccttctcttcttgtcgagtaagcgtctgtccgggatctgaggtgacctcggatgaagtgctccagttccattcatcttcttcattaactttcacatctcggcttatcattactttcttggtgccgggatccaacaacttgtatccctttgtcttttcgtcatatccaatgaaaataaatttcctgctcttgtcttccaacttggttctttgctgatcagggatgtgtgcataagccacactcccgaagactctgaaatggcctactgatggcttctgtcctgtccatgcttcctgtggggtctttccatttagtctggaatgtggacaacggttctgcacatacactgcacacaatacggcctgtccccaaaactccttaggcattttcttcgttttcaacattgaccttaccatgtcaagaatggtactttttttctctcagccactccattttgctgtggtgagtacggtgcagttaagaacctttttatcccatgctcctcgcaataccttttaaatgcaattgatgtgtactccccacctcggtcagaacggattgacttgattcaccttccagttgcattttctatcatcatcttaaaactcttgaatacttcaagcactttggatttttcttccagcaaatacacccaagttttacgactaaaatcgtctataaacgagacaaaatacctctttccactgaaagacccaggagagataggtccacacaagtcggtatgaattagttctagtggttgttgagatctgtatttggccgtcttttgaaatggactcctagcatgcttgcttagcacacattcttcacaaaatctcttttcatagttcaaactcgacaacccttccaccatattcttgttcatcatctcgaccaggcctccgtagtgaagatgcccaaatcggcgatgccataaggatacttcatcactcatctcggcttgcaaacagccgacctgcacactcctcaacattagtttgaacattctatttttcttcatctgaacttgaactatcaaacatccttgtttatccttcaagaagagaactcggtctgtcatgagaaccgagtatccattctccatcagttgtcccacactaagaatattactttttagatcgggcacgtaatagacatcttggatgcgcccaattcgtccatctttctgcaagaaacaaatcgtaccttggcctttgacatcaacctttgaagcatctccaaacgaaacatgtctggcttctatttgtctcagcttactgaacaaatgcttatgcccacacatatgattgcttgcactagtgtctagataccattccatctcgaatccctggttgacttcacttctagtcatcatcagaattcctccatctcttacttctccttctgtaacaaggtttgaaatttcatttcttctttcatcttggcaatctttcgcaaaatgtccgactcgaccacagttgtaacatctgtccgagttgcaatctctcgagtaatgtccatactttccgcatttaaaacattctacatttgattgatttgaatgtcggcctcttccacgatcttggcctcgccaattctgccgacttaatttagcctgctccttattccttccttcttgaccgcggcctctactaccgcgaccgtcttctcgacctccatagccacgccctctacctcgaaagttttgagaatagagtaccttttcgtccttgattgaagcttttgtctggagtgcttgctccagtgactcttctttcttctttttctttctttgtttatgcgcctccaaggaaccagcaagttcgtcaacggtgagtgttgctaggtcttttgactcctcaatcgcacatacaacattctcgaagtcgtcagtgagagacctcagaattttctccaccactttggcatcggacaacacctctctatttctgtttagttgatttaccacggtctagacccgggtgatgaaaccggataccccttctccttccttcattttcattccttccaactctccacggagagtttgaagtcgaacctgtctaactcgatcggctcccttgtagaaatttgcaagaatgtcccacgcctcctttgaagttgttgcaccagcgatcttctcgaacccggaatcatcaacagtccagaataacatgtacaacgcccccttgtctttcaacctcatctccttcaatgtcttgttctgcaccgccgtatatcctgctatggatgttggttccttgaaaccaccatggacaatttcccaactatcatgggctcccaaaagagctttcatctggatactccagttttcgtaattagcctttgtaaggcggggtagtggtagactactacttggcccactctccattttctcaagctctgataccaatttgtgatatcacaggtcctcacacacactggtgcactcacccaaacacacaacaatctctctggtagaatgcttctgatatatttagataatggtctgtcaaactacacattggcctcttacatgagatgttatttataagtaatagcaagtgggatatgactcttcacatgagtaacttctttacctaagaacatgactcttcacatgagtaacttctttatctagggacatgactcttcacatgagtaacttcttcacttatctaatttattattattaaagtttaatccaacataaaaaacttaaccaggatcgtgatggcaattaagagttgatgagagttataacttacagtgtcaaatttccggaaaggcgaatccactttaggcgcaaacttggattcccaaactgaatcagcaatcacaatccccatgttaggcactgcacacaaatcaaagaaagaatatgcatacatgttagaaattatgatggcggtataaatagaaactgctgctgagttatatcattatctactacatccacttcaatcctaatatctggatattaatttaccatgtataacccatagttctggatacagttcctttatagttttctcgagtatctctgaaagcgtatacttcgaccaagtccctgtgcccagttctgctatgcattgcaaaattttgtttgtcagctctaccacagagtcgtcgccactcacgtctatttcttctataaaaggcaaaagcttttcggcttccctaacatgataccttggtacatttttcaaatgagttttcatccgatctataatggcttctctacgatccgatggtggtgcaggctcattgatctcgtccatttcttctgttcctctcagcctcagcaagtttacaaactaatcactttcgtttttgctcaagagacaaataacttgcttctacctaataccatacaattatcatacgcgttgaattcaacgtgtcaataaacaaagcttccacgcaattcttaaaataatcagtattacgtcacagttttagaaagtagattcgttttccactgtaattgccttttaaaattgaaagtcggtgtgattgaaagcaatgcatacgcatttttctagtaccacctgtaataaaatacattatcattcttgtccacaatttcacggcgattacttggttctagaaaatccggtttgactttgaattttcggtgcagcaaaacgcggtttcatattgaattttgccatcaacgccacgatagcattggaaaaaattgttttgagaaggtttgtttggtttcagacttccacatatcatcaagttgggcaaaatttgcagtacgagagagttggattcttgcgctgggaactactaccacatcatgtatgtgcttaatttctcctcggtaagttgtatatgcttacttctatgctgaactgcatcatgatcaaactccattcaactcaaacaaacagagctaatttgttcttaatcattcttatttattattgtttttcacatcgattttgaggaatctgaatatgccaaagtcagcaattgacatatgtatagctatattaattatattttgcagtttgaattgataaatcttgttgcgaaaatcttgaaccaaatgcagaatatccatgaaaaggacgagcatggagatgatgagaagggcaagcttgtatcatcaggacaaatgaatagtttaagtgtaagtttccttcttttcccctctaaactatttaagttataattattgcagaaatgtaatgccattatatatgctaatattaattaaactgcagagcaatcaagatgttcgaatgttagacttagttagagccatgtgcactgtccatgatgagaaatcaggacaacctttagcttttgactacacattgaacacaattatgcattttgttctggttgtgtgtgtagtgagttatttaaccaagcaataatatatggtgtgatgtatattatgtgtattccaggggaaagaggatagtagaacattaatgaacattagttctgaagttgaaagaagaacaccagaaatcatgggaagatgtgcatcgcggtgggccaccaccacattttttgtgtgtttgtgttcattaaaattcagcaagtcacttctccaaccacaaggacccaattcactatggcttgccatagtaggaatgttcagttttactgtgtttgtatctgcttcttctgttatgcttgttgttggagcacagttcactcgtatgcagataactgttatgctcagtgctttcttggctatgctgttagcttaattagacagttttcatatatgtacttatgttttcttcaggcagggttgaacacaagttgttgtaatcttgttatttacataatttcattcgcataacaataaaacaaagtttgggtatgtgatatatcaatatcaacgattaaccagacagaacatattcacggatgaacattgaatggcaggaacatcccttcgtatgaacttttctttctttttcttttaaattcatttagaatcttatgaagcataagcataatacaataagatattaaaagaataacaccagaaatcacaggaagatatgcatcgtggttggcaaccccggatttttttggtgcttgttgtcattaaaattaggcaaaccacttctccaaccacaaggacctcattcactatggcttgccatcctaggaatgttcagttttcctttgtttgtgtcagcggtttctgttatgcttgttgttggagcacgcttcactctgatgcaaataactgttattgtcgttgccttcttcgatctgctgttagcttaatcacgttttcatatatcccttccttaacccactagaatatagtgatggatagggaattgtataatttgcatgggattgctttcaattctataggctgcgtggacacatgagatatatgtttcttgtattttgttatttacagagtttcattagcataccaatgttttatgtatgtgatatatcacaagactccacttgaagttgtttaaggggataagtctagtaccactcgaaccaatgacttaaacctttagtttcataaaagaacattttgtaaatagagaggagttggaagagagttttttggggtcaaagataacatgtgtcttccaatccaccaggtcaaagactaatctagctcatgatgtgtgactatctctaatctaatgaaattttgcacaagataaaaatcaaacaagaattttcctattaaatagatcgttcatctaaacaaagtgaaatctgataccattatgtcaattcttttggatttaaagaaagttttgtttacctaaaaaaaagatgaccaaaaaaaagttttgtttagatagttttaattttttttataatttaattgtatatgttatcaatgtaaaacttttttatataaatatttaataaaaattactttatcaccatatcatattaacatataagaagatatgataaaatattaacatgatccaattttattaaaaagtagtgtgattaaaaaaaagagtttgttttttttcaaatgctaatccaaatcgcattatgtaactgtgtcactgatccaaaaaaaattgcgatagaaatcaaatacgattctttttttcattaaataaattgttctcgtaaatagaatcggacattatactgttaactctaaaatcagttggctttaacttcctacacccgtctaagttctaatacactcagcagttatttgtgaaataccacaaacaacctttgttaaaataataaaacataagcgttttgattttttctaatacaccagtcctttgcgcttctgcttcttctgcttgcttccgcttctgcttctgaaacctgcgattttggttctccgccgcattcaactgtgtgcgctgttgttctctcgggctgtggacggagcttgtggtggtcggcgacgaagctgttctggttttccggcgcgacgaggtaagtgactggctttgagtcgtttcgtcttcggttttgcatgcgtacggattgctaatccataacctctgaagtctatggattatggatccgtattaaggattgaaaatccgtaagcctcatacggatcaccaatccgtaataagaaatttgaaatccgtaggagccttacggattgccaatccgtatcatccttacggattgacaatccgtaaggctcctacggatttatgatccgtaagtcacttacggattgacgatccgtaaggcttacggattgccaatccgtgtgggtatttgcttaattttttactttttgaaattagaagattaagtgtgatgactgaggtattttttacatgaatgaactgaactggtttaatgaattgctatgtgaaaagcatgttgtctaatggtatggacttgctagattgtttttgatcctccacatgcctatggtttgatccacatttgctaggtaatttgaaatgtcatgagcaaaatgaactgtttaaatgaatttttatgtgaaaaccatgttgtctaatggtatggacttggtagattgttgttgatccacatggctatggtttgatccaagattgtttttgaattgttatgagtaaagtgaactgttttattcaattgttatttgaaaagcatgttgtctaatggtatggacctggtagttggtttctgatccacatgggtatgttttaatccagttgttaggtatcgttatatgaaaggtttattttgatcaagatggctatcattgtaatgtaactacttggtttcaatgtcagattatggttaggactataggattacgacgggctttggcaagagatgccagtgaagaagaaccccctgtacgacgaaggcctacagcatcagcacggagacaacgcgagaggactagtgttgttgaggacgtgcctgctgaggtagtcgctgacgatgttgaccttgtggcacctgttgaggctgaggtcgagggaagcatcgatgcctctgatgttgagggatttcctagtgggccccaggacacttcaatgttgactcattacgctgaacatattgcacctagggtttggaggggagaggtacgattgtatttaaatatagttcaatttgagaattattttggcgttagggtgtgcctatgtaactaaccactaataaaggaacggcctgagcttaagttggcatctcatgggaggaaggttcataaattttgcaggcctgcgcctgagattgaaggcttggtcgcaggtaccggattgagcccactgatcacctgttcagtatatacacgtgaccggggtttaatcttgacatttgtggagaggtggcataaggagactagcagtttccatttgccgattggagagatgacgatcacgctggacgacgtcgcatctctatttcatctaccagtcatgggtgcgttgcagacgtacgacctttttgacgttgatgacgcgattgcattacttgtcgagttgcttgaggttacccccgaccaggctgcagcagatacgaaacaatgccatggagcttatgttcgtctgtcatggttgcgggagttgtatgctgagaggtgtgaccagatgcagtggacggtggcagccagagcttatttgctccattcggtggcagtcagagctcctcctaccgtgccagatcccgcacctgatccaccagctagtatggcagagcccacggttgacactggagatgtttaaacttttatgattataatgacctcttacacagttttgtgtaaatttttgcagggcgcatgccaaaccattgctgagaggttacagaggctggcaccgatgcgttcaacattgttgatcactgtatcacattagcacgcagtgtatgcgatgattggagagtttatgtgcggtcgagacgtcctaggagtggaccgcggcgagattgatttgttacattatttgtacttcactatataaacatatgtattaacattattatattgttgaaacatttcatttcatatatgtacatggtttgaacatttcatgttggttatatgtacatggtttgaacattatccatctgcgtcagtattttctttagtgaaataaaataatccggcagttatacaattacataaccccctgtcatataggtgaacacacatgaaaatactacataaagtcacaatgtatgccagtgttgagaccccctatcacagcccagatcaacataagctggtggcaatgttgtcaagctggtatacttctgcattcgactaatgtaactagctggccactcttttgcttgaggtcgacaatgtcttgaccataataaagcaattggtggcataggacagccgtcttgcaatgcaagctgaacaaaatgatcacaattacattgatttgttaatgacatgtgttataatcatctacacaaattaaagcattttgatattacctgtacaaaatggtttccatacacatgcccaataaaaatgacgcgatgtacaacagaatctctaggcggttgggttctaagggggaaaaaggtcatactttgttgcttagaaagacaaacaatgacaaggttataacgtgaaacaattacataacccatgtcagtgatgtccatccatttatcctgtgttacctacaattttacaaatacatcagtatcattatgggcaatttaacaaatactaaattatggaatgcaaatgtaaacaacgaaaaaaacccttacatgtccaattgcttctaccaaaagtgacctctttaactcctcatatctttgtgaccccccgaataaggaaatatagtcttcagaccattctgcaagttcctttaacaaatggttgcgtaccacagcccacgcatcttcaccataaccaagtaaacccgtaatggcacgatagccacagttaccgtcagctttaacatcacatactccaataatataattcctcatgcatggcaaaaattgatctagcattgggatattatccatcggtatacgttctgaagactttgatgcagacttttgttgagatgagcaactatcatgctgagaatataacgcatccacatattcaaaatacgatggatcccgctttgttgatctttcgtgtttggtgacatttttcttttaagcccctttagtcttgaccttttcaggaggaggacacgtggaattttgttcaggatatgctatttctttgagtttagtctttaaagttatcttgccacacatgtcaagactttcgaatcgcttagaaattgcttccatctcgtcagttatcgtcacttcacagtcatcaacaccttggtcagagaaatgtaacctccgccaaaacaggtgtacggtacctagtggtatgctggaaagattatatttggctagctcacatgcacaaggcaaaccacacgtgcttctcaacacacatccacaacgagaactatcaacgcctgtataatgaactcggtcaaactcctcagcaattagatttaacgcataccttgaaaccatgtcgacgagtcttttgtacaatgtcactttaaagaaatgtcctacgacatgcgtgcttgtctcaaaagaagcctttatttcattgtgttgcaaggtgatcatgttgttcatggcatcccaaacattacacagatctccaacgctattttgcagtagccgctttaaggtccaatgagcagactcaaccctgcagtgacaggtgcacaaatattaatttggaaaacataatgtcattcaggtattgcaaaggatttacctgtttgtagtagtgtttccaagatgcatgactttattggtccatgctgtaacaaatctttctttgtggggaatcaaccatgtgtctttaacatactgaacaaatattggccacggtgcgcagacacactcaaacttcataagagagtcttcatatgcattttcataagcacaatctacaagacttccccacgcttccatgacatagtctcatgcattgttttgtcccacaagtgttttacacttagccttcacattcttgtctatgtggaatcgacacaacaagttggtagtgtcagggaaaacctgttggatgacattcatcagggctgcatctctgtctgtgacaatgactttaggaaaaaccccagtctttaaaaaaaggcctctaaatcgttcaagcgcccatacaacattattgatacgttcaccctccaaatatgcataagcagctaaaaatgtcatcccagtaggtgtcacatcaacaatgtccaacaaagggagtttatacctgttcgtcttgtatgtgctgtcaatgagaaataccaaattacatgaatttatcaacttcactgaatcagggtgtgcccagaatatgtctcgtagaacatcttcatccttcagtctatgccaatggatatatctatcgcgttcaagaagcttcattaattgttgcatttcagtattgttgcctctaacagaagtatggtacacatatcttgcattgtatacttgtttcattgttgtataactttcgggattgcgatcttttaaactcagcataatattctttggtttaactcttgactttgtcatttcagcaacaattaccttctcttccgcacttaaacgaccagcataaggatgtccagtcattgacttggccaaacgatgattatgtaacccacatactaacttcaccatccagccttcaccttctacgactggtttccctcgcaacttgaaaggacaaccacactttctcgtgctagtggtcgtccgtactaaatctttgttgaatgctctatactttccactcctttcacaacctatcaaaagaaatgacgttcttcccctctttccagttgttgtgtccgaccgcacaaccacagctacaaatccaatctcatatgcaacactacgagcccattccaataactcctcacgacacttaaacacctacaaaatttgataatacatattttaacagtggttaagtattaaatttactagcttttatgaaatataacatgatacaaatacatttgaagtattgaatgcctccgaacaatcaattgaagagttattattgtcacacgcaaatggttcccctcctttgtcttcactcatattcatttcttgatacatcatatcatcatgcatcaaattatcattatccatcttaacaaacaaattaaaaaataaccatcattaaaaaactaaatgcataataattatactcacaaaaatatcctacaaaaaatattaacttctcaatatatttttaaatgaaaataggacaaatacattaattaataatacttttaacttattaattttttttttaaataaactaccatacggattgacaatccgtaagaatcgtacggattggcaatccgtaaggattatacggattggcaatccttatgatccttacggattagcaatccgaatgatccttacggattgacaatccgtatgatccttacggattgacaatccgtaaggttcgtacgtattgctaatctgtaaggattgtacggattgctaattcgtatcacctttacggattgccaattcgtacaatccttacggatcgccaatccgtacgatcattacggattgccaatcaaacctccgacgacacagaacgtacctccgacgacgccagcgaccaacggaacctccaccaatgctaaccaccacttccgcactcccgacgaagcgacaccaagcgccaacaaataaccacagcagttcacgaacagcgaagaagaagaatgaagaaaacgcatggccatactgcctggtgtatgttaaaaagcaacgaggggtattatcggtatttgatgaaaatgctgggtgtattagaaatccactggtgtaggaagttaatgccaaatcagtttgtccttaaagaaaaaaaaataggataaccaaaaaggctaggaaatgaaagcccaatgcttctgtaacaaaccatttcgtgtttttgtatcgtctctggcattctacatccactctgtaacacaccacacaacaatggcttccagttccaaatcggagacttttctgcagcgatacggttacgattttctgttgggatcaatcgctgctgtctacgtactcactgtgccttacactaaggtcgaagtgagtttcaacgttcaggtctctctcttcatgttcttcgacctctttgcttatttttattagttatttatttatttataaattataattcctttgtgtttcctaaaggcaatgcacgatattctctatcacaggctgaacttagacaatgtgagttcactgtgtctttgctatttttttttattagttggtagtttattgtattttgtatataattgatcttttttttaatgtttctgttaatagtatgatcatctggaattccctggggtggttcctcggactttcctcggtatgtttttgtgttttgtagccattttctctcaaaatatgtatttttatttaagcttattatgtttttcatatctttaatatatctctttttcagtttactacctaaaaatatttttttatgattttactatatgacatatccaaacttttgcttttgacacctactgttagtcttggtccgttaagtgatgatgtgacaagctaacacactagcaaatcatcagttaagtgatgatctgataagttaacagagtgccacatcatcacttaacaaacttagagtaatggtaagtaccaaccaaaagtaaaagttaaaaaatgtcagatagtaaaataaataaataaataaataattaggtagtaaactaaaaaaggagtatattacaagtatgaaaaacatatttaagccttttatttattcatttagtttttggttggttaatgttttcatatttagcactagtcagacctttgtgatttattatggaaaatgataatttactttgtgaagaattaagcaaataaggattacggactcgtttatacagttgcattgcttgtgtgcctgatgtgaatgaatccatttaataatctttgagattgtgatttgttactagcatgtgttacaacggttattcaagtttgtttagtttttagtgctgaaaatttaaccgtgatgatgttctattggattgttaagattgagtttttgcaattgccatgcctcctcggtataagctggtgtgtgctagtgtagtacttctctctttgttatattgttatatgttatctaattgtgttattgttattttgttttaggtgctttgcttgtgtcaataattgcatcaccgtttgtgttaactgcaagtttgctgcatttgccaaagttttatgctcttctcataggtatgtatccttctttcatacttgacaatcaatgcatattttagttttctcatttcagggaatgctgtttttatcttttttccacctctggatttctcctttcctttggtttaaattgaaaaaaagactggcgctaattatcaaacgtgtgtatgtttttttgttgggaggagcccgacatacatatgccaagaagtatctagttattacttttttgtttggatgcctgggaatttgtgcagataagaaaccaatcacactagaagtttaaagtctaaaacactgtctttagctggaaaataatagctcgaaaattaattctgataacgtacttgtttcactgagccattggtctctatgttgttttgcatcattctgttagtctcacaagcattcattacaggaaaatgtctgtgttatatgtacataattgaattgactgatcaaatcaaatcaaataatctacaattcatagttgagtttaaagtcatgaaccatgtcaaaatgctcaaaattaccaagatgatggtgtcaatttttgttctcctgtgctctctgttacttgagaatgtggtgggaagtacattaatatgtcttattgttgatgtctagttcggatggccctagggggcataatactatacacactaagattctttcgacatcaggtatgtacctttggatcatcaataagcaagtaaatttatttatttattttacttatttatgaagtagtgtttattcaactctctgatattatacctatccaattatagctcaagtttcaatacttctatcatgcacccttgcatatttggtgcatggatgcacaaagatgtttaactgttgatcatataataataataataataataataataataataataataattaggagatataaggttggttgggtggttgggtgaaaagttccaagaagggtgggagggagaggtcgtgggtttgaatccctccgctaacaaaaacactaacatttaacaattaactttggccgatcaaaaaaaaaaaatatataaaaataataattcaacagtagagataatttatctcaatttatttggcatgtagtactataatttttaaaattatgaaaatgttctctaaagactaaagcttggccagagcttcagtgagcttcatgactatgaaaaggagattaatcaaacaaaggagtaaattggaaaatttagtgaggaactcaaatgtggttgaaccttctcaagcaatggcaaaaatcttggttatgtaggttgcatatcttttatcagtgtttgttcaagctgtcctaattcatctctataaaacttaactaggtatcccaatggaatggttcatgtgcctccttcaccttgtcgtgcctgccagcattggaaaatctttgagcattgattaaaactttggaaaaggataaaactggtaaaatattgagggaaaaaaaatctgaagggtgcatagcaccaaatttgagagcattgtagaattgttccaagtttcataaggaagcaacaaactttcttttcttatcattctaataatgatctttgctttgcagataatgaataagtttggacatcaagtagaatccttctttgtgatattaactgccactcagtttcattttctgttctattgtacccgtccacttcccaacattcttgctctgggcctaggtaaactacctagcttgttttgcttcttagaatttagtgtgatttttttgggtacatattttagcattgttttattgtcaataagtttgaaatgaaatgattttatgaaagtgattttttttttttaaatatttctactagtttaatatatcatcttttcacacacacacacaatttgacaggaaggaatgttttctggaattagttggcccttttaattcttaatctcgtagtatgcttgatatgctttgcgttttctgaaataatctctttgcacatttggattatcagatttttgctacaactgtactcagacgtgacatgctgttacttctttgcccaattgcgctgcaacttcttttggtatgttgtgcttttctgtaacttgcttttcatttttccgttttctccttaaattcttgagctactgatagagagcttgtcaattctttaatttgtttgtttggaggatatctaatgagttatgatgcttccccctacttgtacctgttcttgttttcctgataaactttcttctatatatttaaaaagaaaaggaaaaaatctggctattctcattattttctgttggatgcttgaatttcttattttccaacaccactccgtgtctattaaactattgcatcctattctttatcttgcttttaataatgttatgctttaagtttgacaagtggagcaagtatttcctggtactggaatatatttattccatatcaactttggacttctgatgtcaatatcttgacaatgaaatgaagatcacgttttctgtaaaaacagataagaaattctattaaactgtcctggcaacagttctaatattgatatcatcatttcccagcataatttaaattaaatctgatttggacttttatcttatcgaatatttcaatttaaaggcttccaatttatgagttggttattattgcattcatgagaatttttgttgctttgtcactatctcagaggctataggatgattggcaaatgtgtttacaatgttcaattgggtcaccttagaatccctatgtttgaaagccacctaaatttcattattgcattcatgaaaatttttgttgctttgtcactatctcagaggctataggatgattggcaaatgtgtttgaaatgttcagttgggtcaccatagaatccctttgctggaaagccgccttaatttcattgttgcattcatgagaatttttgttgctttgccactatctcagacactataggatgattggaaaatgtgtttgaaatgttcaattgggtcacaccatagaatccctatgttggaaagctgccttaatttcagtcacccctagcccaccttaattgcggcctctttgggcttaattgtagccttgttgggggggggggggggggagggggtttgactagagatatgacttaaagagagctcataaagcttgagcaatcctcattttacaaaccggttttgtgggtttgagttaagccttaaacccaaattataaatttataagatggtattagatcctatctaaaatccaatttttggccaacccacatttgtccatgctccagccagatgtgtcttgggtgttattattgcaatacttgcatttgtccatgctctagatggccaatcttgggcatgaggagtgtgttggatagctgccttaattgtggcgactcatcacctgccttaattgtggtggcctggcctctttgggctaccttaattgtggcctcaaggggatttgcttagtctcacattgactaaagatatggttataaagcttggacaatcctcatcttacaagttagttttgtaaggttgagttggactctacgccaatttgtaagaccctattgactctgtgtaattttatttaacatttaaatatagaacttcattgcgttaactatgcttgtgttgttatcaatctttggagaacaatgtttattatatttggcatttgaagttaatagtgctgtttgatgaattttcttatcctcattttctgaccttgcaattttggttgaattattataaaattgaaatttattctgaaaattccatacttatggtaacatgatcatgctttgtgcctaataaaattaatctcttgaagggaagacaaaaaaatttcagtgtgaggtgctctaaaacactgcactgggatggctctcttctgcataggttggtgacatgcttttttactttaattttaatctgttatttaaataaaatcaaaattttatgcatgataggatgctattgttaaaatttgattctaacattatggtcttctgattacaggtctaaccatattggttgattcaattatgtggaaaagattattgtggcctgaatttgaagtcttttggtttaactctgtactaaacaagagctctgaatggggagtatccttcaccaagtttatattgaacaactgtatagctgtagaatatgaatacatgttacacatatttcatgaccagggtattttttaattttcttctggttctgtttggtttgaaaaaacatttttttttcacaaataattacaaaattcccaccttgttttcactttctttcctgtttccaaagatttgtttgtacaggaaaaaagagaacaaaaacccattttcactatttcttgtataaatcattgaaaacaggaaacaaagtgaaaatagaggtattaatttgtgaaagcatataacattttaaccaaacaggcctttgtttgctaccttatatattacttaatgatgaggctaagccctagtttagcaaggagttacaagagaagatcttttgtcacatttcttgttcactttagaggttgatgttctgaattgaatactttcagttgctgtttgtggatagggatatcattcaggatattagagttggtttaccatttagagtaggttatggtcttcatcttaagtttgctgatgatacaattctctttttagaggataatgatacctttggggatgttctttctattaaatttttggaagttatctttgatttttttaggttaacttttctaagagtgatttggctggtttagttgtggatagctatgttctttcttccattgcttctctggatgttgtgaaactttacagtgccccttcacatacttgggagttcctttgggtggtaatccttttctacttcttttggaaatcctgggattactcagatctttgttttgaagtgaatactaaattgtatctaattctgtgcgggctgactttgaaagccatttatttatgtgacccaagaaggactgttcaaagagatttgtacttcctttgtctcttggggtattttagtttgtttcaaaatatttgtcacttttagaaaatcaggaaagcattatttacttttttccatcaataccctcatttgagtatctcacctaaatacttgaatgaattcacaaatacattctaactctttaaataagggtagttttctcaaagaaagttctcttatcatagtaatttgtgtaactaataattttcttaagtgtgcattaccttaaaagacataatatgaaaaagaggaattagtaaatttctgcaacaagaggattttggtgtatttggattagggtttgtatgtttagtggctaatacttgtattcttcattgtttgtgaattttctctctgttttgcccatggacaaggcacactaagcttattatcatgacatcatctaaaatctgcatattgtctgttgtctttgataacactgtcctatttgtatttgataaattattatgttccttgacaatatttttgttagacacatgctttccattggtacttcacctcagctcttcctcgttcactactttgctgcatatccactgtcactagtaagtcttggagtagcaaatatcctctaacgattgcattgttacttatgtagatttctagcctgacatggtggtttcttccacttccagtgatctggttaatattttgtaacttaaatatgaaatattttgggttttctccttaaaattgaactgtctgaaggtagtacttaaagattaatattttccctatgattaatgtttgaactatgaattaatattgaatatttcttacctgttagctttggaaagtatttgttccctatttgttttcagccagttcaaatatcttaaatgcatacacttagtcatgtatattcttgcaaacccagtttaaaagataataggatatttggattgtggatagtcatggtatattgcactgaagaccaggctagttgcatgattagtgtagctgttgccctttaagaatttatcataactattttttggagaaggaaagaaagaaattattttagattaggagaattatatacacatgacaattaaaattgtagtgttatttcattcacattattggtttaacatttcattcctgttattgggatctcatgtgtaaaggattttttttaattatttaacaattagcatactgatgcacaatgaagcagcattaaggattgttttgttatttacaattttttatgttattctcataaagttttgaatttccttttatttgagagatgtcttactattttagggattttattcttcgttatatgttcttaagaaaatattgtcatgttaaaatttcctgtttaatttaattcgattaggatttaacatttaaggaaattaatactatttaattaggatatagctaggattttatattaaactgggattttcataatctttgggttataaacatcttttgagtttcttatgttattgggacttcattataatacaatatttagattttcatttctttagagaatatttttaaactcaaccagaggagtactcttgccttagatgagttctttaggtgaattgatccacatcacatgccctacagtatacgccactttttctaccttcctcgaacggaaaaaaaataaaataaataaaggaaaagaactgggaaaagaaaaagaagaaagcctccttttcatcctaacttcagttacttgacaattgacatgctgtcttctttgcagtttggcctttttgtagacagaagggtacgatctttcacttttccagttcttgccttcattttgctttattctaagcttccccacaaggtattacaattataatctgaaatccattgcctaatatgtgtttgtcttcaaattgaaacatgtatttagtattattctgattgatattgcaggagcttcgttttatcataagttcaattccaatatttaacttgtctgcttcaattgcttctaacagaatgtaagttctcctaatgagtattgtataggtctgtcaaaactaattgggttctatttgtattttctgaaattatttcattgtgtggtcttatatactaaaatacaactgaaaatggatattctgggaaagcaagtaattgagattttaaaatatgaatgaatgtgcattcctttccttgtttctatggcatttgtatgcattctttctcatttgttattggattatgaacaacattcataacttttttaatgtttatcaacctatgttttggcttcaagaatgatgatgctgtgtaacctgtaaggaagctcattagaccaagggattaagttttgctctcaaatgacaatacttgattatgagcacctgagtggcagttgcccctgctttatttcagacaaacttgaattttgcctttaggatgttcatataaggttgttgcgtcgtctatcaaggatcatgcatattccttgtttaaattatgtaaaatctggattgagaagcatgttttcttcatcatccttagtaactcaaaaaattgaataacttgcagttgctaaaatatattggagcaagttagttcaatatacaaagcatcttcctcagtcttctgtatatatttgttcttttgaactgggcttaatttagtgctccaataatacaatatgggttgtgctctaataatacaatatggctgttctattcattttgttagtcaccaagtgaatgcaacaagacagtttgcttcattgcatatttgcatgatccaaaggtatttgacattttgatggcattcataacattaggtggtttgaggtcatttacatgaaatttcattttgcttattggtattttagttttaaattcaatatgacaacagaaaaaaaaaacctttaacctactagttttaaattcaatatgacaacagaaacaacccttaacctactgggtggtgaggcaatatagtatgaatacattattatgatcattgtcatctagtcattttgatacaggaaaaagttaactagatataaaaacaattggaataatgaatacaatatatccatctaaactagaaatctaaaatcaatcgagaataggaaagagaaagctacgaatgccgcacagctgctcaatttctccatgcatgacacaaactgttgaagttgaagctgctgtgacttgtgagttgagtgccaaaaatttgaagagtggtgattgagctgaactgtatttacaatctaaacagagatatgaaaatttcggatgtttccctctttgaagaattatatctgtttccttttaagttactaaccttcagtaatatgcatcctgaaatgtttcagttacaacaataagaaaaagatgatttggaacttgcttttcctcattttgctgggattacttttaatgaggttagttcaccagtcattttgtttcattgtgatggatatatcattctttttttgtacttactctctatcttgcagtcttgcaggcaccatcacaagtttcatggcatcatactggaactatcctagtggccatgccttaaaagaattgcatggaattggtgacatgagtcctgaaatatccatgcattggctgcaaagtattttgctagaattttcctagagagaaattgctccttgtttgtcccttaatttggtaaaatattggagttgcagtttctatttttcataatcaacagtggcaagttctgttaaaattgttaatactgttgtacccgttggatctctgatgaattactaaacatcaatttttaatatggttcagtactgagcttgatattttggtcatggtttattgttctaaaaattatttccatgcgtgctatctgagtgttcatattgtatgacattctggattaaattaaatgatatgtgactttggggtgctttattgaatgatgtgttgtatgacattctctgaatctcttacccgcattccaaaaatatatctcaattctcaaaaggatttctagaataaactacattattcttcgggcagtggtttgagcaaaaaaattagcatgtcataataaatatgccaaagctttccctttggtgtatagagctgatgagttattttttggctaatttatgcatggaccaatatgcagtactggttggtactggtccttctgattttctcaaggacaatacaaaggcagtgtttgtgatactcatacttattgtgatagccaagatgcattccgtattgtctccaatcctgttttctaggggatgaccaagcacaatgatgtagattgtcactctatcagaaaagatccattctggagacatcataacttgttttgtcaatccaaatgctcaccaatcttttgagtggaccttggacaagttgttacttgttatctttgtaaactaactagctctggtcacatatgagctaatgcactagctttgacaggttgtgttaggaaaatttattattctttgtccattgaatggcaactagaaagattgtatcttttttattattacatttatcatataaatatggagtctgtggtgtagttgaaacacatttatggacatatgcttttgttctctctctacatgaacaatgtgtcaattttgggaaaagataaaggattgtgcttattgttactgtggtattaaggaagatttttctttagtgtttggttgatgtaacaggatttgttttgatttgtacgatttcttttcttgcccagaattcctttcatctgaattagaagtcaaatgagcagtcattgagacctaaaacaactccaatccagaaaaccccctcatttaaattataaaaagaaggaatttcattaaaagataaaaaaaattgaccactaaagaagatagtaatgtaacgagttttttatttttatctttttgatactgccccttgtcattacttttgttgtctttgttttgattttttaattgttgcaggctttcataatgatacagatgaacggtgggtgcacattgatacattttcagccatgaatggaatatctcgcttttgtgaaagtgaatttccatggaggtatattttgcttctttcttttgcagtgtactaatgatgcatcgagttgttaattgcatgcatccaaacaaacctttctttgactaaatctcatttttaaggatcaatgaaggcatgcctttctgagcctttatgatttagtatccgcagaaagttacagttctctaatttcattgtcctaattggaatgagatcaactatatttattttcagaagttatgttttcaaaagttatcatcagaatcgcttctttggctgagtaataaattgatggaaagaaaaacttcatcgaattcttcgacttgcacattcacatttttcggttccctccttcaaataaaggagagtgtatttgtttatttgattttatcgttccacagtaaactgtaattcagtgtgacataggtaccttcctgttgggaaggcaggtagtgtaggccttgataatgttgctgaagtatgtaatctgctttctcaagtccagcttttcttagtagagtgacaaggagctggagctatttatagggttagtgaatgtgtttcttattgcactaatgaaaaaacaataaaccttgatctattttcctgatgctcaccatattcaggttcattagttagcaccttatctctagttgaactgagccatcattgtggttatagggtttacatttttaaacatctacttgtaagtgtcttctataccattcgttgctgtagcaagttcaatactggatttgctacatggtctgtaatgtccacatggaaaaattgtggaaaaactacatatacaaatgattaaataagaatgagatagcatgttatggggaagatttaagtgttaacgaggtattagcagtatactactcattatttctgcattcattgctttcaatttccaacgatcagactatgctgctttccactattttccgactacaagtaacttatatactattctctggtgcaggtattctaaagaagaacaaattagcttgcaagaatttcagcagagaaattttacttttcttataaagtgagttatccattaacatattgctagtgttatttctagtactttgtctgacaaacatataaattgtatagcacatgctaatgtttatgatgagagatcctacaaactgttgacagaaagtgtcggtattgtcaaatgagtaagtgtgtgaaataaaattgataaacttcgtacattagatccgcccgcgaagagtacgtgttgttggcaaggactgcatcagcatgtgttgtgtgcataatgatcattgccatgaaccaactatcctaaaaactttagttttaagtgaagacaaatgaatagttttatattacatctcaactacacccattcatgcaaaatcccttatggcttgaagcgtggatagtgtgcaagcccaccaaccttgagctgaaatttaacttcttattacaagaataggtgcagcacagattgaaatctagaacaataagtcatagaatctttgttaccatgtaatggacctactatcccaaaagcttgagctattaaagtgaagacgcatgatgattttatattacatctctaactgccaacaatttggcatttggattaataaatgttgtttgacttctgtcatctttctgtgcttgcgtgtgtagaatgtgagagagaggaaatgaatgtttggagatattgaattggaagtttatatatgatttttcacatgtaaactcactgactaattgcaagaaatttccccctttatctgacctccttgttgatctttaacagtgaacatcctgcgatcaatggattcaagtgcctatttactgaagatggattttcaagagtccgcctgaaacctggctatccaccaattttactggttggtaattgctattagtattcccaacccgctcacacaatgaatgctttgttcattccaaaggtattgatctctgttttgaccctttgcaggttaaagagcccaaagtgtatgttcatggaaatttagaaaaccagaatgcctggctgcccataatgctgtctggtatgatagtctatttgaaatcatcaggtttgaagggtgtgggtatcctttgccacccttttttcttagttaccaatgaggaaggagtggggagcaagtgaaagttggtattgtgacccctactaacagttctgtgttgtcagtattaaagagaatgtacatatgggtacaatagcttttcttgtagcatttcaaatattgattctcggatgtagctatatctgatttttgaattacatatagaagattcggttcaacaatctggtttgggataacttcatttatgctggaaaactttttggctcaattctggtaattggtgtatactgtttgctagagcagaactttctgtattaactagcatgatactcgtgtttttgcacgttttataggcaaataaataatataatatatattcaaattattaaggtcattattaataaggaatcaatgtttaagtggtgagtatattatttacaaaagtctaaaatataattttttttagtcagatataacaataagataaaaacctttgggtaggaaaaaatacaatgtgaatttgtatattagaaaaacatttttttagtgcaatattaaaaaaacatgataccatgcaaaataggagtagtcatatatggagtttaaatacagtatagattaataactatgataatgtttatacttttaaactgttttgtatatattaatataatgttatatgataaattgatttaataagacatcctaacatatcaaagtgaattttgtttaaatttagaaaatataaaataaaatgagagacttataaaagtgtattttaatcttattcaaatatataattatttaacacaaaaagatgttgaaaaagcaaaaaaaaaatgtataagttgtgaataaaaatgacaatagagcagggtgtggatggacatgtcttttgtctccattaggggttgggtatacccaccatgtccccgtcctcactgattcataatataaattcagtccattacaataaaaaaaatattttgagcacaattattcaaaactaaacatgcaggaggtattaaagagatttacctaagttggttaaacacgtgaatgttataaatcctatggcaccatattcaattcctgctcttcaaaaaaaaaaatgcagtaggcgttaagaccattaaattgagagttattccatatatataatgctatgatgctattaatagaactattaaatattttaaattattgattaatatattcttatttttaatttaatattcattcctcacagaactaagatgagtctccttcgaattcatttctctccgcataaacaaaccctcctttctctctctttctcatcttccctctcccattcctcctcactccacttcaaccactcttcttcttcttcttcctccaccactctcacccatgctcacacgtgcttccacccccgcctctcggtgctcacgtgctccatctccaggatccacagctacggcaccgtcgactacgagcgccgccctatcgtgcgctggaacgacgtctaccgcaggatatctctccaccaaaacccccaagtgggttccgcaaaggtgctcaaccaatgggagaacgaagggaggcacctcaccaagtgggagctttcaagagtcgttaaagagttgaggaagtacaagagatttcgaagagccctcgaggtcaaaattctatctttaaaacctttttttttttatcatttgggtctctttttttgtgtgtttttgctttttactgatgaaatgctgatatatatctgtctgtttttggataaacttttctctataagtactttttttaggagaagaaaataagaatgaaagattaattaacttctccaataagttaacttatttagctcatgcatagcatggttttgaattgccgttgcggtcgtgttgtgtgagccagaaaacctttacatcactggaaaatgcggctcatgcggcggtcgtgatgcgattgcggagtgacaaaataccttgacgttgcggaccgtaatttaaaaccatgtgcataggtgaaaaaacacctttttggagaagctaaatgaagagagcttctacaaattaatttgtgctaagctaatatgagcttaaaaggagaagctaatttgtttttcttctcctaaaagtgcttatggagaagtttatccagacagtctagagtttttttcttcttcataaaactaattatttggtcccaggttttaaattgtggtcatggttttgttgtgattattgatattgtgagaatttgcagttgaggaaattccaaaaaacattaacgattccagtgtggcaaaaactgcggttgtggaccgtattttaaaccttgatttggtcccatagttgacatttaggaataaataagaattagccctgtatcagaaaaactaagggtctgtttggtagggtggaacgataacaagggagaaatttgaattttgtttgtagaggttttttccctttcctttttctctccaaccaaacaaatagacgaacataccattaggggttgaagtagaaatagcaattaaattcttagaggaaatacagggtctggtcatacgtcctgaacagcaggatagttggaggtgggagggggccccgaatggtaaatacacggtgggaagtgcctacaagctattaaatagggtgttcactacccagggtcaggaggaggtgtacaaagatctttggaagctacgcatcccttgcaaagcctcttttgcttggaggttactgagagactgtttgccaactaagaggcatttggtttcatcgcaacatgattgttttccataatgaaagctttaatgggtctaagctgttggaggacgctttattcttttgctggtcctggttagcgaatctggagaaaggatttaatattcctttctaccattggtctagtcatattagagatggtttttgttagtaggggaggttttcattttggctctttttgttttctttcggccaacagggtgagcttttcctagggtgacctttggcaccttaattcgagttgttaccatgtattaggttgccaaagctctgctaggcttactgttagctttttctttttcagtactacttgtactcctttctcatatatataatatattatctttgctgatcaaaaaaaaaatcaggggttgaaatatgatgtttaaaaacattttttttttggcactgcaggggctggggggattctcaatttgtgttgttttggtgtcaacaaatacaaaatagttatttaaagggtgaaattattaggtgaacttatcattgaattaacatttccggctactaacttctatagttatgcattttcaaagactaaagcaacacaagtggccaccaaattgtataataacacacacatagggcatattcagtgtgaggtaaaaagtgttaattttgggaatacaactatacatagatggtaaagattgttttagttagtatatataatcaattactgagtggtgggagggttgaatgtttgctgtttgggtatgtttcatttggctttaggaatggttaatatttaaggattttttccttttctgtgttaagaagtttgattttctttggaccctgctaatttagaatttagtggcattcttttttttaggaaaatcctgatccttgagggtgattaacagcttgattcatttctggtttttcattgtgacatgacatgagtatattctacagacttgtgtgtaaataggcagcattcttcaagttcagtagttgataaccagctttaaatacctgagttgaaatatatagcaccgctatttgttcatgatagtggattttgctatacagcattgccatttgatttaagttttgcatgtgattcacgttggaatataaaataccacacaacaattgttacacaattccatgacttcctaaaaacatactaattcagatccataacctcctaaagacatagatgctatacaaacattaataatgcaggcctagtgagtctgggtatgtttcttattccttctcacataagctttccaaggtatatcagagcattagcaggaaaccgaaaaatctgtgaattgaggtttccttcttccacaacagtttataaaagagaaaaagatttccccaagaagcttttgcctaccatctatcatttgtaattttatcttactggatgcaaggaaagtccaatatctttgccacaaatttagcaaatcccctcgccccccctgctcaaccttttccctgctgtctttcccatattttctaataactgtaacagaatattcttagctagaattctattttgtcaacggcgtggaggaactgtatatgtttttacatcattggactatcattccctctgaggaaacaaagattgcttctccccctttggcattgtcagaaaagtgtagcatgtttcaaaagatagtccagggaaaggagtaggggtgggctatatgagactacaacaagacctgattgattccatagaagctatatcagaaatgttatttctttattatttattggggcatttggttttatgggataaatgaacatatacaacataatctgtaccaacatttttagttgttaattctagatttgcaaacactgtcagtaaaatttgaataatctggaaaaccattttttgggccactatatttaatagcatgcttcctttggaatgtatggaattatgaattatgaaatcaattaccatttctgattatgtttacttatttgcttcaaatgtctttatcagccatgtacaattttttgaaagcaatgagaaaatgtttcaggtatatgattggatgaacaatagaccagagcggtttagagtttctgagattgatgcagcaattcagttagatttaattgcaaaagttcgtggagtttctagtgcagaagctttctttctgagtctggaagataagttaaaagataagaagacttatggtgccctgttgaatgtatacgtgcattctagatcgaaggaaaaggcagaatctttatttgatactatgaggagtaaagggtatgtaatacatgccctaccatttaatgtaatgatgactctgtacatgaaccttaatgaatattctaaagttgatatgttggcttctgaaatgatggataaaaacatacggctagacatctatacatataatatctggttatcttcgtgcggatctcaaggatcggtagaaaagatggaacaagtgtttgaacaaatggaaaaggatcctaccattgtccctaactggtctacattcagcacaatggcttcgatgtacattaggatggatcagaatgaaaaagcagaaaagtgcttaagaaaggttgagggtagaattaaaggtcgagatagaataccttttcattatctcctgagtttatatggaagtgttggaaagaaagacgaagtgtatcgtgtgtggaatacttacaaatcaatttttccaagtataccaaacttgggataccatgctattatttcttctctggttaaattggatgataatgagggtgcagaaaaactatacgaggaatggatttcattaaagtcaagttatgatcctagaattggaaaccttctcataagctggtatgttaagaaagatgacacagataaagctttgagtttctttgagcagatgtcgaatgatggctgcattccaaattcaaatacatgggagattctttctgagaggaagatgacatggaaagtgctgaggttttaattggactgctgaggcagtcaaaatttagtagaagtaaagtttatgcatctctcattggctcgcctgatggtaccattgacaatggtgaattgccaagcaagattaatataactgatagaactgatgatgctgttgacagtgaaaacatggatgatgactctcaaatgttgttgaaccaactagatagtagcttttgatactttatttctttttcttttgactagtggaaaatgatttgctagaacatacaggggattttttgtcaaatatgatctcattagtgtttcatggcttctatgtcctcttctcttcataacagttagaaagtggcagctgcttctatgtcattggtggttttctttaacgatttggtgatttgattatgtgtattgaagtgtgaacaaactagagtttttttttttttcgtgagatgcgaattagagtggagtttctttttatttgtaaaatccttgccgctgcacagcaaatgcaagtgtccctgtacggggaggatagaggtgaaggaaggagacggaggggaagatgcagggatacattcggaaatttaggggaaaaaacaaaagaaaaaaaatcttggcgatcaatttataaaactaataaatctaatgagcggtattacttttccacgctattatggtctcccacgctagcctgcgtcatttgaactataatagtttaggttcaatataatgagaggaatgatggattgagcttttggaccattgactctatttgctttagcccctaacgttgcttaaagtattttattttagtcatttttcacttaattctcctactatttttgtaccaaaaatgataaatgtaaggaattaaagtttctcatgttatatgagataaatttttctcctatacactaaaactacgtaattttgaattaaatttttgttattttattgttgaggatatgttagtttatcttttatttagcaacaagaggagaaacatgcgtgagagaagtatctgttttgattgtgtttttttattaaaaagaaaaaaaaattaatccaaaactacttaattttagtgtataggagaaaaatttatctcatataacatgagaaactttaagcagtaggatgatgttttacacatttttatgttcatatggagaggaatacacacaaatacgttattgcttagtttgcaaaaaaatcctttacaattacttaagcgaagtaaacaaaatgaataaaaaatagaataattttttttatcatattgaaccaaaaacttacttttttatctcttgaagataaaacgtctcttattgttgctcatgtagaacttagaagataacaatatgtattttattcaatgatctacatagcatgagaaacattgaaatgaaatttaaattgggtacgatttccaaaaaacaaactttctataaactattatagtcttatagatgtgggttagtaaaaaattgggttaggtacttaaaaaattagggattttcttcatgaaccctgaacccacatagtgaaaaattgtcaatccgtattttcaatattattttaaaaatatattttataaaataatttaaaaataatgatatatgtaggtctcgaatctaaaatcttcatattattaacacgactctctaaccaactgagttaataggtcaattatgtttaaaaaataattaatgtcgctatatataatactaaaatttctaatatatatttaatacacatatacataataaattttgtaaaaattaattttgatctagtaatgtatttttttacatatattaatttttattaaacatatgatttttatttgaaatttatatatgtaaaaaatacattattatatcaaaattaattgctataaaattttttatgtaaccttaccgacattaattattttttaacataattgacctattagctcgtcgtgctaataatgtgaacgttgcatgttcgagactttcatgcgtcattatttttaaattattttgtaaaacatatttttaactaatggtacgaaatccgtatggcccattcgtatagaccttatggattgacgatccctatgaaccttatggattgccaatctttataaaccttatggaaggtggctttccattttactgctgggtgtatgaagaaatttgctagatacaaaaaaaaattccaaaaacttattttcctaggctcaaagcggaaggagtccaatagtgtagagagtgttatatttttttaggttgtcgattcctgcactctaatattgcttcccacatctttcaggattttaattatcttttggattgatcattattctggaagccgaaacgtgtaaccaattttacattccagccaatccataaaagaaaaaaaaaggtgcagcaggcaatttacgtggcaggaagcaacaacctaaacttctgtgtttgttccgaggcccaacaagctaaaatgttattccgtgtgcatgttttaatgggtttaacttcctacacccagcaaagttctaatacacccagcaggtatttgtgaaataccacaaataaccttgcttaaaataataaaacataaggtttttaaaatttctaatacaccagtcactgaaatcgtttggcgcttcttcttcttccttgtgttctctgcttccttgcgcgatttgggttcttcttcttccctgtgcgctactgttctgtcgtgctgtggacggaggttgtggtgttgggcgacgaaggtcttctacttttccggcgcggcgaggtaagttacccaggcctttcttctgtcatttcgtttttcgtttttgaatgcgtacggatgggtaatccgaaatctgtgagttgtacggattatggatacgtaagaggtttggaaatccgaaagcctcatacggattggtgatccgtaataagaaatttgaaattcgtaggtgccttacggattggcaatccgtatcagccttacggattgacgatccgtaagcctcgtacggatcaccaatccgtatgattcttacggattgtcaatccgtatgagtcttacggattgtcaatccgtatgatacttacggattgccaatccgtatgatacttacggattgtcaatccgtatgatactaacggattgcgaatccgtatgatacttacggattgccaatctgtatgatacttacggattgccaatctgtatgatacttacggattgccaatctgtagaagccttacggattgccaatccgtatgggtaataggttaatttttgattttttgaaattagggattagggtttagggatgattgaggtatttttttgatcctccacatgcctatggtttgatccacgtttgctaggtaatttgaaatgtgatgagcaaattgaagtgttttaataaattgttatgtaaaagcatgttgtctaatggtatggacttgctatattgtgtttgatcctccacatgcctataaattgttatgtaaaaccatgttgtctaatggtatcgacttgctatattgttttttatcctccacatgcctataaattgttatgtgaaatgcatgttatgtaatggtatggacttggtatattgtttttgatcctccacatgcctatggtttgatccacgtttgctaggtaatttgaaatgtgatgagcaaattgaactgttttaataaattgttatgtgaaaagcatgttgtctaatggtatggacctgggagctggtttttgatccacatggctatgttttaatccacttgttaagtttcgttatctgaaaggattatttttatccagatgggtatgattgtaatgtaacaacttggtttcaatgtcagattatggctaggactataggattatggttggctttggcaagagatgccagtgaagaagaacccggtgtacggcgaagaccgacagcatcagcacggagacaacgccagaggactggtgttgttgaggacgtgcctgctgaggtagccgctgacgatgttgaccatggtgcacctgttgaggctgaggtcgagggaagcatcgatgcctctgatgttgacggatttcccagtgggccccaggacacttcagtgttgagtcattacgatgaacatattgcacctaggatttggaggggagaggtatgattgtatttaaacatagttgaatttgagaattaatttcgcgttaaggtatgtgtatgtaactaacaactaatacaggaacggccggagcttaagttggcatctcatgggaggaaggtgcataaatttggccggcctgcgcctgagattgaagggttggtggcaggtacaggattaagcccactgatcacctgttcggtagatacaggtgatcggggtttaatctcgacatttgtggagaggtggcataaggagactagcagtttccatttgccgatcgaagagatgacgatcacgctggacgacgtcgcgtctctatttcatctaccggtgatgggtgcgctgcagactaggggtgttcatcaaccgatccaaactgatgcaaaccacggtgaccgaaccgaaaaaccgataaccgaaataaccgaaaaccgaaaaaccgaaaacaaaaaaaaaaaacgaaaattttcattggattggatcggttttcggatcggctggtgaaaaccgatccaatccaaaccgatccaattataaaccttatcttaccctttatctatttgggcctatttcatatcatagcccaaaaaaccaaaaacccaatacattgctcattattttgtccaatgaacgtgtttgtccaatgaaaattagaaaatggtgtttgtttttgttcaatgaatgtaattgtttatgctagatactccttattttgataaaaataagtctcataagttttatttttagactttttaaaaaaccgatccaatccaaaccgaaaaaaattggattggatcggatcggatttgaaaaaaatttcaaaccgaatggatgatcaaatgaccaaaccgaaaggattggatcgaatggttttttgcttcaaaaccgatccaatccagatcgcgaacacccctactgcagacgtacgatgttttggacgttgatgaggcgattgcgttactagtagagttgcttgaggttacgcatgaccagactgcagcagagacgaaacagtgccatggaccttatgttcgtctgtcatggttgcgggagttatatgatgagaggtgtcatcagatgcaatggacggtggcagccagagcttatttgctccacttggttggttgcacgatttttgctaacaagagtgccacacatattggtgtggtattcttggaggcatttcgtgatctccagcagtctggcgggttcagttggggtgcggctgcactccaacacatgtacgataacttgaactactcctgtcagcatcatggtaaacatcttgctggttatattagtctgcttcaggtaaatttttattatataagttaatatttgttatttgtttaatattttggttgaatttcaaccgcttatttgtgtttggttgttttggttacttaagtgttgggtatacgagcactttcctagtgttggcagcatgattgcggatgaggactatgtcgagagccagccacgggcatgtcgctggaagccggggaaggcgttacctgtaagttcctatcgaaggcgactagataggttgacgaccgacgatgtatgttggactccatacactgatcattgtccggtccgtccctttgaggtcatctgtctatactcaggccatattcgatggggtccttactgtgtcaggtaccatccagagaggatactcagacagtttggcttcattcagaccatccccactctcctgagcttgcccaccccgtcagatgaagaggtggatgacagttgggatcattacggccagcatcttaccatcacaggacccccttccagctaccccggacagttcgatactgcatatatggactggttctaccagatatcgcacccctttcatgtgcccgcctcagcctggggaagtcagagcgtctcctcccgtgccagttcccgcagatgatccaccatctactatggcagaccccacgggtgacattgcagctgatcagcctaacatgcctcggcatgcagtggtaacacaaataaaacttcattctagtattaacattattgtttaatctgttatgattataatgaccgcttacacagtttgtctaatttttttgtacggcgcatgccaaaccattgcagagaggttgcagaggcttatagatcttcgcatggtgacctctggcaccgatgtgttcgacactgttgatgactgcattagagtagcacgcagtgtctgcgatgatcggagagtgtatgtgcgctcgagaagtcgtaggactggaccgcgcacagattgatttgttacattatttgtattgtcgaaacatatgtattaacattattgtataacgtggaaacatttcatttgatatacgtacatgcttttaacattatccatctgcgacagtattttcttttgcgaaataaaagaatctagcacttatacaattacataaccccctgctagtgttgacatcccctgtcagaacccagatcaacataagctggtggcaatgttgtcaaccttgtatacttctgcaatcgactaatgtaactagatggccactcttttgcttgaggctgacaatgtcttgaccataatagagcaattggtggcataggacagccatctcgcaatgcaacctgaacacaaacccttttattgttaataacatctgttataagtatgtacacaagtaagatgattggaaacacataaaacgacattagttatacctgaacaaaatggtttccatatacatgaccgatacaaatgacgcgatgtaccgcagcatctctaggcagttggcttctaagcgggaaaaaagtcatactttgttgcttagaaacacaaacaagaaccaggttataacgtgaagcaactacataacccatgtctgtaacatccatccatttatcctgtgttacctgcattttgacaagtataagtgttatgcaccacaacaatatggtaatattatcagaccaacacaagtcacatttaccattgagaatccgtcgacaagtagtgacctctttaagccctcaaatctttgtaacccaccaaacaatagaatatagtcttcagaccatttgttaagttccttaagcaaatggttacgaaccacaggccatccatcttcaccaaaacccagtagagccgcaataacccgatatccacaattaccatcggctttaacatcacatacaccaataatgtgatttctcatgcacggaaggaactgatccagcattggaatatcacccatcggtaaaagacctgaagatgttgaagcaggctatggtacagaggagcaactatcatgctgagaatgtaatgcgtcgacttactcaaagtacaatggatctcgcttggttgatctttcgtgcttcgtaagattttttttctgagccccttttgtcttgaccttctcaggaggaggacacattgatgtttgatcaggatacgctatttctttcaatttagtcttaagtcgagactttcaaatcgcttcgatatcgcttccatctcttctgtgatggtcacctccatctcatcaacacctgagtccgacatatgcaatctccgccaaaacatgtgtaccgtgcccagtggtatgctgccaatattatactttgttaacacacatgcacaaggcaaaccatgcgtacttcttaacacacatccacaacgagaactatctacacctacatgcttaactcagtcaaactcagcggcaatttcatttaaaacatacctggaaaccatgcctacgagtcttttgtataacgtggttttaaacatatgtccaacaacatgtgtgcttgtctcaaaagaagccttaatttcattgtgttgtaacgtgatcatgttgttcatcgcatcacatacattacacagatctccaagactgtcctgcagtagtcgctttaaggtccaatgggcagactaaaccctgtagtaaacggttaaaataacactggttaactaaatcatgaccatgcagtttaatgtaattgtaaactttctgttgtaaaacatttacctgttcgtagtggtatgaccaaaatgcatgactttattagtccatgctgtgacaaatcgttctttgtgagggatcaaccacgtgtgtttcacatattgaacaaatattggtcatggtgcacaaacacattcaaacctcatcagatactcattccattcattttcaaaagcacaatctagaagacttctccatgcttccatgacatactcccatgaattgttttgtcccacgagcattttacattttgtcttgacattcttgtcaatgtggaaccgacacaacaagttcattgtgccagggaaaacatctttgatcgcattcatgagggttgtatctctgtctgtcacaatgacattagggaaaaattcagtcttcaaaaaaagccctctaaatcgttcaaatgcccatacaagattgttcgtacgttcgccctccaaatatgcaaaggcagctgaaaaagtcatcccggttggtgtcactccgactatgtcaaccaaaaggagtttatacctatttgttttgtatgtactatcaattaggaaaaccaaatgacatgaatttatcaacttcactgcatccggatgtgcccagaacatgtctcggacaacattttcgtccttcactctatgccagtggacatacatatcacgatgaagaagctttattaaatgttgcatttcagtattgttgcctcttatagaagtacggtatgcatatcttgcattgtacacttgtttcattgttgtataacttttacgattgtgatcttttaaagtgagcataatattctttggtttaacccttgacttagtcatatcagcaacaattaccttctctttctcagttacacgaccagcatatggatgtccagtgaatgacttagccaaacaatggttgtgtaacccgcagattaacttgaccatccacccttcatcacctacgacgggtttccctcgcaacttaaaaggacaaccgcattttctactgctagtggtcatgcgtactaaatctttcttgaatgctctatagtttccactcctttcacaacctatcaaaataaatgaggttattcccctcttaccagttgttgtgtcagaccttattatcacagctacaaaaccaatctcatatgcaacactacgagcccattgcaatacctcgtcacgagaattaaacacctacaacatttcaaaatacatgctcaatctcatgaaattatttatttactgacattcaatatgctactttatcacataaatacctttgaagtcttaaatgcatcagaacaatcaatcacagagttgttattcttaaatgcaaacggttcacctctgttgtcttcacccatattattttcttcctccataatatcatcatgcatccaatcatcgttgtccatcttaataaacaaacaaattcaaaacaaataatcattaacaaaaagtatctttatattcatttcacaacgttgtgattaattcaaaacaaatttccatcataataaaaaacttaactgttattttaattcaaaaaggaaaaatacattaattattaatacctttaacaaaaacaacacaaatatttaaaaaaaaaaaacaaaaatcaaaacaaacaggaaaaaaaaacccatacggattgccaatccgtacggtttatacggattggcaatccgtacggtttatacggattggcaatccgtacggtttatacggattgacaatccgtataccccaatccgtatgcgtgtccccagaaacagtagaacttacctccgatgacgacgatcccggccaacgaaacctccacgactgctaacaacgacctcaacccaacaacccacaacaatccaagctagaaccaacaagccagaaccaacaaagccataacacggaagaagaagacgagaaggaagaatattcagcagctgggtaatattactgggtgtaggaagaagaaaataatgaatcacaaggtaaagtaaagcaaaacgtacaagggtaatattgtcttttttacaaaagtgctgggtgtattagaactttgttgggtgtaggaagttagccccttgcatctgctgtgtaattggttttgattgaacattggagaaacataataaattatgctatggtaaattgataagctaaaaattataagtttgtatgtaaggtggaagtatttttgagaattttttaatagaaatattaatttttttaattaaagaaactcttaaaaatgtttttaaccttttatccaaacaaagtgtggtgtaaccaaaatttagcggattttctccatgattttttcatgtggcaattttttttctagtttacttttttacaagtaaaactttatttatttttactagatgtgactagatgtagggaatttgtaattctattttatatctttatgtagatactactggtagtacgaaaaaataacactatgcactttcgagggtatgatagaggttgcttttgcacaactatttattattaggggtgttataggatatatacttgcgagaaatatttgactaaacaattataaaagttataattaagtaactaataagtaatttttatcaaaacttaagatatcagtaaaaaaattaaaattactataaaaatatttaaagaatgaaaaatacatgcatgcagcatgccttttatataattcttattgtaattaatacatgaatgccttttatagaataaataatacatgcatgatttttacaataattgtaattaatgcatgcataatgataaaaaaaaataatgtcatgcattcttttatatatagaataaataattaatgtgagtgcttgtgactattttattttatatttttggtggtacaacaaaaaatggatggtcttctaagttgaaacgggtaataagtttttgtctatacatgctaacactggtggtccctggcaattcaattataaggaacaaaaatatataagatagtaactaaaaaaattagttaatatatttaaaaatattttttaacttttaatttttgatgaattttatttcaataaattaatttggtgcatttaatttttaacttttaaaaaaattatgaattttatcttatgtcattttactcctaatataattacaccttttactcaactttttatttttttgaaaaaaaatttcctaatttttggtaaattttacccctaattttttgcttggttgggataaaatttacaagtttgttatgccaaattaaaaagggcaaggttcaccgaaaattaaaaagtttggataaaaaaatgcaattaagtccgaataatatagagtttatgctatgaaaggaagattaatagaaaaaataagaagtattaatgtgatatttaaaacaaaaagagaatttcaatattaagatttttcatttaaaaagcgtcttgggccacttctaaagaagttgcaaagaaactgcttttcagaaatttcatttcaaaaatcaactactatcaaatactcaaacttttttatttcaaaggcaaccacgacctaaaaagaaataaaatagataaattggttctagatccaaaataccagattccaaacttacattcaagtgcatgatttcaggtatcctggatcgtgatgatacactcaggcgagtgcttcagttagttctcctccaacatcatttagagaggagcaccatgcagcatagagcaccccgatgattatgatgctaagaagtagtagcattaagcctgtggcccctccaaacacaaacaataggcctgatattttagctgaaccaccccatgaaacactatatccagacgttgcaactattatctctaaaatgtaatagagtaaagaaaatcccagcaatgcgtatgccgatatatgaaaaggcaatgacgaggtatgcactgctgcataaataatgttcaggaaaaatagtgatgcccatactgggctggaacatgctttccccatacttaattccattgggtcaatgcttgtggattgggacaacttgtcaagaggattgcccaaagcagatagttgggtccatagcagtccatataaggcaacacataaagtacgatcagtagaatgtgatcggaatagcctgttcccataaaacttcagaaatctgttcaaactcttcttctttcctttatctactttcaacaccccattatcgtcaatctttacaacatggtttgggggatgagaatggcgatttttcttttcttcctgttgatagtcaatcacaatggagaaatatggttggtgagtgagaattttgatgaaataatatatggtctttctatggaactttatttttggtattttcatgcaaatttaaagatcttatattttttagatgatgttaaaaatttaggttaaaatatatttgtgattcttaataaatattcaaattttgttgtgtttacttcccaataaatgttttctttgtatttgatccttgatatttgaatttttttgtttctggtccctaacattaggttaagaatgttaagtgtctgttaaatgtgatttctgatgatttctaaaacaaccagtatttactttaaattttaaatgacaatttatggcatctaaaaatgactagaatttgcttcaccgaggcacaccaacaacttgccatcacgatccatttcaggggacaaaaagaacaaaatgatacatggctcattttgctcccatgttgagatatgtgacacacatgcacaccatatatatataattttagatatgttgaggcagcaacggatccaggaatattcttttatgttcgcacacttagtatttttttgtaaatttttttaaaaaaaacactttaaatcaactaaaaatggaatcaacaaattgttagtgaaactaaacagtgacaacaatcacatcggcacatactggagattatgcaaacacagttagaggaacttacattatattgatcaatcttttttatcatctcatccagatgataacgtaggctgaagtccctaaaagcctgtagaagctcgtcactgcttatgtgaccactatcatctttatcaaaataggcaaaagcagcagccaaatcctcccctccttccatcttattcaagtgcagtgtagcagcaggaagttcaccatagtctattgatccattgttgtctataccagcctgccaaagttcatacataaggcaaggcaattaatgatcacccaactcagcttacacaaaagaagacttatttgaaataacattaaaacactatgtgttacattctatgataatggaaatctacctaccgcttccataagggatttaatttcatattccatgagattagagcccacacttttcaaccccttcttgagttcctcaaaagctattgtcccactattgttttaatgtatcagggtgtgatttatatgaataccagactatatataaataacagagtataaaactttataaaattgatatgaattgatattctgaaacgaaaagactatataatatatttaatcaccattaacgggaacaaacataaatcttacaaaggataaaaatatccatcaatgcagaaagctctaacctctaatatgagagaaatggatccatagaaattgacaaataaataactagctgacatgacatctgtattcagatacccacattttcttatgtaatatactttcatcatatctcttgacctcacaatccttagatttatgatatcacaaaagcattctaagatctacaatcctatatgaaagtattggctcataggaattcagaacaaatcatgtggtaattaataataattcagaacaaatcatgtgaaaaaagcttgaatcagcttcaatggcggtgacgacgcaggcacgaacgcagaacgcagaacgcagattggaggtcgaggccgaggccaagcaagccgtgagcgtgacgacggcgcggccaacacgagggtgtcgacgcagggaggtgttgcacggtggacacgtgacgacggggtggcggcagatgactcggtgcggctcgacgtgaggctcccgtttttggacaaacagaagacaaaggcaaattagatgtttaaatttatttgtaaaaaaaaatacttaaaataaacaacttttttatttttagtgtgtttgtctaaattgattgtgcttaaaaaaccagtttcctcttattttaagaaactaattatatctacttagaaaaaatgctgataaaaaacgcttattttaaaaggattttctaactttaaaaacaaacgcacccaatgccttaccctcggtataaattggtgttttatgtagtttggtcctgttttcataaatttggatacttaaatgctcaattaattctttaagatactacacttttattttacctaaaatgtaacttttgtcttcctattttttaaaatttattatttttgtcctcataatttttaattgagacattttattttttattttaaaaaaaaattataattttagtctccctatttttgattgagacattttgcttttcacttttaaaattttgtgattttagtccctttttgtcactttcaagacttgtaaaaataatatgaaccaccatggttgtacttaaacagtggatagaatgaaattaatagaaacaaattgaacggatccaatgaaataaaataaattaatatttattgtgtcaatattttaatgataaaatgatcaaagagattgattccatctcatagtagctaaattagaaaagaagaaaaaataatagtattggatgatatgaaataaaatacgtataacaaagtataacaaagtcatgtttagttctattccgaacatatttaaaaccaatcatgactcaaaagaaagagaaaaaaatagcaccttccattttattgtatttctaggtgtcaattgggaacacaactccgaatatttcaggaattaatgattacaaatattgaaatcattttgcaactcaaagtaatctagttaattgatttaactttattttactacttttacaagagaaaatcaaaggaaaacaaaaggagggcattttcattagacaacattatcacgaacactgttagcaaaatcgcgatctggatcgtaagatcggacgattttacgatctttgcggccaacggcaatccggatcggtgctaggatcgtagccgagtgggatcgtggtgagatcgcgagtttgatcgtaggatcgtaagatcctcgatccgtcacgatcctgtttcagcactgaaattaaaagccctaaccttggattcgaaaacgacccaaatcacccagtagctgcgcgacccagcaacgccgcaacatcccgacctccccccgcgcgatttcagacgacggtggcacctcccgacctcccctcttctgcgatcccgaacgatccaaggtcgacgacccgaaggaggaggagaaggaggagatcgacgccgattttgacggaggagtggaaaacttgttcggcgatctcggcggcgagaggaaggctctgattttcagcaacgaaacgcggaggacggaggtgcttcacagaggcgccgcagagctcctctagtttcagcaataacacacgcgaccaggagaagatgaaacacgcgaccaggagcagaggagaagaggaaacatgcggggagaagagaagaggaaacacgctacaggagaagaggagaagaaaagaaaatagggttttctactaatttgttttttgtactcactttttttctttctgcagtatcatataatatttttgaatggacttttgagttttaatttacgtaatttctatttaagtctttccatacccacttttttatttctgcagtttcatagtaggatctacgatccgatctacgatccgatcttacgatcctcgatttacgatctttcatcccccctcccgatcctaagtaaaatctcgattttaacaacaatgatcacgaatcaaattatttttttatttctcttccgattttgaatgaaaaactatatttaagcattttttttttgtctacacacgaggaacaaaaaccctcggtcaattttgaggatcattgaagaataaaggaaaaatctagggaggataaaataattaaattagggttactaattataatttgaaattaaagataatagtgacgatttttcaaaattgtcaaaaattgtattttgaatttttaacggtcatgtcatcatttaataaaggaatccttacaacagggactatgaataaattttttcaaatatcaaagatcaaacacaaagaaaaaataaattaggaatcaaacacaaaatttaaatatttattagggatcgaaaatatattttaaccaaaaatttattataaaaaaattgactaatgcaactttcaaaatcttggagaacttgcaaatatttattattatttcttacgataaatgttagctaaaaaagcaaaaaaaaaatatcaaacaatcaacataataaaaaggacaagcacatggtaaacgtttaagtttgtcttggtttaatccaaggtccattttattaatgtatggagaaggatctcctatagtgaaagttaacatgagaggatcatgtgaactaatcacatccatctatatcttttaaatatgagtttataataattttttataaaataaattaaataatttcaatggtttagatctcatgcaatggaaatgtcattgtaggggacaaaagaacaaaatgatacatggctcattttgctcccatgttgagatatgtgacacacatgcacaccatatatataattttagatatgttgaggtatgtgaaagatgtgagatatttcaaaaataaaattgtgagatatttgaaagacatatcctctctatgtgagcaaaaagtgtgtctaaagctcaatgacttgttatttttaactaattctatatgtaccttgcacattctctctaccaactttgcatgttcatacccttcatccggttgccgatatagttctttgacttctttcggttgttgtagttgcagttgctgtacaaaaaagtaacaaatgttaggtacttgagaagtaaaggtccaattaagttacattaaggaaaaatatgcatatgcatctctttattaaatttccaagccagttagtattgatcatcaaaatggacaaaaagaaaagacaatgatttttcctacatggctcattttgctcccatgttgagttatgtgggatgtttcaagccgagcaaaaaataaaaatatacttgaaagacacatcttgtttatgtgagtagaaagtttgtctaacaatatgtagttagacaacaaatgtttcgttatgagatgacactacactttgcgccattgaaaatataatgtgtcttttctcttaatgtataagacgactttgcaaataatctacgtaagacaaaaattgtactttttttttcacaagacaaggccattgcattagcttttttacatcaaaattgttattataatgttccatttgacaataaaataataatgttaaatgataagtccaaaaaaaattatgttttggatgaagaaaactcaatgacttgtaattaattagttctatatgtatcttgtttttctccttttgagtctcacttgcttgtttttgtgcataaaagcaagaaaggttaggtacttgagaagtaaattaaggtgcaattcaacttatttgttcaaataaatagaatgatttgccctagcttatggatcatatagttagatattatgttgagatacgtcaaaaaaaatgtgagatatttgaaggatatactttgtattgtggatttgtgagtagatattatgtctaaaaatttaagatgatttgatttctgcactagacaaagttttgtgtatttcttggtcatatctctttatcaaatttgttattacgatgttccatttgtgattaaaataataatggtattgataagacttgggaaaaattatgttttggattaagagataacaatggttggctgttataattagttctatatgtaccttaaactttctttcatcctcttgccacttcacaaaacaacgttctacgtcaatgtcagtatatacttgcagccacttctgtacataaaagcaatagagattaattaggcacttagtagaggtgcaattcaaatacattaaggaaaaataagcatgcctttattatatttaatctcatgtcacttagtatcgagcattaaaaatggacaaaaggtagagaaagaaaattatatgattcatttagcttccgtttttggacaaacggaagacaaaggcaaattagatgtttaaatttatttgttaaaaaaacttaaaataaacaactttttatctttagtgtatttgtctaaattgcttatgcttaaaaaatcagtttcctcttattttaagaaactaattatatctacttaaaaaaatgctgataaaaaatgcttattttaaaattgattttctaactttaaaaacaaacacacccaatgccttaccctcggtagaaattggtgttttatgtagtttggtcctgttttcataaatttggatacttaaatgctcaattacttctttaagatactacacttttatttattttacctaaaatgtaatttcgtcttcctattttttaattcattatttttgtcctcataatttttaattgagacattttgttttttttttttaaattacaattttagtccctttgtcactttcaagatttgtaaaaataatatgaaccatgattgtacttaaatagtggatagaattaacaattatgaaccatgattgtacttaaatagtggatagaattaacaacaatgaaccatggttgtacttaaatagtggatagaatgaaattaatagaaaaaaattgaacggatccaatgaaataaaataaattaatatttattgtgtcaatattttaatgataaaatgatcaaagagattgatgccatctcatagtagctaaattagaaaagaagaaaaaataatagtattggatgatatgaaataaaatacttataataaagtcatgtttagttctgttccaacatatttaaaaccaatcatgactcaaaagaaaaaaatagcaccttccatttaattgtatttctaggtgtcaattgggaacacaactccgaatatttcaggaattaatgattataaatattgaaatcattttgcaactcaaagtaatctagttaattgatttaactttattttactactattacaagagaaaatgaaaatatgacatttcatcgatagtttctttttcaatattggtagatatgttatagatatttgaaagatgtgagatatttaaaagaaaaagtgagattttgaaagacacacaagaaattaacttttattacaagaaactagctaggaaacttgttaaaaaataattacttgtcaaccaagttataataaggcatagctattatttctaacaggcagaagcctttatgtatgggtaaagagtgtgtttgacaatgtagagttaggcaacacgtgtttagttatgatgacactgcattttgttgaaaacataattatgtctatttgccttaatgtgagacaaattttccaatcatctatacaagtaacaagactaaggttgtgtttttcttgtttacaaagtggttgcattatcttctctagctatatcaaatttgttattataatgatccaattgacagtaaaatattaatagcaatgaatagcactatagaaattatgatttgaacgaagagatcataacttgttgtatcaattagttctatacttacatgatttttcacctgaccaagatatatttgataaaggtaaccttgtagtatcaatccctgtacataaaagcaataaaagttaggtacctaacaagtgaaggtgcaattcaattactgtaaggaaaaacatgcgtatttttagtaaacttctatgccaattagtatttaataggaactggttcagaaaatgcagagaacgactgaagttttattcaatagtgaagaacacagattacgagcacagtgattttcgaggcaatcacaacctcccaactctccaagagagtctaaccttaccttctctccccttcttatacattcacgcccccctctcagttagttataactattcctccaacattgttactgtaatgttagtggcattaaaatacaaaggtggtatgtttggtggatagcactgacatggagcatttggttccaccgcaacaggatcgtcttccacaatgaaaacttcaatggatctaagttgatggaggatgctgtatttttttgttggtcatggctagttaatctggagaaaggatttaatatacctttctaccattggtctagtcagttgagtgttgggttctgctagatagttttttatcttgttttttaacttccttatgagtctaaccttaggctgttctttggcatcttaatgcgggtcgttatcatgtattaggctgccaaagttctggttggctccctgggatggtttctttttcagtattacttgtactcagctctatatatatatatatatatatattatctttgctgatcaaaaaaaaaaaattatcttttagttatactaactgcctaactattaaaatgctatctgaaccctctgtgatatgtttcaccccacctagcactcctatcaataccctcctcttcaagctggaccttgtccccaaggtcctcttcagaaaaagcagtcttcaactcttccagcagctcccagctgttctcatgatgtggcagtccttgccatttgatcttgacagaaaattaatgatgaattaaaagttatcaaaaaaaattgagggactaaaatcttaattaaccctttagattaatatttattacaaataattctgaaattaaaaaaaaaaaaattgaatccctgataagggccaagaaaaatttgaatggagccaataataattctcaaaagtgtgacatttacttgtcattttaagatatcccttcacatctcaaaatatttcacaaattaaaggttacagctccagttcaaagtaatttacatagcataattgcaaatttgaatgcaagaaggaaaaaattattgctttagaagtaaaattacttttgagttcttccaactgaaatctaaatatgcagtaaactacttaatgaggatcgtgatagcaattaagagtttatgagagttataacttacagcgtaaaatttcgggaaagacgaattcacattaggcgcaaacttggattcccaaactgaatcagcaatcacaatccccatgccatgcactgcacacaaatcaaagaaagaatatgcatacatgttagaaattatgatggcgtattaacacaaactgctgctgagttaatttaccatgtacaacccagagttctggatacagttccgttatagttttctcgagtgcccgtaaaagataaggctccaaagtccttgtttccgtttctgatatggattgcaaaattttgcgtatcagctcttccagagagtcgtctccactcacttctatttcttctataaaatgaaacttcttttcggcttccccaactggaaagtaccgtagtagatttttcaaatgaaatttcatccgatctataatggcttctctacgatcggatggaggtgcctgttcattgatctcgttcatttcttctgttcctctcagcctcagctagtttacaaactaatcaatttcggttttgctcaagagttgcttctactgccagaggcggcagaagtagggtggaaaaccctaatttgcgggtgaagttacctatatacccttcattttttatatttttcccaaagtaccctaagacttggtcagaaaatatcagcccatttttttgttagtgcctctgttctcctcgctgttctctcatttcacgctttccccctggccctttctctcgtttcgctgcagcgccgcctgtgcctgttgcctgctgcccacggcgctccgcgcctccgcctgctgcctgcgcctccgcggatctccagcttccaggttcacttaatcacttttatttttcaattttccccaatccaatttacaatcactcaacgataacaaaaataaaaaagtagggataaaaaggaactgcaatgtgctttgtttgaagctatagaacatgtgaacaaattttcattgctttatacatttttaaagcttttaattagtagtttggttggtacttggtaataactagaatatcacattttagtttctatttttcttataaaaaactgacacatacactgatacagtgatacacatgaaatctcatttctttagaaacatattatgagttatgaattaatattcaatgtaagatggataccttaaaattaaacaaatgccacaaaactaaagtgtgacaaaactatgaataaaatgcagtacttttttttttggtttcccacaagtatcatcctttaaaagatgtatttgaaagcattgataataacgtaattatgaaggaattccaaaaaataaggcatagaatgatgatattgtaattatttttctggatatgtatttgaaattttgagtgcccccttgatttattttctggatctgtccctgcctaacaccatacaagtttgagaaggtgatgagaaatcaggacaaccttttagcttttgagtacacattgaacacaattatgcattttgttctggttgcgtgtgtaagtgagttatttaacttatgcaatgatatatggtgtgatgtatattatgtgtattccaggggaaagaggatagtagaacatcaatgaacattagttctgaagttgaaagaagaacaccagaaatcatgggaagatgtgcatcgtggtgggccaccttcacattttttttgtgtttgtgttcattaaaattgagcaagccacttctccaaccacaaggacccaattcactatgccttgcgatagtaggaatgttcagttttaccgtgtttgtatctgcttcttctgttatgcttgttgttggagcacagttcactcgtatgcagataactgttatgctcagtgctttcttggctatgctgttagtttaattagacagttttcatatatgtagttatgtttccttcaggcagggttgaacacaagttgttgtaatcttgttatttacataatttcattcgcataacaataaaacaaagtttgggtatgtgatatatcaatatcaacgattaaccagacagaacatattcacggatgaacattgaatgacaggaacatcccttcgtatgaacttttctttctttttcttttaagtttatttagaatcttatgaagcataagcataatacaataagatattaaaagaataacaccagaaatcacaggaagatatgcatcgtggttggcaaccccggatttttttggtgcttgtcattaaaattaggcaaaccacttctccaaccacaaggacctcattcactatggcttgccatcctaggaatgttcagtttcctttgtttgtgtctgcggtttctgttatgcttgttgttctagcacgcttcaatctgatgcaaataactgttattgtcgttgccttcttcgatctgctgttagctttatcacatgttttcatatatcccttccttaacccactagaatatagtgatggatagggaattgtataatttgcatgggattgctttcaattctataggctgcgtggacacatgagatatatgtttcttgtattttgttatttacagagtttcattagcataccaatgttttatgtatgtgatatatcacaagactcgacttgaagttgtttaaggggataagtctagtaccactcgaaccaatgacttaaacatttaatttcataaaagaacattttgtaaatagagaggagttggaagagagtttttttggggtcaaagataacatgtgtcttccaatccaccaggtcaaagactaatctagttcatgatgtgtgactatctctaatccaatgaaattttgcacaagataaaaatcaaacacgaattttcctattaaatagatcgttcatctaaacaaagtgaaatatgataccattatgtcaattcttttggatttaaagaaagttttgtttacctaaaaaaaagatgaccaaaaaaaaagttttgtttagatagttttaattttttataatttaattgtatatgttatcaatgtaaaactttttttatataaatatttaataaaaattactttatcaccatatcatattaacatataagaagatatgataaaatattaacatgatccaattttattaaaaagtagtgtgattaaaaaaaagagtttgttttttttcaaatgctaatccaaatcgcattatgtaactgtgtcactgatccaaaaaaaattgcgatagaaatcaaatatgattctttttttcattaaataaatttttctcgtaaatagaatcggacattatattaactctaaaatcagttggctttaacttcctacacccgtctaagttctaatgcacccagcagttatttgtgaaataccacaaataacctttgttaaaataataaaacataagcgttttgattttttctaatacaccagtcctttgcgcttctgcttcttctgcttacttccgcttctgcttctgaaacctgggattttggttctccgtcgcattcaaccgtgtgcgatgctgttctctcgggctgtggacggagcttgtggtggtcggcgacgaagctgttctggttttccggcgcgacgaggtaagtgactggctttgagtcgtttcgtcttcggttttgcatgcgtacggattgctaatctgtaacctctgaagtctacggattatggatccgtattaaagattggaaatccgtaataagaaatttgaaatccgtaggagccttacggattgccaatctgtatcatccttacggattgtcaatccgtaaggctcctacggattgtcaatccgtaaggctcctacggatttatgatccgtaagtcacttacggattgacgatccgtaaggcttacggattgccaatccgtatgggtatttgcttaatttttgacttttttgaaattagaagattaagtgtgatgactgaggtattttttacatgaatgaactgaactggtttaatgaattgctatgtgaaaagcatgttgtctaatggtatggacttgctagattgtgtttgatcctccacatgcctatggtttgatccacatttgctaggtaatttgaaatgtcatgagcaaaatgaactgtttaaatgaatttttatgtgaaaaccatgttgtctaatggtatggacttggtagattgttgttgatccacatggctatggtttgatccaagattgtttttgaattgttatgagtaaagtgaattgttttattcaattgttatttgaaaagcatgttgtctgatggtatggacctggtagttggtttctgatccacatgggtatgttttaatccagttgttaggtatcgttatctgaaaggtttattttgattaagatggctatcattgtaatgtaactacttgggttcaatgtcagattatggttaggactataggattacgacgggctttggcaagagatgccagtgaagaagaaccccctgtacagcgaaggcctacagcatcagcacggagacaacgcgagaggactagtgttgttgaggacgtgcctgctgaggtagtcgctgacgatgttgaccttgtggcacctgttgaggctgaggtcgagggaagcatcaatgcctctgatattgagggatttcctggtgggccccaggacacttcagtgttgactcattacgctgaacatattgcacctagggtttggaggggagaggtacgattgtatttaaatataattcaatttgagaattattttggcgttagggtgtgcctatgtaactaaccactaataaaggaacggcccgagcttaagttggcatctcatgggaggaaggttcataaatttggcaggcttgcgcctgagattgaaggcttggtcgcaggtaccggattgagcccactgatcacctgttcggtatatacaggtgaccggggtttaatctcggcatttgtggagaggtggcataaggagactagcagtttccatttgccgattggagagatgacgatcacgctggacgacgtcgcatctctatttcatctaccagtcatgggtgcgttgcagacgtacgaccttttggacgttgatgacgcgattgcgttacttgtcgagttgcttgaggttacccccgaccaggttgcagcagagacgaaatagtgccatggagcttatgttcgtctgtcatggttgcgggagttgtatgctgagaggtgtgaccagatgcagtggacggtggcagctagagcttatttgctccattcgatggcagttagagctcctcctgccgtgccagatcccgcacctgatccaccagctagtatggcagagcccacggttgacactggagatgatcagcctgacatgcctcgacatgcagtggtaaatacaattaaaacttctttctagtattcagaaatgtttaaacttttatgattataatgacctcttacacagttttgtgtaactttttgcagggcgcatgccaaaccattgctgagaggttacagaggctgatagattttcgcatggtgaccgctggcaccgatgcgttcgacattgttgatcactgtatcacattagcacgcagtgtatgcgatgatcagagagtttatgtgcggtcgagacgtcctaggagtggaccgcggcgagattgatttgttacattatttgtacttcactatataaacatatgtattaacattattatattgttgaaacatttcatttcatatatgtacatggtttgaacatttcatgttggttatatgtacatggtttgaacattatccaactgcgtcagtattttctttagtgaaataaaataatccggcagttatacaattacataaccccctgtcatataggtgaacacacatgaaaatactacataaagtcacaatgtatgccagtgttgagaccccctatcacagcccagatcaacataagttggtggcaatgttgtcaagctggtatacttctgcattcgactaatgtaactagctggccactcttttgcttgaggtcgacaatgtcttgaccataataaagcaattggtggcataggacagccgtcttgcaatgcaagctgaacaaaatgatcacaattacattgatttgttaatgacatgtgttataatcatctacacaaattaaagcattttgatattacctgtacaaaatggtttccatacacatgcccaataaaaatgacgcgatgtacagcagaatctctaggcggttgggttctaagggggaaaaaggtcatactttgttgcttagaaagacaaacaatgacaaggttataacgtgaaacaattacataacccatgtcagtgatgtccatccatttatcctgtgttacctgcaattttacaaatacatcagtatcattatgggcaatttaacaaatactaaattatggaatgcaaatgtaaacaacgaaaaaaacccttacatgtccaattgcttctaccaaaagtgacctctttaactcctcatatctttgtgaccccccgaataaggcaatatagtcttcagaccattctgcaagttcctttaacaaatggttgcgtaccacagcccacgcgtcttcaccataaccaagtaaacccgcaatggcacgatagtcacagttaccgtcagctttaacatcacatactccaataatataattcctcatgcatggcaaaaattgatctagcattgggatattacccatcggtatacgttctgaagactttgatgcagacttttgttgagatgagcaactgtcatgctgagaatataacgcatccacatattcaaaatacaatggatcccgctttgttgatctttcgtgtttggtgacatttttcttttgagcccctttagtcttgaccttttcaggaggaggacacatggaattttgttcaggatatgctatttctttgagcttagtctttaaagttatcttgccacacatgtcaagactttcgaatcgcttagaaattgcttccatctcgtcagttatcgtcacttcacagtcatcaacaccttggtcagagaaatgtaacctccgccaaaacatgtgtatggtacctagtggtatgctggaaagattatatttggctagctcacacgtacaaggcaaaccacacgtgcttctcaacacacatccacaacgagaactatcaacgcctgcataatgaactcggtcaaactcctcagcaattagatttaacgcataccttgaaaccatgtcgacgagtcttttgtacaatgtcactttaaaggaatgtcctacgacatgcgtacttgtctcaaaagaagcctttatttcattgtgttgcaaggtgatcatgttgctcatggcatcccaaacattacacagatctccaacgctattttgcagtagccgctttaaggtccaatgagcagactcaaccctgcagtgaaaggtgcacaaatattaatttggaaaacatgatgtcattcaggtattgcaaaggatttacttgtttgtagtagtgtttccaagatgcatgactttattggtccatgctgtaacaaatctttctttgtggggaatcaaccatgtgtctttaacatactgaacaaatattggccatggtgcgcaggcacactcaaacttcataagacagtcttcatatgcattttcagaagcacaatctacaagacttccccacgcttccatgacatagtcccatgcattgttttgtcccacaagcgttttacacttagccttcacattcttgtcaatgtggaattgacacaacaagttggtagtgtcaaggaaaacctgttggatgacattcatcagggctgcatctctgtctgtgacaatgactttaggaaaaacctcagtctttaaaaaaggcctctaaatcgttcaagggcccatacaacattattgatacgttcaccctccaaatatgcataagcagctgaaaatgtcatcccagtaggtggcacaccaactatgtccaacaaagggagtctatacatgttcgtcttgtatgtgctgtcaatgagaaataccaaattacatgaatttatcaacttcactgaatcagggtgtgcccagaatatgtctcgtagaacatcttcatccttcagtctatgccaatggatatatctatcgcattcaagaagcttcatcaattgttgcattcagtattgttgcctctaacagaagtacggtacgcatatcttgcattgtatacttgtttcattgttgtataactttcgggattgcgatcttttaaactcagcataatattctttggtttaactcttgactttgtcatttcagcaacaattaccttctcttccgcacttaaacgaccagcataaggatgtccagtcattgacttggccaaacgatgattatgtaacccacatactaacttcatcatccagccttcacctcctacgactggtttccctcgcaacttgaaaggacaaccacactttctcgtgctagtggtcgtccgtactaaatctttgttgagtgctctatactttccactcctttcacaacctatcaaaagaaatgacgttcttcccctctttccagttgctgtgtccgaccgcacaaccacagctacaaatccaatctcatgcaacactacgagcccattccaataactcctcacgacacttaaacacctacaaaatttgataatacatattttaacagtggttaagtattaaatttactagcttttatgaaatataacatgatacaaatacctttgaagtattgaatgcctccgaacaatcaattgaagagttattattgtcacacgcaaatggttcccctcccttgtcttcactcatattcatttcttgatacatcatatcatcatgcatcaaattatcattatccatcttaacaaacaaattaaaaaataaccatcattaaaaaactaaatgcataataattatactcacaaaaatatcctacaaaaaatattaacttcacaatatatttttaaatgaaaataggacaaatacattaattaataatacttttaacttattaaaaataattttttttaaataaactaccatacggattggcaatctgtaagaatcgtactgattggcaatccgtaaggattatacggattagcaatccgtatgatccttacggattagcaatccgaatgatccttacggattgacaatccgtatgatccttacggattgacaatccgtaaggttcgtacggattgctaatccgtaaggtccatacggattgctaatccgtaaggatcgtacggattgctaattcgtatcacctttacggattgccaattcgtacgatccttacggatcgccaatccgtacgatcattatggattgccaatcaaacctccgacgacacagaacgtacctctggcgacgccagcggccaacggaacctccaccaatgctaaccaccacttccgcactcccgacgaagcgataccaagcgccaacgaataaccacagcagttcacgaacagcgaagaagaagaatgaagaaaacgcatggccatactgcctggtgtatgttaaaaagcaacgaggggtattatcggtatttgatgaaaatgctgggtgtattagaaatcctactggtgtaggaagttaatgccaaatcagtttggtgttgtgcttcatacaccagatataattattctacaccattttttttcataataccaaaactgcccttaatgaatgaaaaaacaaataaatggtgaaggcgctttacttactgtgtttggtttgttggaataccgtttttgaggcggaagcttggttggaacggattgtggggtggtttttttgcatttggaagtcgttttgaggtgtgcattccacatttggaaggaaaagctgggctgggtgtcgccggagtgaaagcgccgcagttgcaggtgagttttggtagttttttgggccgtacggatccgagaatccgtaagaattgatggtgcatgaccttacggattgggcaatccgtataggtagtacggattacacaatccgtataggtcatacggattgtgtaatccgtactacctatacggattgcctaatccgtaaggtaaaaaaagtttaaaaaagtgtaaaaaatgaaatttaatttgcaaaaaattaaaattaaatgtaaataaggacattttaattataagaagtgttttaaaaatgttatgtgaaagaatgacaaagcatgtagggtgtgtcaggcatgtgttgtgttttggatttgtttgcccatcagtgttttgaatgtcattactgtttaatgaatacgattttggtgtgtgattttaatgttagtaaatgttattatttagagtatatgtgtttttacatggtatgttaaattcttttttgttttgttagaatggacagcgatgagtggatgtacgagagtataatggctgatgatgtttacatgaatgatgacattggactagagcctgatgacttggacgagaatgattgttttgttgattgttctgcagcgttccaaacaaatgaggtgattatgtggattttaatgtagttgaagttgttaaatgattatattttaaacattgatgagtaatgacttggctgatgatgtggatttcaaacaaatgacttggtgtaggtgtttgaatcgcgtgatgccgtattgaagtgggctcgtaccgttgcgtttgacaatggttttgtggcggttatcatgaggagtgacaccaaaactggaaaaataggaagaacgtcatttgtgttaattggttgtgaaagaagtggaaagtatagagcatggaaaaaaggtttcacaccttcaatcactagtagtagaaaatgcgagtgtccatttaagttgcgggccaaaccaattacgggaggtgaagggtggatggtgaagcgactatgtggtaatcataatcatgtattggccaagacatttttcggacacccatatgctggtcgaataactgaggaggaaaagaccattgttgctgatatgacaagggcgatggtcaaaccgaaaaatatcctcctaacattgaaggagcacaatgcagataatgtaacaagtttgaaacaaatttacaatgcaagatatgcataccggacacgtacaagaggcggtaatactgaaatgcaacagctaatgaaacttcttgaacgcgacatgtatattcattggcatcgattgaaagatgaaagtgttgtacgtgacttgttttgggcacttctaggtgcagtgaaactgagcaatgcatatcatatggtgttcttgatagacagtacctataaaacaaacaagtatagacttcctttactagacattgttggtgtaacaccaactggcatgacattttcagctgcctttgcctatttagaggttgaacgtataaataatcttgtgtgggctcttgaacggtttataggccttttttataagattgaattttcccaaaggtgattgttacagataaggatgcagcattgatgagtgctgtgaaaactgtttttccagaggccaaaaatttattgtgtcggtttcacattgacaaaaatgtgaaggccaaatgtaaaacttatgttaggcagaataatgcatgggattatattatggaagcatggggaagtctacttgattgtgctactgagaagcagtatgatgatacccttatgcaatttgaagttttttgtgcaccgtggcctacttttgttgattatgttaaggacacttggcttaatccccacaaagagaaatttgtgactgcatggactaataaagttatgcattttgggaatacaacaacaaataggtaactttgttatttcacattgttcaatctgacatttgtttatgtttattggcttgcaaaataatttatttgtttgtcatacaatacagggttgagtctgctcattggaccttaaaaatgcttttgcagaatagtgttggagacttatgcactgtttgggatgccatgaataatatgatcacctagcaacatactgagatcaaggcatctttagagaaaagcacacatgtggtaggacatgtttttaaagttactttatacaagaaacttgttggcatggtgtctaggtacgcgttaaatgagattgctgctgagtatgagcgagtccattatgcaggaaaaaatccttctcgttgtgggtgtgtaatgaggacaacgcatggtttgccttgtgcatgtgacctagcgaaatatgtacttgggagcattccgattgatactgttcatgttttttggcggagattaaacttttcagatgaatgtatggatgaatctgaagtcaccatcactaaagagtgggaagtcatatctacccggtttgaaaaacttgatttgtgtgggaaaatgacactaaagactaagcttaaagaaatcgcgtaccctgattcaaattcgatgtgtcctcctcctgataaggtaaaaactaaaggtgccccaaagaagaaaatgacaaagcacgaaaggtcaacgagaagggacccgtcttactttgagtatgtagacgagttacattccgtgcataatagttgccctagtccaagacaatccgtttctgaaactgatgttatgcagcaacacatgatggttccaatgttagatcaatttcatccatttatgcagaagtttatccagaatatcatcgatgtcaaagcggatggtaactgtggatatcgtgtcattgctgccttattaggttatggtgaagaatcttggtctttggtacgcaatcatttgctcaaagagcttacagcatggactgatggatatatagccttgttcggcgggatacatagatttgaaagcttgaagcggtcgcttttggttgatggattgtctaaggtaagacaactaaagatgtcggtttcgtaagcctttactaccaaatgcatgggatctttaatgtttttcattaactgtatatttacaggtaactaaggataaatggatggatctcacggatatgggtcatgttattgcatcaagatataacgtgattctagtctcgctttcacttaaacaaagtatgacattcttccctctacatagccaacctccgagggatctgcaagttcatcgcatactatgcatcggtcatgtttttggtaaccactttgttcaggtatgattgacattttttgccttcatttgttgttactttaattacgttcaacctacctaattttaatatgatatgaattgcaaacaggtatttttacgagacaattgtccgttgccatgcacggccttgttgtgggctactcattgtaccatggaagctgaatcgtggcctagttattatattgggcgtatgcaaatgtatacaaaatgtatgaaagtacgtacagaacacgttgacttaggagatgcttagacaatttctggttaatgaaagttctatttaaattcataaaagttcaaatattaaaatgctacaattcatatgcatccataaatgttcaactgcaaagtacatgaataatataaacaaaggtcatagtcgcctgcgctgtctctgtctaggccgtgtagtgcgcgtatacactaagccgtcacctgtcacacctcgagcaatacgcagcagttgctccaccatagagtatgcatcagtgccctctatgactatctcagagtcaagcatatgttgcaacccctcaaccatgcgttcgcaagcaacctgaaataagcacatatattattatttaaatgttactatatcattagacgggttctgcattgcatatacaaaaaaaagttaaatttactaccgctgcatgtgcatgggtatgccctgtcgaagatatagtagactctaagcctgctgcagcatcgtcacgaggctgatgcaccaggggagccacaggtactggtgtactagttgcctctgaacaaaaacgtgaacgagcagcctcatactgtgcaggagtcatgattggatgtgacagacggtaaaaccactccatatagccagcaacatgttgacccgaatgatcaaccaagtccccagtcgcaacaatgtaatcagtgaagtgcatccatctgtcatccacatccgctgcagaaggcatgtcgcacgaaggtacctctggaatgtgctgtacatatccaaactgcctcaggacccggtctggacaatacatgacactatgtggaccccaacggatggccccaaagtataaacatatcgtctcaaactctctgtgcaaacggtggtcttcatacggagtccagcaaacatcagcagaagtcaacctatctatacgcaatcgataggttgacagaggtaacgccttcgccatcttccaccggcaggcacgtggctgaacctcctgatagtcatcgctcggaacaacattccctaacatagggaaatgctcgtatatccagcactgtatatgacaataaaatataaattaacacataatcaataacataaattaacaaatataaattaacacatcatcattactaaccacctatacctgtaacagtgtaatgtagcctgccagatgcttcccatggtgcttacatgcatagttcaagttttcatacatgtgcatcagagcaccagctccccaagcgtatctggcagcctcatcgatgtcgcggaaagcttccaagaataccacacctatatgtgtggcactcttgttagcaaacagagtgcaccccaccagatgaagcaaataagccctcgcagctaatgtccactgcctggcttcacacttctgataatacaactctcgtagccacgataggcgaacgtatgccccatggcattgggtcgtctcagcagtggcttcgtcaactgtcacatgtagcaaatcaaccagcatgctaatggcctctcctacgtcgataacctcaaaggactgcagcgctcccataaccggaagatgcaagatcgaagccacgtcatccagcgtcactgtcagctcccccataggcaggtggaaactactggtttcagggtgccatctctcaacaaaagcagatagcaatcccctgtccccagtgtccacacagcaagaaacaagaggagccagccccgatgcagccactatgccctggatctctgcagctggcttcccaaacttaaccactttccgtccgtgagaagccaacttcagctcaggacgctcctgcagtaaagactatatcagttttcatgtatgttatcaataattgaatatgcaaaaccgatgaaaattataaaaatgtacctctccggcccagactctaacggcaatatgatctgcataagatgtcaacaacgaacaatcctctagtccccctggaaatgaatcaatctgatcatcaacatcctgtgcctcagcatcaacatgctgtgcctcagcatcaacatcctgtgtccgatcatcctgtgtcggagggatatccgtatccacctgaggctcctcaataacgggatcctcgacagcaacctcctctaacctttgcctacggacggatgccgtaggcctacgcctctgcccaggatcatcttcactagctcgactaactccacccactttctgaggcattcctctagtcctagccatgatctctgcgtatcatttgtacacatgtcagccatcaatccataaccatcacatgtcattcatgactagaaaccaaaccaaaccaagattggtacacagccaccagatgtcattcatcaattcaaaccacacaaaaccaaacaaacatcaatccataactattacatatcattcatcacatactacataaccactacatctaatacttcacttcataaccactacataaccactacatcaatccaacattactacataaccactacatctaatacttgacttcaacccagaccaagccaaaccaacaataacacttaaatatgcattccagctcatactacacaagcatgaccatatcttaacttactgaaaaagcaaaattatattcaaatatgtccaaaaccaaagcaacaataacccttaaacattgcaagtcatgttacactaaaaaattcacctaaatccaacaaaaaattcaaaaaaaagcatccatggattgaccaatccgtaaacaacctacggatcacataatccgcagtaatccgtaagcttcgtacggattatgtgatccgtatgctgtttacggattgtctgatccaaagcacctttacggattgtctgatccagagcacctttacggattgtctgatccgagagcattgtacggattatctaatccgtatacatattacggattcagtaatccgtaatatgcatacggattacataatccgtacaatgctcccggatcagacaatccgtgcggcctttctccacggattcccgattccgtttccgccatagccggcctgcaacagcctccacacaccaccatacccaagtttcaaccaataaatcactcaccggaagcaggaacgaccaatgcggtgcttgtcggaatacaatgcctcactgaacaataaccctcgccgccgattaaaaacaaaggagcctgcggtgcttgtcggaaaagctcactgaaccaatgttgctcggatagtggtgcaggaaacagggaagagggaagtaaacaacgaattggtgcagcaaatggtgtaggaagtatggcggagacagcgcagagaaggaggaggacgaagtgggaagaagagggaaggacaaatttttaaattttttttatataattaaattttaatttaagggtataaatggaaattaactgtataaatatggtgtataagattttattatggtgtaggaagctgtagccatcagtttgtccttaaagaaaaaaaaataggataaccaaaaaggctaggaaatgaaagcccaatgcttctgtaacaaaccatttcgtgtttttgtatcgtctctggcattctacatccactctgtaacacaccacacaacaatggcttccagttccaaatcggagacttttctgcagcgatacggttactattttctgttgggatcaatcgctgctgtctacgtactcactgtgccttacactaaggtcgaagtgagtttcaacgttcaggtctctctcttcatcttcttcgacctctttgcttatttttattagttatttatttatttataaattataattcctttgtgtttcctaaaggcaatgcacgatattctctatcacaggctgaacttagacaatgtgagttcactgtgtctttgctattttttttattagttggtagtttattgtattttgtatataattgatctttttttttaatgtttctgttaatagtatgatcatctggaattccctggggtggttcctcggactttcctcggtatgtttttgtgttttgtagccattttctctcaaaatatgtatttttatttaagcttattatgtttttcatatctttaatatatctctttttcagtttactacctaaaattatttttttatgattttactatatgacatatccaaacttttgcttttgacacctactattagtcttggtctgttaagtgatgatgtgacaagctaacacactagcaaatcatcagttaagtgatgatctgataagttaacagagtgccacatcatcacttaacaaacttagagtaatggtaagtagtaagtaccaaccaaaagtaaaagtttaaaaatgtcagatagtaaaataaataaataaataaaaaattaggtagtaaactaaaaaaggagtatattacaagtatgaaaaacatatttaagccttttatttattcatttagtttttggttggttaatgttttcatatttagcactagtcagacctttgtgatttattatggaaaatgataatttactttgtgaagaattaagcaaataaggattacggactcgtttatacagttgcattgcttgtgtgcctgatgtgaatgaatccatttaataacctttgagattgtgatttgttactagcatgtgttacaacggttattcaagtttgtttagtttttagtgctgaaaatttaaccgtgatgatgttctattggattgttaagattgagtttttgcaattgccatgcctcctcggtataagctggtgtgtgctagtgtagtacttctctctttgttatattgttatatgttatctaattgtgttattgttattttgttttaggtgctttgcttgtgtcaataattgcatcaccgtttgtgttaactgcaagtttgctgcatttgccaaagttttatgctcttctcataggtatgtatccttctttcatacttgacaatcaatgcatattttagttttctcatttcagggaatgctgtttttatcttttttccacctctggatttctcctttcctttggtttaaattgaaaaaaagactggcgctaatgatcaaacgtgtgtatgtttttttgttgggaggagcccgacatacatatgccaagaagtatctagttattacttttttgtttggatgcctgggaatttgtgcagataagaaaccaatcacactagaagtttaaagtctaaaacactgtctttagctggaaaataatagctcgaaaattaattctgataacgtacttgtttcactgagccattggtctctatgttgttttgcatcattctgttagtctcacaagcattcattacaggaaaatgtctgtgttatatgtacataattgaattgactgatcaaatcaaatcaaataatctacaattcatagttgagtttaaagtcatgaaccatgtcaaaatgctcaaaattaccaagatgatggtgtcaatttttgttctcctgtgctctctgttacttgagaatgtggtgggaagtacattaacatgtcttattgttgatgtctagttcggatggccctagggggcataatactatacacactaagattctttcgacatcaggtatgtacctttggatcatcaataagcaagtaaatttatttatttattttacttatttatgaagtagtgtttattcaactctctgatattatacctatccaattatagctcaagtttcaatacttctatcatgcacccttgcatatttggtgcatggatgcacaaagatgtttaactgttgatcatataaaaataataattcaacagtagagataatttatctcaatttatttggcatgtagtactataatttttaaaattatgaaaatgttctctaaagactaaagcttggccagagcttcagtgagcttcatgactatgaaaaggagattaatcaaacaaaggagtaaattggaaaatttagtgaggaactcaaatgtggttgaaccttctcaagcaatggcaaaaatcttggttatgtaggttgcatatcttttatcagtgtttgttcaagctgtcctaattcatctctataaaaacttaactaggtgtcccaatggaatggttcatgtgcctccttcaccttgtcgtgcctgccagcattggaaaatctttgagcattgattaaaactttggaaaaggataaaactggtaaaatattgagagagaaaaaaatctgaagggtgcatggcaccaaatttgagagcattgtagaattgttccaagtttcataaggaagcaacaaactttcttttcttatcattctaataatgatctttgctttgcagataatgaataagtttggacatcaagtagaatccttctttgtgatattaactgccactcagtttcattttctgttctattgtacccgtccacttcccaacattcttgctctgggcctaggtaaactacctagcttgttttgcttcttaatattcctgatagtgtttttcttgtttttaatgatgaatggcagtgtgaaaccttctagtcaatgttatggtgctgtactttctcttcatttattttgcagtgaatttagcatatggatactggttcgagggatgtttttatgcagctctaaaatctctggtatgtatgttgtggttttatttcaggttcatctcaatactaaatttttcttttgaatattataagctcttatgtattttttcttcacttcatataatatgcatattttgccaagccagaatttcgtgattaaatattctgtctcagaattagttttttaaactagtcttttaccgttaaattactatttttaaaaaagaaaagtccaacttctcatattatcaagtttacaacaaccaagcttttttccattaggtggggccagttacatggatactatttctttattttttttttccagaaatatttacttaatatttgattccaaacctattccattagattaacaaaacttaattctcatttgtatgtgtattgtgtgttgtgtatgtcaatgtgaactagttgcctgtgtgataacacatgtgaattccgtggaggcagtattggttacctcccaatgattattatttagtagggtctatcggcatctatgtttttcattttgttctcacagagacaaattcaatgcccttgatttgaatttctatgattggaatctcttgcacaactaaatgactcctccttttgcatatctacaaagttcagttaatagcttataatttaatgaaaagagattttgaaattaacttcttaagcatataaatatatgcaacttcttagccccaacttttttaaaggaaagggaaattagaaagaaaggggtgaaggtacaattaagatgagaatgaaatgtttgtggtccattttgaccagaatagaggtaaaaataaaatttatccgcaattttataatattttataagtgttaaaagtgaactgtacatggcatttagatgtttttgaaaacttgcaagggcaagaatatcttgtgaatatatatatatatatatatatatatatatatatatatatatatatatatatatatatgatatttttgctcctaatcaactgtgagagaaaaattcatgaactcctaaagagggttgttagagtagtgtaggttgcggtgtgatgacagtagtgcagtgtcagtggtgtgtggttagagtggaggggcaagtggtaaggggtgatcaatgatgtctctctatacattccattctcaataatgaaataaaacccacctccctgcttcactcagaatgtctttagctgaaacacgagaatactgaattatcagggcctataaccttcctgaaatattttttaaaatttgaactagatgtggggattatacaatgtgcataacattcttaattagtagctcacttaccctttatattatttatgctattatctatttaaaatcatagaatttagtgtgatttttttgggtacatattttagcattgttttattgtcaataagtttgaaatgaaatgattttatgaaagtgatttttttttttaaatatttctactagtttaatatatcatcttttcacacacacacacacacacaatttgacaggaaggaatgttttctggaattagttggcccttttaattcttaatatcgtagtatgcttgatatgctttgccttttctgaaataatctctttgcacatttggattatcagatttttgctacaactgtactcagacgtgacatgctgttacttctttgccccattgcgctgcaacttcttttggtatgttgtgcttttctgtaacttgcttttcatttttccgttttctccttaaattcttgagctactgatagagagcttgtcaattctttaatttatttgtttggaggatatctaatgagttatgatgcttccccctacttgtacctgttcttgttttcctgataaactttcttctatatatttaaaaagaaaaggaaaaaatctggctattctcattattttctgttggatgcttgaatttcttattttccaacaccactccgtgtctattaaactattgcatcctattctttatcttgcttttaataatgttatgctttaagtttgacaagtggagcaagtatttcctggtactggaatatatttattccatatcaactttggacttctgatgtcaatatcttgacaatgaaatgaagatcacgttttctgtaaaaacagataagaaattctattgaactgtcctggcaacagttctaatattgatatcatcatttcccagcataatttaaattaaatctgatttggacttttatcttatcgaatatttcaatttaaaggcttccaatttatgagttggttattattgcattcatgagaatttttgttgctttgtcactgtctcagaggctataggatgattggcaaatgtgtttacaatgttcaattgggtcaccttagaatccctatgtttgaaagccacctaaatttcattattgcattcatgaaattttttgttgctttgtcactatctcagaggctataggatgattggcaaatgtgtttgaaatgttcagttgggtcaccatagaatccctttgctggaaagccgccttaatttcattgttgcattcatgagaatttttgttgctttgccactatctcagacactataagatgattggaaaatgtgtttgaaatgttcaattgggtcacaccatagaatccctatgttggaaagctgccttaatttcagtcacccctagcccaccttaattgcggcctctttgggctgtcttaattgtagccttgtggggggggttgactagagatatgacttaaagagagctcataaagcttgagcaatcctcattttacaaaccggttttgtgggtttgagttaagccttaaacccaaattataaatttataaggtggtattagatcctatctaaaatccaatttttggccaacccacatttgtccatgctccagccagatgtgtcttgggtgttattattgcgatacttgcatttgtccatgctctagatggccaatcttgggcatgaggattgtgttggatagctgccttaattgtggcgactcatcacctgccttaattgtggtggcctggcctctttgggctaccttaattgtggcctcaaggggatttgcttagtctcacattgactaaagatatggttataaagcttggacaatcctcatcttacaagttagttttgtaaggttgagttggactctacgccaatttgtaagaccctattgactctgtaattttatttaacatttaaatatagaacttcattgcgttaactatgcttgtgttgttatcaatctttggagaacaatgtttattatatttggcatttgaagttaatacttaatagtgctgtttgatgaattttcttatcctcattttctgaccttgcaattttggttgaattattataaaattgaaatttattctgaaaattccatacttatggtaacatgatcatgctttgtgcctaataaaattaatctcttgaagggaagacaaaaaaaatttcagtgtgaggtgctctaaaacactgcactgggatggctctcttctgcataggttggtgacatgcttttttactttaattttaatctgttatttaaataaaatcaaaattttatgcatgataggatgctattgttaaaatttgattctaacattatggtcttctgattacaggtctaaccatattggttgattcaattatgtggaaaagattattgtggcctgaatttgaagtcttttggtttaactctgtactaaacaagagctctgaatggggagtatccttcaccaagtttatattgaacaactgatagctgtagaatatgaatacatgttatacatatttcatgaccagggtattttttaatttttttctggttctgtttggtttgaaaaaacatttttttttcacaaataattacaaaattctcaccttgttttcactttctttcctgtttccaaagatttgtttgtacaggaaaaaagagaacaaaaacccattttcactattttttgtataaatcattgaaaacaggaaacaaagtgaaaatagaggtattaatttgtgaaagcatataacattttaaccaaacaggcctttgtttgctaccttatatattacttaatgatgaggctaagccctagtttagcaaggagttacaagagaagatcttttgtcacgtttcttgttcactttagaggttgatgttctgaattgaatactttcagttgctgtttgtggatagggatatcattcaggatattagagttggtttaccatttacagtaggttatggtcttcatcttaagtttgctgatgatacaattctctttttagaggataatgatacctttggggatgttctttctattaaatttttggaagttatctttgatttttttaggttaacttttctaagagtgatttggctggtttaattgtggatagctatgttctttcttccattgcttctctggatgttgtgaaactttacagtgccccttcacatacttgggagttcctttgggtggtaatccttttccacttcttttggaaatcctgggattactcagatctttgttttgaagtgaatactaaattgtatctaattctgtgtgggctgactttgaaagccatttatttatgtgacccaagaaggactgttcaaagagatttgtacttcctttgtctcttggggtattttagtttgtttcaaaatatttgtcacttttagaaaatcaggaaagcattatttacttttttcaatcaataccctcatttgagtatctcacctaaatacttgaatgaattcacaaatacattctaactctttaaataagggtagttttctcaaagaaagttctcttatcatagtaatttgtgtaactaataattttcttaagtgtgcattaccttaaaagacataatatgaaaaagaggaattagtaaatttctgcaacaagaggattttggtgtatttggattagggtttgtatgtttagtggctaatacttgtattcttcattgtttgtgaattttctctctgttttgcccatggacaaggcacactaagcttattatcatgacatcatctaaaatctgcatattgtctgttgtctttgataacactgtcctatttgtatttgataaattattatgttccttgacaatatttttgttagacacatgctttccattggtacttcacctcagctcttcctcgttcactactttgctgcatatccactgtcactagtaagtcttggattagcaaatatcctctaacgattgcattgttacttatgtagatttctagcctgacatggtggtttcttccacttccagtgatctggttaatattttgtaacttaaatatgaaatattttgggttttctccttaaaattgaactgtctgaaggtagtacttaaagattaatattttccctatgattaatgtttgaactatgaattaatattgaatatttcttacctgttagctttggaaagtatttgttccctatttgttttcagccagttcaaatatcttaaatgcatacacttagtcatgtatattcttgcaaacccagtttaaaagataataggatatttggattgtggatagtcatggtatattgcactgaagaccaggctagttgcatgattagtgtagctgttgccctttaagaatttatcataactattttttagagaaggaaagaaagaaattattttagattaggagaattatatacacatgacaattaaaattgtagtgttatttcattcacattattggtttaacatttcattcctgttattgggatctcatgtgtaaaggattttttttaattatttaacaattagcatactgatgcacaatgaagcagcattaaggattgttttgttatttacaattttttatgttattctcataaagttttgaatttccttttatttgagagatgtcttactattttagggattttattcttcgttatatgttcttaagaaaatattgtcatgttaaaatttcctgtttaatttaattcgattaggatttaacatttaaggaaattaatactatttaattaggatatagctaggattttatattaaactgggattttcataatctttgggttataaacatcttttgagtttcttatgttattgggacttcattataatacaatatttagattttcatttctttagagaatatttttaaactcaaccagaggagtactcttgccttagatgagttctttaggtgaattgatccacatcacatgccctacagtatacgccactttttctaccttcctcgaacggaaaaaaaataaaataaataaaggaaaagaactgggaaaagaaaaagaagaaagcctccttttcatcctaacttcagttacttgacaattgacatgctgtcttctttgcagtttggcctttttgtagacagaagggtacgatctttcacttttccagttcttgccttcattttgctttattctaagcttccccacaaggtattacaattataatctgaaatccattgcctaatatgtgtttgtcttcaaattgaaacatgtatttagtattattctgattgatattgcaggagcttcgttttatcataagttcaattccaatatttaacttgtctgcttcaattgcttctaacagaatgtaagttctcctaatgagtattgtataggtctgtcaaaactaattgggttctatttgtattttctgaaattatttcattgtgtggtcttatatactaaaatacaactgaaaatggatattctgggaaagcaagtaattgagattttaaaatatgaatgaatgtgcattcctttccttgtttctatggcatttgtatgcattctttctcatttgttattggattatgaacaacattcataacttttttaatgtttatcaacctatgttttggcttcaagaatgatgatgctgtgtaacctgtaaggaagctcattagaccaagggattaagttttgctctcaaatgacaatacttgattatgagcacctgagtggcagttgcccctgctttatttcagacaaacttgaattttgcctttaggatgttcatataaggttgttgcgtcgtctatcaaggatcatgcatattccttgtttaaattatgtaaaatctggattgagaagcatgttttcttcatcatccttagtaactcaaaaaattgaataacttgcagttgctaaaatatattggagcaagttagttcaatatacaaagcatcttcctcagtcttctgtatatatttgttcttttgaactgggcttaatttagtgctccaataatacaatatgggttgtgctctaataatacaatatggctgttctattcattttgttagtcaccaagtgaatgcaacaagacagtttgcttcattgcatatttgcatgatccaaaggtatttgacattttgatggcattcataacattaggtggtttgaggtcatttacatgaaatttcattttgcttattggtattttagttttaaattcaatatgacaacagaaaaaaaaaacctttaacctactagttttaaattcaatatgacaacagaaacaacccttaacctactgggtggtgaggcaatatagtatgaatacattattatgatcattgtcatctagtcattttgatacaggaaaaagttaactagatataaaaacaattggaataatgaatacaatatatccatctaaactagaaatctaaaatcaatcgagaataggaaagagaaagctacgaatgccgcacagctgctcaatttctccatgcatgacacaaactgttgaagttgaagctgctgtgacttgtgagttgagtgccaaaaatttgaagagtggtgattgagctgaactgtatttacaatctaaacagagatatgaaaatttcggatgtttccctctttgaagaattatatctgtttccttttaagttactaaccttcagtaatatgcatcctgaaatgtttcagttacaacaataagaaaaagatgatttggaacttgcttttcctcattttgctgggattacttttaatgaggttagttcaccagtcattttgtttcattgtgatggatatatcattctttttttgtacttactctctatcttgcagtcttgcaggcaccatcacaagtttcatggcatcatactggaactatcctagtggccatgccttaaaagaattgcatggaattggtgacatgagtcctgaaatatccatgcattggctgcaaagtattttgctagaattttcctagagagaaattgctccttgtttgtcccttaatttggtaaaatattggagttgcagtttctatttttcataatcaacagtggcaagttctgttaaaattgttaatactgttgtacccgttggatctctgatgaattactaaacatcaatttttaatatggttcagtactgagcttgatattttggtcatggtttattgttctaaaaattatttccatgcgtgctatctgagtgttcatattgtatgacattctggattaaattaaatgatatgtgactttggggtgctttattgaatgatgtgttgtatgacattctctgaatctcttacccgcattccaaaaatatatctcaattctcaaaaggatttctagaataaactacattattcttcgggcagtggtttgagcaaaaaaattagcatgtcataataaatatgccaaagctttccctttggtgtatagagctgatgagttattttttggctaatttatgcatggaccaatatgcagtactggttggtactggtccttctgattttctcaaggacaatacaaaggcagtgtttgtgatactcatacttattgtgatagccaagatgcattccgtattgtctccaatcctgttttctaggggatgaccaagcacaatgatgtagattgtcactctatcagaaaagatccattctggagacatcataacttgttttgtcaatccaaatgctcaccaatcttttgagtggaccttggacaagttgttacttgttatctttgtaaactaactagctctggtcacatatgagctaatgcactagctttgacaggttgtgttaggaaaatttattattctttgtccattgaatggcaactagaaagattgtatcttttttattattacatttatcatataaatatggagtctgtggtgtagttgaaacacatttatggacatgcttttgttctctctacatgaacaatgtgtcaattttgggaaaagataaaggattgtgcttattgttactgtggtattaaggaagatttttctttagtgtttggttgatgtaacaggatttgttttgatttgtacgatttcttttcttgcccagaattcctttcatctgaattagaagtcaaatgagcagtcattgagacctaaaacaactccaatccagaaaaccccctcatttaaattataaaaagaaggaatttcattaaaagataaaaaaaattgaccactaaagaagatagtaatgtaacgagttttttatttttatctttttgatactgccccttgtcattacttttgttgtctttgttttgattttttaattgttgcaggctttcataatgatacagatgaacggtgggtgcacattgatacattttcagccatgaatggaatatctcgcttttgtgaaagtgaatttccatggaggtatattttgcttctttcttttgcagtgtactaatgatgcatcgagttgttaattgcatgcatccaaacaaacctttctttgactaaatctcatttttaaggatcaatgaagccatgcctttctgagcctttatctatgatttagtatccacagaaagttacagttctctaatttcattgtcctacttggaatgagatcaactatatttattttcagaagttatgttttcaaaagttatcatcagaattgcttctttggctgagtaataaattgatgcaaagaaaaacttcatcgaattcttcgacttgcacattcacatttttcggttccccccttcaaataaaggatagtgtatttgtttatttgattttatcgttccacagtaaactgtaattcagtatgacataggtaccttcctgttgggaaggcaggtagtgtaggccttgataatgttgcttaagtacgtaatctgctttctcaagtccagcttttcttagtagagtgacaaggagctggagctatttatagggttagtgaatgtgtttcttattgcactaatgaaaaaacaataaaccttgatctattttcctgatgctcaccatattcaggttcattagttagcaccttatctctagttgaactgagccatcattgtggttatagggtttacatttttaaacatctacttgtaagtgtcttctataccattcgttgctgtagcaagttcaatactggatttgctacatggtctgtaatgtctacatggaaaaattgtggaaaaactacatatacaaatgattaaataagaatgagatagcatgttatggggaagatttaagtgttaacgaggtattagcagtatacttctcattatttctgcattcattgctttcaatttccaacgatcagactatgctgctttccactattttccgactacaagtaacttatatactattctctggtgcaggtattctaaagaagaacaaattagcttgcaagaatttcagcagagaaattttacttttcttataaagtgagttatccattaacatattgctagtgttatttctagtactttgtctgacaaacatataaattgtatagcacatgctaatgtttatgatgagagatcctacaaactgttgacagaaagtgtcggtattgtcaagtgagtaagtgtgtgaaataaaattgataaacttcatacattagatccgcccgcgaagagtatatgttgttggcaaggactgcatcagcatgtgttgtgtgcataatgatcattgccatgaaccaactatcctaaaaactttagttttaagtgaagacaaatgaatagttttatattacatctcaactacacccattcatgcaaaatcccttatggcttgaagcgtggatagtgtgcaagcccaccaaccttgagctgaaatttaacttcttattacaagaataggtgcagcacagattgaaatctagaacaataagtcatagaatctttgttatcatgtaatggacctactatcccaaaagcttgagctattaaagtgaagacgcatgatgattttatattacatctctaactgcgaacaatttggcatttggatgaataaatgttgtctgacgtctgtcatctttctgtgcttgcatgtgtagaatgtgtgtgtgagagaggaaatgaatgtttggagatattgaattggaagtttatatatgatttttcacatgtcaactcactgactaattgcaagaaatttccccctttatctgacctccttgttgatctttaacagtgaacatcctgcgatcaatggattcaagtgcctatttactgaagatgggttttcaagagtccgcctgaaacctggctatccaccaattttactggttggtaattgctattagtattcccaaccagctcaaacaatgaatgctttgttcattccaaaggtattgatctctgttttgaccctttgcaggttaaagagcccaaagtgtatgttcatggaaatttagaaaaccagaatattttcagccaaaattggcctggctgcccataatgctgtctggtatgatagtctatttgaaatcatcaggtttgaagggtgtgggtatcctttgccacccttttttcttaattaccaatgaggaaggagggggagcaagtgaaagttggtattgtgacccctactaacagttctgtgttgtcaatattaaagagaatgtacatatgggtacaatagtttttcttgtagcatttcaaatattgattctcggatgtagctatatctgatttttgaattacatatagaagattcggttcaacaatctggtttgggataacttcatttatgctggaaaactttttggctcaattctggtaattggtgtatactgtttgctagagtagaactttctgtattaactagcatgatactcgtgtttttgcacgttttatagacaaataaataatataatatatattcaaattattaaggtcattattaataaggaatcaatgtttaagtggtgagtatattatttacaaaagtctaaaatataattttttgtagtcagatataacaataagataaaaaacctttgggtaggcaaataaataatataatatatattcaaattattaaggtcattattaatacggaatcaatgtttaagtggtgagtatattatttacaaaagtctaaaatataattttttgtagtaagatataacaataagacaaaaacctttgggtaggaaaaaatacaatgtgaatttgtatattagaaaaactttttttagtgcaatattaaaaaaacaagataccatgcaagataagagtagtcatatatggagtttaaatacagtatagagcaggggtgtggatggacatgtcttttgtctccattaggggttgggtatacccaccatgtccccgtcctcagagtgtttcttcatacaccattagtaatttctaatacaccattatttttaaaaaaataccaaaactgcccttgctggttgtggacgggtatatatctgagctgagcagcatttgatcaccatttcatacatctctttcataagcattggtgattttccgttagtcccagttgcagttgatacgcattggtgattttggtgccggagtcgtggttgtcggagctgttggtggttggcggagccgcgaagacgcggctgaactgtgcaaggtaagtggaggcagagtggatcgtacggattcggtaatccgtatggatgatacggattaaccaatccgtataacccatacggattacttaatccgtacaatgtgtacggattacttaatccgtacaatgtgtacggattacttaatccgtacaatgtgtacggattaagtaatccgtatgggttatacggattggttaatccgtatcatccatacggattaccgaatccgtacgcgttgtacggattacataatccgtatgtgtggtacggattaaccaatccgtatggattggtttgctgaattttactgttttctaaatgttggaaatatgtgtttgtataaaattgagtttagtttgataaatttgttgtattgaatgaatatattgatttaattgttgtgtaaacataacaataaataaaataaaacaatatgaatgcaaataaacataacttagcagaatgtttcatgctgaatgagtttgaaatcttattggatttgttaggatggacaacgatgaatggatttatgacagtataatgtcagaagaagttaatatggatgaagacattggagcagacgctgatgcgtttgaattcaatgaaaacaacggtgtggaccccgatgcgattgaggataatggttgtttaattgattgttctgcaactttccaaaccaatgaggtaattatgtgtatgttaatgtatttaaagttgttatatcattgtatttgggatattaaatatgaattatccatttgtgtaggtgtttgcatcacgtgaggcggtattgaaatgggctcgtaccactgcgtttgaaaatggttttgtagcggttataatgaggagcgacaccaaaactggaaaaataggaagaacgtcctttgtattaattggttgtgaaaggagtggaaaatatagagcatggaaaaaaggtttcacaccgtcaaacaccagcactagaaaatgtgagtgtccttttaagttgcggggtaaaccgatccctggaggacaggggtggatggtgaaggtaatttgtggcaatcataatcatgaattggcgaagtcattttgtggacacccatatgctggtcgaataactgaggaggagaagaccattgttggtgatatgacaaggtcgatggtgaaaccaagaaatattttgttaactttgaaggggcacaatgccgacaatgtaacaagtttgaagcaagtttataatgcaagatatgcataccgaacgcgtacaagaggcgataattctgaaatgcaacagctaatgaaactcctagaacgcgacatgtatattcattggcatcgagtaaaggatgacaatgttgtacgagatttgttttgggcacatccagatgcagtacagttgactaatgcatatcatatggtgttcctcattgatagtacctacaaaacaaacaggtacagacttcctttacttgacattgttggtgtcactcctactgggctgacattttcagctgcatttgcctatttagaggctgaacgcatcaacaatgttgtctgggcccttgaacggtttagagggatttttatgaaaagtgaattcttcccaaaggtgattgttaccgatagagattcagcgttgatgaatgctgtgaaatgtgtttttccggattcaaccaatttattgtgtcggtttcacatagacaaaaatgttaaggcaaaatgtaaaacttatgttaggcaaagccatgtatgggattatgttatggaagcatggggaagtcttgttgactgtgctactgaaaaacaatttggtgaatgccttatgcagtttgatgttgcatgtgcaccgtggcctacatttgttgattacgtgaaggacacttggttgaatcttcacaaagaaaaatttgtgaaagcatggacaaacaaagtaatgcattttggaaatacgacaacaaacaggtacatctgttattagacattaattacattgacatttccattagttaattgtattttaattaattcattatatgacctatatgcagggtggagtctgcacattccactttaaagcgcctgttgcagaatagcattggagacttatgcactgtttgagatgccatgaacaacatgatcatcttacaacataatgaaataagggcatctttggagaaaagcacgcatgttgttgggcatgcttttaaagttactttatacaagcaacttgttggcactgtatcgaggtatgctttaaatcaaattgctgatgagtatgaccgagtgcattatgcaggaaaaaacccttctcggtgtggatgtgtgctgagaacaacgaatggtttgccttgtgcttgtgaacttgctaagtatactcttcagagcattccaattaacgccgttcatatattttggcggagattaaactttgcaagtgaaggtatgcatgattctgaagtcacgataactgaagagattcaagccatttcaaaccgttttcaaaagcttgatttggttgggaaactgacagttaaaagtaaacttaaagaaattgcgtaccctgatttgaattccatgtgccctccccctgataaggtgaaaactaaaggtgctccgaagaagaagatgacaaagcacagaaggtccactagaagggacccatcatactttgagtatgtagacgaatttcattcagtgaagaatagttgctcaacgccacaacagtccaattcgtcatgtgatgttcagatgcaacacgggatagttccaatgttagatcaatttcatccatgtatgcggcagttcatcttgaacgtgacggatgtcaaagcagatggtaattgcggataccgtgtaattgctgccttattaggttatggtgaagaatcttggtcattagtacgcagtcatttgctccgagaacttggagactggacagatggatatatagccttgtttggtggtatacatagatttgaaacgttgaagagatccctactggttgacggattatccggggtaaaccttttttttatttaatgattatggctttaataacaaagtaattactactcattgtgtctaacagaatatgtgtttatgtacaggttactatggataagtggatggatattacggacatgggtcatgtgattgcatcaagatataacgtcattgtggtgtccctttcacttaaacaaagtatgacattttttcctctacgaagccaaccccctagagacccccacgttcatcgcattttgtgtattggtcatgtctttggtaaccactttgtgcaggtatcatatttatttgtttatttaatgataatacaataatctgaaaatgtagactgattgtatgatgttattatttgtaccaggttttcttaaaggacaactgccctttgccgttgacagcatttttgtgggatagacactgtaccaacgaagcaagacattggccctatcattacattagtagaatgcaaatgtatgttagatgtatgaattttaccactgaacatgtagacttatgcgatgtgtagaaacgtctaaaatgcaataaataaagtgttctggaaagcataactgtatgtacatatatttataagtatccaggtctgtacaatatgaaaaatacgacgcaaaagtaatactaaataagtgttatcaagaaagcatgactagtagtctactgaaggcgcctccgctgtctatctcgtgctcgtgtgcctcggcggtagactaggccgtggtccatcacactacgagctaggagcaacatgtcttgcaccctagagtatgcttcggtgccctctgtgaccatcccgtcatctatgtaagcctgcaaacccccggccaatcgttggcaaataacttgcaattacaatggcttgaacaataaattacccaacgtaatacaatcatatatcaaaatgcgtaaataaatacatatatttttaccaatgcatgtactggatcagaaggaggagctggatcagatacgtgtggtgaagggggatcaacatctgctgaagggggaggtgtagggtcagaaggaggacctggaggagaacgaggcgcgtatggggcctgtgaccgggcctgtgcatactgtgtcggagtgagaaatggatgtgagatccgatagaaccactctaaatatccagcaacacagtgacttggataatgtaccaagtcaccggtcgatataaggtaatctgtgaagtgggcccatcggtcgtcaacatcgtctgcagataggactcccgacaacggcaagtttggaatatgctgaatctgtccaaactgcctcactacccgatccggcaagtacctaaccacgtgcggaccccatctgatgaaacctctgaataaactgatcggctcaaaatctctaaagggtcggtgctccgcatatggtgtccaacatatgtcataaggtctcagcctctctatgcggacacggtacgtcatcaacggcaacgccttcgcacccttccagcgacaggcacgcggctgcgtctcatcgtatgtagccatagcacccccatccaccacagtgggaaaatactcatatatccagcactgcaagtagaaatgattaatctctgaaataattcaaaacaaaacaaacccacacacacagaaagaggcacaacattacctgtaatagtgaaacatatccagctagatgctttccatggtgttttgatgcataactaagattctcatacatatgcaccaaaacaccagctccccatgcaaatcgtccagcctgatcgatgtcccggaaaggctctaagaactggacgcctatatgtgtcgcactcttgttggcaaacaaagtgcagccaacaagatgcaaaagataagcccgggctgccagcagccactgcctagcgccacacagcctcaaatactggtctcgaagccaggacaagcgaacatatgccccgtggcactgtgtcgtctcggcttcagcctcatctacggtgacgttcagcagctcaaccagcaatgtaatggcctctgcaacatctatctggtcatagtcatggactactcccgtgatagggatgtgcaatagagaggcgacgtcatcgagtgtaacagtcaactcgccgactgggagatgaaaactcgaagtctcagcatgccatctctcgacaaatgctgagagcagtccccgatctccagtctccatcgagcaccgtatcagcgggctcaggccagtagcagccactaacccctcaatctcaggagctggcttcccaaatttaataaccttacgcccatgggaagccaacttcagctctgcacgttcctgcaataattaaatgaacatatgtttacaatggctaagaagcacaaatatcattttgtcataaagtgcttgtcttatatttacctgtcctgaccatactctaacagccacatggtcggcataagcagtcaacactgaacagtttgctggacctcctggaaaaccatcagcctccatatcatctgtcaccggtacatcctgcaccagaggatcctgaaccggagcatcctcaaccggaggatcctgaatcagaggatcctgaaccggagcatcctcaatcggaggatcctgaatcggaggatcctgaaccagaggatcctcacgaagactaacacccgccctttcacggcgaaccgatgctgtcggcctacgccgatcatgaagtccacctcgagtcctagccataatctgtacaacatttgtatatatcttacattatcgcttcagtctaaatgacatacacactatctgatttggggtttaccaagttggattcacaggcatacaaaccaataaagtccaattatatgaaaacaatttcatatcggtttcaacaagtatgattcaaagcctcaggaacatataaagacaaattctaacaaaaccatatcatttggggttcaacaagtttgcatcacaggcatagaaaccagtaaagtccaagtatatggaaacaatcccataccggattcaacaactgtgattcaaaaccataatcacctataaagtttgattcacaagtttcttgttcaaatcacatgtaaagaagcacatacaatcaataccatataatcatcaatgcatggataactaaacaatcacatacacttgaaaattattgttcctaattacttcaaaatcatgccatcatcaataattggatacctgttgtcttcaattcttttcataattttcattctactaaattcaatgaaaggaaatacatggccattaccacatacactaatcaacatgtatggctaacaaaatacaaaatcatcttcctaatattttaaaaaccatgtcatcatcaacatttgcataaccatagataaatgatattcctaattattttaaaaagcatgttttccaaaaatatagtctgatacggattacgtgatccgtaaaccccatacggattggacaatccgtacggggtttacggattcggtaatccgtataacccatacggattgtccaatccgtacggggtttacggattatcgaatccgtatcatgcccaagcccccatggacgtaacagaccaagaaaaaaccagagggaagaagaaacttacctcaacgccctcgtcaccagtcgcggaccacgcccccgcccaatgccaccaccaaagccaccaccaaagcctcaccgagaatggatgccggatgaagtgagggagacgaaacggaagaagtgaacagtgagtgagagaagggaagtggagcgggtttttttattttaaaaagctaagggcagaatgggaaatgcactgtaaatgaatggtgtaggagaaattttaatggtgtatgaagttgctctcccgtcctcactgattcataatataaattcagtccattacaataaaaaaaatattttgagcacaattagtcaaaactaaacatgcagtaggtattaaagagatttaactaagttggttaaacacgcgaatgttataaatcctatggcaccatattcaattcctgctcttcaaaaaaaaaaatgcagtaggcgttaagaccattaaattgagagttattccatatatattcacattgaagtaagatgagtctccttcgaattcattcctcacagaactaggatgagtctccttcgaattcattcctctgcgcataaccaaaccctcctttctgtctctttctcatcttccctctcccattcctcctcactctacttcaaccactcttcttcttcttcctccaccactctcacccatgctcacacgtgcttccacccccgcctctcggtgctcacgtgctccatctccaagatccacagctacggcaccgtcgactacgagcgccgccccatcgtgcgctggaacgacgtctaccgcaggatatctctccaccaaaacccccaagtggggtccgcagaggtgctcaaccaatgggagaacgaagggaggcacctcaccaagtgggagctttcaagagtcgttaaagagttgaggaagtacaagagatttcgaagagccctcgaggtcaaaattctatctttaaaaccttttttttttatcatttgggtctctttttttgtatgtttttgctttttactgatgaaatgctgatatatatgtctgtttttggataaacttttctctgtaagtactttttttaggagaagaaaataagaatgaaagattaattaacttctccaataagttaatttatttagctcatgcatagcatggttttgaattgccgttgcggtcgtgttgtgtgagccagaaaacctttacatcactggaaaatgcagctcatgcggcggtcgtgatgcgattgcagagtgacaaaataccttgacgttgcggaccgtaatttaaaaccatgtgcataggtgagaaaacacctttttggagaagctaaatgaagagagcttctacaaattaatttgtgctaagctaatatgagcttaaaaggagaagctaatttgttttttcttctcctaaaagtgcttatggagaagtttatccagacagcctagagtttttttgttcttcataaaactaattatttggtcccaggttttaaattgtggtcatggttttgttgtgattattgatattgtgagaatttgcagttgaggaaattcaaaaaaacattaacgattccagtgtggcaaaaactgcggttgtggaccgtattttaaaccttgatttggtcccatagttgacatttaggaataaataagaattagtcctgtatcagaaaaactaagggtctgtttggtagggtggaacgaaaacaagggagaaatttgaattttgtttgtagaggttttttccctttcctttttctctccaaccaaacaaatagacgaacataccattaggggttgaagtagaaataacaattaaattcttaccattaggggttgaagtagaaataacaattaaattcttagaggaaatacagggtctggtcatacgtcctgaacagcaggatagttggaggtgggagggggccccgaatggtaaatacatggtgggaagtgcgtacaagctattaaatagggtgttggatacccagggtcaggaggaggtgtacaaagatctttggaagctacacatcccttgcaaagcctcttttgcttggaggttactgagagactgtttgccaactaaaaagaatttggttcatagaaatataaatattggtgatcatagttgtcctttctgttgtaactttgttgaggatgcatcacacgtgttcttgagctgccacaagattatgccgctgtggtgggaatctatgagctggataaatgtggtaggggcatccgcggttaacccaattggtcatttccttcaacacagttactgcaactttactggcattaaagttcagaggtggtatatttggtggatagccttgacatggagcatttagtttcatctcaacatgattgttttccataatgaaagctttgatgggtctaagctgttggaggacgctttattcttttactggttctggttagcgaatctggagaaaggatttaatattcctttctaccattggtctagtcatattagagatggtttttgttagtaggggaggttttcattttggctctttttgttttctttcggccaacagggtgagcttttcctagggtgacctttggcaccttaattcgagttgttaccatgtattaggttgccaaagctctgctaggcttactgttagctttgtctttttcagtactacttgtactcctttctcatatatataatatattatctttgctgataaaaaaaaaattaggggttgaagtatgatgtttaaaaactttttttttttggcactgcaggggctggggggattctcaatttgtgttgttttggtgtcaacaaatacaaaatagttatttaaagggtgaaattattaggtgaacttatcattgaattaacatttccggttactaatttctatagttatgcattttcaaagactaaagcaacacaagtggccaccaaattgtataataacacacacatagggcatattcagtgtgaggtaaaaagtgttaatcttgggaatacaactatacatagatggtaaagattgttttagttagtatatataatcaattactgagtggtgggagggttgaatgtttgctgtttgggtatgtttcatttggctttaggaatggttaatatttaaggattttttccttttctgtgttaagaagtttgattttctttggaccctgctaatttagaatttagatggcattcttctttttaggaaaatcctgatccttgagggtgattaacaacttgattcatttctggtttttcattgtgacatgacatgagtatattctacagacttgtgtgtaaataggcagcattcttcaagttcagtagttgataaccatctttaaatacctgagttgaaatatatagcactgctatttgttcatgatagtggattttgctatacagcattgccatttgatttaagttttgcatgtgattcaagttggaatataaaataccacacaacaattgttacacaattccatgacttcctaaaaacatactaattcagatccataacctcctaaagacatagatgctatacaaacattaataatgcaggcctagtgagtctgggtatgtttcttattccttctcacataagctttccaaggtatatcagagcattagcaggaaccgaaaaatctgtgaattgaggtttccttcttccacaacagtttataaaagagaaaaagatttcccgaagaagcttttgcctaccatctatcatttgtaattttatcttactggatgcaaggaaagtccaatatctttgccacaaatttagcaaatcccctcgccccccctgctcaaccttttccctgctgtctttcccatattttctaataactgtaacagaatattcttagctagaattctattttgtcaatggcgtggaggaactgtatatgtttttacatcattggactatcattcactctgaggaaacaaagattgcttcgccccctttggcattgtcagaaaagtgtagcatgtttcaaaagatagtccagggaaaggagtaggggtgggctatatgagactacaacaagacctgattgattccatagaagctatatcagaaatgttctttctttattatttatttggggcatttggttttatgggataaatgaacatatacaataacataatctgtaacagcatttttagttgttaattctagagttgcaaacactgtcagtaaaatttgaataatctggaaaaccattttttgggccactatatttaatggcatgcttcctttggaatgtatggaattatgaattatgaaatcaattactatttctgattatgtttacttatttgcttcaaatgtctttatcaggcatgtacaattttttgaaagcaatgagaaaatgtttcaggtatatgattggatgaacaatagaccagagcggtttagagtttctgagagtgatgcagcaattcagttagatttaattgcaaaagttcgtggagtttctagtgcagaagctttctttctgagtctggaagataagttaaaagataagaagacttatggtgccctgttgaatgtatacgtgcattctagatcgaaggaaaaggcagaatctttatttgatactatgaggagtaaagggtatgtaatacatgccctaccatttaatgtaatgatgactctgtacatgaaccttaatgaatattctaaagttgatatgttgacttctgaaatgatggaaaaaaacatacggctagacatctatacatataatatctggttatcttcgtgcggatctcaaggatcggtagaaaagatggaacaagtgtttgaacaaatggaaaaggatcctaccattgtccctaactggtctacattcagcacaatggcttcgatgtacattaggatggatcagaatgaaaaagcagaaaagtgcttaagaaaagttgagggtagaattaaaggtcgagatagaataccttttcattatctcctgagtttatatggaagtgttggaaagaaagacgaagtgtatcgtgtgtggaatacttacaaatcaatttttccaagtataccaaacttgggataccatgctattatttcttctctggttaaattggatgataatgagggtgcagaaaaattatatgaggaatggatttcagtaaagtcaagttatgatcctagaattggaaaccttctcataggctggtatgttaagaaagatgacacagataaagctttgagtttctttgagcagatgtcgaatgatggctgcattccaaattcaaatacatgggagattctttctgagggccacattgcagataagaggatttctgaagccctgtcctgcttgaaagaagcatttatggttgctggtggttcagagagttggagaccaaagccctcatacttgtctgcattccttgagctttgtcaagagcaagatgacatggaaagtgctgaggttttaattggactgctgaggcagtcaaaatttagtaaaagtaaagtttatgcatctctcattggctcacctgatggtaccattgacaatggtgaattgccaagcaagattgatataactgatagaactgatgatgctgttgacagtgaaaatatggatgatgactctcaaatgttgttgaaccaactagatagtagcttttgatactttatttctttttcttttgactagtggaaaatgatatgctagaacatacaggggattttttgtcaaatatgatctcatcagagtttcatggcttctatgtcttattctcttcatcacagttagaaagtggcagctgcttctatgtcattggtgttttttttaacgatttggtgatttgattatgtgtattgaagtgtgaacgtgtgtgtgtgtcaacagtgaaaatatggatgatgactctcaaatgttgttaaaccaactagatagtagcttttgatactttatttctttttcttttgactagtggaaaatgatatgctagaacatacaggggatttttttgtcaaatatgatgtcatcagagtttcatggcttctatgtcttattctcttcatcacagttagaaagtggcagctgcttctatgtcattggttggtgttttttttaacgatttggtgatttgattatgtgtattgaagtgtgaacgtgtgtgtgtgcgtgtgtgtgtgcgtgtgcgcgtgtgtgtgtgttgtgcgtgcgcgcaccgtgcgtgcgtgcggtttaggttcaatataatgagaggaatgatggattgagcttttggagcattaactctatttgctttagcccctaacgttgcttaaagtattttattttagtcatttttcacttaattgtgcgtgcgcgcgtgcgtgcgcgtgcgtgcgtgtgtgtgcgtgcggtttaggttcaatataatgagaggaatgatggattgagcttttggagcattaactctatttgctttagcccctaacattgctgaaagtattttattttagtcatttttcacttaattctcttctactatttttgtaccaaaaatgataaatgtaaatgccatttatagataggaactaaaatatatttttttaacttttaatttttgatgaattttatttcaataaattaatttggtgcatttaaattttaacttttaaaaaaattatgaattttatcttctgtcattttactcctaatataattacacctttactcaactttttattttttgaaaaaatttatcctaatttttggtaaattttactcctaatttttttgcttggttgggataaaatttacaagtttattatgtcaaattaaaaagggaaaggttcaccgaaaattaaaaagtttggataaaaaatgcaattaagtccgagtaatatagagtttatgctatgaaaggaagattaatagaaaaaataacaagtattaatgtgatatttaaaacaaaagagaatttcaatagtaagatttttcatttaaaaagcgtcttgggccacttctaaagaagttgcaaagaaactgcttttcagaaatttcatttcaaagaccaattactatcaaatactcaaactttgatatttcaaaggcaaccacgacctaaaaagaaataaaacagataaattgtttctggatccaaaataccatattccagacttacattcaagtgcgtcatttcaggtatcctggatcttgatgatacactcaggcgagtgtttctgagttacttctcctccaacatcatttggataggagcaccatatgcagctagcatagagcacccccatgattatgatgctaagaagtagcattaaggatgtggcccctccaaacacaaacaataggcctgagattttagctgaaccaccccatgaaacactatatccagacactgcaactattatcgctaaaatgtaatagagtaaattaaatcccagcattgagtatgcaggtatacgatgaggcaatgacaaggtaagcatggctgcaaaaataaagttcaggaaaaatattgatgcccatactgtgatggaacatgctttccccatacttaattccattgggtcaatgcctctggattgggataacttctcaagaggattgcccaaaacagatagttgggcccattgcattccatataaggcagcccatgtagtaagacgatttgcatcagtagaatgtgattggaatagcctgttcccataaaacttcaaaaatgtgctcaaactcttcgtctttcctttatctacttttaacaccccattatcgtcaatctttacaacatggtttgggggatgagaacggcgattttccttttcttcctgttgatagtcaatcacaatggagaaatatggttggtgagtgagaattttgatgaaataatagatggtctttctatggaactttatttttggtattttcttgtaaatgtaaagatctcatgcaatggaaatgtcattgtaggggacaaaataacaaaatgatatatggctcattttgcacccatgttgagatatgtgacacacatgcacaccatatatataattttagatatgttgaggtatgtgaaagatgtgagatatttcaaaaataaaattgtgagatatttgaaagacatatcctctctatgtgagcaaaaagtgtgtctaaagcttaatgacttgttattattacctaattctatatgttaccttgcattttttcagttgctgatatagttctttctgtggctgtacaaaaaagcaacaaatgttaggtacttgagaagtaaagatccaatcaagttacattaaggaaaaatatgcatatgcatctctttattaaattttcaagccaattagtattgatcatcaaaatggacaaaaggaaaagacaatgattttccctacatggctcattttgctcctatgttgagttatgtgagatatttcaagccgagaaaaaacaaaaatatatttgaaagacacatctttttatgtgagtagaaagttagtctaacaatatggagttagacaccaaatgtttcgttacgagatgacactacactttgcgccattgaaatataatgtgtcttttctcccagtgcataagacgattttgcaaataattacataagacataaattgtactttttttgtcacgagacaaggtcattgaattagcttttttacatcaaaattgttattataatgttccatttgacaataaaataataatgttaatgataagtcccaaaatttttttgttttggatgaagaaaacacaataacctgtaattaattagttctatatgtaccttgttctcctcttgactctcgcatgcttgtttttgtgcataaaagcaaggaaggttaggtacttgatcatatcattagatattatgttaaattaaggtgcaattcaacttatttgttaaaataaaataccatgattttccctagcttatggatcatatcattagatattatgttgagatacatcaaaaacaaaaatgtgacatatttgaaggatatactttgtattgtgagtagaaattctgtctaaaaatgtaagatgatttgatttctgcactagacaaagttttgtgtttttcttgatcacatctctatatcaaatttgttattacaatgttccatttgtcattaaaataataatggtattgataagacttgggaaaaattatgtacctttgatattatttcattctctggccttttcacaaaacaacgttctaggtcaatgtcagtatatacttgcagccgcttctcctgtacataaaagaaatagaggttaattaggcacttagtagaggtgaaattcaaatacattaaggaaaaataagcatgcctttattatatttaatctcatgtcaattagtattgagcattaaaaatggacaaaaggaagagaaagaaaattatatgattcatttagctcccgctttggatacaaacggaagaaaggcaaattaaatgtttaaatttatttgttaaaaaaaactaaaaataaacaactttttatttttagtgtgattgtctaaattgcttatgcttaaaaaataactttcctcttattttaagaaactaattatatctacttaaaaaaatgctgataaaaaatgcttattttaaaattgattttctaactttaaaaacaaacacacccaatgccttaccctcggtagaaattgatgtttttttgtagtttggtcctgttttcataaatttggatacttaaatgctcaattacctctttaagatactacacttttatttattttatctaaaatgtaactttcgtcttcttatttttttaaatccattattttttctcataatttttaattgagacattttgttttttatttttaattaattgcaattttagtcctcctatttttaattgagacatttcgcttttaaaaatttgtgattttagtccctcactttcaagacttgtaaaaataatatgaactatgattgtacttagatagtggatagaattaacaataataaaccatggttgtactttaatagtggatagaaagaaattaatagaaaaaaattgaacggatccaatggaataaaataaattaatatttattgtgtcaatattttaatgataaaaggatcaaagagattgattccatctcatagtagctaaattagaaaagaagaaaaaataatagtattgggtgatatgaaataaaatacgtataataaagtcatgtttagttctattccaacatatttaaaaccaatcatgactcaaaagaaaaaaaatagcaccttccattttattgtatttctaggtgtcaattggaacacaactccgaatatttcaggaattaatgattataaatattgaaatcattttgcaactcaaagtaatctaggtaattgatttaactttattttactacttttacaagagaaaacgaaaatatgacattttatcgatagtttctttttctatattggtagatatgttatagatatttgaaagatgtgagatatttaaaaaaaaagttagattttgaaagacacacaagaaattaacttttattacgagaaactagctaagaaacttgttaaaaaataattacttgtcaaccaagttataataaggcataactattatttctaacaagcacaagcctttatgtatgggtaaagagtgtgtttgacaatgtagacttaggcaacatgtgtttagttatgatgacactacattttgttgaaaacataattatgtctattttccttaatgtgagacaaattttccaatcatctatacaagtaacaagactaaggttgtgtttttcttgttcacaaagtggttgcattatcttctctatatcaaatttgttattataatgatccaattgacagtaaaatattaatagcaatgaatagcactatagaaaattatgttttgaacgaagagatcataacttgttgtatcaattagttctatacttacatgatttttcaccatttgcatcggataatactgatcaagatatattccattaaggtaacgttgtagtatgactctctgtacataaaagcaataaaagttaggtacctaacaagtgaaggtgcaattcaattactgtaaggaaaaacatgcatatttttagtaaacttctatgccaattagtattgatcataaaaatggacaacaggaagagaatcacaattacttgccttggtttatttcgttgccatgtctggacaaaaggcaaacaacgacaaggacttgcccttggtagaaaaattgttgtttttcgtagcttggtacaggtttcacaaagttggaaacttaaaaagataaattatttctttaagatactatccattaatgtaatgtcactaattataatcaatgtaatttgggtgatatttgcataattaagagacttagtattaataaaagagaattaatgaaagaatggacattttggtatttgtttagaggttagggtgtgaataagagattgctgaatgcacccccctcctcatgttaaaaactgtggctgaccatggttgtctcactttctccttcttcttcctttatgtccatgtctctccttcacacccaagcaccatttctcttcactccttgattagtggctggaaacataaatcaagcaagaaaggaagctcttctccttgtcctttattgttgttgagttttggttgtgagcaatttgaggaaaagagtagagctaggagctgcccacttcattcggagtagctgggcagaattggaagctatagagagctggtccactctccaatttgatcaattgaactctaaggttggtactctcctcctatttctccttgattcttagaaatcataaggttagggtttggtttgataaatgttgagtattggaagggtgaaacagttttaaggtgtaatttggagtgtgtagaagttgattcaaggttgaaatggtgttccaagcatgaaacatgcatgttcatgacatcaaggaacatcgatctggtcaaccctgatgttttgtgcataacaggagctatcggtatccaaatggtctaaaaacagtgtcatttgaaagttaagagattcaagtaggttctggaattttcgtgctatttttggaggttcctagaatttattgtgcagagttataacaggctgttatactctgtttttagcagattttactggtgtttagcagattttactggtgcagagggcaatcttgaagaaattttatctcagtctaggatgatccaaatttagtgatttcagtggaaatagaaagatgtgtatgtttaatttatacctgtaaaatctcaggtcatttggatgagtatcctggtgttggcgggtgtttgaaaattgctggttgaactgatatgttattgtcagcactgttatagtgcgtaacagttctggtattttaagtgttacagaggttgtggtttatgaaattggatgaaaccagttttgtttgaaagataggagataaatatagatgcctctaaattcccttgattttaggagcttcctacagggatttatgatttttataagtgggtacaaaatctgcataattttatgcagaaaattctgtttttccgtgtgttttggtataattaaatgccttgtacggataggatgattgttgattcatgcctgtgtgcctttatatagggcatgacaacgagggtgttagtggcggagaccacagttcagggattgaatagcagagcacggccttgcctggtaagttcgtggttcttgggagtgttttccactgcttgagtttcaccacactccgagggagtgatggttgcatacatgattcatatgcattaaattggatgttacatctttcctcatgataatatgtatgcatattctgtgctttgtgtttggttgcctttcggatagatcttctggcaagttagatagatatagacctccctaggagcagagcacaactcctagttggtttgctcacgtaaagggtatatgatcccttaacaccagagatacgtggtcccgtcgtggttgaagcctattctcggaggtgtcatatacgtgcatccatgcgtaaaggggtatctcccgatgggatgacaagggtgtgagttccggcctaacactgaggactgagagtcctgatgctgatgatgatgcatctttggcaggcttagtaccgcagactcctgatgctgatgatgatgcatcattggcagcgtagttccgcagatacacattagagctaaagaatgtttagttgatttaccacggtctggacccgggtgatgaaaccggataccccttctccttccttcattttcattccttccaactctccacggagagtttgaagtcgaacctgtctaactcgatcggctcccttgtagacatttgcaagaatgtcccacgcctcctttgaatttgttgcaccagcgatcttctaaaacccagaatcatcaacagcccggaataacatgtacaacacccctttgtctttcaacctcgtctccttcaatgtcttgttctgcgccatcgtatatcctgctgtggatgttggtttctcgaaaccaccatggacaacttcccaactatcatgggctcccaaaagagctttcatatggatactccaattttcataattagcctttgtaaggcggggtagtggcagactactacttgacccactctccattttctcaagctctgaaaccaatttgtgatatcacatgtcctcacacacactggtgcactcacataaacacacaacaatctctctagtagaatacttctgatgtatttagataatggtctgccaagccacacattggcctcttacatgagatgctatttataagtaatagcaagtgagatatgactcttcacatgagtaacttatttacctagggacatgactcttcacatgagtaacttcttcacctagggacatgactcttcacatgagtaacttcttcacctatctaatttattattattaaagtttaatccaacaaaatctccccataaactttaaatatttctttcgtctttcatccccaacttttttttttctttttctcttataaacccatttaactccaatgggctgacttccttttggcagttcggtcaactcccaagtttcattccgttcaatgaccttgattttttcattcatggcggttttccatttttcatctcggattgcctctttaaatgttatttcttcagaatctgtcaacaaacaaataaggtaagcttcattagttgaatcatacaggtcatgcaaactcctcattcggggttgtctcatctcgtcttcatcagatgtctcacttcttgccgggatccaacttggtgattctccttccttctcttcttgtcgagtaagcgtctgtccgggatctgaggtgaccttggatgaagtgctccagttccattcatcttcttcattaactttcacatctcggcttatcattactttcttggtgccgggatccaacaacttgtatccctttgtcttttcgtcatatccaatgaaaataaatttcctgctcttgtcttccaacttggttctttgctgatcagggatgtgtgcataagccacactcccgaagactctgaaatggcctactgatggcttctgtcctgtccatgcttcctgtggggtctttccatttagactggaatgtggacaacggttctgcacatacactgcacacaatacggcctgcccccaaaactccttaggcattttcttcgttttcaacattgaccttaccatatcaagaatgatacggtttttctctcagccactccattttgctgtggtgagtacggtgcagttaaaaacctttttatcccatgctcctcgcaataccttttaaatgcaattgatgtgtactccccacctcggtcagaacggattgacttgattcaccttccagttgcattttctatcatcatcttaaaactcttgaatacttcaagcactttggatttttcttccagcaaatacacccaagttttacgactaaaatcgtctataaacgagacaaaatacctctttccactgaaagacccaggagagatgggtccacacaagtcggtatgaattagttctagtggttgttgagatctgtatttggccgtcttttgaaatggactcctaacatgcttgcttagcacacattcttcacaaatctcttttcacagttcaaactcggcaactcttccaccatattcttgttcatcatctcgaccaggcctccgtagtgaagatgcccaaatcggcgatgccataaggatacttcatcactcatctcggcttgcaaacagtcgacctgcacactcctcaacattagtttgaacattctatttttcttcatctgaacttgaactatcaaacatccttgtttatccttcaagaagagaactcggtctgtcatgagaaccgagtatccattctccatcagttgtcccacactaagaatattactttttagatcgggcacgtaatagacatcttggatgcgcccaattcgtccatctttctgcaagaaacaaatcgtacctcagcctttgacatcaacctttgaaacatctccaaacgaaacatgtatggcttctatttgtctcagattactgaacaaatgcttatgcccacacatatgattgcttgcactagtgtctagataccattccatctcgaatccctggttgacgtcacttctagtcatcatcagaattcctccatctcttacttctccttctgtaacaaggtttgaagtttcatttcttctttcatcttggcaatccttcgcaaaatgtccgactcgaccacagttgtaacatctgtccgagttgcaatctcttgagtaatgtccatactttcctcatttaaaacattctacatttgattgatttgaatgtcggcctcttccacgaccttggcctcgccaattctgccgacttaatttagcctgctccttattccttccttcttgaccgcggcctctactaccgcgaccgtctcctcgacctccatagtcatgccctctacctcgaaagttttgagaataaagtaccttttcgtccttgattgaagcttttgtctggagtgcttgctccagtgactcttctttcttctttttctttctttgttcatgcgcctccaaggaaccagcaagttcgtcaacggtgagcgttgctaggtcctttgactcctcaatcgcacatacaacattctcgaagtcgtcagtgagagacctcagaattttctccaccactttggcatcggacaacacctctccatttctgtttagttgatttaccacggtctagacccgggtgatgaaaccggataccccttctccttccttcattttcattccttccaactctccacggagagtttgaagtcgaacctgtctaactcgatcggctcccttgtagaaatttgcaagaatgtcccacgcctcctttgaagttgttgcaccagcgatcttctcgaacccggaatcatcaacagtccagaataacatgtacaacgcccccttgtctttcaacctcatctccttcaatgtcttgttctgcaccgccgtatatcctgctatggatgttggttccttgaaaccaccatggacaacttcccaactatcatgggctcccaaaagagctttcatctggatactccagttttcgtaattagcctttgtaaggcggggtagtggtagactactacttggcccactctccattttctcaagctctgataccaatttgtgatatcacaggtcctcacacacactggtgcactcacacaaacacacaacaatctctctggtagaatacttctgatatatttagataatggtctgccaagccacacattgacctcttacatgagatgttatttataagtaatagcaagtgggacatgactcttcacatgagtaacttctttacctagggacatgactcttcacatgagtaacttcttcacctatctaatttattattattaaagtttaatccaacataaattacttaatcaggatcgtgatggcaattaagagttgatgagagttataacttacagtgtcaaatttccggaaaggcgaatccactttaggcgcaaacttggattcccaaactgaatcagcaatcacaatccccatgttaggcactgcacacaaatcaaagaaagaatatgcatacatgttagaaattatgatggcggtataaacagaaactgctgctgagttatatcattatctactacttccacttcaatcctaatatctggatattaatttaccttgtataacccatagttctggatacagttcctctatagtttcctcgagtatccatgaaagcatattcttaaaccaaatctgtgtgtccagttctgctatgcattgcaaaattttgtttgtcagctctaccacagagtcgtcgccactgatcacgtttatttcttctatcaaatgcgcaatattctcggcttccataactgaataacatggtagatttttcaaatgagttttcatccgatctataatggcttctccacgatccgatggtggtgcaggctcattgatctcgtccatttcttctgttcctctcagtctcagcaagtttacaaactactcactttcttttttgctcaagagacacataacttgcttctacctaataccatacaattatcatacgcgttgaattcaacgtttcaataacacaccttccacgcaattcttaaaataatcagcattacgtcacagttctagaatgtataatcgttttccacggtaagtgccttttaaagttgaaagttggtgtggttgacagcaatgcatatgcatttttctagtaccacgtgtaataaaatacattatcattctagaaaagccggtttgactttgaattttcggtgcagcaaaacgcggtttcatattgaatttcgccatcaacgccacgatagcattggaaaaaaatgttttgagaaggtgtgtttggtttcagactttcacatatcatccaggtggagcaaaatttgcagtacgagagagttggattcttgggctgggaactactaccaaatcatgtatatgcctaatttctcctcggtaagttgtatatgcttacttctatgctgaactgcatcatgatcaaactccattcaactcaaacaaaccgagctaatttgttcttaatcattcttatttattatcgtttttcacatcgattttgaggaatctgaatatgccaaagtcagcagttgacacatgtatagctatattaattatattttgcagtttccatcgacaaatcttgttgcgaaaatcttgaaccaaaggcagaatatccatgaaaaggaggagcatggagatgatgagaagggcaagctggtatcattaggacaaatgaatagtttaagtgtaagtttccttcttttcccctctaaactatataagttgtaattattgcagaaatgtaatgccattatattatgctaatactaattaaactgcagagcaagcaagatgttccaatgttagacttagttagagccatgtgcactgtccatgatgagaaatcaggacaacctttagcttttgagtacacattgaacacaattatgcattttgttctggttgtgtgtagtgagttatttaactaagctaatatatggtgtgatgtatattatgtgtattccagaggaaagaggatagtagaacatcaatgaacattagttctgaagttgaaagaagaacaccagaaatcatgggaagatgtgcatcgtggtgggccacctccacattttttttgtgtttgtgttcattaaaattcagcaagtcacttctccaaccacaaggacccaaatcactatggcttgccatagtaggaatgttcagttttactgtgtttgtatctgcttcttctgttatgctagttgttggagcacagttcactcgtatgcagataactgttatgctcagtgctttcttggctatgctgttagcttaattagatagttttcatatatgtacttatgttttcttcaggcagggttgacaagttgttttaatcttgttatttacataatttcattcgcataacattaaaacaaagtttgggtatgtgatatatcaatatcaacgattaaccagacagaacatattcacggatgaacattga

SEQ ID NO: 19 - DNA, 12154 bp, Glycine argyea
  source 1..12154: mol_type = genomic DNA
aaatactcaaactttgatatttcaaaggcaaccacgacctaaaaagaaataaaacagataaattgtttctggatccaaaataccagattccagacttacattcaagtgcgtcatttcaggtatcctggatcttgatgatacactcaggcgagtgtttctgagttacttctcctccaacatcatttggataggagcaccatatgcagctagcatagagcacccccatgattatgatgctaagaagtagcattaaggatgtggcccctccaaacacaaacaataggcctgagattttagctgaaccaccccatgaaacattatatccagacactgcaactattatcgctaaaatgtaatagagtaaattaaatcccagcattgagtatgcgggtatacgatgaggcaatgacaaggtaagcatggctgcaaaaataaagttcaggaaaaatattgatgcccatactgtgatggaacatgctttccccatacttaattccattgggtcaatgcctctggattgggataacttctcaagaggattgcccaaaacagatagttgggcccattgcattccatataaggcagcccatgtagtaagacgatttgcatcagtagaatgtgattggaatggcctgttcccataaaacttcaaaaatgtgttcaaactcttcgtctttcctttatctacttttaacaccccattatcgtcaatctttacaacatggtttgggggatgagaacggcgattttccttttcttcctgttgatagtcaatcacaatggagaaatatggttggtgagtgagaattttgatgaaataatatatggtctttctatggaactttatttttggtattttcttgtaaatgtaaagatctcatgcaatggaaatgtcattgtaggggacaaaataacaaaatgatatatggctcattttgcacccatgttgagatatgtgacacacatgcacaccatatatataattttagatatgttgaggtatgtgaaagatgtgagatatttcaaaaataaaattgtgagatatttgaaagacatatcctctctatgtgagcaaaaagtgtgtctaaagcttgatgacttgttattattacctaattctatatgttaccttgcattttttcagttgctgatatagttctttctgtggctgtacaaaaaagcaacaaatgttaggtacttgagaagtaaagatccaatcaagttaaattaaggaaaaatatgcatatgcatctctttattaaattttcaacccaattagtattgatcatcaaaatggacaaaaggaaaagacaatgattttccctacatggctcattttgctcctatgttgagttatgtgagatatttcaagccgagaaaaaacaaaaatatatttgaaagacacatcttttttatgtgagtagaaagttagtctaacaatatggagttagacaccaaatgtttcgttacgagatgacactacactttgcgccattgaaaatataatgtgtcttttctccccgtgcataagacgattttgaaaataattacataagacataaattgtactttttttgtcacgagacaaggtcattgaattagcttttttacatcaaaattgttattataatgttccatttgacaataaaataataatgttaatgataagtcccaaaaaaattatgttttggatgaagaaaacacaataacctgtcattaattagttctatatgtaccttgttctcctcttgactctcgcatgcttgtttttgtgcataaaagcaaggaaggttaggtacttgatcatatcattagatattatgttaaattaaggtgcaattcaacttatttgttaaaataaatacaatgattttccctagcttatggatcatatcattagatactatgttgagatacatcaaaaacaaaatgtgacatatttgaaggatatactttgtattgtgagtagaaattctgtctaaagatgtaagatgatttgatttctgcactagacaaagttttgtgtttttcttgatcacatctctatatcaaatttgttattacaatgttccatttgtcattaaaataataatggtattgataagacttgggaaaaattatgtacctttgatattatttcattctctggccttttcacaaaacaactttctaggtcaatgccagtatatacttgcagccgcttctgctgtacataaaagaaatagatgttaattaggcacttagtagaggtgaaattcaaatacattaaggaaaaataagcatgcctttattatatttaatctgatgtcaattagtatcgagcattaaaaatggacaaaaggaagagaaagaaaattatatgattcatttagctcccgcttttggacacaaaccgaagaaaggaaaattaaatgtttaaatttttgttaaaaaaaacaaaaaataaacaactttttatttttagtgtgattgtctaaattacttatgcttaaaaaataactttcctcttattttaagaaactaattatatctacttaaaaaaatgctgataaaaaatgcttattttaaaattgattttctaactttaaaaacaaacacacccaatgccttaccctcggtagaaattgatgttttatgtagtttggtcctgttttcataaatttggatacttaaatgctcaattatctctttaagatactacacttttatttattttacctaaaatgtaactttcgtcttcttatttttttaaatccattatttttctcataatttttaattgagacattttattttttatttttaattaattgtaattttagtcctcctatttttaattgagacatttcacttttcacttttaaaaatttgtgattttagtccctcactttcaagacttgtaaaaataatatgaactatgattgtacttagatagtggatagaaagaaattaatagaaaaaaattgaacggatccaatgaaataaaataaattaatatttattgtgtcaatattttaatgataaaatgatcaaagagattgattccatctcatagtagctaaattagaaaagaagaaaaaataatagtattggatgatatgaaataaaatacgtataataaagtcatgtttagttctattccaacatatttaaaaccaatcatgactcaaaagaaaaaaaatagtaccttccattttattgtatttctaggtgtcaattgggaacacaactccgaatatttcaggaattaatgattataaatattgaaatcattttgcaactcaaagtaatctaggtaattgatttaactttattttactacttttacaagagaaaacgaaaatatgacattttatcgatagtttctttttctatattggtagatatgttatagatatttgaaagatgtgagatatttaaaaaaaaagtgagattttgaaagacacacaagaaattaacttttattacaagaaactagctaggaaacttgttaaaaaataattacttgtcaaccaagttataataaggcatagctattatttctaacaggcacaagcctttatgtatgggtaaagagtgtgtttgacaatgtagacttaggcaacacgtgtttagttatgatgacactacattttgttgaaaacataattatgtctatttgccttaatgtgagacaaattttccaatcatctatacaagtaacaagactaaggttgtgtttttcttgttcacaaagtggttgcattatcttctctatataaaatttgttattataatgatccaattgacagtaaaatattaatagcaatgaatagcactatagaaagttatgttttgaacgaagagatcataacttgttgtatcaattagttctatacttacatgatttttcaccatttgcatcggataatactgatcaagatatattcgattaaggtaacgttgtagtatgactctctgtacataaaagcaataaaagttaggtacctaacaagtgaaggtgcaattcaattactgtaaggaaaaacatgcatatttttagtaaacttctatgccaattagtattgatcataaaaatggacaacaggaagagaatcacaattacttgccctggtttatttcgttgccatgtttggacaaaaggcaaacaacgacaaggacttgcccttggtagaaaaattgttgtttttcgtagcttggtacaggtttcacaaagttggaaacttaaaaagctaaattatttctttaagatactatgcattaatgtaatgtcactaattataatcaatgtaatttgggtgatatttgcataattaagagacttagtattaataaaagaggattaatgaaagaagggacattttggtatttgtttagaggttagggtgtgaataagagattgctgaatgcacccccctcctgatgttaaaaactgtggctgaccatggttctctcactttctccttcttcttcctttatgtccatgtctctccttcacacccaagcaccatttctcttcactccttgattagtggctggaaacataaatcaagcaagaaaggaagctcttctccttgtcctttattgttgttgagtttggttgtgagaaatttgaggaaaagagtaaagctaggagctgcccacttcattcggagtagctgggcagaattggaagctatagagagctggtccactctccaatttgatcaattgaactctaaggttggtactctcctcctatttctccttgattcttagaaatcataaggttagggtttggtttgatgagggtttggagtgaaattgataaatgttgagtattggaagggtgaaacagttttaaggtgtaatttggagtgtgtagaagttgattcaaggttgaaatggtgttccaagcatgaaacatgcatgttcatgacagcaaggaacatcgatctggtcagccctgatgttttgtgcataacaggagctatcggtatccaaatggtctaaaaacagtgtcatttgaaagttaagagattcaagtaggttctggaattttcgtgctatttttggaggttcctagaatttattgtgcagagttataacaggctgttatactctgtttttagcagattttactggtgcagaggcaatcttgcggaaattttatctcagtctaggatgatccaaatttagtgctttcagtggaaatagaaagatgtgtatgtttaatttatacctgtaaaagctcaggtcatttggatgagtatcctggtgttggcgggagtttgaaaatggctggttgaactgatatgttactgtcagcactgttatagtgcgtaacagttctggtattttaagtgttatagaggctgtggtttatgaaattggatgaaaccagttttgtttgaaagataggagataaatatagatgcctctaaattcccttgattttaggagctttctacagggatttatgatttttataagtgggtacaaaatctgcataattttatgcagaaaattctgtttttccgtgtgttttggtataattaaatgccttgtacagataggatgattgttgattcatgcctgtgtgcctttgtataggcgtgacaccgagggtgttagtggcggagaccacagttcagggattgaacagcagagcacggcctcgcctggtaagttcgtggttcttgggagtgtttcccactgcttgagtttcaccacactccgagggagtgatggttgcatacatgattcatatgcattaaattggatgttacagctttcctcatgataatatgtatgcatattctgtgctttgtgtttggttgcctttcggatagatcttctggcaagttagatagatatggacctacctaggagcagagcacaactcctagttggtttgctcacataaagggtatatgatcccttaacaccagagatacgtggtcccgtcgcggttgaagcctattctcggaggtgtcatatacgtgcatccatgcgtaaaagggtatctcccgatgggatgacaagggtgtgagttccggcctcacactgaggactgagagtcctgatgctgatgatgatgcatctttggcaggcttagtaccgcagactcctgatgctgatgatgatgcatcattggcggcgtagttccgcagatacacattagagctaaagaatgtttagttgatttaccacggtctggacccgggtgatgaaaccggataccccttctccttccttcattttcattccttccaactctccacggagagtttgaagtcgaacctgtctaactcgatcagctcccttgtagacatttgcaagaatgtcccacgcctcctttgaatttgttgcaccagcgatcttcttaaacccaaaatcatcaacagcccggaataacatgtacaacgcccctttgtctttcaacctcgtctccttcaatgtcttgttctgcgccgtcgtatatcctactgtggatgttggtttctcaaaaccaccatggacaacttcccaactatcatgggctcccaaaagagctttcatctggatactccagtttttgtaattagcctttgtaaggcggggtagtggcagactactacttggcccactctctattttctcaagctctgaaaccaatttgtgatatcacaggtcctcacacacactagtgcactcacacaaacacacaacaatctctctggtagaatacttctgatgtatttagataatggtctgccaagccacacattggcctcttacatgagatgctatttataagtaatagcaagtgggatatgactcttcacatgagtaacttatttacctagggacatgactcttcacatgagtaacttcttcacctatctaatttattattattaaagtttaatccaacaaaatctctccataaactttaaatatttttttcgtctttcatccccaactttttttttctttttctcttataaacccatttaactccaatgggctgacttccttttggcagttcggtcaactcccaagtttcattccgttcaatggccttgatttcttcattcatggcggttttccatttttcatctcggattgcctctttaaatgttatttcttcagaatctgtcaacaaacaaataaggtaagcttcattagttgaatcatacaggtcctgcaaactcctcattcggggttgtctcatctcgtcttcatcagatgtctcacttcttgccgggatccaacttggtgattctccttccttctcttcttgtcgagtaagcgtctgtccgggatctgaggtgacctcggatgaagtgctccagttccattcatcttcttcattaactttcacatctcggcttatcattactttcttggtgccgggatccaacaacttgtatccctttgtcttttcgtcatatccaatgaaaataaatttcctgctcttgtcttccaacttggttctttgctgatcagggatgtgtgcataagccacactcccgaagactctgaaatggcctactgatggcttctgtcctgtccatgcttcctgtggggtctttccatttagtctggaatgtggacaacggttctgcacatacactgcacacaatacggcctgtccccaaaactccttaggcattttcttcgttttcaacattgaccttaccatgtcaagaatggtactttttttctctcagccactccattttgctgtggtgagtacggtgcagttaagaacctttttatcccatgctcctcgcaataccttttaaatgcaattgatgtgtactccccacctcggtcagaacggattgacttgattcaccttccagttgcattttctatcatcatcttaaaactcttgaatacttcaagcactttggatttttcttccagcaaatacacccaagttttacgactaaaatcgtctataaacgagacaaaatacctctttccactgaaagacccaggagagataggtccacacaagtcggtatgaattagttctagtggttgttgagatctgtatttggccgtcttttgaaatggactcctagcatgcttgcttagcacacattcttcacaaaatctcttttcatagttcaaactcgacaacccttccaccatattcttgttcatcatctcgaccaggcctccgtagtgaagatgcccaaatcggcgatgccataaggatacttcatcactcatctcggcttgcaaacagccgacctgcacactcctcaacattagtttgaacattctatttttcttcatctgaacttgaactatcaaacatccttgtttatccttcaagaagagaactcggtctgtcatgagaaccgagtatccattctccatcagttgtcccacactaagaatattactttttagatcgggcacgtaatagacatcttggatgcgcccaattcgtccatctttctgcaagaaacaaatcgtaccttggcctttgacatcaacctttgaagcatctccaaacgaaacatgtctggcttctatttgtctcagcttactgaacaaatgcttatgcccacacatatgattgcttgcactagtgtctagataccattccatctcgaatccctggttgacttcacttctagtcatcatcagaattcctccatctcttacttctccttctgtaacaaggtttgaaatttcatttcttctttcatcttggcaatctttcgcaaaatgtccgactcgaccacagttgtaacatctgtccgagttgcaatctctcgagtaatgtccatactttccgcatttaaaacattctacatttgattgatttgaatgtcggcctcttccacgatcttggcctcgccaattctgccgacttaatttagcctgctccttattccttccttcttgaccgcggcctctactaccgcgaccgtcttctcgacctccatagccacgccctctacctcgaaagttttgagaatagagtaccttttcgtccttgattgaagcttttgtctggagtgcttgctccagtgactcttctttcttctttttctttctttgtttatgcgcctccaaggaaccagcaagttcgtcaacggtgagtgttgctaggtcttttgactcctcaatcgcacatacaacattctcgaagtcgtcagtgagagacctcagaattttctccaccactttggcatcggacaacacctctctatttctgtttagttgatttaccacggtctagacccgggtgatgaaaccggataccccttctccttccttcattttcattccttccaactctccacggagagtttgaagtcgaacctgtctaactcgatcggctcccttgtagaaatttgcaagaatgtcccacgcctcctttgaagttgttgcaccagcgatcttctcgaacccggaatcatcaacagtccagaataacatgtacaacgcccccttgtctttcaacctcatctccttcaatgtcttgttctgcaccgccgtatatcctgctatggatgttggttccttgaaaccaccatggacaatttcccaactatcatgggctcccaaaagagctttcatctggatactccagttttcgtaattagcctttgtaaggcggggtagtggtagactactacttggcccactctccattttctcaagctctgataccaatttgtgatatcacaggtcctcacacacactggtgcactcacccaaacacacaacaatctctctggtagaatgcttctgatatatttagataatggtctgtcaaactacacattggcctcttacatgagatgttatttataagtaatagcaagtgggatatgactcttcacatgagtaacttctttacctaagaacatgactcttcacatgagtaacttctttatctagggacatgactcttcacatgagtaacttcttcacttatctaatttattattattaaagtttaatccaacataaaaaacttaaccaggatcgtgatggcaattaagagttgatgagagttataacttacagtgtcaaatttccggaaaggcgaatccactttaggcgcaaacttggattcccaaactgaatcagcaatcacaatccccatgttaggcactgcacacaaatcaaagaaagaatatgcatacatgttagaaattatgatggcggtataaatagaaactgctgctgagttatatcattatctactacatccacttcaatcctaatatctggatattaatttaccatgtataacccatagttctggatacagttcctttatagttttctcgagtatctctgaaagcgtatacttcgaccaagtccctgtgcccagttctgctatgcattgcaaaattttgtttgtcagctctaccacagagtcgtcgccactcacgtctatttcttctataaaaggcaaaagcttttcggcttccctaacatgataccttggtacatttttcaaatgagttttcatccgatctataatggcttctctacgatccgatggtggtgcaggctcattgatctcgtccatttcttctgttcctctcagcctcagcaagtttacaaactaatcactttcgtttttgctcaagagacaaataacttgcttctacctaataccatacaattatcatacgcgttgaattcaacgtgtcaataaacaaagcttccacgcaattcttaaaataatcagtattacgtcacagttttagaaagtagattcgttttccactgtaattgccttttaaaattgaaagtcggtgtgattgaaagcaatgcatacgcatttttctagtaccacctgtaataaaatacattatcattcttgtccacaatttcacggcgattacttggttctagaaaatccggtttgactttgaattttcggtgcagcaaaacgcggtttcatattgaattttgccatcaacgccacgatagcattggaaaaaattgttttgagaaggtttgtttggtttcagacttccacatatcatcaagttgggcaaaatttgcagtacgagagagttggattcttgcgctgggaactactaccacatcatgtatgtgcttaatttctcctcggtaagttgtatatgcttacttctatgctgaactgcatcatgatcaaactccattcaactcaaacaaacagagctaatttgttcttaatcattcttatttattattgtttttcacatcgattttgaggaatctgaatatgccaaagtcagcaattgacatatgtatagctatattaattatattttgcagtttgaattgataaatcttgttgcgaaaatcttgaaccaaatgcagaatatccatgaaaaggacgagcatggagatgatgagaagggcaagcttgtatcatcaggacaaatgaatagtttaagtgtaagtttccttcttttcccctctaaactatttaagttataattattgcagaaatgtaatgccattatatatgctaatattaattaaactgcagagcaatcaagatgttcgaatgttagacttagttagagccatgtgcactgtccatgatgagaaatcaggacaacctttagcttttgactacacattgaacacaattatgcattttgttctggttgtgtgtgtagtgagttatttaaccaagcaataatatatggtgtgatgtatattatgtgtattccaggggaaagaggatagtagaacattaatgaacattagttctgaagttgaaagaagaacaccagaaatcatgggaagatgtgcatcgcggtgggccaccaccacattttttgtgtgtttgtgttcattaaaattcagcaagtcacttctccaaccacaaggacccaattcactatggcttgccatagtaggaatgttcagttttactgtgtttgtatctgcttcttctgttatgcttgttgttggagcacagttcactcgtatgcagataactgttatgctcagtgctttcttggctatgctgttagcttaattagacagttttcatatatgtacttatgttttcttcaggcagggttgaacacaagttgttgtaatcttgttatttacataatttcattcgcataacaataaaacaaagtttgggtatgtgatatatcaatatcaacgattaaccagacagaacatattcacggatgaa

SEQ ID NO: 20 - DNA, 10416 bp, Glycine argyea
  source 1..10416: mol_type = genomic DNA
ttttgccttttatagaagaaatagacgtgagtggcgacgactctgtggtagagctgacaaacaaaattttgcaatgcatagcagaactgggcacagggacttggtcgaagtatacgctttcagagatactcgagaaaactataaaggaactgtatccagaactatgggttatacatggtaaattaatatccagatattaggattgaagtggatgtagtagataatgatataactcagcagcagtttctatttataccgccatcataatttctaacatgtatgcatattctttctttgatttgtgtgcagtgcctaacatggggattgtgattgctgattcagtttgggaatccaagtttgcgcctaaagtggattcgcctttccggaaatttgacactgtaagttataactctcatcaactcttaattgccatcacgatcctggttaagttttttatgttggattaaactttaataataataaattagataagtgaagaagttactcatgtgaagagtcatgtccctagataaagaagttactcatgtgaagagtcatgttcttaggtaaagaagttactcatgtgaagagtcatatcccacttgctattacttataaataacatctcatgtaagaggccaatgtgtagtttgacagaccattatctaaatatatcagaagcattctaccagagagattgttgtgtgtttgggtgagtgcaccagtgtgtgtgaggacctgtgatatcacaaattggtatcagagcttgagaaaatggagagtgggccaagtagtagtctaccactaccccgccttacaaaggctaattacgaaaactggagtatccagatgaaagctcttttgggagcccatgatagttgggaaattgtccatggtggtttcaaggaaccaacatccatagcaggatatacggcggtgcagaacaagacattgaaggagatgaggttgaaagacaagggggcgttgtacatgttattctggactgttgatgattccgggttcgagaagatcgctggtgcaacaacttcaaaggaggcgtgggacattcttgcaaatttctacaagggagccgatcgagttagacaggttcgacttcaaactctccgtggagagttggaaggaatgaaaatgaaggaaggagaaggggtatccggtttcatcacccgggtctagaccgtggtaaatcaactaaacagaaatagagaggtgttgtccgatgccaaagtggtggagaaaattctgaggtctctcactgacgacttcgagaatgttgtatgtgcgattgaggagtcaaaagacctagcaacactcaccgttgacgaacttgctggttccttggaggcgcataaacaaagaaagaaaaagaagaaagaagagtcactggagcaagcactccagacaaaagcttcaatcaaggacgaaaaggtactctattctcaaaactttcgaggtagagggcgtggctatggaggtcgagaagacggtcgcggtagtagaggccgcggtcaagaaggaaggaataaggagcaggctaaattaagtcggcagaattggcgaggccaagatcgtggaagaggccgacattcaaatcaatcaaatgtagaatgttttaaatgcggaaagtatggacattactcgagagattgcaactcggacagatgttacaactgtggtcgagtcggacattttgcgaaagattgccaagatgaaagaagaaatgaaatttcaaaccttgttacagaaggagaagtaagagatggaggaattctgatgatgactagaagtgaagtcaaccagggattcgagatggaatggtatctagacactagtgcaagcaatcatatgtgtgggcataagcatttgttcagtaagctgagacaaatagaagccagacatgtttcgtttggagatgcttcaaaggttgatgtcaaaggccaaggtacgatttgtttcttgcagaaagatggacgaattgggcgcatccaagatgtctattacgtgcccgatctaaaaagtaatattcttagtgtgggacaactgatggagaatggatactcggttctcatgacagaccgagttctcttcttgaaggataaacaaggatgtttgatagttcaagttcagatgaagaaaaatagaatgttcaaactaatgttgaggagtgtgcaggtcggctgtttgcaagccgagatgagtgatgaagtatccttatggcatcgccgatttgggcatcttcactacggaggcctggtcgagatgatgaacaagaatatggtggaagggttgtcgagtttgaactatgaaaagagattttgtgaagaatgtgtgctaagcaagcatgctaggagtccatttcaaaagacggccaaatacagatctcaacaaccactagaactaattcataccgacttgtgtggacctatctctcctgggtctttcagtggaaagaggtattttgtctcgtttatagacgattttagtcgtaaaacttgggtgtatttgctggaagaaaaatccaaagtgcttgaagtattcaagagttttaagatgatgatagaaaatgcaactggaaggtgaatcaagtcaatccgttctgaccgaggtggggagtacacatcaattgcatttaaaaggtattgcgaggagcatgggataaaaaggttcttaactgcaccgtactcaccacagcaaaatggagtggctgagagaaaaaaagtaccattcttgacatggtaaggtcaatgttgaaaacgaagaaaatgcctaaggagttttggggacaggccgtattgtgtgcagtgtatgtgcagaaccgttgtccacattccagactaaatggaaagaccccacaggaagcatggacaggacagaagccatcagtaggccatttcagagtcttcgggagtgtggcttatgcacacatccctgatcagcaaagaaccaagttggaagacaagagcaggaaatttattttcattggatatgacgaaaagacaaagggatacaagttgttggatcccggcaccaagaaagtaatgataagccgagatgtgaaagttaatgaagaagatgaatggaactggagcacttcatccgaggtcacctcagatcccggacagacgcttactcgacaagaagagaaggaaggagaatcaccaagttggatcccggcaagaagtgagacatctgatgaagacgagatgagacaaccccgaatgaggagtttgcaggacctgtatgattcaactaatgaagcttaccttatttgtttgttgacagattctgaagaaataacatttaaagaggcaatccgagatgaaaaatggaaaaccgccatgaatgaagaaatcaaggccattgaacggaatgaaacttgggagttgaccgaactgccaaaaggaagtcagcccattggagttaaatgggtttataagagaaaaagaaaaaaaaagttggggatgaaagacgaaaaaaatatttaaagtttatggagagattttgttggattaaactttaataataataaattagataggtgaagaagttactcatgtgaagagtcatgtccctaggtaaataagttactcatgtgaagagtcatatcccacttgctattacttataaatagcatctcatgtaagaggccaatgtgtggcttggcagaccattatctaaatacatcagaagtattctaccagagagattgttgtgtgtttgtgtgagtgcactagtgtgtgtgaggacctgtgatatcacaaattggtttcagagcttgagaaaatagagagtgggccaagtagtagtctgccactaccccgccttacaaaggctaattacaaaaactggagtatccagatgaaagctcttttgggagcccatgatagttgggaagttgtccatggtggttttgagaaaccaacatccacagtaggatatacgacggcgcagaacaagacattgaaggagacgaggttgaaagacaaaggggcgttgtacatgttattccgggctgttgatgattttgggtttaagaagatcgctggtgcaacaaattcaaaggaggcgtgggacattcttgcaaatgtctacaagggagctgatcgagttagacaggttcgacttcaaactctccgtggagagttggaaggaatgaaaatgaaggaaggagaaggggtatccggtttcatcacccgggtccagaccgtggtaaatcaactaaacattctttagctctaatgtgtatctgcggaactacgccgccaatgatgcatcatcatcagcatcaggagtctgcggtactaagcctgccaaagatgcatcatcatcagcatcaggactctcagtcctcagtgtgaggccggaactcacacccttgtcatcccatcgggagatacccttttacgcatggatgcacgtatatgacacctccgagaataggcttcaaccgcgacgggaccacgtatctctggtgttaagggatcatataccctttatgtgagcaaaccaactaggagttgtgctctgctcctaggtaggtccatatctatctaacttgccagaagatctatccgaaaggcaaccaaacacaaagcacagaatatgcatacatattatcatgaggaaagctgtaacatccaatttaatgcatatgaatcatgtatgcaaccatcactccctcggagtgtggtgaaactcaagcagtgggaaacactcccaagaaccacgaacttaccaggcgaggccgtgctctgctgttcaatccctgaactgtggtctccgccactaacaccctcggtgtcacgcctatacaaaggcacacaggcatgaatcaacaatcatcctatctgtacaaggcatttaattataccaaaacacacggaaaaacagaattttctgcataaaattatgcagattttgtacccacttataaaaatcataaatccctgtagaaagctcctaaaatcaagggaatttagaggcatctatatttatctcctatctttcaaacaaaactggtttcatccaatttcataaaccacagcctctataacacttaaaataccagaactgttacgcactataacagtgctgacagtaacatatcagttcaaccagccattttcaaactcccgccaacaccaggatactcatccaaatgacctgagcttttacaggtataaattaaacatacacatctttctatttccactgaaagcactaaatttggatcatcctagactgagataaaatttccgcaagattgcctctgcaccagtaaaatctgctaaaaacagagtataacagcctgttataactctgcacaataaattctaggaacctccaaaaatagcacgaaaattccagaacctacttgaatctcttaactttcaaatgacactgtttttagaccatttggataccgatagctcctgttatgcacaaaacatcagggctgaccagatcgatgttccttgctgtcatgaacatgcatgtttcatgcttggaacaccatttcaaccttgaatcaacttctacacactccaaattacaccttaaaactgtttcacccttccaatactcaacatttatcaatttcactccaaaccctcatcaaaccaaaccctaaccttatgatttctaagaatcaaggagaaataggaggagagtaccaaccttagagttcaattgatcaaattggagagtggaccagctctctatagcttccaattctgcccagctactccgaatgaagtgggcagctcctagctttactcttttcctcaaatttctcacaaccaaactcaacaacaataaaggacaaggagaagagcttcctttcttgcttgatttatgtttccagccactaatcaaggagtgaagagaaatggtgcttgggtgtgaaggagagacatggacataaaggaagaagaaggagaaagtgagagaaccatggtcagccacagtttttaacatcaggaggggggtgcattcagcaatctcttattcacaccctaacctctaaacaaataccaaaatgtcccttctttcattaatcctcttttattaatactaagtctcttaattatgcaaatatcacccaaattacattgattataattagtgacattacattaatgcatagtatcttaaagaaataatttagctttttaagtttccaactttgtgaaacctgtaccaagctacgaaaaacaacaatttttctaccaagggcaagtccttgtcgttgtttgccttttgtccaaacatggcaacgaaataaaccagggcaagtaattgtgattctcttcctgttgtccatttttatgatcaatactaattggcatagaagtttactaaaaatatgcatgtttttccttacagtaattgaattgcaccttcacttgttaggtacctaacttttattgcttttatgtacagagagtcatactacaacgttaccttaatcgaatatatcttgatcagtattatccgatgcaaatggtgaaaaatcatgtaagtatagaactaattgatacaacaagttatgatctcttcgttcaaaacataactttctatagtgctattcattgctattaatattttactgtcaattggatcattataataacaaattttatatagagaagataatgcaaccactttgtgaacaagaaaaacacaaccttagtcttgttacttgtatagatgattggaaaatttgtctcacattaaggcaaatagacataattatgttttcaacaaaatgtagtgtcatcataactaaacacgtgttgcctaagtctacattgtcaaacacactctttacccatacataaaggcttgtgcctgttagaaataatagctatgccttattataacttggttgacaagtaattattttttaacaagtttcctagctagtttcttgtaataaaagttaatttcttgtgtgtctttcaaaatctcactttttttttaaatatctcacatctttcaaatatctataacatatctaccaatatagaaaaagaaactatcgataaaatgtcatattttcgttttctcttgtaaaagtagtaaaataaagttaaatcaattacctagattactttgagttgcaaaatgatttcaatatttataatcattaattcctgaaatattcggagttgtgttcccaattgacacctagaaatacaataaaatggaaggtactattttttttcttttgagtcatgattggttttaaatatgttggaatagaactaaacatgactttattatacgtattttatttcatatcatccaatactattattttttcttcttttctaatttagctactatgagatggaatcaatctctttgatcattttatcattaaaatattgacacaataaatattaatttattttatttcattggatccgttcaatttttttctattaatttctttctatccactatctaagtacaatcatagttcatattatttttacaagtcttgaaagtgagggactaaaatcacaaatttttaaaagtgaaaagtgaaatgtctcaattaaaaataggaggactaaaattacaattaattaaaaataaaaaataaaatgtctcaattaaaaattatgagaaaaataatggatttaaaaaaataagaagacgaaagttacattttaggtaaaataaataaaagtgtagtatcttaaagagataattgagcatttaagtatccaaatttatgaaaacaggaccaaactacataaaacatcaatttctaccgagggtaaggcattgggtgtgtttgtttttaaagttagaaaatcaattttaaaataagcattttttatcagcatttttttaagtagatataattagtttcttaaaataagaggaaagttattttttaagcataagtaatttagacaatcacactaaaaataaaaagttgtttattttttgttttttttaacaaaaatttaaacatttaattttcctttcttcggtttgtgtccaaaagcgggagctaaatgaatcatataattttctttctcttccttttgtccatttttaatgctcgatactaattgacatcagattaaatataataaaggcatgcttatttttccttaatgtatttgaatttcacctctactaagtgcctaattaacatctatttcttttatgtacagcagaagcggctgcaagtatatactggcattgacctagaaagttgttttgtgaaaaggccagagaatgaaataatatcaaaggtacataatttttcccaagtcttatcaataccattattattttaatgacaaatggaacattgtaataacaaatttgatatagagatgtgatcaagaaaaacacaaaactttgtctagtgcagaaatcaaatcatcttacatctttagacagaatttctactcacaatacaaagtatatccttcaaatatgtcacattttgtttttgatgtatctcaacatagtatctaatgatatgatccataagctagggaaaatcattgtatttattttaacaaataagttgaattgcaccttaatttaacataatatctaatgatatgatcaagtacctaaccttccttgcttttatgcacaaaaacaagcatgcgagagtcaagaggagaacaaggtacatatagaactaattaatgacaggttattgtgttttcttcatccaaaacataatttttttgggacttatcattaacattattattttattgtcaaatggaacattataataacaattttgatgtaaaaaagctaattcaatgaccttgtctcgtgacaaaaaaagtacaatttatgtcttatgtaattattttcaaaatcgtcttatgcacggggagaaaagacacattatattttcaatggcgcaaagtgtagtgtcatctcgtaacgaaacatttggtgtctaactccatattgttagactaactttctactcacataaaaaagatgtgtctttcaaatatatttttgttttttctcggcttgaaatatctcacataactcaacataggagcaaaatgagccatgtagggaaaatcattgtcttttccttttgtccattttgatgatcaatactaattgggttgaaaatttaataaagagatgcatatgcatatttttccttaatttaacttgattggatctttacttctcaagtacctaacatttgttgcttttttgtacagccacagaaagaactatatcagcaactgaaaaaatgcaaggtaacatatagaattaggtaataataacaagtcatcaagctttagacacactttttgctcacatagagaggatatgtctttcaaatatctcacaattttatttttgaaatatctcacatctttcacatacctcaacatatctaaaattatatatatggtgtgcatgtgtgtcacatatctcaacatgggtgcaaaatgagccatatatcattttgttattttgtcccctacaatgacatttccattgcatgagatctttacatttacaagaaaataccaaaaataaagttccatagaaagaccatatattatttcatcaaaattctcactcaccaaccatatttctccattgtgattgactatcaacaggaagaaaaggaaaatcgccgttctcatcccccaaaccatgttgtaaagattgacgataatggggtgttaaaagtagataaaggaaagacgaagagtttgaacacatttttgaagttttatgggaacaggccattccaatcacattctactgatgcaaatcgtcttactacatgggctgccttatatggaatgcaatgggcccaactatctgttttgggcaatcctcttgagaagttatcccaatccagaggcattgacccaatggaattaagtatggggaaagcatgttccatcacagtatgggcatcaatatttttcctgaactttatttttgcagccatgcttaccttgtcattgcctcatcgtatacccgcatactcaatgctgggatttaatttactctattacattttagcgataatagttgcagtgtctggatataatgtttcatggggtggttcagctaaaatctcaggcctattgtttgtgtttggaggggccacatccttaatgctacttcttagcatcataatcatgggggtgctctatgctagctgcatatggtgctcctatccaaatgatgttggaggagaagtaactcagaaacactcgcctgagtgtatcatcaagatccaggatacctgaaatgacgcacttgaatgtaagtctggaatctggtattttggatccagaaacaatttatctgttttatttctttttaggtcgtggttgcctttgaaatatcaaagtttgagtattt

SEQ ID NO: 21 - DNA, 433 bp, Glycine argyea
  source 1..433: mol_type = genomic DNA
gcttttcggcttccctaacatgataccttggtacatttttcaaatgagttttcatccgatctataatggcttctctacgatccgatggtggtgcaggctcattgatctcgtccatttcttctgttcctctcagcctcagcaagtttacaaactaatcactttcgtttttgctcaagagacaaataacttgcttctacctaataccatacaattatcatacgcgttgaattcaacgtgtcaataaacaaagcttccacgcaattcttaaaataatcagtattacgtcacagttttagaaagtagattcgttttccactgtaattgccttttaaaattgaaagtcggtgtgattgaaagcaatgcatacgcatttttctagtaccacctgtaataaaatacattatcattcttgtccacaatttcacggcgatta

SEQ ID NO: 22 - DNA, 1305 bp, Glycine argyea
  source 1..1305: mol_type = genomic DNA
cttggttctagaaaatccggtttgactttgaattttcggtgcagcaaaacgcggtttcatattgaattttgccatcaacgccacgatagcattggaaaaaattgttttgagaaggtttgtttggtttcagacttccacatatcatcaagttgggcaaaatttgcagtacgagagagttggattcttgcgctgggaactactaccacatcatgtatgtgcttaatttctcctcggtaagttgtatatgcttacttctatgctgaactgcatcatgatcaaactccattcaactcaaacaaacagagctaatttgttcttaatcattcttatttattattgtttttcacatcgattttgaggaatctgaatatgccaaagtcagcaattgacatatgtatagctatattaattatattttgcagtttgaattgataaatcttgttgcgaaaatcttgaaccaaatgcagaatatccatgaaaaggacgagcatggagatgatgagaagggcaagcttgtatcatcaggacaaatgaatagtttaagtgtaagtttccttcttttcccctctaaactatttaagttataattattgcagaaatgtaatgccattatatatgctaatattaattaaactgcagagcaatcaagatgttcgaatgttagacttagttagagccatgtgcactgtccatgatgagaaatcaggacaacctttagcttttgactacacattgaacacaattatgcattttgttctggttgtgtgtgtagtgagttatttaaccaagcaataatatatggtgtgatgtatattatgtgtattccaggggaaagaggatagtagaacattaatgaacattagttctgaagttgaaagaagaacaccagaaatcatgggaagatgtgcatcgcggtgggccaccaccacattttttgtgtgtttgtgttcattaaaattcagcaagtcacttctccaaccacaaggacccaattcactatggcttgccatagtaggaatgttcagttttactgtgtttgtatctgcttcttctgttatgcttgttgttggagcacagttcactcgtatgcagataactgttatgctcagtgctttcttggctatgctgttagcttaattagacagttttcatatatgtacttatgttttcttcaggcagggttgaacacaagttgttgtaatcttgttatttacataatttcattcgcataacaataaaacaaagtttgggtatgtgatatatcaatatcaacgattaaccagacagaacatattcacggatgaa

SEQ ID NO: 23 - DNA, 1221 bp, Glycine argyea
  source 1..1221: mol_type = unassigned DNA
ttttgccttttatagaagaaatagacgtgagtggcgacgactctgtggtagagctgacaaacaaaattttgcaatgcatagcagaactgggcacagggacttggtcgaagtatacgctttcagagatactcgagaaaactataaaggaactgtatccagaactatgggttatacatgtgcctaacatggggattgtgattgctgattcagtttgggaatccaagtttgcgcctaaagtggattcgcctttccggaaatttgacactagagtcatactacaacgttaccttaatcgaatatatcttgatcagtattatccgatgcaaatggtgaaaaatcatcagaagcggctgcaagtatatactggcattgacctagaaagttgttttgtgaaaaggccagagaatgaaataatatcaaagccacagaaagaactatatcagcaactgaaaaaatgcaaggaagaaaaggaaaatcgccgttctcatcccccaaaccatgttgtaaagattgacgataatggggtgttaaaagtagataaaggaaagacgaagagtttgaacacatttttgaagttttatgggaacaggccattccaatcacattctactgatgcaaatcgtcttactacatgggctgccttatatggaatgcaatgggcccaactatctgttttgggcaatcctcttgagaagttatcccaatccagaggcattgacccaatggaattaagtatggggaaagcatgttccatcacagtatgggcatcaatatttttcctgaactttatttttgcagccatgcttaccttgtcattgcctcatcgtatacccgcatactcaatgctgggatttaatttactctattacattttagcgataatagttgcagtgtctggatataatgtttcatggggtggttcagctaaaatctcaggcctattgtttgtgtttggaggggccacatccttaatgctacttcttagcatcataatcatgggggtgctctatgctagctgcatatggtgctcctatccaaatgatgttggaggagaagtaactcagaaacactcgcctgagtgtatcatcaagatccaggatacctgaaatgacgcacttgaatgtaagtctggaatctggtattttggatccagaaacaatttatctgttttatttctttttaggtcgtggttgcctttgaaatatcaaagtttgagtattt

SEQ ID NO: 24 - protein, 367 aa, Glycine argyea
MPFIEEIDVSGDDSVVELTNKILQCIAELGTGTWSKYTLSEILEKTIKELYPELWVIHVPNMGIVIADSVWESKFAPKVDSPFRKFDTRVILQRYLNRIYLDQYYPMQMVKNHQKRLQVYTGIDLESCFVKRPENEIISKPQKELYQQLKKCKEEKENRRSHPPNHVVKIDDNGVLKVDKGKTKSLNTFLKFYGNRPFQSHSTDANRLTTWAALYGMQWAQLSVLGNPLEKLSQSRGIDPMELSMGKACSITVWASIFFLNFIFAAMLTLSLPHRIPAYSMLGFNLLYYILAIIVAVSGYNVSWGGSAKISGLLFVFGGATSLMLLLSIIIMGVLYASCIWCSYPNDVGGEVTQKHSPECIIKIQDT

SEQ ID NO: 25 - DNA, 788 bp, Glycine argyea
  source 1..788: mol_type = unassigned DNA
cttggttctagaaaatccggtttgactttgaattttcggtgcagcaaaacgcggtttcatattgaattttgccatcaacgccacgatagcattggaaaaaattgttttgagaaggtttgtttggtttcagacttccacatatcatcaagttgggcaaaatttgcagtacgagagagttggattcttgcgctgggaactactaccacatcatgtatgtgcttaatttctcctcgaatatccatgaaaaggacgagcatggagatgatgagaagggcaagcttgtatcatcaggacaaatgaatagtttaagtgggaaagaggatagtagaacattaatgaacattagttctgaagttgaaagaagaacaccagaaatcatgggaagatgtgcatcgcggtgggccaccaccacattttttgtgtgtttgtgttcattaaaattcagcaagtcacttctccaaccacaaggacccaattcactatggcttgccatagtaggaatgttcagttttactgtgtttgtatctgcttcttctgttatgcttgttgttggagcacagttcactcgtatgcagataactgttatgctcagtgctttcttggctatgctgttagcttaattagacagttttcatatatgtacttatgttttcttcaggcagggttgaacacaagttgttgtaatcttgttatttacataatttcattcgcataacaataaaacaaagtttgggtatgtgatatatcaatatcaacgattaaccagacagaacatattcacggatgaa

SEQ ID NO: 26 - protein, 136 aa, Glycine argyea
MYVLNFSSNIHEKDEHGDDEKGKLVSSGQMNSLSGKEDSRTLMNISSEVERRTPEIMGRCASRWATTTFFVCLCSLKFSKSLLQPQGPNSLWLAIVGMFSFTVFVSASSVMLVVGAQFTRMQITVMLSAFLAMLLA

SEQ ID NO: 27 - DNA, 11111 bp, Glycine argyea
  source 1..11111: mol_type = genomic DNA
agaaaaaataacaagtattaatgtgatatttaaaacaaaagagaatttcaatagtaagatttttcatttaaaaagcgtcttgggccacttctaaagaagttgcaaagaaactgcttttcagaaatttcatttcaaagaccaattactatcaaatactcaaactttgatatttcaaaggcaaccacgacctaaaaagaaataaaacagataaattgtttctggatccaaaataccatattccagacttacattcaagtgcgtcatttcaggtatcctggatcttgatgatacactcaggcgagtgtttctgagttacttctcctccaacatcatttggataggagcaccatatgcagctagcatagagcacccccatgattatgatgctaagaagtagcattaaggatgtggcccctccaaacacaaacaataggcctgagattttagctgaaccaccccatgaaacactatatccagacactgcaactattatcgctaaaatgtaatagagtaaattaaatcccagcattgagtatgcaggtatacgatgaggcaatgacaaggtaagcatggctgcaaaaataaagttcaggaaaaatattgatgcccatactgtgatggaacatgctttccccatacttaattccattgggtcaatgcctctggattgggataacttctcaagaggattgcccaaaacagatagttgggcccattgcattccatataaggcagcccatgtagtaagacgatttgcatcagtagaatgtgattggaatagcctgttcccataaaacttcaaaaatgtgctcaaactcttcgtctttcctttatctacttttaacaccccattatcgtcaatctttacaacatggtttgggggatgagaacggcgattttccttttcttcctgttgatagtcaatcacaatggagaaatatggttggtgagtgagaattttgatgaaataatagatggtctttctatggaactttatttttggtattttcttgtaaatgtaaagatctcatgcaatggaaatgtcattgtaggggacaaaataacaaaatgatatatggctcattttgcacccatgttgagatatgtgacacacatgcacaccatatatataattttagatatgttgaggtatgtgaaagatgtgagatatttcaaaaataaaattgtgagatatttgaaagacatatcctctctatgtgagcaaaaagtgtgtctaaagcttaatgacttgttattattacctaattctatatgttaccttgcattttttcagttgctgatatagttctttctgtggctgtacaaaaaagcaacaaatgttaggtacttgagaagtaaagatccaatcaagttacattaaggaaaaatatgcatatgcatctctttattaaattttcaagccaattagtattgatcatcaaaatggacaaaaggaaaagacaatgattttccctacatggctcattttgctcctatgttgagttatgtgagatatttcaagccgagaaaaaacaaaaatatatttgaaagacacatctttttatgtgagtagaaagttagtctaacaatatggagttagacaccaaatgtttcgttacgagatgacactacactttgcgccattgaaatataatgtgtcttttctcccagtgcataagacgattttgcaaataattacataagacataaattgtactttttttgtcacgagacaaggtcattgaattagcttttttacatcaaaattgttattataatgttccatttgacaataaaataataatgttaatgataagtcccaaaatttttttgttttggatgaagaaaacacaataacctgtaattaattagttctatatgtaccttgttctcctcttgactctcgcatgcttgtttttgtgcataaaagcaaggaaggttaggtacttgatcatatcattagatattatgttaaattaaggtgcaattcaacttatttgttaaaataaaataccatgattttccctagcttatggatcatatcattagatattatgttgagatacatcaaaaacaaaaatgtgacatatttgaaggatatactttgtattgtgagtagaaattctgtctaaaaatgtaagatgatttgatttctgcactagacaaagttttgtgtttttcttgatcacatctctatatcaaatttgttattacaatgttccatttgtcattaaaataataatggtattgataagacttgggaaaaattatgtacctttgatattatttcattctctggccttttcacaaaacaacgttctaggtcaatgtcagtatatacttgcagccgcttctcctgtacataaaagaaatagaggttaattaggcacttagtagaggtgaaattcaaatacattaaggaaaaataagcatgcctttattatatttaatctcatgtcaattagtattgagcattaaaaatggacaaaaggaagagaaagaaaattatatgattcatttagctcccgctttggatacaaacggaagaaaggcaaattaaatgtttaaatttatttgttaaaaaaaactaaaaataaacaactttttatttttagtgtgattgtctaaattgcttatgcttaaaaaataactttcctcttattttaagaaactaattatatctacttaaaaaaatgctgataaaaaatgcttattttaaaattgattttctaactttaaaaacaaacacacccaatgccttaccctcggtagaaattgatgtttttttgtagtttggtcctgttttcataaatttggatacttaaatgctcaattacctctttaagatactacacttttatttattttatctaaaatgtaactttcgtcttcttatttttttaaatccattattttttctcataatttttaattgagacattttgttttttatttttaattaattgcaattttagtcctcctatttttaattgagacatttcgcttttaaaaatttgtgattttagtccctcactttcaagacttgtaaaaataatatgaactatgattgtacttagatagtggatagaattaacaataataaaccatggttgtactttaatagtggatagaaagaaattaatagaaaaaaattgaacggatccaatggaataaaataaattaatatttattgtgtcaatattttaatgataaaaggatcaaagagattgattccatctcatagtagctaaattagaaaagaagaaaaaataatagtattgggtgatatgaaataaaatacgtataataaagtcatgtttagttctattccaacatatttaaaaccaatcatgactcaaaagaaaaaaaatagcaccttccattttattgtatttctaggtgtcaattggaacacaactccgaatatttcaggaattaatgattataaatattgaaatcattttgcaactcaaagtaatctaggtaattgatttaactttattttactacttttacaagagaaaacgaaaatatgacattttatcgatagtttctttttctatattggtagatatgttatagatatttgaaagatgtgagatatttaaaaaaaaagttagattttgaaagacacacaagaaattaacttttattacgagaaactagctaagaaacttgttaaaaaataattacttgtcaaccaagttataataaggcataactattatttctaacaagcacaagcctttatgtatgggtaaagagtgtgtttgacaatgtagacttaggcaacatgtgtttagttatgatgacactacattttgttgaaaacataattatgtctattttccttaatgtgagacaaattttccaatcatctatacaagtaacaagactaaggttgtgtttttcttgttcacaaagtggttgcattatcttctctatatcaaatttgttattataatgatccaattgacagtaaaatattaatagcaatgaatagcactatagaaaattatgttttgaacgaagagatcataacttgttgtatcaattagttctatacttacatgatttttcaccatttgcatcggataatactgatcaagatatattccattaaggtaacgttgtagtatgactctctgtacataaaagcaataaaagttaggtacctaacaagtgaaggtgcaattcaattactgtaaggaaaaacatgcatatttttagtaaacttctatgccaattagtattgatcataaaaatggacaacaggaagagaatcacaattacttgccttggtttatttcgttgccatgtctggacaaaaggcaaacaacgacaaggacttgcccttggtagaaaaattgttgtttttcgtagcttggtacaggtttcacaaagttggaaacttaaaaagataaattatttctttaagatactatccattaatgtaatgtcactaattataatcaatgtaatttgggtgatatttgcataattaagagacttagtattaataaaagagaattaatgaaagaatggacattttggtatttgtttagaggttagggtgtgaataagagattgctgaatgcacccccctcctcatgttaaaaactgtggctgaccatggttgtctcactttctccttcttcttcctttatgtccatgtctctccttcacacccaagcaccatttctcttcactccttgattagtggctggaaacataaatcaagcaagaaaggaagctcttctccttgtcctttattgttgttgagttttggttgtgagcaatttgaggaaaagagtagagctaggagctgcccacttcattcggagtagctgggcagaattggaagctatagagagctggtccactctccaatttgatcaattgaactctaaggttggtactctcctcctatttctccttgattcttagaaatcataaggttagggtttggtttgataaatgttgagtattggaagggtgaaacagttttaaggtgtaatttggagtgtgtagaagttgattcaaggttgaaatggtgttccaagcatgaaacatgcatgttcatgacatcaaggaacatcgatctggtcaaccctgatgttttgtgcataacaggagctatcggtatccaaatggtctaaaaacagtgtcatttgaaagttaagagattcaagtaggttctggaattttcgtgctatttttggaggttcctagaatttattgtgcagagttataacaggctgttatactctgtttttagcagattttactggtgtttagcagattttactggtgcagagggcaatcttgaagaaattttatctcagtctaggatgatccaaatttagtgatttcagtggaaatagaaagatgtgtatgtttaatttatacctgtaaaatctcaggtcatttggatgagtatcctggtgttggcgggtgtttgaaaattgctggttgaactgatatgttattgtcagcactgttatagtgcgtaacagttctggtattttaagtgttacagaggttgtggtttatgaaattggatgaaaccagttttgtttgaaagataggagataaatatagatgcctctaaattcccttgattttaggagcttcctacagggatttatgatttttataagtgggtacaaaatctgcataattttatgcagaaaattctgtttttccgtgtgttttggtataattaaatgccttgtacggataggatgattgttgattcatgcctgtgtgcctttatatagggcatgacaacgagggtgttagtggcggagaccacagttcagggattgaatagcagagcacggccttgcctggtaagttcgtggttcttgggagtgttttccactgcttgagtttcaccacactccgagggagtgatggttgcatacatgattcatatgcattaaattggatgttacatctttcctcatgataatatgtatgcatattctgtgctttgtgtttggttgcctttcggatagatcttctggcaagttagatagatatagacctccctaggagcagagcacaactcctagttggtttgctcacgtaaagggtatatgatcccttaacaccagagatacgtggtcccgtcgtggttgaagcctattctcggaggtgtcatatacgtgcatccatgcgtaaaggggtatctcccgatgggatgacaagggtgtgagttccggcctaacactgaggactgagagtcctgatgctgatgatgatgcatctttggcaggcttagtaccgcagactcctgatgctgatgatgatgcatcattggcagcgtagttccgcagatacacattagagctaaagaatgtttagttgatttaccacggtctggacccgggtgatgaaaccggataccccttctccttccttcattttcattccttccaactctccacggagagtttgaagtcgaacctgtctaactcgatcggctcccttgtagacatttgcaagaatgtcccacgcctcctttgaatttgttgcaccagcgatcttctaaaacccagaatcatcaacagcccggaataacatgtacaacacccctttgtctttcaacctcgtctccttcaatgtcttgttctgcgccatcgtatatcctgctgtggatgttggtttctcgaaaccaccatggacaacttcccaactatcatgggctcccaaaagagctttcatatggatactccaattttcataattagcctttgtaaggcggggtagtggcagactactacttgacccactctccattttctcaagctctgaaaccaatttgtgatatcacatgtcctcacacacactggtgcactcacataaacacacaacaatctctctagtagaatacttctgatgtatttagataatggtctgccaagccacacattggcctcttacatgagatgctatttataagtaatagcaagtgagatatgactcttcacatgagtaacttatttacctagggacatgactcttcacatgagtaacttcttcacctagggacatgactcttcacatgagtaacttcttcacctatctaatttattattattaaagtttaatccaacaaaatctccccataaactttaaatatttctttcgtctttcatccccaacttttttttttctttttctcttataaacccatttaactccaatgggctgacttccttttggcagttcggtcaactcccaagtttcattccgttcaatgaccttgattttttcattcatggcggttttccatttttcatctcggattgcctctttaaatgttatttcttcagaatctgtcaacaaacaaataaggtaagcttcattagttgaatcatacaggtcatgcaaactcctcattcggggttgtctcatctcgtcttcatcagatgtctcacttcttgccgggatccaacttggtgattctccttccttctcttcttgtcgagtaagcgtctgtccgggatctgaggtgaccttggatgaagtgctccagttccattcatcttcttcattaactttcacatctcggcttatcattactttcttggtgccgggatccaacaacttgtatccctttgtcttttcgtcatatccaatgaaaataaatttcctgctcttgtcttccaacttggttctttgctgatcagggatgtgtgcataagccacactcccgaagactctgaaatggcctactgatggcttctgtcctgtccatgcttcctgtggggtctttccatttagactggaatgtggacaacggttctgcacatacactgcacacaatacggcctgcccccaaaactccttaggcattttcttcgttttcaacattgaccttaccatatcaagaatgatacggtttttctctcagccactccattttgctgtggtgagtacggtgcagttaaaaacctttttatcccatgctcctcgcaataccttttaaatgcaattgatgtgtactccccacctcggtcagaacggattgacttgattcaccttccagttgcattttctatcatcatcttaaaactcttgaatacttcaagcactttggatttttcttccagcaaatacacccaagttttacgactaaaatcgtctataaacgagacaaaatacctctttccactgaaagacccaggagagatgggtccacacaagtcggtatgaattagttctagtggttgttgagatctgtatttggccgtcttttgaaatggactcctaacatgcttgcttagcacacattcttcacaaatctcttttcacagttcaaactcggcaactcttccaccatattcttgttcatcatctcgaccaggcctccgtagtgaagatgcccaaatcggcgatgccataaggatacttcatcactcatctcggcttgcaaacagtcgacctgcacactcctcaacattagtttgaacattctatttttcttcatctgaacttgaactatcaaacatccttgtttatccttcaagaagagaactcggtctgtcatgagaaccgagtatccattctccatcagttgtcccacactaagaatattactttttagatcgggcacgtaatagacatcttggatgcgcccaattcgtccatctttctgcaagaaacaaatcgtacctcagcctttgacatcaacctttgaaacatctccaaacgaaacatgtatggcttctatttgtctcagattactgaacaaatgcttatgcccacacatatgattgcttgcactagtgtctagataccattccatctcgaatccctggttgacgtcacttctagtcatcatcagaattcctccatctcttacttctccttctgtaacaaggtttgaagtttcatttcttctttcatcttggcaatccttcgcaaaatgtccgactcgaccacagttgtaacatctgtccgagttgcaatctcttgagtaatgtccatactttcctcatttaaaacattctacatttgattgatttgaatgtcggcctcttccacgaccttggcctcgccaattctgccgacttaatttagcctgctccttattccttccttcttgaccgcggcctctactaccgcgaccgtctcctcgacctccatagtcatgccctctacctcgaaagttttgagaataaagtaccttttcgtccttgattgaagcttttgtctggagtgcttgctccagtgactcttctttcttctttttctttctttgttcatgcgcctccaaggaaccagcaagttcgtcaacggtgagcgttgctaggtcctttgactcctcaatcgcacatacaacattctcgaagtcgtcagtgagagacctcagaattttctccaccactttggcatcggacaacacctctccatttctgtttagttgatttaccacggtctagacccgggtgatgaaaccggataccccttctccttccttcattttcattccttccaactctccacggagagtttgaagtcgaacctgtctaactcgatcggctcccttgtagaaatttgcaagaatgtcccacgcctcctttgaagttgttgcaccagcgatcttctcgaacccggaatcatcaacagtccagaataacatgtacaacgcccccttgtctttcaacctcatctccttcaatgtcttgttctgcaccgccgtatatcctgctatggatgttggttccttgaaaccaccatggacaacttcccaactatcatgggctcccaaaagagctttcatctggatactccagttttcgtaattagcctttgtaaggcggggtagtggtagactactacttggcccactctccattttctcaagctctgataccaatttgtgatatcacaggtcctcacacacactggtgcactcacacaaacacacaacaatctctctggtagaatacttctgatatatttagataatggtctgccaagccacacattgacctcttacatgagatgttatttataagtaatagcaagtgggacatgactcttcacatgagtaacttctttacctagggacatgactcttcacatgagtaacttcttcacctatctaatttattattattaaagtttaatccaacataaattacttaatcaggatcgtgatggcaattaagagttgatgagagttataacttacagtgtcaaatttccggaaaggcgaatccactttaggcgcaaacttggattcccaaactgaatcagcaatcacaatccccatgttaggcactgcacacaaatcaaagaaagaatatgcatacatgttagaaattatgatggcggtataaacagaaactgctgctgagttatatcattatctactacttccacttcaatcctaatatctggatattaatttaccttgtataacccatagttctggatacagttcctctatagtttcctcgagtatccatgaaagcatattcttaaaccaaatctgtgtgtccagttctgctatgcattgcaaaattttgtttgtcagctctaccacagagtcgtcgccactgatcacgtttatttcttctatcaaatgcgcaatattctcggcttccataactgaataacatggtagatttttcaaatgagttttcatccgatctataatggcttctccacgatccgatggtggtgcaggctcattgatctcgtccatttcttctgttcctctcagtctcagcaagtttacaaactactcactttcttttttgctcaagagacacataacttgcttctacctaataccatacaattatcatacgcgttgaattcaacgtttcaataacacaccttccacgcaattcttaaaataatcagcattacgtcacagttctagaatgtataatcgttttccacggtaagtgccttttaaagttgaaagttggtgtggttgacagcaatgcatatgcatttttctagtaccacgtgtaataaaatacattatcattctagaaaagccggtttgactttgaattttcggtgcagcaaaacgcggtttcatattgaatttcgccatcaacgccacgatagcattggaaaaaaat

SEQ ID NO: 28 - DNA, 1509 bp, Glycine argyea
  source 1..1509: mol_type = genomic DNA
ctaataccatacaattatcatacgcgttgaattcaacgtttcaataacacaccttccacgcaattcttaaaataatcagcattacgtcacagttctagaatgtataatcgttttccacggtaagtgccttttaaagttgaaagttggtgtggttgacagcaatgcatatgcatttttctagtaccacgtgtaataaaatacattatcattctagaaaagccggtttgactttgaattttcggtgcagcaaaacgcggtttcatattgaatttcgccatcaacgccacgatagcattggaaaaaaatgttttgagaaggtgtgtttggtttcagactttcacatatcatccaggtggagcaaaatttgcagtacgagagagttggattcttgggctgggaactactaccaaatcatgtatatgcctaatttctcctcggtaagttgtatatgcttacttctatgctgaactgcatcatgatcaaactccattcaactcaaacaaaccgagctaatttgttcttaatcattcttatttattatcgtttttcacatcgattttgaggaatctgaatatgccaaagtcagcagttgacacatgtatagctatattaattatattttgcagtttccatcgacaaatcttgttgcgaaaatcttgaaccaaaggcagaatatccatgaaaaggaggagcatggagatgatgagaagggcaagctggtatcattaggacaaatgaatagtttaagtgtaagtttccttcttttcccctctaaactatataagttgtaattattgcagaaatgtaatgccattatattatgctaatactaattaaactgcagagcaagcaagatgttccaatgttagacttagttagagccatgtgcactgtccatgatgagaaatcaggacaacctttagcttttgagtacacattgaacacaattatgcattttgttctggttgtgtgtagtgagttatttaactaagctaatatatggtgtgatgtatattatgtgtattccagaggaaagaggatagtagaacatcaatgaacattagttctgaagttgaaagaagaacaccagaaatcatgggaagatgtgcatcgtggtgggccacctccacattttttttgtgtttgtgttcattaaaattcagcaagtcacttctccaaccacaaggacccaaatcactatggcttgccatagtaggaatgttcagttttactgtgtttgtatctgcttcttctgttatgctagttgttggagcacagttcactcgtatgcagataactgttatgctcagtgctttcttggctatgctgttagcttaattagatagttttcatatatgtacttatgttttcttcaggcagggttgacaagttgttttaatcttgttatttacataatttcattcgcataacattaaaacaaagtttgggtatgtgatatatcaatatcaacgattaaccagacagaacatattcacggatgaacattga

SEQ ID NO: 29 - DNA, 106 bp, Glycine argyea
  source 1..106: mol_type = genomic DNA
ctaataccatacaattatcatacgcgttgaattcaacgtttcaataacacaccttccacgcaattcttaaaataatcagcattacgtcacagttctagaatgtata

SEQ ID NO: 30 - DNA, 204 bp, Glycine argyea
  source 1..204: mol_type = genomic DNA
tataatcgttttccacggtaagtgccttttaaagttgaaagttggtgtggttgacagcaatgcatatgcatttttctagtaccacgtgtaataaaatacattatcattctagaaaagccggtttgactttgaattttcggtgcagcaaaacgcggtttcatattgaatttcgccatcaacgccacgatagcattggaaaaaaat

SEQ ID NO: 31 - DNA, 88 bp, Glycine argyea
  source 1..88: mol_type = genomic DNA
tataatcgttttccacggtaagtgccttttaaagttgaaagttggtgtggttgacagcaatgcatatgcatttttctagtaccacgtg

SEQ ID NO: 32 - DNA, 1221 bp, Glycine argyea
  source 1..1221: mol_type = unassigned DNA
atggacgagatcaatgagcctgcaccaccatcggatcgtggagaagccattatagatcggatgaaaactcatttgaaaaatctaccatgttattcagttatggaagccgagaatattgcgcatttgatagaagaaataaacgtgatcagtggcgacgactctgtggtagagctgacaaacaaaattttgcaatgcatagcagaactggacacacagatttggtttaagaatatgctttcatggatactcgaggaaactatagaggaactgtatccagaactatgggttatacaagtgcctaacatggggattgtgattgctgattcagtttgggaatccaagtttgcgcctaaagtggattcgcctttccggaaatttgacactagagtcatactacaacgttaccttaatggaatatatcttgatcagtattatccgatgcaaatggtgaaaaatcatgagaagcggctgcaagtatatactgacattgacctagaacgttgttttgtgaaaaggccagagaatgaaataatatcaaagccacagaaagaactatatcagcaactgaaaaaatgcaaggaagaaaaggaaaatcgccgttctcatcccccaaaccatgttgtaaagattgacgataatggggtgttaaaagtagataaaggaaagacgaagagtttgagcacatttttgaagttttatgggaacaggctattccaatcacattctactgatgcaaatcgtcttactacatgggctgccttatatggaatgcaatgggcccaactatctgttttgggcaatcctcttgagaagttatcccaatccagaggcattgacccaatggaattaagtatggggaaagcatgttccatcacagtatgggcatcaatatttttcctgaactttatttttgcagccatgcttaccttgtcattgcctcatcgtatacctgcatactcaatgctgggatttaatttactctattacattttagcgataatagttgcagtgtctggatatagtgtttcatggggtggttcagctaaaatctcaggcctattgtttgtgtttggaggggccacatccttaatgctacttcttagcatcataatcatgggggtgctctatgctagctgcatatggtgctcctatccaaatgatgttggaggagaagtaactcagaaacactcgcctgagtgtatcatcaagatccaggatacc

SEQ ID NO: 33 - DNA, 1032 bp, synthetic construct
  source 1..1032: mol_type = other DNA
atggacgagatcaatgagcctgcaccaccatcggatcgtggagaagccattatagatcggatgaaaactcatttgaaaaatctaccatgttattcagttatggaagccgagaatattgcgcatttgatagaagaaataaacgtgatcagtggcgacgactctgtggtagagctgacaaacaaaattttgcaatgcatagcagaactggacacacagatttggtttaagaatatgctttcatggatactcgaggaaactatagaggaactgtatccagaactatgggttatacaagtgcctaacatggggattgtgattgctgattcagtttgggaatccaagtttgcgcctaaagtggattcgcctttccggaaatttgacactagagtcatactacaacgttaccttaatggaatatatcttgatcagtattatccgatgcaaatggtgaaaaatcatgagaagcggctgcaagtatatactgacattgacctagaacgttgttttgtgaaaaggccagagaatgaaataatatcaaagccacagaaagaactatatcagcaactgaaaaaatgcaaggaagaaaaggaaaatcgccgttctcatcccccaaaccatgttgtaaagattgacgataatggggtgttaaaagtagataaaggaaagacgaagagtttgagcacatttttgaagttttatgggaacaggctattccaatcacattctactgatgcaaatcgtcttactacatgggctgccttatatggaatgcaatgggcccaactatctgttttgggcaatcctcttgagaagttatcccaatccagaggcattgacccaatggaattaagtatggggaaagcatgttccatcacagtatgggcatcaatatttttcctgaactttatttttgcagccatgcttaccttgtcattgcctcatcgtatacctgcatactcaatgctgggatttaatttactctattacattttagcgataatagttgcagtgtctggatatagtgtttcatgg

SEQ ID NO: 34 - protein, 344 aa, synthetic construct
MDEINEPAPPSDRGEAIIDRMKTHLKNLPCYSVMEAENIAHLIEEINVISGDDSVVELTNKILQCIAELDTQIWFKNMLSWILEETIEELYPELWVIQVPNMGIVIADSVWESKFAPKVDSPFRKFDTRVILQRYLNGIYLDQYYPMQMVKNHEKRLQVYTDIDLERCFVKRPENEIISKPQKELYQQLKKCKEEKENRRSHPPNHVVKIDDNGVLKVDKGKTKSLSTFLKFYGNRLFQSHSTDANRLTTWAALYGMQWAQLSVLGNPLEKLSQSRGIDPMELSMGKACSITVWASIFFLNFIFAAMLTLSLPHRIPAYSMLGFNLLYYILAIIVAVSGYSVSW

SEQ ID NO: 35 - DNA, 939 bp, synthetic construct
  source 1..939: mol_type = other DNA
atggacgagatcaatgagcctgcaccaccatcggatcgtggagaagccattatagatcggatgaaaactcatttgaaaaatctaccatgttattcagttatggaagccgagaatattgcgcatttgatagaagaaataaacgtgatcagtggcgacgactctgtggtagagctgacaaacaaaattttgcaatgcatagcagaactggacacacagatttggtttaagaatatgctttcatggatactcgaggaaactatagaggaactgtatccagaactatgggttatacaagtgcctaacatggggattgtgattgctgattcagtttgggaatccaagtttgcgcctaaagtggattcgcctttccggaaatttgacactagagtcatactacaacgttaccttaatggaatatatcttgatcagtattatccgatgcaaatggtgaaaaatcatgagaagcggctgcaagtatatactgacattgacctagaacgttgttttgtgaaaaggccagagaatgaaataatatcaaagccacagaaagaactatatcagcaactgaaaaaatgcaaggaagaaaaggaaaatcgccgttctcatcccccaaaccatgttgtaaagattgacgataatggggtgttaaaagtagataaaggaaagacgaagagtttgagcacatttttgaagttttatgggaacaggctattccaatcacattctactgatgcaaatcgtcttactacatgggctgccttatatggaatgcaatgggcccaactatctgttttgggcaatcctcttgagaagttatcccaatccagaggcattgacccaatggaattaagtatggggaaagcatgttccatcacagtatgggcatcaatatttttcctgaactttatttttgcagccatgcttaccttgtcattgcct

SEQ ID NO: 36 - protein, 313 aa, synthetic construct
MDEINEPAPPSDRGEAIIDRMKTHLKNLPCYSVMEAENIAHLIEEINVISGDDSVVELTNKILQCIAELDTQIWFKNMLSWILEETIEELYPELWVIQVPNMGIVIADSVWESKFAPKVDSPFRKFDTRVILQRYLNGIYLDQYYPMQMVKNHEKRLQVYTDIDLERCFVKRPENEIISKPQKELYQQLKKCKEEKENRRSHPPNHVVKIDDNGVLKVDKGKTKSLSTFLKFYGNRLFQSHSTDANRLTTWAALYGMQWAQLSVLGNPLEKLSQSRGIDPMELSMGKACSITVWASIFFLNFIFAAMLTLSLP

SEQ ID NO: 37 - DNA, 870 bp, synthetic construct
  source 1..870: mol_type = other DNA
atggacgagatcaatgagcctgcaccaccatcggatcgtggagaagccattatagatcggatgaaaactcatttgaaaaatctaccatgttattcagttatggaagccgagaatattgcgcatttgatagaagaaataaacgtgatcagtggcgacgactctgtggtagagctgacaaacaaaattttgcaatgcatagcagaactggacacacagatttggtttaagaatatgctttcatggatactcgaggaaactatagaggaactgtatccagaactatgggttatacaagtgcctaacatggggattgtgattgctgattcagtttgggaatccaagtttgcgcctaaagtggattcgcctttccggaaatttgacactagagtcatactacaacgttaccttaatggaatatatcttgatcagtattatccgatgcaaatggtgaaaaatcatgagaagcggctgcaagtatatactgacattgacctagaacgttgttttgtgaaaaggccagagaatgaaataatatcaaagccacagaaagaactatatcagcaactgaaaaaatgcaaggaagaaaaggaaaatcgccgttctcatcccccaaaccatgttgtaaagattgacgataatggggtgttaaaagtagataaaggaaagacgaagagtttgagcacatttttgaagttttatgggaacaggctattccaatcacattctactgatgcaaatcgtcttactacatgggctgccttatatggaatgcaatgggcccaactatctgttttgggcaatcctcttgagaagttatcccaatccagaggcattgacccaatggaattaagtatggggaaagcatgttcc

SEQ ID NO: 38 - protein, 290 aa, synthetic construct
MDEINEPAPPSDRGEAIIDRMKTHLKNLPCYSVMEAENIAHLIEEINVISGDDSVVELTNKILQCIAELDTQIWFKNMLSWILEETIEELYPELWVIQVPNMGIVIADSVWESKFAPKVDSPFRKFDTRVILQRYLNGIYLDQYYPMQMVKNHEKRLQVYTDIDLERCFVKRPENEIISKPQKELYQQLKKCKEEKENRRSHPPNHVVKIDDNGVLKVDKGKTKSLSTFLKFYGNRLFQSHSTDANRLTTWAALYGMQWAQLSVLGNPLEKLSQSRGIDPMELSMGKACS

SEQ ID NO: 39 - DNA, 645 bp, synthetic construct
  source 1..645: mol_type = other DNA
atggaagaaaaggaaaatcgccgttctcatcccccaaaccatgttgtaaagattgacgataatggggtgttaaaagtagataaaggaaagacgaagagtttgagcacatttttgaagttttatgggaacaggctattccaatcacattctactgatgcaaatcgtcttactacatgggctgccttatatggaatgcaatgggcccaactatctgttttgggcaatcctcttgagaagttatcccaatccagaggcattgacccaatggaattaagtatggggaaagcatgttccatcacagtatgggcatcaatatttttcctgaactttatttttgcagccatgcttaccttgtcattgcctcatcgtatacctgcatactcaatgctgggatttaatttactctattacattttagcgataatagttgcagtgtctggatatagtgtttcatggggtggttcagctaaaatctcaggcctattgtttgtgtttggaggggccacatccttaatgctacttcttagcatcataatcatgggggtgctctatgctagctgcatatggtgctcctatccaaatgatgttggaggagaagtaactcagaaacactcgcctgagtgtatcatcaagatccaggatacc

SEQ ID NO: 40 - protein, 215 aa, synthetic construct
MEEKENRRSHPPNHVVKIDDNGVLKVDKGKTKSLSTFLKFYGNRLFQSHSTDANRLTTWAALYGMQWAQLSVLGNPLEKLSQSRGIDPMELSMGKACSITVWASIFFLNFIFAAMLTLSLPHRIPAYSMLGFNLLYYILAIIVAVSGYSVSWGGSAKISGLLFVFGGATSLMLLLSIIIMGVLYASCIWCSYPNDVGGEVTQKHSPECIIKIQDT